(19) United States
(12) Patent Application Publication
(10) Pub. No.: US 2025/0270187 A1
(43) Pub. Date: Aug. 28, 2025

(54) MOLECULAR EDITING OF MULTIPLE C-H BONDS LEVERAGING RECOGNITION OF DISTANCE, GEOMETRY AND CHIRALITY

(71) Applicant: THE SCRIPPS RESEARCH INSTITUTE, La Jolla, CA (US)

(72) Inventors: Jin-Quan YU, San Diego, CA (US); Zhoulong FAN, San Diego, CA (US)

(21) Appl. No.: 18/859,759

(22) PCT Filed: Apr. 25, 2023
(86) PCT No.: PCT/US2023/066193

§ 371 Date: Oct. 24, 2024

(51) Int. Cl. C07D 401/12; B01J 23/44; B01J 31/22; C07B 39/00; C07B 43/08; C07D 213/81

(52) U.S. Cl. CPC C07D 401/12; B01J 23/44; B01J 31/2295; C07B 39/00; C07B 43/08; C07D 213/81; C07B 2200/07

(57) ABSTRACT

This disclosure provides functional templates that direct Pd to functionalize multiple C—H bonds in polycyclic aza-arenes such as quinolines and related heterocycles at locations that are difficult to isolate and reach for substitution. Herein disclosed are two conceptually distinct directing templates (T) that enable site-selective C6 and C7-H activation of polycyclic aza-arenes. These catalytic pyridine-based templates recruit the aza-arene substrate through N-coordination, enabling the directing arm to deliver the catalyst and precisely activate remote and adjacent C6 or C7-H bond (FIG. 1d). In parallel, we discovered that the use of a simple and readily prepared template chaperone (TC) can turn over the directing template, allowing it to be used catalytically for the first time. Notably, chiral recognition is vital in the granular discrimination between competing C3 and C7-H bonds when the differentiation via distance and geometry is insufficient. Thus, precise recognition of a directing template's distance, geometry and chirality now enables the iterative C—H editing of quinoline and related pharmacophores at any desired site and order. The methods disclosed herein can also be used for diverse and late-stage modification of heterocycle-based drug molecules and pharmacophores.

### STATEMENT OF GOVERNMENT SUPPORT

This invention was made with government support under grant number GM102265 awarded by the National Institutes of Health. The government has certain rights in the invention.

### FIELD OF THE INVENTION

This disclosure provides functional templates that direct Pd to functionalize multiple C—H bonds in quinoline and related heterocycles at the locations that are difficult to reach. This method can be used for diverse modification of heterocycle-based drug molecules and pharmacophores.

### BACKGROUND OF THE INVENTION

The efficient generation of diverse analogues with structural modifications at various sites forms a perennial synthetic challenge that underpins drug discovery¹. For a given molecular scaffold or pharmacophore, the selective and iterative activation of multiple inert C—H bonds at different sites represents the most direct strategy for the expeditious generation of structural diversity^{2,3}. For example, functionalization with ten coupling partners at five different sites of quinoline scaffold could generate up to 100,000 structurally unique analogues using a unified editing strategy challenging through de novo synthesis (FIG. 1a). This notionally ideal ‘molecular editing’ approach, however, is marred by a lack of reliable methods for their late-stage selective functionalization, curtailing broader realization in a translational context¹. As privileged pharmacophores for diverse biological targets, aza-arene heterocycles are particularly dominant within the realm of drug discovery. Within the azine component, leveraging a substrate's intrinsic electronic properties have enabled the now-established site-selective functionalizations of C2-H^{4,5} and C4-H⁶⁻⁹ under a nucleophilic metallation regimen, and at C3-H^{10,11} via the corresponding electrophilic metallation process.

In contrast, selective functionalization of multiple C—H bonds on the benzocyclic component of bicyclic aza-arene heterocycles remains to be realized. For these chemically-similar C—H bonds, leverage of proximity-driven effects to selectively direct the catalyst has been limited to benzocyclic C8-H bonds adjacent to Lewis basic heteroatoms¹²⁻¹⁴ Notably, the selective editing of remote positions, such as C5-C7 on quinoline-type scaffolds, remain inaccessible to established electronically-driven or substrate-directed approaches described above (FIG. 1a).

It was surmised that this eminent problem could be solved using reversibly-binding directing templates capable of selectively positioning the catalyst proximate to a target remote C—H bond via a macrocyclophanic pre-transition state¹⁵⁻¹⁹. In this context, bicyclic aza-arene scaffolds pose further obstacles for template-directed remote regioselection; in addition to suppressing functionalization at activated (C2-C4) sites, the multiple adjacent, yet minutely inequivalent and unactivated remote benzocyclic positions (C5-C7) demand stringent regiochemical precision for their discrimination and selective activation.

The feasibility of this template-directed approach for the activation of remote benzocyclic C—H bonds was first indicated in 2017, where stoichiometric template loadings to overcome deleterious azine binding have enabled the singular C5-H palladation and functionalization of quinoline^{20,21}. In combination with norbornene relay, an indirect C6-selective arylation can also be realized based on this C5-H palladation, though this strategy only permits arylation with electron-deficient aryl iodides, requires a vacant C5 position, and fundamentally does not provide a solution for selective C6-H palladation and diverse functionalization necessary to achieve molecular editing (FIG. 1b)²². The direct activation of C6 and C7 positions, as compared with the marginally more nucleophilic C5 and C8 positions, is particularly difficult both for the chemical inertness and electronic similarity of the two C—H bonds.

Beyond their chemical similarity, executing a general template strategy to achieve site-selective C—H palladation at C6 and C7 positions is confounded by their spatial similarity relative to the anchoring N atom. Careful analysis revealed subtle differences between C6 and C7-H in both distance (one bond difference) and geometry (meta vs. para), suggesting the possibility of precise template design to differentiate between these two C—H bonds. The same analysis also revealed that the sterically-similar and more activated C3-H possesses a similar distance (one bond difference) but identical geometry (meta vs. meta) to our desired C7-H bond. The latter challenge alluded that judicious tuning of template distance and two-dimensional geometry may not be sufficient to impart selectivity for the remote C7 position.

To address these pitfalls, it was further inspired by chiral catalyst-controlled regioselective functionalization of chiral polyols²³⁻²⁵. This led to the design of a chiral template, which upon interaction with a matched chiral catalyst, is capable of distinguishing between C3 and C7-H positions thereby providing the desired C7-selective functionalization (FIG. 1c).

We herein disclose two conceptually distinct directing templates that enable site-selective C6 and C7-H activation of bicyclic aza-arenes. These catalytic pyridine-based templates recruit the aza-arene substrate through N-coordination, enabling the directing arm to deliver the catalyst and precisely activate remote and adjacent C6 or C7-H bond (FIG. 1d). In parallel, we discovered that the use of a simple and readily prepared template chaperone (TC) can turn over the directing template, allowing it to be used catalytically for the first time. Notably, chiral recognition is vital in the granular discrimination between competing C3 and C7-H bonds when the differentiation via distance and geometry is insufficient. Thus, precise recognition of a directing template's distance, geometry and chirality now enables the iterative C—H editing of quinoline pharmacophores at any desired site and order.

### SUMMARY OF THE INVENTION

The instant application provides a palladium-coordinating template compound for directing C6 selective functionalization of polycyclic aza-arenes having a hydrogen atom disposed on the 6-position thereof, comprising the structure of Formula I

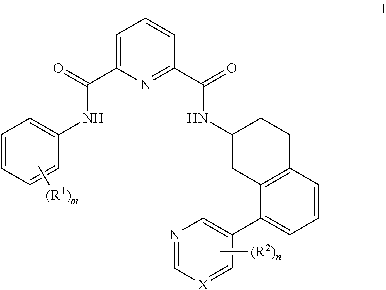

wherein:
each R¹ and R² is independently selected from halo, trifluoromethyl, nitro, optionally substituted (C₁-C₁₂)alkyl, and optionally substituted (C₁-C₁₂)alkoxy;
m is 0, 1, 2, 3, 4 or 5;
n is 0, 1, 2, or 3; and
X is CH, CR², or N.

The instant application provides the palladium-coordinating template compound having the structure of Formula II:

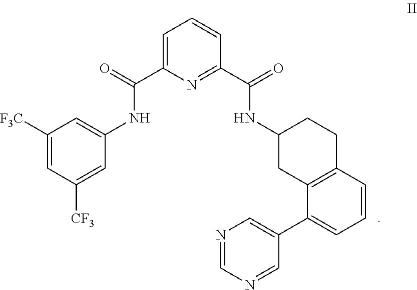 [SMILES: O=C(Nc1cc(C(F)(F)F)cc(C(F)(F)F)c1)c1cccc(C(=O)NC2CCc3cccc(-c4cncnc4)c3C2)n1]

The instant application provides the palladium-coordinating template compound having the structure of Formula III:

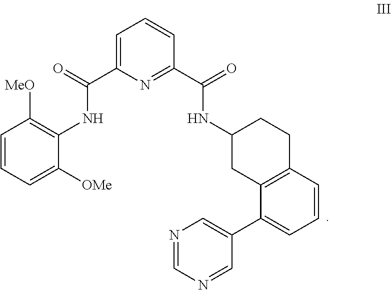 [SMILES: COc1cccc(OC)c1NC(=O)c1cccc(C(=O)NC2CCc3cccc(-c4cncnc4)c3C2)n1]

The instant application provides the palladium-coordinating template chaperone compound for catalytic conversion of Formula I after C6 selective functionalization of polycyclic aza-arenes having a hydrogen atom disposed on the 6-position thereof, comprising the structure of Formula IV

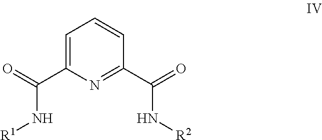

wherein:
each R¹ and R² is independently selected from (C₁-C₁₂)alkyl, Bn, and phenyl, each of which is optionally mono-, di-, tri-, tetra-, or penta-substituted with one or more substituents including, but not limited to, halo, trifluoromethyl, nitro, (C₁-C₁₂)alkyl, and (C₁-C₁₂)alkoxy.

The instant application provides the palladium-coordinating template chaperone compound having the structure of Formula V:

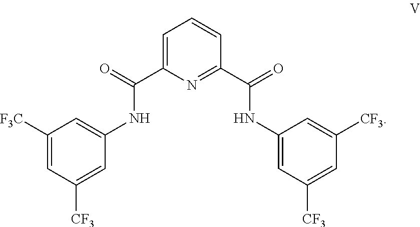 [SMILES: O=C(Nc1cc(C(F)(F)F)cc(C(F)(F)F)c1)c1cccc(C(=O)Nc2cc(C(F)(F)F)cc(C(F)(F)F)c2)n1]

The instant application provides the template palladium complex of Formula VI for directing C6 selective olefination or allylation of polycyclic aza-arenes having a hydrogen atom disposed on the 6-position thereof, comprising the template chaperone compound of Formula V, an atom of Pd(II), and a molecule of acetonitrile (L):

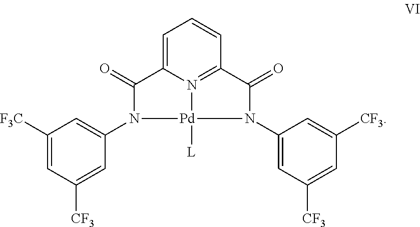

The instant application provides the palladium-coordinating template chaperone compound having the structure of Formula VII:

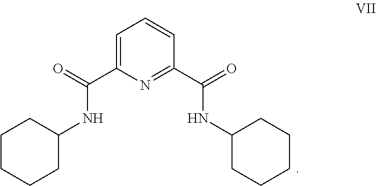 [SMILES: O=C(NC1CCCCC1)c1cccc(C(=O)NC2CCCCC2)n1]

The instant application provides the template palladium complex of Formula VIII for directing C6 selective olefination or allylation of polycyclic aza-arenes having a hydrogen atom disposed on the 6-position thereof, comprising the template chaperone compound of Formula VII, an atom of Pd(II), and a molecule of acetonitrile(L):

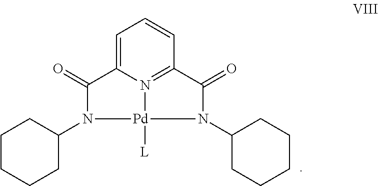

The instant application provides a method of directing diverse C6 selective functionalization of a polycyclic aza-arene having a hydrogen atom disposed on the 6-position thereof, including, but not limited to, olefination, allylation, alkynylation, arylation, iodination and cyanation, comprising mixing a polycyclic aza-arene with the palladium-coordinating template compound of Formula I with the template chaperone compound of Formula II.

The instant application provides a method comprising i) mixing the polycyclic aza-arene and the template compound of Formula I with the template chaperone compound of Formula II and a Pd catalyst; and ii) addition of an acrylate, additional Pd catalyst, an N-acylamino acid and an Ag salt.

The instant application provides a method comprising i) mixing the polycyclic aza-arene and the template compound of Formula I with the template chaperone compound of Formula II and Pd(OAc)₂ and ii) addition of an acrylate, additional Pd(OAc)₂, Ac-Gly-OH and Ag₂CO₃.

The instant application provides a method comprising i) mixing the polycyclic aza-arene with the template compound of Formula I, the template chaperone compound of Formula II, and a Pd catalyst; and ii) addition The instant application provides the above method wherein the Pd catalyst is Pd(II)(OAc)₂, the N-acylamino acid is Ac-Gly-OH, the olefin is (E)-4-octene, the Ag salt is Ag₂CO₃, and the Cu salt is Cu(OH)₂.

The instant application provides a template palladium complex of Formula IX for directing C6 selective functionalization of polycyclic aza-arenes having a hydrogen atom disposed on the 6-position thereof, comprising the template compound of Formula I, an atom of Pd(II), and a second molecule of the template compound of Formula I (L)

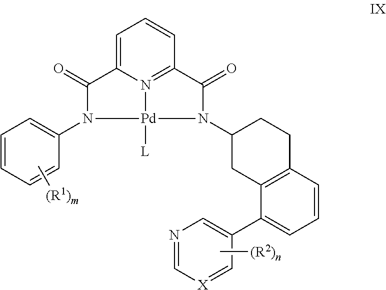

wherein:
each R¹ and R² is independently selected from halo, trifluoromethyl, nitro, optionally substituted (C₁-C₁₂)alkyl, and optionally substituted (C₁-C₁₂)alkoxy;
m is 0, 1, 2, 3, 4 or 5;
n is 0, 1, 2, or 3; and
X is CH, CR², or N.

The instant application provides the template palladium complex of Formula X for directing C6 selective functionalization of polycyclic aza-arenes having a hydrogen atom disposed on the 6-position thereof, comprising the template compound of Formula II, an atom of Pd(II), and a second molecule of the template compound of Formula II (L):

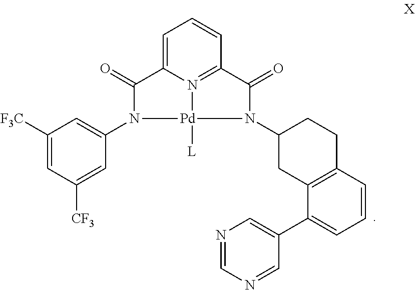

The instant application provides a template palladium complex of Formula XI for directing C6 selective functionalization of polycyclic aza-arenes having a hydrogen atom disposed on the 6-position thereof, comprising the template compound of Formula III, an atom of Pd(II), and a second molecule of the template compound of Formula III (L)

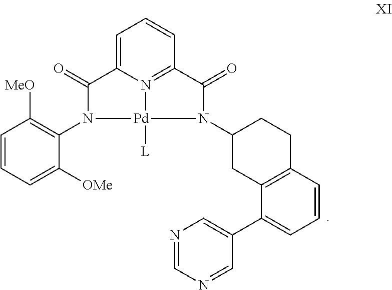

The instant application provides a method for directing C6 selective alkynylation of polycyclic aza-arenes having a hydrogen atom disposed on the 6-position thereof, comprising i) mixing the polycyclic aza-arene with the template compound of Formula I, the template chaperone compound of Formula II, and a Pd catalyst; and ii) addition of an alkynyl functional group, additional Pd catalyst, an N-acylamino acid, an Ag salt and a Cu salt.

The instant application provides a method for directing C6 selective alkynylation of polycyclic aza-arenes having a hydrogen atom disposed on the 6-position thereof, comprising i) mixing the polycyclic aza-arene with the template compound of Formula I, the template chaperone compound of Formula II, and Pd(OAc)₂; and ii) addition of alkynyl functional group, additional Pd(OAc)₂, Ac-Gly-OH, Ag₂CO₃ and Cu(OH)₂.

The instant application provides the above method, wherein the alkynyl functional group is triisopropylsilyl acetylene bromide.

The instant application provides a palladium-coordinating template compound for directing C7 selective functionalization of polycyclic aza-arenes having a hydrogen atom disposed on the 7-position thereof, having the structure of Formula XII

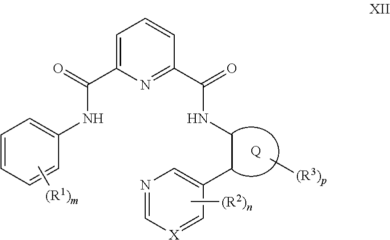

wherein:
each R¹, R², and R³ is independently selected from halo, trifluoromethyl, nitro, (C₁-C₁₂)alkyl, and (C₁-C₁₂)alkoxy;
X is CH, CR², or N;
m is 0, 1, 2, 3, 4 or 5;
n is 0, 1, 2, or 3;
p is 0, 1, 2, 3, or 4; and
Q is selected from the following:

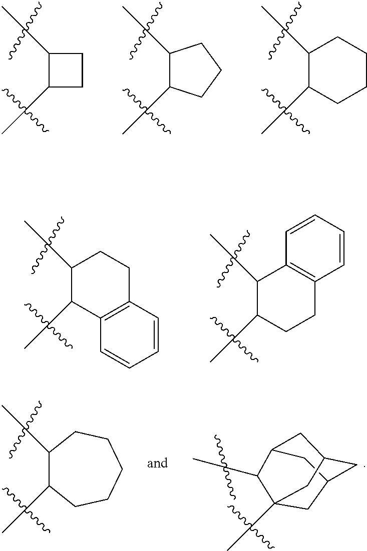

The instant application provides a palladium-coordinating template compound for directing C7 selective functionalization of polycyclic aza-arenes having the structure of Formula XIII:

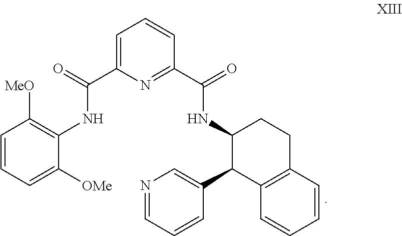 [SMILES: COc1cccc(OC)c1NC(=O)c1cccc(C(=O)N[C@H]2CCc3ccccc3[C@H]2c2cccnc2)n1]

The instant application provides a method of directing diverse C7 selective functionalization of C2, C3, and C4-substituted polycyclic aza-arenes having a hydrogen atom disposed on the 7-position thereof, including, but not limited to, olefination and alkynylation, comprising mixing the polycyclic aza-arene with the palladium-coordinating template compound of Formula XII with the template chaperone compound of Formula IV.

The instant application provides a method of directing diverse C7 selective olefination of C2, C3, and C4-substituted polycyclic aza-arenes having a hydrogen atom disposed on the 7-position thereof comprising mixing the polycyclic aza-arene with the palladium-coordinating template compound of Formula XII with the template chaperone compound of Formula IV.

The instant application provides a method of directing diverse C7 selective olefination of C2, C3, and C4-substituted polycyclic aza-arenes having a hydrogen atom disposed on the 7-position thereof comprising mixing the polycyclic aza-arene with the palladium-coordinating template compound of Formula XII with the template chaperone compound of Formula IV, further comprising addition of a Pd catalyst, an N-acylamino acid, an olefin, and an Ag salt.

The instant application provides a method of directing diverse C7 selective olefination of C2, C3, and C4-substituted polycyclic aza-arenes having a hydrogen atom disposed on the 7-position thereof comprising mixing the polycyclic aza-arene with the palladium-coordinating template compound of Formula XII with the template chaperone compound of Formula IV, further comprising addition of Pd(OAc)₂, Ac-DL-Phe-OH, an olefin, and Ag₂CO₃.

The instant application provides a method of directing diverse C7 selective alkynylation of C2, C3, and C4-substituted polycyclic aza-arenes having a hydrogen atom disposed on the 7-position thereof comprising mixing the polycyclic aza-arene with the palladium-coordinating template compound of Formula XII with the template chaperone compound of Formula IV, further comprising addition of a Pd catalyst, an N-acylamino acid, an olefin, an Ag salt, and a Cu salt.

The instant application provides a method of directing diverse C7 selective alkynylation of C2, C3, and C4-substituted polycyclic aza-arenes having a hydrogen atom disposed on the 7-position thereof comprising mixing the polycyclic aza-arene with the palladium-coordinating template compound of Formula XII with the template chaperone compound of Formula IV, further comprising addition of Pd(OAc)₂, Ac-DL-Phe-OH, an alkynyl functional group, Ag₂CO₃, and Cu(OH)₂.

The instant application provides the above method, wherein the alkynyl functional group is triisopropylsilyl acetylene bromide.

The instant application provides a palladium-coordinating enantiopure template compound for directing C7 selective functionalization of non-, C5, C6 or C8-substituted polycyclic aza-arenes having the structure of Formula XIVa or XIVb:

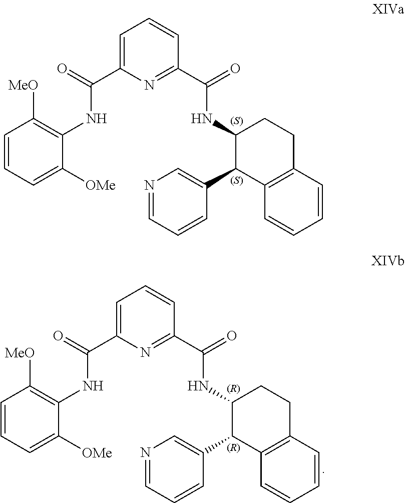

The instant application provides a method of olefination of the C7 position of non-, C5, C6 or C8-substituted polycyclic aza-arene having a hydrogen atom disposed on the 7-position thereof, comprising i) mixing the polycyclic aza-arene with the template compound of Formula XIVa or XIVb, the template chaperone compound of Formula IV, and a Pd catalyst and ii) mixing the product of step i) with additional Pd catalyst, an N-acylamino acid, an olefin functional group, and an Ag salt.

The instant application provides a method of olefination of the C7 position of non-, C5, C6 or C8-substituted polycyclic aza-arene having a hydrogen atom disposed on the 7-position thereof, comprising i) mixing the polycyclic aza-arene with the template compound of Formula XIVa or XIVb, the template chaperone compound of Formula IV, and Pd(OAc)₂ and ii) mixing the product of step i) with Pd(OAc)₂, Ac-L-Leu-OH or Ac-D-Leu-OH, an olefin functional group, and Ag₂CO₃.

The instant application provides a method of alkynylation of the C7 position of non-, C5, or C6-substituted polycyclic aza-arene having a hydrogen atom disposed on the 7-position thereof, comprising i) mixing the polycyclic aza-arene with the template compound of Formula XII, the template chaperone compound of compound of Formula IV, and Pd catalyst; and ii) addition of a Pd catalyst, an N-acylamino acid, an alkynyl functional group, an Ag salt, and a Cu salt.

The instant application provides a method of alkynylation of the C7 position of non-, C5, or C6-substituted polycyclic aza-arene having a hydrogen atom disposed on the 7-position thereof, comprising i) mixing the polycyclic aza-arene with the template compound of Formula XII, the template chaperone compound of compound of Formula IV, and Pd(OAc)₂; and ii) addition of Pd(OAc)₂, Ac-L-Leu-OH or Ac-D-Leu-OH, an alkynyl functional group, Ag₂CO₃, and Cu(OH)₂.

The instant application provides the above method, wherein the alkynyl functional group is triisopropylsilyl acetylene bromide.

The instant application provides any of the above methods, wherein the polycyclic aza-arene has the structure of any one of Formulae XV-XXIX:

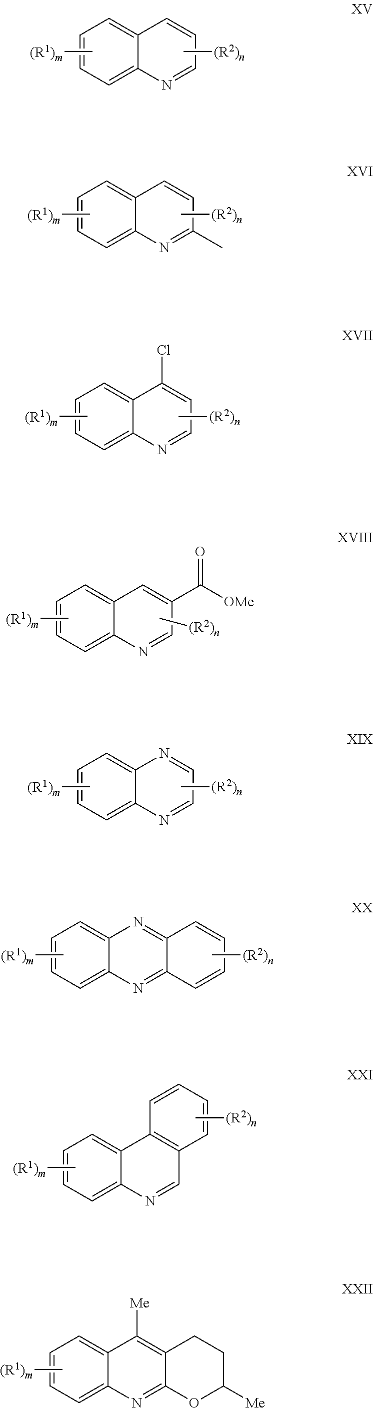

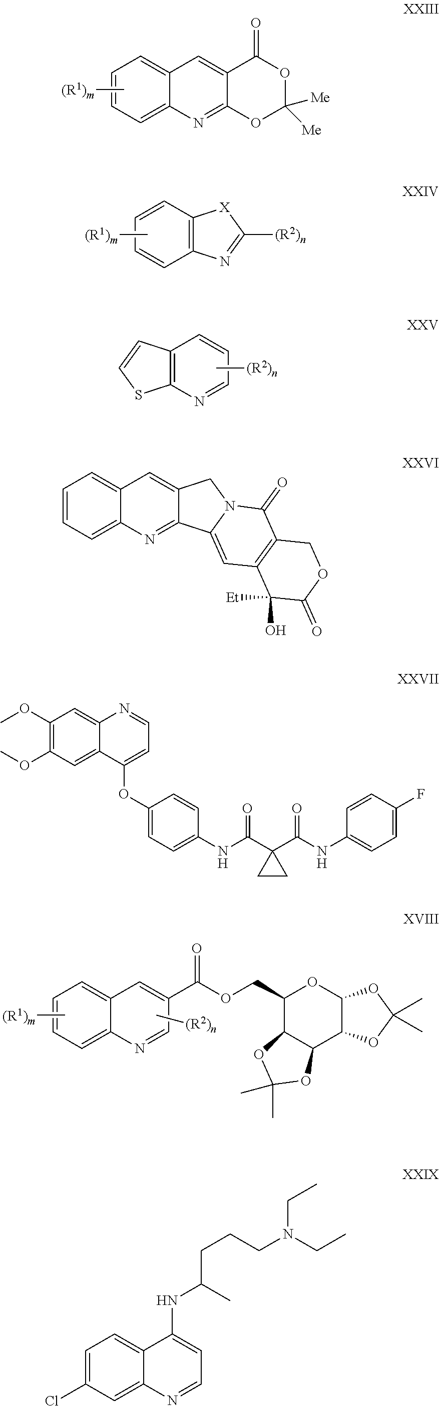

wherein:
each R¹ and R² is independently selected from H, F, Cl, C₁-C₆ alkyl, C₃-C₆ cycloalkyl, 3,5-dimethylC₆H₃, O(C₁-C₆ alkyl), CF₃, C(═O)OH, C₁-C₆ alkylC(═O)OH, C(═O)OC₁-C₆ alkyl) and C₁-C₆ alkylC(═O)OC₁-C₆ alkyl;
m is 0, 1, 2, or 3;
n is 0, 1, 2, or 3; and
X is S, O, NR², or C(R²)₂.

The instant application provides any of the above methods, wherein the polycyclic aza-arene is an optionally substituted bicyclic aza-instant application provides any of the above methods, wherein the polycyclic aza-arene is an optionally substituted tricyclic aza-arene.

The instant application provides any of the above methods, wherein the polycyclic aza-arene is an optionally substituted quinoline.

The instant application provides any of the above methods, wherein the polycyclic aza-arene is optionally substituted 3-methylquinoline.

The instant application provides any of the above methods, wherein the polycyclic aza-arene is an optionally substituted tetracyclic or pentacyclic quinoline.

The instant application provides any of the above methods, wherein the polycyclic aza-arene is an optionally substituted quinoxaline.

The instant application provides any of the above methods, wherein the polycyclic aza-arene is an optionally substituted benzothiophene.

The instant application provides any of the above methods, wherein the polycyclic aza-arene is an optionally substituted phenazine.

The instant application provides any of the above methods, wherein the polycyclic aza-arene is an optionally substituted thieno[2,3-b]pyridine.

The instant application provides a process for iterative C7 and C6 C—H activation and selective substitution of polycyclic aza-arenes comprising i) mixing the polycyclic aza-arene with the C7 directing template Formula of XII, Formula XIVa or Formula XIVb, and the template chaperone compound of Formula IV in the presence of Pd(OAc)₂, the first functional group to be added at position C7, Ag(OAc)₂, and Ac-DL-Phe-OH, Ac-D-Leu-OH, or Ac-L-Leu-OH and ii) mixing the polycyclic aza-arene product of step i) with the template compound of Formula I and the template chaperone compound of Formula IV in the presence of Pd(OAc)₂, the second functional group to be added at position C6, Ag(OAc)₂, and Ac-Gly-OH.

The instant application provides the above process, wherein the first functional group is an olefin and the second functional group is an olefin.

The instant application provides the above process, wherein the first functional group is an olefin and the second functional group is an allyl in the additional presence of Cu(OH)₂.

The instant application provides the above process, wherein the first functional group is an olefin and the second functional group is an alkyne in the additional presence of Cu(OH)₂.

The instant application provides the above process, wherein the first functional group is an alkyne in the additional presence of Cu(OH)₂ and the second functional group is an olefin.

The instant application provides the above process, wherein the first functional group is an alkyne in the additional presence of Cu(OH)₂ and the second functional group is an allyl in the additional presence of Cu(OH)₂.

The instant application provides the above process, wherein the first functional group is an alkyne in the additional presence of Cu(OH)₂ and the second functional group is an alkyne in the additional presence of Cu(OH)₂.

The instant application provides a process for iterative C7 and C6 C—H activation and selective olefination of polycyclic aza-arenes comprising i) mixing the polycyclic aza-arene with the C7 directing template Formula of XII and the template chaperone compound of Formula IV in the presence of Pd(OAc)₂, the first olefin functional group to be added at position C7, and Ac-DL-Phe-OH, Ac-D-Leu-OH, or Ac-L-Leu-OH, and Ag(OAc)₂, and ii) mixing the polycyclic aza-arene product of step i) with the template compound of Formula I and the template chaperone compound of Formula IV in the presence of Pd(OAc)₂, the second olefin functional group to be added at position C6, Ag(OAc)₂, and Ac-Gly-OH.

The instant application provides a process for iterative C7 and C6 C—H activation and selective alkynylation of polycyclic aza-arenes comprising i) mixing the polycyclic aza-arene with the C7 directing template Formula of XII and the template chaperone compound of Formula IV in the presence of Pd(OAc)₂, the first alkynyl functional group to be added at position C7, and Ac-DL-Phe-OH, Ac-D-Leu-OH, or Ac-L-Leu-OH, Ag(OAc)₂, and Cu(OH)₂ and ii) mixing the polycyclic aza-arene product of step i) with the template compound of Formula I and the template chaperone compound of Formula IV in the presence of Pd(OAc)₂, the second alkynyl functional group to be added at position C6, Ag(OAc)₂, Cu(OH)₂ and Ac-Gly-OH.

The instant application provides any palladium-coordinating template compound, palladium-coordinating template chaperone compound, template palladium complex, template chaperone palladium complex, method of functionalization including, but not limited to, olefination, allylation, alkynylation, arylation, iodination and cyanation, or process for iterative C7, C6 or related positional C—H activation and substitution of polycyclic aza-arenes as herein described.

### BRIEF DESCRIPTION OF THE DRAWINGS

FIG. 1. Molecular editing of heterocycles.

FIG. 2. C6 (and related) selective C—H olefination reactions of quinoline and related heterocycles.

FIG. 3. C7 (and related) selective C—H olefination reactions of quinoline and related heterocycles.

FIG. 4. Other transformations and synthetic applications.

### DETAILED DESCRIPTION OF THE INVENTION

The molecular editing methods as described herein can be used to modify polycyclic aza-arene molecules at the locations that are difficult to reach otherwise. Heterocycle containing natural products or drug candidates can directly be modified to improve the properties of a molecule. Two templates have been developed to activate C6 and C7 C—H bonds on the C6 and C7 positions of polycyclic aza-arenes. Olefination, allylation, and alkynylation methods have been developed as disclosed hereinbelow.

Through consecutive selective C—H functionalization at multiple sites, the direct molecular editing of heteroarene carbon-hydrogen (C—H) bonds has the potential to achieve rapid access into diverse molecular space; a valuable but often challenging goal to accomplish in medicinal chemistry. Contrasting with a handful of electronically-biased heterocyclic C—H bonds, remote benzocyclic C—H bonds on bicyclic aza-arenes are especially difficult to differentiate due to lack of intrinsic steric/electronic biases. We herein report a unified catalytic directing template strategy that enables the modular functionalization of chemically-similar and adjacent remote positions on polycyclic aza-arene scaffolds through careful modulation of distance, geometry and previously-unconsidered chirality in template molecule design. Differentiated by two structurally distinct catalytic directing templates, this method enables direct C—H olefination, alkynylation, and allylation at previously thought inaccessible adjacent C6 and C7 positions of quinolines and other polycyclicaza-arenes in the presence of a competing C3 position that is spatially similar to C7. Notably, such site-selective, iterative, and late-stage C—H editing of quinoline-containing pharmacophores can be modularly performed in different orders to provide diverse synthetic routes to suit bespoke synthetic applications. This report, in combination with previously reported complementary methods, now fully establishes a unified late-stage ‘molecular editing’ strategy to directly modify aza-arene heterocycles at any given site and in any order.

The instant application provides a palladium-coordinating template compound for directing C6 selective functionalization of polycyclic aza-arenes having a hydrogen atom disposed on the 6-position thereof, comprising the structure of Formula I

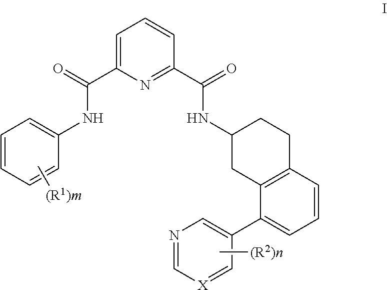

wherein:
each R¹ and R² is independently selected from halo, trifluoromethyl, nitro, optionally substituted (C₁-C₁₂)alkyl, and optionally substituted (C₁-C₁₂)alkoxy;
m is 0, 1, 2, 3, 4 or 5;
n is 0, 1, 2, or 3; and
X is CH, CR², or N.

The instant application provides the above palladium-coordinating template compound, wherein X is N, m is 2, both R¹ are —CF₃, and n is 0.

The instant application provides the above palladium-coordinating template compound, having the structure of Formula II:

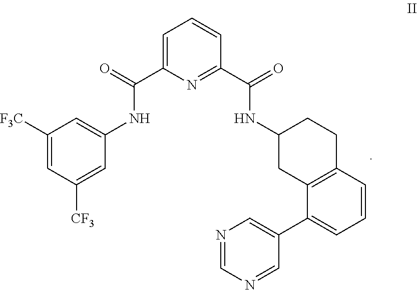 [SMILES: O=C(Nc1cc(C(F)(F)F)cc(C(F)(F)F)c1)c1cccc(C(=O)NC2CCc3cccc(-c4cncnc4)c3C2)n1]

Alternatively, the instant application provides the palladium-coordinating template compound, wherein X is N, m is 2, both R¹ are —OMe, and n is 0.

The instant application provides the above the palladium-coordinating template compound, having the structure of Formula III:

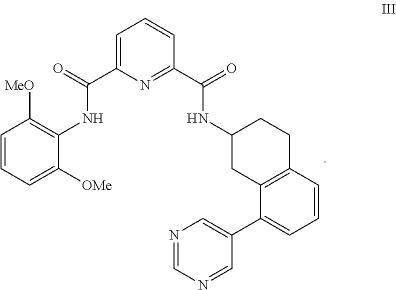 [SMILES: COc1cccc(OC)c1NC(=O)c1cccc(C(=O)NC2CCc3cccc(-c4cncnc4)c3C2)n1]

The instant application provides a palladium-coordinating template chaperone compound for catalytic conversion of Formula I after C6 selective functionalization of polycyclic aza-arenes having a hydrogen atom disposed on the 6-position thereof, comprising the structure of Formula IV

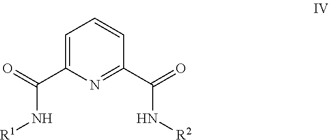

wherein:
each R¹ and R² is independently selected from (C₁-C₁₂)alkyl, Bn, and phenyl, each of which is optionally mono-, di-, tri-, tetra-, or penta-substituted with one or more substituents including, but not limited to, halo, trifluoromethyl, nitro, (C₁-C₁₂)alkyl, and C₁-C₁₂)alkoxy.

The instant application provides the above palladium-coordinating template chaperone compound, wherein both R¹ and R² are optionally substituted phenyl.

The instant application provides the above palladium-coordinating template chaperone compound, having the structure of Formula V:

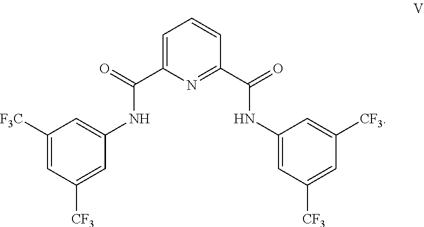 [SMILES: O=C(Nc1cc(C(F)(F)F)cc(C(F)(F)F)c1)c1cccc(C(=O)Nc2cc(C(F)(F)F)cc(C(F)(F)F)c2)n1]

The instant application provides a template palladium complex of Formula VI for directing C6 selective olefination or allylation of polycyclic aza-arenes having a hydrogen atom disposed on the 6-position thereof, comprising the template chaperone compound of Formula V, an atom of Pd(II), and a molecule of acetonitrile (L):

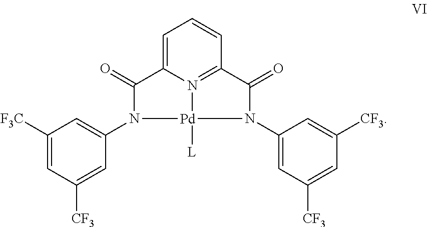

The instant application provides the above palladium-coordinating template chaperone compound, wherein both R¹ and R² are optionally substituted cyclohexyl.

The instant application provides the above palladium-coordinating template chaperone compound, having the structure of Formula VII:

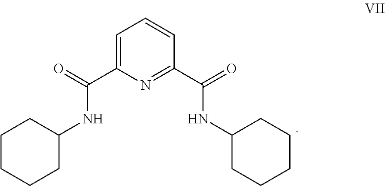 [SMILES: O=C(NC1CCCCC1)c1cccc(C(=O)NC2CCCCC2)n1]

The instant application provides a template palladium complex of Formula VIII for directing C6 selective olefination or allylation of polycyclic aza-arenes having a hydrogen atom disposed on the 6-position thereof, comprising the template chaperone compound of Formula VII, an atom of Pd(II), and a molecule of acetonitrile (L):

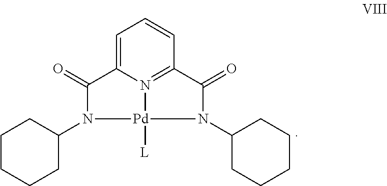

The instant application provides a method of directing diverse C6 selective functionalization of a polycyclic aza-arene having a hydrogen atom disposed on the 6-position thereof, including, but not limited to, olefination, allylation, alkynylation, arylation, iodination and cyanation, comprising mixing a polycyclic aza-arene with the palladium-coordinating template compound of Formula I with the template chaperone compound of Formula II.

The instant application provides the above method, wherein the selective functionalization is olefination.

Alternatively, the instant application provides the above method, wherein the selective functionalization is allylation.

Alternatively, the instant application provides the above method, wherein the selective functionalization is alkynylation.

Alternatively, the instant application provides the above method, wherein the selective functionalization is arylation.

Alternatively, the instant application provides the above method, wherein the selective functionalization is iodination.

Alternatively, the instant application provides the above method, wherein the selective functionalization is cyanation.

The instant application provides a method of olefination, comprising i) mixing the polycyclic aza-arene and the template compound of Formula I with the template chaperone compound of Formula II and a Pd catalyst; and ii) addition of an acrylate, additional Pd catalyst, an N-acylamino acid and an Ag salt.

The instant application provides the above method, wherein the Pd catalyst is Pd(OAc)₂.

The instant application provides the above method, wherein the N-acylamino acid is Ac-Gly-OH.

The instant application provides the above method, wherein the Ag salt is Ag₂CO₃.

The instant application provides a method of allylation, comprising i) mixing the polycyclic aza-arene with the template compound of Formula I, the template chaperone compound of Formula II, and a Pd catalyst; and ii) addition of additional Pd catalyst, an N-acylamino acid, an olefin, an Ag salt, and a Cu salt.

The instant application provides a method of allylation, comprising i) mixing the polycyclic aza-arene with the template compound of Formula I, the template chaperone compound of Formula II, and Pd(OAc)₂; and ii) addition of additional Pd(OAc)₂, an N-acylamino acid, an olefin, an Ag salt, and a Cu salt.

The instant application provides a method of allylation, comprising i) mixing the polycyclic aza-arene with the template compound of Formula I, the template chaperone compound of Formula II, and a Pd catalyst; and ii) addition of additional Pd catalyst, Ac-Gly-OH, an olefin, an Ag salt, and a Cu salt.

The instant application provides a method of allylation, comprising i) mixing the polycyclic aza-arene with the template compound of Formula I, the template chaperone compound of Formula II, and Pd(OAc)₂; and ii) addition of additional Pd(OAc)₂, Ac-Gly-OH, an olefin, an Ag salt, and a Cu salt.

The instant application provides a method of allylation, comprising i) mixing the polycyclic aza-arene with the template compound of Formula I, the template chaperone compound of Formula II, and a Pd catalyst; and ii) addition of additional Pd catalyst, an N-acylamino acid, an olefin, Ag₂CO₃, and a Cu salt.

The instant application provides a method of allylation, comprising i) mixing the polycyclic aza-arene with the template compound of Formula I, the template chaperone compound of Formula II, and Pd(OAc)₂; and ii) addition of additional Pd(OAc)₂, an N-acylamino acid, an olefin, Ag₂CO₃, and a Cu salt.

The instant application provides a method of allylation, comprising i) mixing the polycyclic aza-arene with the template compound of Formula I, the template chaperone compound of Formula II, and a Pd catalyst; and ii) addition of additional Pd catalyst, Ac-Gly-OH, an olefin, Ag₂CO₃, and a Cu salt.

The instant application provides a method of allylation, comprising i) mixing the polycyclic aza-arene with the template compound of Formula I, the template chaperone compound of Formula II, and Pd(OAc)₂; and ii) addition of additional Pd(OAc)₂, Ac-Gly-OH, an olefin, Ag₂CO₃, and a Cu salt.

The instant application provides a method of allylation, comprising i) mixing the polycyclic aza-arene with the template compound of Formula I, the template chaperone compound of Formula II, and a Pd catalyst; and ii) addition of additional Pd catalyst, an N-acylamino acid, an olefin, an Ag salt, and Cu(OH)₂.

The instant application provides a method of allylation, comprising i) mixing the polycyclic aza-arene with the template compound of Formula I, the template chaperone compound of Formula II, and Pd(OAc)₂; and ii) addition of additional Pd(OAc)₂, an N-acylamino acid, an olefin, an Ag salt, and Cu(OH)₂.

The instant application provides a method of allylation, comprising i) mixing the polycyclic aza-arene with the template compound of Formula I, the template chaperone compound of Formula II, and a Pd catalyst; and ii) addition of additional Pd catalyst, Ac-Gly-OH, an olefin, an Ag salt, and Cu(OH)₂.

The instant application provides a method of allylation, comprising i) mixing the polycyclic aza-arene with the template compound of Formula I, the template chaperone compound of Formula II, and a Pd catalyst; and ii) addition of additional Pd catalyst, an N-acylamino acid, (E)-4-octene, an Ag salt, and a Cu salt.

The instant application provides a method of allylation, comprising i) mixing the polycyclic aza-arene with the template compound of Formula I, the template chaperone compound of Formula II, and Pd(OAc)₂; and ii) addition of additional Pd(OAc)₂, an N-acylamino acid, (E)-4-octene, an Ag salt, and a Cu salt.

The instant application provides a method of allylation, comprising i) mixing the polycyclic aza-arene with the template compound of Formula I, the template chaperone compound of Formula II, and a Pd catalyst; and ii) addition of additional Pd catalyst, Ac-Gly-OH, (E)-4-octene, an Ag salt, and a Cu salt.

The instant application provides a method of allylation, comprising i) mixing the polycyclic aza-arene with the template compound of Formula I, the template chaperone compound of Formula II, and a Pd catalyst; and ii) addition of additional Pd catalyst, Ac-Gly-OH, (E)-4-octene, an Ag salt, and a Cu salt.

The instant application provides a method of allylation, comprising i) mixing the polycyclic aza-arene with the template compound of Formula I, the template chaperone compound of Formula II, and a Pd catalyst; and ii) addition of additional Pd catalyst, an N-acylamino acid, (E)-4-octene, Ag₂CO₃, and a Cu salt.

The instant application provides a method of allylation, comprising i) mixing the polycyclic aza-arene with the template compound of Formula I, the template chaperone compound of Formula II, and a Pd catalyst; and ii) addition of additional Pd catalyst, an N-acylamino acid, (E)-4-octene, an Ag salt, and Cu(OH)₂.

The instant application provides a method of allylation, comprising i) mixing the polycyclic aza-arene with the template compound of Formula I, the template chaperone compound of Formula II, and Pd(OAc)₂; and ii) addition of additional Pd(OAc)₂, Ac-Gly-OH, (E)-4-octene, an Ag salt, and a Cu salt.

The instant application provides a method of allylation, comprising i) mixing the polycyclic aza-arene with the template compound of Formula I, the template chaperone compound of Formula II, and Pd(OAc)₂; and ii) addition of additional Pd(OAc)₂, Ac-Gly-OH, (E)-4-octene, Ag₂CO₃, and a Cu salt.

The instant application provides a method of allylation, comprising i) mixing the polycyclic aza-arene with the template compound of Formula I, the template chaperone compound of Formula II, and Pd(OAc)₂; and ii) addition of additional Pd(OAc)₂, Ac-Gly-OH, (E)-4-octene, an Ag salt, and Cu(OH)₂.

The instant application provides a method of allylation, comprising i) mixing the polycyclic aza-arene with the template compound of Formula I, the template chaperone compound of Formula II, and Pd(OAc)₂; and ii) addition of additional Pd(OAc)₂, Ac-Gly-OH, (E)-4-octene, Ag₂CO₃, and Cu(OH)₂.

The instant application provides a template palladium complex of Formula IX for directing C6 selective functionalization of polycyclic aza-arenes having a hydrogen atom disposed on the 6-position thereof, comprising the template compound of Formula I, an atom of Pd(II), and a second molecule of the template compound of Formula I (L)

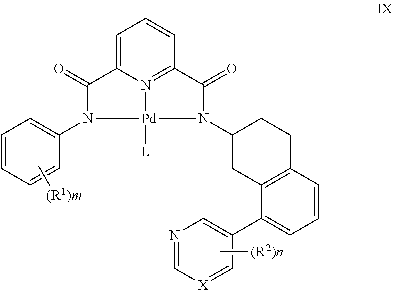

wherein:
each R¹ and R² is independently selected from halo, trifluoromethyl, nitro, optionally substituted (C₁-C₁₂)alkyl, and optionally substituted (C₁-C₁₂)alkoxy;
m is 0, 1, 2, 3, 4 or 5;
n is 0, 1, 2, or 3; and
X is CH, CR², or N.

The instant application provides a template palladium complex of Formula X for directing C6 selective functionalization of polycyclic aza-arenes having a hydrogen atom disposed on the 6-position thereof, comprising the template compound of Formula II, an atom of Pd(II), and a second molecule of the template compound of Formula II (L):

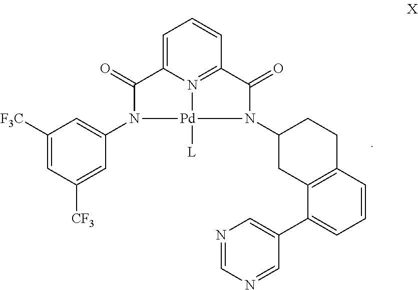

The instant application provides a template palladium complex of Formula XI for directing C6 selective functionalization of polycyclic aza-arenes having a hydrogen atom disposed on the 6-position thereof, comprising the template compound of Formula III, an atom of Pd(II), and a second molecule of the template compound of Formula III (L)

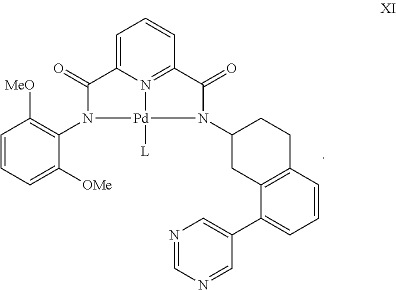

The instant application provides a method for directing C6 selective alkynylation of polycyclic aza-arenes having a hydrogen atom disposed on the 6-position thereof, comprising i) mixing the polycyclic aza-arene with the template compound of Formula I, the template chaperone compound of Formula II, and a Pd catalyst; and ii) addition of an alkynyl functional group, additional Pd catalyst, an N-acylamino acid, an Ag salt and a Cu salt.

The instant application provides a method for directing C6 selective alkynylation of polycyclic aza-arenes having a hydrogen atom disposed on the 6-position thereof, comprising i) mixing the polycyclic aza-arene with the template compound of Formula I, the template chaperone compound of Formula II, and Pd(OAc)₂; and ii) addition of an alkynyl functional group, additional Pd(OAc)₂, an N-acylamino acid, an Ag salt and a Cu salt.

The instant application provides a method for directing C6 selective alkynylation of polycyclic aza-arenes having a hydrogen atom disposed on the 6-position thereof, comprising i) mixing the polycyclic aza-arene with the template compound of Formula I, the template chaperone compound of Formula II, and a Pd catalyst; and ii) addition of an alkynyl functional group, additional Pd catalyst, Ac-Gly-OH, an Ag salt and a Cu salt.

The instant application provides a method for directing C6 selective alkynylation of polycyclic aza-arenes having a hydrogen atom disposed on the 6-position thereof, comprising i) mixing the polycyclic aza-arene with the template compound of Formula I, the template chaperone compound of Formula II, and Pd(OAc)₂; and ii) addition of an alkynyl functional group, additional Pd(OAc)₂, Ac-Gly-OH, an Ag salt and a Cu salt.

The instant application provides a method for directing C6 selective alkynylation of polycyclic aza-arenes having a hydrogen atom disposed on the 6-position thereof, comprising i) mixing the polycyclic aza-arene with the template compound of Formula I, the template chaperone compound of Formula II, and Pd(OAc)₂; and ii) addition of an alkynyl functional group, additional Pd(OAc)₂, an N-acylamino acid, Ag₂CO₃ and a Cu salt.

The instant application provides a method for directing C6 selective alkynylation of polycyclic aza-arenes having a hydrogen atom disposed on the 6-position thereof, comprising i) mixing the polycyclic aza-arene with the template compound of Formula I, the template chaperone compound of Formula II, and a Pd catalyst; and ii) addition of an alkynyl functional group, additional Pd catalyst, Ac-Gly-OH, Ag₂CO₃ and a Cu salt.

The instant application provides a method for directing C6 selective alkynylation of polycyclic aza-arenes having a hydrogen atom disposed on the 6-position thereof, comprising i) mixing the polycyclic aza-arene with the template compound of Formula I, the template chaperone compound of Formula II, and Pd(OAc)₂; and ii) addition of an alkynyl functional group, additional Pd(OAc)₂, Ac-Gly-OH, Ag₂CO₃ and a Cu salt.

The instant application provides a method for directing C6 selective alkynylation of polycyclic aza-arenes having a hydrogen atom disposed on the 6-position thereof, comprising i) mixing the polycyclic aza-arene with the template compound of Formula I, the template chaperone compound of Formula II, and Pd(OAc)₂; and ii) addition of an alkynyl functional group, additional Pd(OAc)₂, an N-acylamino acid, an Ag salt and Cu(OH)₂.

The instant application provides a method for directing C6 selective alkynylation of polycyclic aza-arenes having a hydrogen atom disposed on the 6-position thereof, comprising i) mixing the polycyclic aza-arene with the template compound of Formula I, the template chaperone compound of Formula II, and a Pd catalyst; and ii) addition of an alkynyl functional group, additional Pd catalyst, Ac-Gly-OH, an Ag salt and Cu(OH)₂.

The instant application provides a method for directing C6 selective alkynylation of polycyclic aza-arenes having a hydrogen atom disposed on the 6-position thereof, comprising i) mixing the polycyclic aza-arene with the template compound of Formula I, the template chaperone compound of Formula II, and Pd(OAc)₂; and ii) addition of an alkynyl functional group, additional Pd(OAc)₂, Ac-Gly-OH, an Ag salt and Cu(OH)₂.

The instant application provides a method for directing C6 selective alkynylation of polycyclic aza-arenes having a hydrogen atom disposed on the 6-position thereof, comprising i) mixing the polycyclic aza-arene with the template compound of Formula I, the template chaperone compound of Formula II, and Pd(OAc)₂; and ii) addition of an alkynyl functional group, additional Pd(OAc)₂, an N-acylamino acid, Ag₂CO₃ and Cu(OH)₂.

The instant application provides a method for directing C6 selective alkynylation of polycyclic aza-arenes having a hydrogen atom disposed on the 6-position thereof, comprising i) mixing the polycyclic aza-arene with the template compound of Formula I, the template chaperone compound of Formula II, and a Pd catalyst; and ii) addition of an alkynyl functional group, additional Pd catalyst, Ac-Gly-OH, Ag₂CO₃ and Cu(OH)₂.

The instant application provides a method for directing C6 selective alkynylation of polycyclic aza-arenes having a hydrogen atom disposed on the 6-position thereof, comprising i) mixing the polycyclic aza-arene with the template compound of Formula I, the template chaperone compound of Formula II, and Pd(OAc)₂; and ii) addition of an alkynyl functional group, additional Pd(OAc)₂, Ac-Gly-OH, Ag₂CO₃ and Cu(OH)₂.

The instant application provides a method for directing C6 selective alkynylation of polycyclic aza-arenes having a hydrogen atom disposed on the 6-position thereof, comprising i) mixing the polycyclic aza-arene with the template compound of Formula I, the template chaperone compound of Formula II, and a Pd catalyst; and ii) addition of triisopropylsilyl acetylene bromide, additional Pd catalyst, an N-acylamino acid, an Ag salt and a Cu salt.

The instant application provides a method for directing C6 selective alkynylation of polycyclic aza-arenes having a hydrogen atom disposed on the 6-position thereof, comprising i) mixing the polycyclic aza-arene with the template compound of Formula I, the template chaperone compound of Formula II, and Pd(OAc)₂; and ii) addition of triisopropylsilyl acetylene bromide, additional Pd(OAc)₂, an N-acylamino acid, an Ag salt and a Cu salt.

The instant application provides a method for directing C6 selective alkynylation of polycyclic aza-arenes having a hydrogen atom disposed on the 6-position thereof, comprising i) mixing the polycyclic aza-arene with the template compound of Formula I, the template chaperone compound of Formula II, and a Pd catalyst; and ii) addition of triisopropylsilyl acetylene bromide, additional Pd catalyst, Ac-Gly-OH, an Ag salt and a Cu salt.

The instant application provides a method for directing C6 selective alkynylation of polycyclic aza-arenes having a hydrogen atom disposed on the 6-position thereof, comprising i) mixing the polycyclic aza-arene with the template compound of Formula I, the template chaperone compound of Formula II, and Pd(OAc)₂; and ii) addition of triisopropylsilyl acetylene bromide, additional Pd(OAc)₂, Ac-Gly-OH, an Ag salt and a Cu salt.

The instant application provides a method for directing C6 selective alkynylation of polycyclic aza-arenes having a hydrogen atom disposed on the 6-position thereof, comprising i) mixing the polycyclic aza-arene with the template compound of Formula I, the template chaperone compound of Formula II, and Pd(OAc)₂; and ii) addition of triisopropylsilyl acetylene bromide, additional Pd(OAc)₂, an N-acylamino acid, Ag₂CO₃ and a Cu salt.

The instant application provides a method for directing C6 selective alkynylation of polycyclic aza-arenes having a hydrogen atom disposed on the 6-position thereof, comprising i) mixing the polycyclic aza-arene with the template compound of Formula I, the template chaperone compound of Formula II, and a Pd catalyst; and ii) addition of triisopropylsilyl acetylene bromide, additional Pd catalyst, Ac-Gly-OH, Ag₂CO₃ and a Cu salt.

The instant application provides a method for directing C6 selective alkynylation of polycyclic aza-arenes having a hydrogen atom disposed on the 6-position thereof, comprising i) mixing the polycyclic aza-arene with the template compound of Formula I, the template chaperone compound of Formula II, and Pd(OAc)₂; and ii) addition of triisopropylsilyl acetylene bromide, additional Pd(OAc)₂, Ac-Gly-OH, Ag₂CO₃ and a Cu salt.

The instant application provides a method for directing C6 selective alkynylation of polycyclic aza-arenes having a hydrogen atom disposed on the 6-position thereof, comprising i) mixing the polycyclic aza-arene with the template compound of Formula I, the template chaperone compound of Formula II, and Pd(OAc)₂; and ii) addition of triisopropylsilyl acetylene bromide, additional Pd(OAc)₂, an N-acylamino acid, an Ag salt and Cu(OH)₂.

The instant application provides a method for directing C6 selective alkynylation of polycyclic aza-arenes having a hydrogen atom disposed on the 6-position thereof, comprising i) mixing the polycyclic aza-arene with the template compound of Formula I, the template chaperone compound of Formula II, and a Pd catalyst; and ii) addition of triisopropylsilyl acetylene bromide, additional Pd catalyst, Ac-Gly-OH, an Ag salt and Cu(OH)₂.

The instant application provides a method for directing C6 selective alkynylation of polycyclic aza-arenes having a hydrogen atom disposed on the 6-position thereof, comprising i) mixing the polycyclic aza-arene with the template compound of Formula I, the template chaperone compound of Formula II, and Pd(OAc)₂; and ii) addition of triisopropylsilyl acetylene bromide, additional Pd(OAc)₂, Ac-Gly-OH, an Ag salt and Cu(OH)₂.

The instant application provides a method for directing C6 selective alkynylation of polycyclic aza-arenes having a hydrogen atom disposed on the 6-position thereof, comprising i) mixing the polycyclic aza-arene with the template compound of Formula I, the template chaperone compound of Formula II, and Pd(OAc)₂; and ii) addition of triisopropylsilyl acetylene bromide, additional Pd(OAc)₂, an N-acylamino acid, Ag₂CO₃ and Cu(OH)₂.

The instant application provides a method for directing C6 selective alkynylation of polycyclic aza-arenes having a hydrogen atom disposed on the 6-position thereof, comprising i) mixing the polycyclic aza-arene with the template compound of Formula I, the template chaperone compound of Formula II, and a Pd catalyst; and ii) addition of triisopropylsilyl acetylene bromide, additional Pd catalyst, Ac-Gly-OH, Ag₂CO₃ and Cu(OH)₂.

The instant application provides a method for directing C6 selective alkynylation of polycyclic aza-arenes having a hydrogen atom disposed on the 6-position thereof, comprising i) mixing the polycyclic aza-arene with the template compound of Formula I, the template chaperone compound of Formula II, and Pd(OAc)₂; and ii) addition of triisopropylsilyl acetylene bromide, additional Pd(OAc)₂, Ac-Gly-OH, Ag₂CO₃ and Cu(OH)₂.

The instant application provides a palladium-coordinating template compound for directing C7 selective functionalization of polycyclic aza-arenes having a hydrogen atom disposed on the 7-position thereof, having the structure of Formula XII

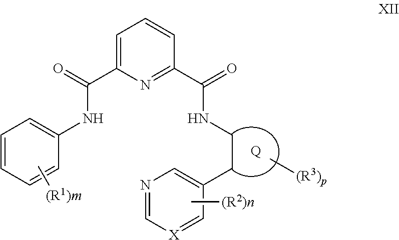

wherein:
each R¹, R², and R³ is independently selected from halo, trifluoromethyl, nitro, (C₁-C₁₂)alkyl, and (C₁-C₁₂)alkoxy;
X is CH, CR², or N;
m is 0, 1, 2, 3, 4 or 5;
n is 0, 1, 2, or 3;
p is 0, 1, 2, 3, or 4; and
Q is selected from the following:

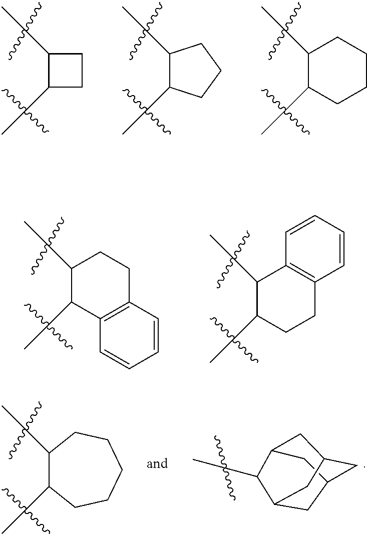

The instant application provides a palladium-coordinating template compound for directing C7 selective functionalization of polycyclic aza-arenes having the structure of Formula XIII:

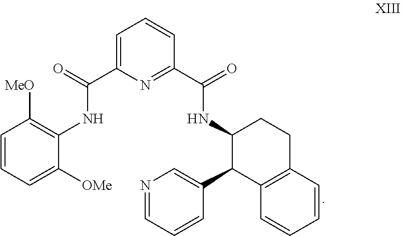 [SMILES: COc1cccc(OC)c1NC(=O)c1cccc(C(=O)N[C@H]2CCc3ccccc3[C@H]2c2cccnc2)n1]

The instant application provides a method of directing diverse C7 selective functionalization of C2, C3, and C4-substituted polycyclic aza-arenes having a hydrogen atom disposed on the 7-position thereof, including, but not limited to, olefination and alkynylation, comprising mixing the polycyclic aza-arene with the palladium-coordinating template compound of Formula XII with the template chaperone compound of Formula IV.

The instant application provides a method of directing C7 selective olefination of C2, C3, and C4-substituted polycyclic aza-arenes having a hydrogen atom disposed on the 7-position thereof, including, but not limited to, olefination and alkynylation, comprising mixing the polycyclic aza-arene with the palladium-coordinating template compound of Formula XII with the template chaperone compound of Formula IV.

The instant application provides a method of directing C7 selective olefination of C2, C3, and C4-substituted polycyclic aza-arenes having a hydrogen atom disposed on the 7-position thereof, including, but not limited to, olefination and alkynylation, comprising mixing the polycyclic aza-arene with the palladium-coordinating template compound of Formula XII with the template chaperone compound of Formula IV, further comprising addition of a Pd catalyst, an N-acylamino acid, an olefin, and an Ag salt.

The instant application provides a method of directing C7 selective olefination of C2, C3, and C4-substituted polycyclic aza-arenes having a hydrogen atom disposed on the 7-position thereof, including, but not limited to, olefination and alkynylation, comprising mixing the polycyclic aza-arene with the palladium-coordinating template compound of Formula XII with the template chaperone compound of Formula IV, further comprising addition of a Pd catalyst, an N-acylamino acid, an olefin, and an Ag salt, wherein the Pd catalyst is Pd(OAc)₂.

The instant application provides a method of directing C7 selective olefination of C2, C3, and C4-substituted polycyclic aza-arenes having a hydrogen atom disposed on the 7-position thereof, including, but not limited to, olefination and alkynylation, comprising mixing the polycyclic aza-arene with the palladium-coordinating template compound of Formula XII with the template chaperone compound of Formula IV, further comprising addition of a Pd catalyst, an N-acylamino acid, an olefin, and an Ag salt, wherein the N-acylamino acid is Ac-DL-Phe-OH.

The instant application provides a method of directing C7 selective olefination of C2, C3, and C4-substituted polycyclic aza-arenes having a hydrogen atom disposed on the 7-position thereof, including, but not limited to, olefination and alkynylation, comprising mixing the polycyclic aza-arene with the palladium-coordinating template compound of Formula XII with the template chaperone compound of Formula IV, further comprising addition of a Pd catalyst, an N-acylamino acid, an olefin, and an Ag salt, wherein the Ag salt is Ag₂CO₃.

The instant application provides a method for directing C7 selective olefination of C2, C3, and C4-substituted polycyclic aza-arenes having a hydrogen atom disposed on the 7-position thereof, comprising i) mixing the polycyclic aza-arene, the template compound of Formula XIII, the template chaperone compound of Formula IV, and Pd(II)(OAc)₂; and ii) addition of Pd(II)(OAc)₂, Ac-DL-Phe-OH, an olefin, and Ag₂CO₃.

The instant application provides a method of directing C7 selective alkynylation of C2, C3, and C4-substituted polycyclic aza-arenes having a hydrogen atom disposed on the 7-position thereof, including, but not limited to, olefination and alkynylation, comprising mixing the polycyclic aza-arene with the palladium-coordinating template compound of Formula XII with the template chaperone compound of Formula IV.

The instant application provides a method of directing C7 selective alkynylation of C2, C3, and C4-substituted polycyclic aza-arenes having a hydrogen atom disposed on the 7-position thereof, including, but not limited to, olefination and alkynylation, comprising mixing the polycyclic aza-arene with the palladium-coordinating template compound of Formula XII with the template chaperone compound of Formula IV, further comprising addition of a Pd catalyst, an N-acylamino acid, an alkynyl functional group, an Ag salt, and a Cu salt.

The instant application provides a method of directing C7 selective alkynylation of C2, C3, and C4-substituted polycyclic aza-arenes having a hydrogen atom disposed on the 7-position thereof, including, but not limited to, olefination and alkynylation, comprising mixing the polycyclic aza-arene with the palladium-coordinating template compound of Formula XII with the template chaperone compound of Formula IV, further comprising addition of a Pd(OAc)₂, an N-acylamino acid, an alkynyl functional group, an Ag salt, and a Cu salt.

The instant application provides a method of directing C7 selective alkynylation of C2, C3, and C4-substituted polycyclic aza-arenes having a hydrogen atom disposed on the 7-position thereof, including, but not limited to, olefination and alkynylation, comprising mixing the polycyclic aza-arene with the palladium-coordinating template compound of Formula XII with the template chaperone compound of Formula IV, further comprising addition of a Pd catalyst, Ac-DL-Phe-OH, an alkynyl functional group, an Ag salt, and a Cu salt.

The instant application provides a method of directing C7 selective alkynylation of C2, C3, and C4-substituted polycyclic aza-arenes having a hydrogen atom disposed on the 7-position thereof, including, but not limited to, olefination and alkynylation, comprising mixing the polycyclic aza-arene with the palladium-coordinating template compound of Formula XII with the template chaperone compound of Formula IV, further comprising addition of a Pd catalyst, an N-acylamino acid, an alkynyl functional group, Ag₂CO₃, and a Cu salt.

The instant application provides a method of directing C7 selective alkynylation of C2, C3, and C4-substituted polycyclic aza-arenes having a hydrogen atom disposed on the 7-position thereof, including, but not limited to, olefination and alkynylation, comprising mixing the polycyclic aza-arene with the palladium-coordinating template compound of Formula XII with the template chaperone compound of Formula IV, further comprising addition of a Pd(OAc)₂, an N-acylamino acid, an alkynyl functional group, Ag₂CO₃, and a Cu salt.

The instant application provides a method of directing C7 selective alkynylation of C2, C3, and C4-substituted polycyclic aza-arenes having a hydrogen atom disposed on the 7-position thereof, including, but not limited to, olefination and alkynylation, comprising mixing the polycyclic aza-arene with the palladium-coordinating template compound of Formula XII with the template chaperone compound of Formula IV, further comprising addition of a Pd catalyst, Ac-DL-Phe-OH, an alkynyl functional group, Ag₂CO₃, and a Cu salt.

The instant application provides a method of directing C7 selective alkynylation of C2, C3, and C4-substituted polycyclic aza-arenes having a hydrogen atom disposed on the 7-position thereof, including, but not limited to, olefination and alkynylation, comprising mixing the polycyclic aza-arene with the palladium-coordinating template compound of Formula XII with the template chaperone compound of Formula IV, further comprising addition of a Pd catalyst, an N-acylamino acid, an alkynyl functional group, an Ag salt, and Cu(OH)₂.

The instant application provides a method of directing C7 selective alkynylation of C2, C3, and C4-substituted polycyclic aza-arenes having a hydrogen atom disposed on the 7-position thereof, including, but not limited to, olefination and alkynylation, comprising mixing the polycyclic aza-arene with the palladium-coordinating template compound of Formula XII with the template chaperone compound of Formula IV, further comprising addition of a Pd(OAc)₂, an N-acylamino acid, an alkynyl functional group, an Ag salt, and Cu(OH)₂.

The instant application provides a method of directing C7 selective alkynylation of C2, C3, and C4-substituted polycyclic aza-arenes having a hydrogen atom disposed on the 7-position thereof, including, but not limited to, olefination and alkynylation, comprising mixing the polycyclic aza-arene with the palladium-coordinating template compound of Formula XII with the template chaperone compound of Formula IV, further comprising addition of a Pd catalyst, Ac-DL-Phe-OH, an alkynyl functional group, an Ag salt, and Cu(OH)₂.

The instant application provides a method of directing C7 selective alkynylation of C2, C3, and C4-substituted polycyclic aza-arenes having a hydrogen atom disposed on the 7-position thereof, including, but not limited to, olefination and alkynylation, comprising mixing the polycyclic aza-arene with the palladium-coordinating template compound of Formula XII with the template chaperone compound of Formula IV, further comprising addition of a Pd catalyst, an N-acylamino acid, an alkynyl functional group, Ag₂CO₃, and Cu(OH)₂.

The instant application provides a method of directing C7 selective alkynylation of C2, C3, and C4-substituted polycyclic aza-arenes having a hydrogen atom disposed on the 7-position thereof, including, but not limited to, olefination and alkynylation, comprising mixing the polycyclic aza-arene with the palladium-coordinating template compound of Formula XII with the template chaperone compound of Formula IV, further comprising addition of a Pd(OAc)₂, an N-acylamino acid, an alkynyl functional group, Ag₂CO₃, and Cu(OH)₂.

The instant application provides a method of directing C7 selective alkynylation of C2, C3, and C4-substituted polycyclic aza-arenes having a hydrogen atom disposed on the 7-position thereof, including, but not limited to, olefination and alkynylation, comprising mixing the polycyclic aza-arene with the palladium-coordinating template compound of Formula XII with the template chaperone compound of Formula IV, further comprising addition of a Pd catalyst, Ac-DL-Phe-OH, an alkynyl functional group, Ag₂CO₃, and Cu(OH)₂.

The instant application provides a method of directing C7 selective alkynylation of C2, C3, and C4-substituted polycyclic aza-arenes having a hydrogen atom disposed on the 7-position thereof, including, but not limited to, olefination and alkynylation, comprising mixing the polycyclic aza-arene with the palladium-coordinating template compound of Formula XII with the template chaperone compound of Formula IV, further comprising addition of a Pd catalyst, an N-acylamino acid, triisopropylsilyl acetylene bromide, an Ag salt, and a Cu salt.

The instant application provides a method of directing C7 selective alkynylation of C2, C3, and C4-substituted polycyclic aza-arenes having a hydrogen atom disposed on the 7-position thereof, including, but not limited to, olefination and alkynylation, comprising mixing the polycyclic aza-arene with the palladium-coordinating template compound of Formula XII with the template chaperone compound of Formula IV, further comprising addition of a Pd(OAc)₂, an N-acylamino acid, triisopropylsilyl acetylene bromide, an Ag salt, and a Cu salt.

The instant application provides a method of directing C7 selective alkynylation of C2, C3, and C4-substituted polycyclic aza-arenes having a hydrogen atom disposed on the 7-position thereof, including, but not limited to, olefination and alkynylation, comprising mixing the polycyclic aza-arene with the palladium-coordinating template compound of Formula XII with the template chaperone compound of Formula IV, further comprising addition of a Pd catalyst, Ac-DL-Phe-OH, triisopropylsilyl acetylene bromide, an Ag salt, and a Cu salt.

The instant application provides a method of directing C7 selective alkynylation of C2, C3, and C4-substituted polycyclic aza-arenes having a hydrogen atom disposed on the 7-position thereof, including, but not limited to, olefination and alkynylation, comprising mixing the polycyclic aza-arene with the palladium-coordinating template compound of Formula XII with the template chaperone compound of Formula IV, further comprising addition of a Pd catalyst, an N-acylamino acid, triisopropylsilyl acetylene bromide, Ag₂CO₃, and a Cu salt.

The instant application provides a method of directing C7 selective alkynylation of C2, C3, and C4-substituted polycyclic aza-arenes having a hydrogen atom disposed on the 7-position thereof, including, but not limited to, olefination and alkynylation, comprising mixing the polycyclic aza-arene with the palladium-coordinating template compound of Formula XII with the template chaperone compound of Formula IV, further comprising addition of a Pd(OAc)₂, an N-acylamino acid, triisopropylsilyl acetylene bromide, Ag₂CO₃, and a Cu salt.

The instant application provides a method of directing C7 selective alkynylation of C2, C3, and C4-substituted polycyclic aza-arenes having a hydrogen atom disposed on the 7-position thereof, including, but not limited to, olefination and alkynylation, comprising mixing the polycyclic aza-arene with the palladium-coordinating template compound of Formula XII with the template chaperone compound of Formula IV, further comprising addition of a Pd catalyst, Ac-DL-Phe-OH, triisopropylsilyl acetylene bromide, Ag₂CO₃, and a Cu salt.

The instant application provides a method of directing C7 selective alkynylation of C2, C3, and C4-substituted polycyclic aza-arenes having a hydrogen atom disposed on the 7-position thereof, including, but not limited to, olefination and alkynylation, comprising mixing the polycyclic aza-arene with the palladium-coordinating template compound of Formula XII with the template chaperone compound of Formula IV, further comprising addition of a Pd catalyst, an N-acylamino acid, triisopropylsilyl acetylene bromide, an Ag salt, and Cu(OH)₂.

The instant application provides a method of directing C7 selective alkynylation of C2, C3, and C4-substituted polycyclic aza-arenes having a hydrogen atom disposed on the 7-position thereof, including, but not limited to, olefination and alkynylation, comprising mixing the polycyclic aza-arene with the palladium-coordinating template compound of Formula XII with the template chaperone compound of Formula IV, further comprising addition of a Pd(OAc)₂, an N-acylamino acid, triisopropylsilyl acetylene bromide, an Ag salt, and Cu(OH)₂.

The instant application provides a method of directing C7 selective alkynylation of C2, C3, and C4-substituted polycyclic aza-arenes having a hydrogen atom disposed on the 7-position thereof, including, but not limited to, olefination and alkynylation, comprising mixing the polycyclic aza-arene with the palladium-coordinating template compound of Formula XII with the template chaperone compound of Formula IV, further comprising addition of a Pd catalyst, Ac-DL-Phe-OH, triisopropylsilyl acetylene bromide, an Ag salt, and Cu(OH)₂.

The instant application provides a method of directing C7 selective alkynylation of C2, C3, and C4-substituted polycyclic aza-arenes having a hydrogen atom disposed on the 7-position thereof, including, but not limited to, olefination and alkynylation, comprising mixing the polycyclic aza-arene with the palladium-coordinating template compound of Formula XII with the template chaperone compound of Formula IV, further comprising addition of a Pd catalyst, an N-acylamino acid, triisopropylsilyl acetylene bromide, Ag₂CO₃, and Cu(OH)₂.

The instant application provides a method of directing C7 selective alkynylation of C2, C3, and C4-substituted polycyclic aza-arenes having a hydrogen atom disposed on the 7-position thereof, including, but not limited to, olefination and alkynylation, comprising mixing the polycyclic aza-arene with the palladium-coordinating template compound of Formula XII with the template chaperone compound of Formula IV, further comprising addition of a Pd(OAc)₂, an N-acylamino acid, triisopropylsilyl acetylene bromide, Ag₂CO₃, and Cu(OH)₂.

The instant application provides a method of directing C7 selective alkynylation of C2, C3, and C4-substituted polycyclic aza-arenes having a hydrogen atom disposed on the 7-position thereof, including, but not limited to, olefination and alkynylation, comprising mixing the polycyclic aza-arene with the palladium-coordinating template compound of Formula XII with the template chaperone compound of Formula IV, further comprising addition of a Pd catalyst, Ac-DL-Phe-OH, triisopropylsilyl acetylene bromide, Ag₂CO₃, and Cu(OH)₂.

The instant application provides a method for directing C7 selective alkynylation of C2, C3, and C4-substituted polycyclic aza-arenes having a hydrogen atom disposed on the 7-position thereof, comprising i) mixing the polycyclic aza-arene, the template compound of Formula XII, the template chaperone compound of Formula II, and Pd(II)(OAc)₂; and ii) addition of Pd(II)(OAc)₂, Ac-DL-Phe-OH, triisopropylsilyl acetylene bromide, Ag₂CO₃, and Cu(OH)₂.

The instant application provides a palladium-coordinating enantiopure template compound for directing C7 selective functionalization of non-, C5, C6 or C8-substituted polycyclic aza-arenes having the structure of Formula XIVa or XIVb:

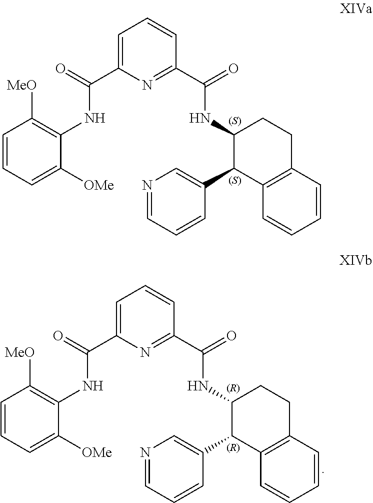

The instant application provides a method of olefination of the C7 position of non-, C5, C6 or C8-substituted polycyclic aza-arene having a hydrogen atom disposed on the 7-position thereof, comprising i) mixing the polycyclic aza-arene with the template compound of Formula XIVa or XIVb, the template chaperone compound of Formula IV, and a Pd catalyst and ii) mixing the product of step i) with additional Pd catalyst, an N-acylamino acid, an olefin functional group, and an Ag salt.

The instant application provides a method of olefination of the C7 position of non-, C5, C6 or C8-substituted polycyclic aza-arene having a hydrogen atom disposed on the 7-position thereof, comprising i) mixing the polycyclic aza-arene with the template compound of Formula XIVa or XIVb, the template chaperone compound of Formula IV, and Pd(OAc)₂ and ii) mixing the product of step i) with additional Pd(OAc)₂, an N-acylamino acid, an olefin functional group, and an Ag salt.

The instant application provides a method of olefination of the C7 position of non-, C5, C6 or C8-substituted polycyclic aza-arene having a hydrogen atom disposed on the 7-position thereof, comprising i) mixing the polycyclic aza-arene with the template compound of Formula XIVa or XIVb, the template chaperone compound of Formula IV, and a Pd catalyst and ii) mixing the product of step i) with additional Pd catalyst, Ac-L-Leu-OH or Ac-D-Leu-OH, an olefin functional group, and an Ag salt.

The instant application provides a method of olefination of the C7 position of non-, C5, C6 or C8-substituted polycyclic aza-arene having a hydrogen atom disposed on the 7-position thereof, comprising i) mixing the polycyclic aza-arene with the template compound of Formula XIVa or XIVb, the template chaperone compound of Formula IV, and Pd(OAc)₂ and ii) mixing the product of step i) with additional Pd(OAc)₂, Ac-L-Leu-OH or Ac-D-Leu-OH, an olefin functional group, and an Ag salt.

The instant application provides a method of olefination of the C7 position of non-, C5, C6 or C8-substituted polycyclic aza-arene having a hydrogen atom disposed on the 7-position thereof, comprising i) mixing the polycyclic aza-arene with the template compound of Formula XIVa or XIVb, the template chaperone compound of Formula IV, and a Pd catalyst and ii) mixing the product of step i) with additional Pd catalyst, an N-acylamino acid, an olefin functional group, and Ag₂CO₃.

The instant application provides a method of olefination of the C7 position of non-, C5, C6 or C8-substituted polycyclic aza-arene having a hydrogen atom disposed on the 7-position thereof, comprising i) mixing the polycyclic aza-arene with the template compound of Formula XIVa or XIVb, the template chaperone compound of Formula IV, and Pd(OAc)₂ and ii) mixing the product of step i) with additional Pd(OAc)₂, an N-acylamino acid, an olefin functional group, and Ag₂CO₃.

The instant application provides a method of olefination of the C7 position of non-, C5, C6 or C8-substituted polycyclic aza-arene having a hydrogen atom disposed on the 7-position thereof, comprising i) mixing the polycyclic aza-arene with the template compound of Formula XIVa or XIVb, the template chaperone compound of Formula IV, and a Pd catalyst and ii) mixing the product of step i) with additional Pd catalyst, Ac-L-Leu-OH or Ac-D-Leu-OH, an olefin functional group, and Ag₂CO₃.

The instant application provides a method of olefination of the C7 position of non-, C5, C6 or C8-substituted polycyclic aza-arene having a hydrogen atom disposed on the 7-position thereof, comprising i) mixing the polycyclic aza-arene with the template compound of Formula XIVa or XIVb, the template chaperone compound of Formula IV, and Pd(OAc)₂ and ii) mixing the product of step i) with additional Pd(OAc)₂, Ac-L-Leu-OH or Ac-D-Leu-OH, an olefin functional group, and Ag₂CO₃.

The instant application provides a method of alkynylation of the C7 position of non-, C5, or C6-substituted polycyclic aza-arene having a hydrogen atom disposed on the 7-position thereof, comprising i) mixing the polycyclic aza-arene with the template compound of Formula XII, the template chaperone compound of compound of Formula IV, and Pd catalyst; and ii) additional Pd catalyst, an N-acylamino acid, an alkynyl functional group, an Ag salt, and a Cu salt.

The instant application provides a method of alkynylation of the C7 position of non-, C5, or C6-substituted polycyclic aza-arene having a hydrogen atom disposed on the 7-position thereof, comprising i) mixing the polycyclic aza-arene with the template compound of Formula XII, the template chaperone compound of compound of Formula IV, and Pd(OAc)₂; and ii) additional Pd(OAc)₂, an N-acylamino acid, an alkynyl functional group, an Ag salt, and a Cu salt.

The instant application provides a method of alkynylation of the C7 position of non-, C5, or C6-substituted polycyclic aza-arene having a hydrogen atom disposed on the 7-position thereof, comprising i) mixing the polycyclic aza-arene with the template compound of Formula XII, the template chaperone compound of compound of Formula IV, and Pd catalyst; and ii) additional Pd catalyst, Ac-L-Leu-OH or Ac-D-Leu-OH, an alkynyl functional group, an Ag salt, and a Cu salt.

The instant application provides a method of alkynylation of the C7 position of non-, C5, or C6-substituted polycyclic aza-arene having a hydrogen atom disposed on the 7-position thereof, comprising i) mixing the polycyclic aza-arene with the template compound of Formula XII, the template chaperone compound of compound of Formula IV, and Pd(OAc)₂; and ii) additional Pd(OAc)₂, Ac-L-Leu-OH or Ac-D-Leu-OH, an alkynyl functional group, an Ag salt, and a Cu salt.

The instant application provides a method of alkynylation of the C7 position of non-, C5, or C6-substituted polycyclic aza-arene having a hydrogen atom disposed on the 7-position thereof, comprising i) mixing the polycyclic aza-arene with the template compound of Formula XII, the template chaperone compound of compound of Formula IV, and Pd catalyst; and ii) additional Pd catalyst, an N-acylamino acid, an alkynyl functional group, Ag₂CO₃, and a Cu salt.

The instant application provides a method of alkynylation of the C7 position of non-, C5, or C6-substituted polycyclic aza-arene having a hydrogen atom disposed on the 7-position thereof, comprising i) mixing the polycyclic aza-arene with the template compound of Formula XII, the template chaperone compound of compound of Formula IV, and Pd(OAc)₂; and ii) additional Pd(OAc)₂, an N-acylamino acid, an alkynyl functional group, Ag₂CO₃, and a Cu salt.

The instant application provides a method of alkynylation of the C7 position of non-, C5, or C6-substituted polycyclic aza-arene having a hydrogen atom disposed on the 7-position thereof, comprising i) mixing the polycyclic aza-arene with the template compound of Formula XII, the template chaperone compound of compound of Formula IV, and Pd catalyst; and ii) additional Pd catalyst, Ac-L-Leu-OH or Ac-D-Leu-OH, an alkynyl functional group, Ag₂CO₃, and a Cu salt.

The instant application provides a method of alkynylation of the C7 position of non-, C5, or C6-substituted polycyclic aza-arene having a hydrogen atom disposed on the 7-position thereof, comprising i) mixing the polycyclic aza-arene with the template compound of Formula XII, the template chaperone compound of compound of Formula IV, and Pd(OAc)₂; and ii) additional Pd(OAc)₂, Ac-L-Leu-OH or Ac-D-Leu-OH, an alkynyl functional group, Ag₂CO₃, and a Cu salt.

The instant application provides a method of alkynylation of the C7 position of non-, C5, or C6-substituted polycyclic aza-arene having a hydrogen atom disposed on the 7-position thereof, comprising i) mixing the polycyclic aza-arene with the template compound of Formula XII, the template chaperone compound of compound of Formula IV, and Pd catalyst; and ii) additional Pd catalyst, an N-acylamino acid, triisopropylsilyl acetylene bromide, an Ag salt, and a Cu salt.

The instant application provides a method of alkynylation of the C7 position of non-, C5, or C6-substituted polycyclic aza-arene having a hydrogen atom disposed on the 7-position thereof, comprising i) mixing the polycyclic aza-arene with the template compound of Formula XII, the template chaperone compound of compound of Formula IV, and Pd catalyst; and ii) additional Pd catalyst, an N-acylamino acid, triisopropylsilyl acetylene bromide, an Ag salt, and Cu(OH)₂.

The instant application provides a method of alkynylation of the C7 position of non-, C5, or C6-substituted polycyclic aza-arene having a hydrogen atom disposed on the 7-position thereof, comprising i) mixing the polycyclic aza-arene with the template compound of Formula XII, the template chaperone compound of compound of Formula IV, and Pd catalyst; and ii) additional Pd catalyst, an N-acylamino acid, triisopropylsilyl acetylene bromide, Ag₂CO₃, and Cu(OH)₂.

The instant application provides a method of alkynylation of the C7 position of non-, C5, or C6-substituted polycyclic aza-arene having a hydrogen atom disposed on the 7-position thereof, comprising i) mixing the polycyclic aza-arene with the template compound of Formula XII, the template chaperone compound of compound of Formula IV, and Pd(OAc)₂; and ii) additional Pd(OAc)₂, an N-acylamino acid, triisopropylsilyl acetylene bromide, Ag₂CO₃, and Cu(OH)₂.

The instant application provides a method of alkynylation of the C7 position of non-, C5, or C6-substituted polycyclic aza-arene having a hydrogen atom disposed on the 7-position thereof, comprising i) mixing the polycyclic aza-arene with the template compound of Formula XII, the template chaperone compound of compound of Formula IV, and Pd catalyst; and ii) additional Pd catalyst, Ac-L-Leu-OH or Ac-D-Leu-OH, triisopropylsilyl acetylene bromide, Ag₂CO₃, and Cu(OH)₂.

The instant application provides a method of alkynylation of the C7 position of non-, C5, or C6-substituted polycyclic aza-arene having a hydrogen atom disposed on the 7-position thereof, comprising i) mixing the polycyclic aza-arene with the template compound of Formula XII, the template chaperone compound of compound of Formula IV, and Pd(OAc)₂; and ii) additional Pd(OAc)₂, Ac-L-Leu-OH or Ac-D-Leu-OH, triisopropylsilyl acetylene bromide, Ag₂CO₃, and Cu(OH)₂.

The instant application provides any one of the above methods, wherein the polycyclic aza-arene has the structure of any one of Formulae XV-XXIX

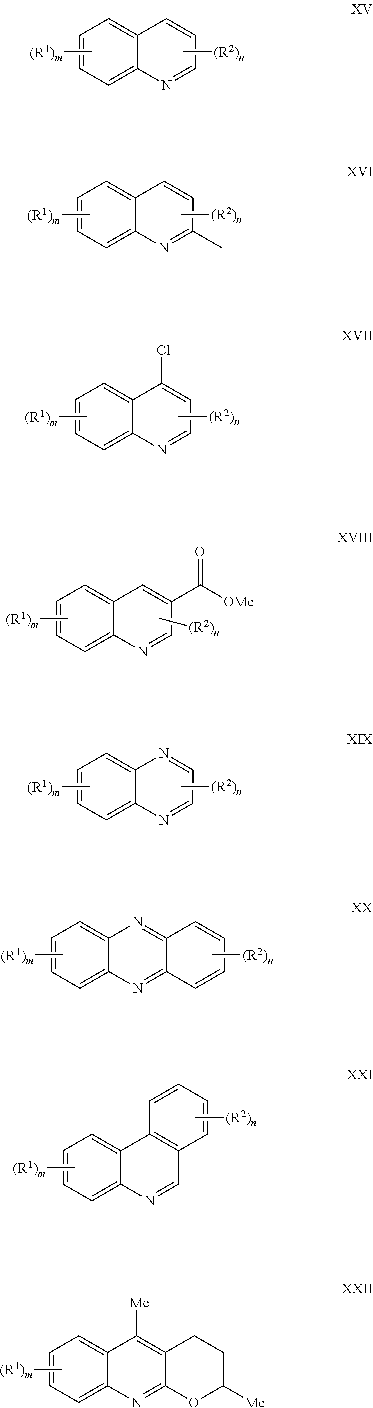

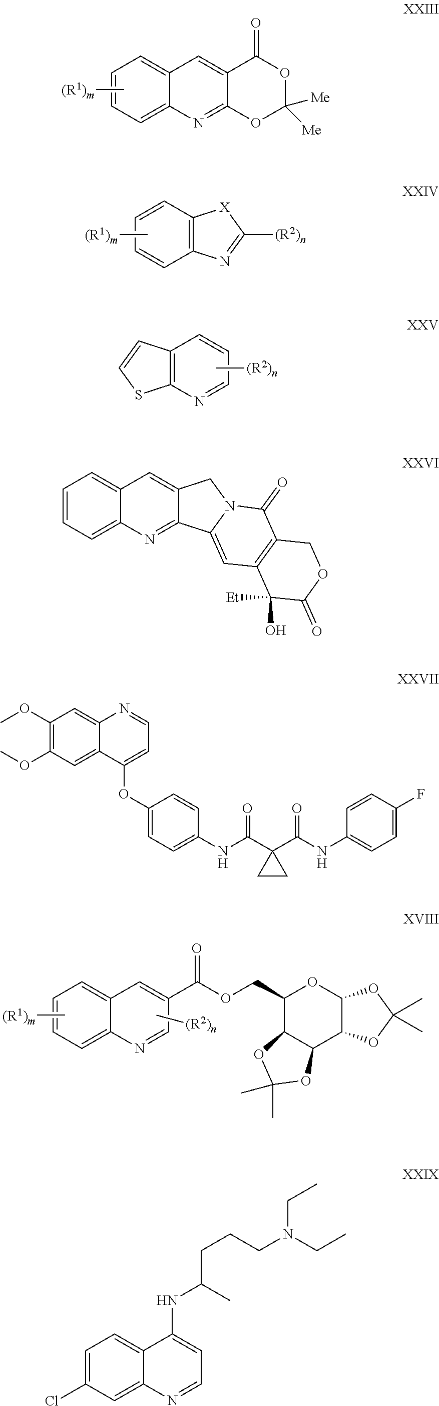

wherein:
each R¹ and R² is independently selected from H, F, Cl, C₁-C₆ alkyl, C₃-C₆ cycloalkyl, 3,5-dimethylC₆H₃, O(C₁-C₆ alkyl), CF₃, C(═O)OH, C₁-C₆ alkylC(═O)OH, C(═O)OC₁-C₆ alkyl) and C₁-C₆ alkylC(═O)OC₁-C₆ alkyl;
m is 0, 1, 2, or 3;
n is 0, 1, 2, or 3; and
X is S, O, NR², or C(R²)₂.

The instant application provides any one of the above methods, wherein the polycyclic aza-arene is an optionally substituted bicyclic aza-arene.

The instant application provides any one of the above methods, wherein the polycyclic aza-arene is an optionally substituted tricyclic aza-arene.

The instant application provides any one of the above methods, wherein the polycyclic aza-arene is an optionally substituted quinoline.

The instant application provides any one of the above methods, wherein the polycyclic aza-arene is optionally substituted 3-methylquinoline.

The instant application provides any one of the above methods, wherein the polycyclic aza-arene is an optionally substituted tetracyclic or pentacyclic quinoline.

The instant application provides any one of the above methods, wherein the polycyclic aza-arene is an optionally substituted quinoxaline.

The instant application provides any one of the above methods, wherein the polycyclic aza-arene is an optionally substituted benzothiophene.

The instant application provides any one of the above methods, wherein the polycyclic aza-arene is an optionally substituted phenazine.

The instant application provides any one of the above methods, wherein the polycyclic aza-arene is an optionally substituted thieno[2,3-b]pyridine.

The instant application provides a process for iterative C7 and C6 C—H activation and selective substitution of polycyclic aza-arenes comprising i) mixing the polycyclic aza-arene with the C7 directing template Formula of XII, Formula XIVa or Formula XIVb, and the template chaperone compound of Formula IV in the presence of Pd(OAc)₂, the first functional group to be added at position C7, Ag(OAc)₂, and Ac-DL-Phe-OH, Ac-D-Leu-OH, or Ac-L-Leu-OH and ii) mixing the polycyclic aza-arene product of step i) with the template compound of Formula I and the template chaperone compound of Formula IV in the presence of Pd(OAc)₂, the second functional group to be added at position C6, Ag(OAc)₂, and Ac-Gly-OH.

The instant application provides the above process, wherein the first functional group is an olefin and the second functional group is an olefin.

The instant application provides the above process, wherein the first functional group is an olefin and the second functional group is an allyl in the additional presence of Cu(OH)₂.

The instant application provides the above process, wherein the first functional group is an olefin and the second functional group is an alkyne in the additional presence of Cu(OH)₂.

The instant application provides the above process, wherein the first functional group is an alkyne in the additional presence of Cu(OH)₂ and the second functional group is an olefin.

The instant application provides the above process, wherein the first functional group is an alkyne in the additional presence of Cu(OH)₂ and the second functional group is an allyl in the additional presence of Cu(OH)₂.

The instant application provides the above process, wherein the first functional group is an alkyne in the additional presence of Cu(OH)₂ and the second functional group is an alkyne in the additional presence of Cu(OH)₂.

The instant application provides a process for iterative C7 and C6 C—H activation and selective olefination of polycyclic aza-arenes comprising i) mixing the polycyclic aza-arene with the C7 directing template Formula of XII and the template chaperone compound of Formula IV in the presence of Pd(OAc)₂, the first olefin functional group to be added at position C7, and Ac-DL-Phe-OH, Ac-D-Leu-OH, or Ac-L-Leu-OH, and Ag(OAc)₂, and ii) mixing the polycyclic aza-arene product of step i) with the template compound of Formula I and the template chaperone compound of Formula IV in the presence of Pd(OAc)₂, the second olefin functional group to be added at position C6, Ag(OAc)₂, and Ac-Gly-OH.

The instant application provides a process for iterative C7 and C6 C—H activation and selective alkynylation of polycyclic aza-arenes comprising i) mixing the polycyclic aza-arene with the C7 directing template Formula of XII and the template chaperone compound of Formula IV in the presence of Pd(OAc)₂, the first alkynyl functional group to be added at position C7, and Ac-DL-Phe-OH, Ac-D-Leu-OH, or Ac-L-Leu-OH, Ag(OAc)₂, and Cu(OH)₂ and ii) mixing the polycyclic aza-arene product of step i) with the template compound of Formula I and the template chaperone compound of Formula IV in the presence of Pd(OAc)₂, the second alkynyl functional group to be added at position C6, Ag(OAc)₂, Cu(OH)₂ and Ac-Gly-OH.

The instant application provides any palladium-coordinating template compound, palladium-coordinating template chaperone compound, template palladium complex, template chaperone palladium complex, method of functionalization including, but not limited to, olefination, allylation, alkynylation, arylation, iodination and cyanation, or process for iterative C7, C6 or related positional C—H activation and substitution of polycyclic aza-arenes as herein described.

### Definitions

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “as defined herein above” refers to the broadest definition for each group as provided in the Summary of the Invention, the Detailed Description of the Invention, the Experimentals, or the broadest claim. In all other embodiments provided below, substituents which can be present in each embodiment and which are not explicitly defined retain the broadest definition provided in the Summary of the Invention.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

As used herein, unless specifically indicated otherwise, the word “or” is used in the “inclusive” sense of “and/or” and not the “exclusive” sense of “either/or”.

The term “independently” is used herein to indicate that a variable is applied in any one instance without regard to the presence or absence of a variable having that same or a different definition within the same compound. Thus, in a compound in which “R” appears twice and is defined as “independently selected from” means that each instance of that R group is separately identified as one member of the set which follows in the definition of that R group. For example, “each R¹ and R² is independently selected from carbon and nitrogen” means that both R¹ and R² can be carbon, both R¹ and R² can be nitrogen, or R¹ or R² can be carbon and the other nitrogen or vice versa.

When any variable occurs more than one time in any moiety or formula depicting and describing compounds employed or claimed in the present invention, its definition on each occurrence is independent of its definition at every other occurrence. Also, combinations of substituents and/or variables are permissible only if such compounds result in stable compounds.

The symbols “*” at the end of a bond or a line drawn through a bond or “˜˜˜˜” drawn through a bond each refer to the point of attachment of a functional group or other chemical moiety to the rest of the molecule of which it is a part.

A bond drawn into ring system (as opposed to connected at a distinct vertex) indicates that the bond may be attached to any of the suitable ring atoms.

The term “optional” or “optionally” as used herein means that a subsequently described event or circumstance may, but need not, occur, and that the description includes instances where the event or circumstance occurs and instances in which it does not. For example, “optionally substituted” means that the “optionally substituted” moiety may incorporate a hydrogen or a substituent.

The phrase “optional bond” means that the bond may or may not be present, and that the description includes single, double, or triple bonds. If a substituent is designated to be a “bond” or “absent”, the atoms linked to the substituents are then directly connected.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Certain compounds of Formulae I-XXIX may exhibit tautomerism. Tautomeric compounds can exist as two or more interconvertable species. Prototropic tautomers result from the migration of a covalently bonded hydrogen atom between two atoms. Tautomers generally exist in equilibrium and attempts to isolate an individual tautomers usually produce a mixture whose chemical and physical properties are consistent with a mixture of compounds. The position of the equilibrium is dependent on chemical features within the molecule. For example, in many aliphatic aldehydes and ketones, such as acetaldehyde, the keto form predominates while; in phenols, the enol form predominates. Common prototropic tautomers include keto/enol (—C(═O)—CH—⇄—C(—OH)═CH—), amide/imidic acid (—C(═O)—NH—⇄—C(—OH)═N—) and amidine (—C(═NR)—NH—⇄—C(—NHR)═N—) tautomers. The latter two are particularly common in heteroaryl and heterocyclic rings and the present invention encompasses all tautomeric forms of the compounds.

The term “template compound” or “directing template” (T) as used herein refers to compounds that enable the modular differentiation and functionalization of adjacent remote (for example C6 vs. C7) and positionally-similar positions (for example C3 vs. C7) on polycyclic aza-arene scaffolds through careful modulation of either distance and geometry or previously unconsidered chirality in template design and are capable of selectively positioning a catalyst proximate to a target remote C—H bond via a macrocyclophanic pre-transition state.¹⁵⁻¹⁹ Chiral recognition is vital in the granular discrimination between competing C3 and C7-H bonds when the differentiation via distance and geometry is insufficient. Thus, precise recognition of a directing template's distance, geometry and chirality enables the iterative C—H editing of quinoline pharmacophores at any desired site and order.

As used herein “template chaperone” (TC) refers to companion compounds used with the directing template compounds and are used to turn over the directing template compounds, allowing them to be used catalytically.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10^{th} Ed., McGraw Hill Companies Inc., New York (2001). Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

The definitions described herein may be appended to form chemically-relevant combinations, such as “heteroalkylaryl,” “haloalkylheteroaryl,” “arylalkylheterocyclyl,” “alkylcarbonyl,” “alkoxyalkyl,” and the like. When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” refers to an alkyl group having one to two phenyl substituents, and thus includes benzyl, phenylethyl, and biphenyl. An “alkylaminoalkyl” is an alkyl group having one to two alkylamino substituents. “Hydroxyalkyl” includes 2-hydroxyethyl, 2-hydroxypropyl, 1-(hydroxymethyl)-2-methylpropyl, 2-hydroxybutyl, 2,3-dihydroxybutyl, 2-(hydroxymethyl), 3-hydroxypropyl, and so forth. Accordingly, as used herein, the term “hydroxyalkyl” is used to define a subset of heteroalkyl groups defined below. The term -(ar)alkyl refers to either an unsubstituted alkyl or an aralkyl group. The term (hetero)aryl or (het)aryl refers to either an aryl or a heteroaryl group.

The term “acyl” as used herein denotes a group of formula —C(═O)R wherein R is hydrogen or lower alkyl as defined herein. The term or “alkylcarbonyl” as used herein denotes a group of formula C(═O)R wherein R is alkyl as defined herein. The term C₁₋₆ acyl refers to a group —C(═O)R contain 6 carbon atoms. The term “arylcarbonyl” as used herein means a group of formula C(═O)R wherein R is an aryl group; the term “benzoyl” as used herein an “arylcarbonyl” group wherein R is phenyl.

The term “alkyl” as used herein denotes an unbranched or branched chain, saturated, monovalent hydrocarbon residue containing 1 to 12 carbon atoms. The term “lower alkyl” or “C₁-C₆ alkyl” as used herein denotes a straight or branched chain hydrocarbon residue containing 1 to 6 carbon atoms. “C₁₋₁₂ alkyl” as used herein refers to an alkyl composed of 1 to 12 carbons. Examples of alkyl groups include, but are not limited to, lower alkyl groups include methyl, ethyl, propyl, i-propyl, n-butyl, i-butyl, t-butyl or pentyl, isopentyl, neopentyl, hexyl, heptyl, and octyl.

When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” denotes the radical R′R″—, wherein R′ is a phenyl radical, and R″ is an alkylene radical as defined herein with the understanding that the attachment point of the phenylalkyl moiety will be on the alkylene radical. Examples of arylalkyl radicals include, but are not limited to, benzyl, phenylethyl, 3-phenylpropyl. The terms “arylalkyl” or “aralkyl” are interpreted similarly except R′ is an aryl radical. The terms “(het)arylalkyl” or “(het)aralkyl” are interpreted similarly except R′ is optionally an aryl or a heteroaryl radical.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” refers to a radical of a straight-chain or branched saturated hydrocarbon group having from 1 to 20 carbon atoms (“C₁₋₂₀ alkyl”). In some embodiments, an alkyl group has 1 to 15 carbon atoms (“C₁₋₁₅ alkyl”). In some embodiments, an alkyl group has 1 to 14 carbon atoms (“C₁₋₁₄ alkyl”). In some embodiments, an alkyl group has 1 to 13 carbon atoms (“C₁₋₁₃ alkyl”). In some embodiments, an alkyl group has 1 to 12 carbon atoms (“C₁₋₁₂ alkyl”). In some embodiments, an alkyl group has 1 to 11 carbon atoms (“C₁₋₁₁ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C), and n-hexyl (C₆). Additional examples of alkyl groups include n-heptyl (C₇), n-octyl (C₈) and the like.

“Alkenyl” or “olefin” refers to a radical of a straight-chain or branched hydrocarbon group having from 2 to 10 carbon atoms and 1, 2, 3, or 4 carbon-carbon double bonds (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₆ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C), pentadienyl (C₅), hexenyl (C₆), and the like. Additional examples of alkenyl include heptenyl (C7), octenyl (C), octatrienyl (C), and the like.

“Alkynyl” refers to a radical of a straight-chain or branched hydrocarbon group having from 2 to 10 carbon atoms and one or more carbon-carbon triple bonds (e.g., 1, 2, 3, or 4 triple bonds) (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₄ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃). 2-propynyl (C3), 1-butynyl (C₄), 2-butynyl (C₄), and the like. Examples of C2-6 alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like.

The terms “haloalkyl” or “halo-lower alkyl” or “lower haloalkyl” refers to a straight or branched chain hydrocarbon residue containing 1 to 6 carbon atoms wherein one or more carbon atoms are substituted with one or more halogen atoms.

The term “alkylene” or “alkylenyl” as used herein denotes a divalent saturated linear hydrocarbon radical of 1 to 10 carbon atoms (e.g., (CH₂)ₙ) or a branched saturated divalent hydrocarbon radical of 2 to 10 carbon atoms (e.g., —CHMe- or —CH₂CH(i-Pr)CH₂—), unless otherwise indicated. Except in the case of methylene, the open valences of an alkylene group are not attached to the same atom. Examples of alkylene radicals include, but are not limited to, methylene, ethylene, propylene, 2-methyl-propylene, 1,1-dimethyl-ethylene, butylene, 2-ethylbutylene.

The term “alkoxy” as used herein means an —O-alkyl group, wherein alkyl is as defined above such as methoxy, ethoxy, n-propyloxy, i-propyloxy, n-butyloxy, i-butyloxy, t-butyloxy, pentyloxy, hexyloxy, including their isomers. “Lower alkoxy” as used herein denotes an alkoxy group with a “lower alkyl” group as previously defined. “C₁₋₁₀ alkoxy” as used herein refers to an —O-alkyl wherein alkyl is C₁₋₁₀.

The term “hydroxyalkyl” as used herein denotes an alkyl radical as herein defined wherein one to three hydrogen atoms on different carbon atoms is/are replaced by hydroxyl groups.

The terms “alkylsulfonyl” and “arylsulfonyl” as used herein refers to a group of formula —S(═O)₂R wherein R is alkyl or aryl respectively and alkyl and aryl are as defined herein. The term “heteroalkylsulfonyl” as used herein refers herein denotes a group of formula —S(═O)₂R wherein R is “heteroalkyl” as defined herein.

The terms “alkylsulfonylamino” and “arylsulfonylamino” as used herein refers to a group of formula —NR′S(═O)₂R wherein R is alkyl or aryl respectively, R′ is hydrogen or C₁₋₃ alkyl, and alkyl and aryl are as defined herein.

The term “cycloalkyl” as used herein refers to a saturated carbocyclic ring containing 3 to 8 carbon atoms, i.e. cyclopropyl, cyclobutyl, cyclopentyl, cyclohexyl, cycloheptyl or cyclooctyl. “C₃₇ cycloalkyl” as used herein refers to an cycloalkyl composed of 3 to 7 carbons in the carbocyclic ring.

The term carboxy-alkyl as used herein refers to an alkyl moiety wherein one, hydrogen atom has been replaced with a carboxyl with the understanding that the point of attachment of the heteroalkyl radical is through a carbon atom. The term “carboxy” or “carboxyl” refers to a —CO₂H moiety.

The term “heteroaryl” or “heteroaromatic” as used herein means a monocyclic or bicyclic radical of 5 to 12 ring atoms having at least one aromatic ring containing four to eight atoms per ring, incorporating one or more N, O, or S heteroatoms, the remaining ring atoms being carbon, with the understanding that the attachment point of the heteroaryl radical will be on an aromatic ring. As well known to those skilled in the art, heteroaryl rings have less aromatic character than their all-carbon counter parts. Thus, for the purposes of the invention, a heteroaryl group need only have some degree of aromatic character. Examples of heteroaryl moieties include monocyclic aromatic heterocycles having 5 to 6 ring atoms and 1 to 3 heteroatoms include, but is not limited to, pyridinyl, pyrimidinyl, pyrazinyl, pyrrolyl, pyrazolyl, imidazolyl, oxazol, isoxazole, thiazole, isothiazole, triazoline, thiadiazole and oxadiaxoline which can optionally be substituted with one or more, preferably one or two substituents selected from hydroxy, cyano, alkyl, alkoxy, thio, lower haloalkoxy, alkylthio, halo, lower haloalkyl, alkylsulfinyl, alkylsulfonyl, halogen, amino, alkylamino, dialkylamino, aminoalkyl, alkylaminoalkyl, and dialkylaminoalkyl, nitro, alkoxycarbonyl and carbamoyl, alkylcarbamoyl, dialkylcarbamoyl, arylcarbamoyl, alkylcarbonylamino and arylcarbonylamino. Examples of bicyclic moieties include, but are not limited to, quinolinyl, isoquinolinyl, benzofuryl, benzothiophenyl, benzoxazole, benzisoxazole, benzothiazole and benzisothiazole. Bicyclic moieties can be optionally substituted on either ring; however the point of attachment is on a ring containing a heteroatom.

The term “heterocyclyl”, “heterocycloalkyl” or “heterocycle” as used herein denotes a monovalent saturated cyclic radical, consisting of one or more rings, preferably one to two rings, including spirocyclic ring systems, of three to eight atoms per ring, incorporating one or more ring heteroatoms (chosen from N,O or S(O)₀₋₂), and which can optionally be independently substituted with one or more, preferably one or two substituents selected from hydroxy, oxo, cyano, lower alkyl, lower alkoxy, lower haloalkoxy, alkylthio, halo, lower haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonylamino, arylcarbonylamino, unless otherwise indicated. Examples of heterocyclic radicals include, but are not limited to, azetidinyl, pyrrolidinyl, hexahydroazepinyl, oxetanyl, tetrahydrofuranyl, tetrahydrothiophenyl, oxazolidinyl, thiazolidinyl, isoxazolidinyl, morpholinyl, piperazinyl, piperidinyl, tetrahydropyranyl, thiomorpholinyl, quinuclidinyl and imidazolinyl.

“Heterocyclyl” or “heterocyclic” refers to a group or radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiiranyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 3 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydro-benzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

“Aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl ((α-naphthyl) and 2-naphthyl (β-naphthyl)). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system.

“Heteroaryl” refers to a radical of a 5-14 membered monocyclic or polycyclic (e.g., bicyclic, tricyclic) 4n+2 aromatic ring system (e.g., having 6, 10, or 14 pi electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

“Saturated” refers to a ring moiety that does not contain a double or triple bond, i.e., the ring contains all single bonds.

Alkyl, cycloalkyl, heterocyclyl, aryl, and heteroaryl groups may be optionally substituted. Optionally substituted refers to a group which may be substituted or unsubstituted. In general, the term “substituted” means that at least one hydrogen present on a group is replaced with a non-hydrogen substituent, and which upon substitution results in a stable compound, e.g., a compound which does not spontaneously undergo transformation such as by rearrangement, cyclization, elimination, or other reaction. Heteroatoms such as nitrogen, oxygen, and sulfur may have hydrogen substituents and/or non-hydrogen substituents which satisfy the valencies of the heteroatoms and results in the formation of a stable compound.

Exemplary non-hydrogen substituents wherein a moiety is “optionally substituted” as used herein means the moiety may be substituted with any additional moiety selected from, but not limited to, the group consisting of halogen, —CN, —NO₂, —N₃, —SO₂H, —SO₃H, —OH, —OR^{aa}, —N(R^{bb})₂, —N(OR^{cc})R^{bb}, —SH, —SR^{aa}, —C(═O)R^{aa}, —CO₂H, —CHO, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —S(═O)R^{a}, —OS(═O)R^{aa}, —B(OR^{cc})₂, C₁₋₁₀ alkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, wherein each alkyl, alkenyl, alkynyl, carbocyclyl, heterocyclyl, aryl, and heteroaryl is independently substituted with 0, 1, 2, 3, 4, or 5 Rid groups, or two geminal hydrogens on a carbon atom are replaced with the group ═O; each instance of R^{aa} is, independently, selected from the group consisting of C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{aa} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{bb} is, independently, selected from the group consisting of hydrogen, —OH, —OR^{aa}, —N(R^{cc})₂, —CN, —C(═O)R^{a}, —C(═O)N(R^{cc})₂, —CO₂R^{aa}, —SO₂R^{aa}, —SO₂N(R^{cc})₂, —SOR^{aa}, C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{bb} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{cc} is, independently, selected from the group consisting of hydrogen, C₁₋₁₀ alkyl, C₁₋₁₀ perhaloalkyl, C₂₋₁₀ alkenyl, C₂₋₁₀ alkynyl, C₃₋₁₄ carbocyclyl, 3- to 14-membered heterocyclyl, C₆₋₁₄ aryl, and 5- to 14-membered heteroaryl, or two R^{cc} groups are joined to form a 3- to 14-membered heterocyclyl or 5- to 14-membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; and each instance of R^{dd} is, independently, selected from the group consisting of halogen, —CN, —NO₂, —N₃, —SO₂H. —SO₃H, —OH, —OC₁₋₆ alkyl, —ON(C₁₋₆ alkyl)₂, —N(C₁₋₆ alkyl)₂, —N(OC₁₋₆ alkyl)(C₁₋₆ alkyl), —N(OH)(C₁₋₆ alkyl), —NH(OH), —SH, —SC₁₋₆ alkyl, —C(═O)(C₁₋₆ alkyl), —CO₂H. —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₄ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —B(OH)₂, —B(OC₁₋₆ alkyl)₂, C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3- to 10-membered heterocyclyl, and 5- to 10-membered heteroaryl; or two geminal R^{dd} substituents on a carbon atom may be joined to form ═O.

“Halo” or “halogen” refers to fluorine (fluoro, —F), chlorine (chloro, —Cl), bromine (bromo, —Br), or iodine (iodo, —I).

As used herein, the term “composition” is intended to encompass a product comprising the specified ingredients, as well as any product which results, directly or indirectly, from combination of the specified ingredients.

“Salt” includes any and all salts. “Pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts include those derived from inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N+(C-alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

Unless otherwise indicated, compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC). Compounds described herein can be in the form of individual isomers substantially free of other isomers, and alternatively, as mixtures of various isomers.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, replacement of a carbon by a ¹³C- or ¹⁴C-enriched carbon, and/or replacement of an oxygen atom with ¹⁸O, are within the scope of the disclosure. Other examples of isotopes include ¹⁵N, ¹⁸O, ³¹P, ³²P, ³⁵S, ¹⁸F, ³⁶Cl and ¹²³I. Compounds with such isotopically enriched atoms are useful, for example, as analytical tools or probes in biological assays.

Certain isotopically-labelled compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability.

Certain isotopically-labelled compounds of Formula (I) can be useful for medical imaging purposes, for example, those labeled with positron-emitting isotopes like ¹¹C or ¹⁸F can be useful for application in Positron Emission Tomography (PET) and those labeled with gamma ray emitting isotopes like ¹²³I can be useful for application in Single Photon Emission Computed Tomography (SPECT). Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Further, substitution with heavier isotopes such as deuterium (i.e., ²H) may afford certain therapeutic advantages resulting from greater metabolic stability (e.g., increased in vivo half-life or reduced dosage requirements), and hence, may be preferred in some circumstances. Additionally, isotopic substitution at a site where epimerization occurs may slow or reduce the epimerization process and thereby retain the more active or efficacious form of the compound for a longer period of time. Isotopically labeled compounds of Formula (I), in particular those containing isotopes with longer half-lives (t_{1/2}>1 day), can generally be prepared by following procedures analogous to those disclosed in the Schemes and/or in the Examples herein below, by substituting an appropriate isotopically labeled reagent for a non-isotopically labeled reagent.

### DETAILED DESCRIPTION OF THE DRAWINGS

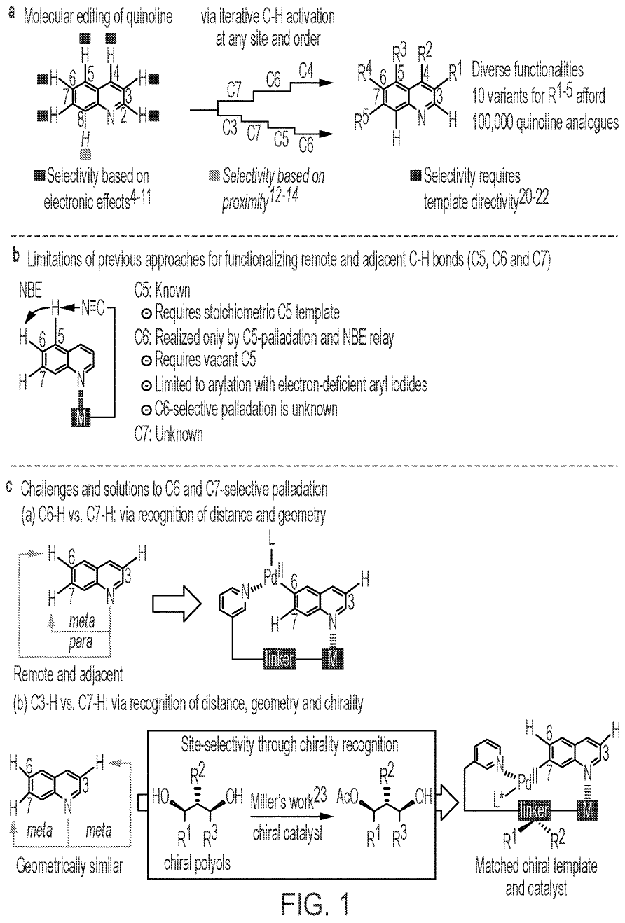

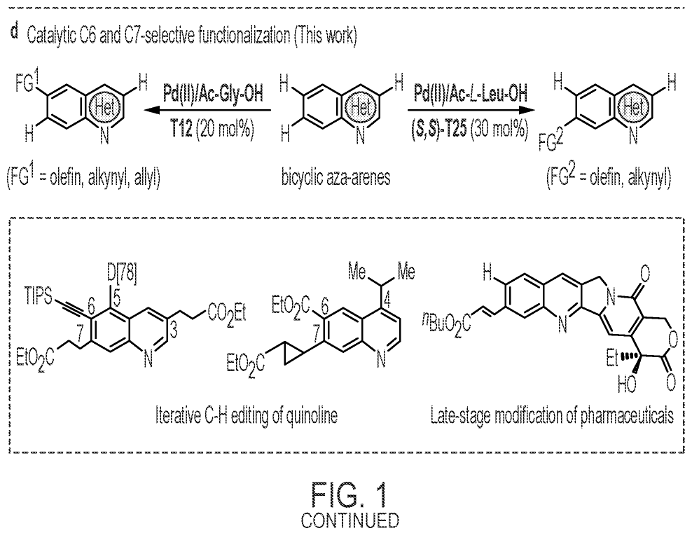

FIG. 1. Molecular editing of heterocycles, a, Molecular editing of quinoline, b, Limitations of previous approaches for functionalizing remote and adjacent C—H bonds (C5, C6 and C7), c, Challenges and solutions to C6 and C7-selective palladation, d, Catalytic C6 and C7-selective functionalization (This work). NBE, norbornene; M, metal; L, ligand; FG, functional group.

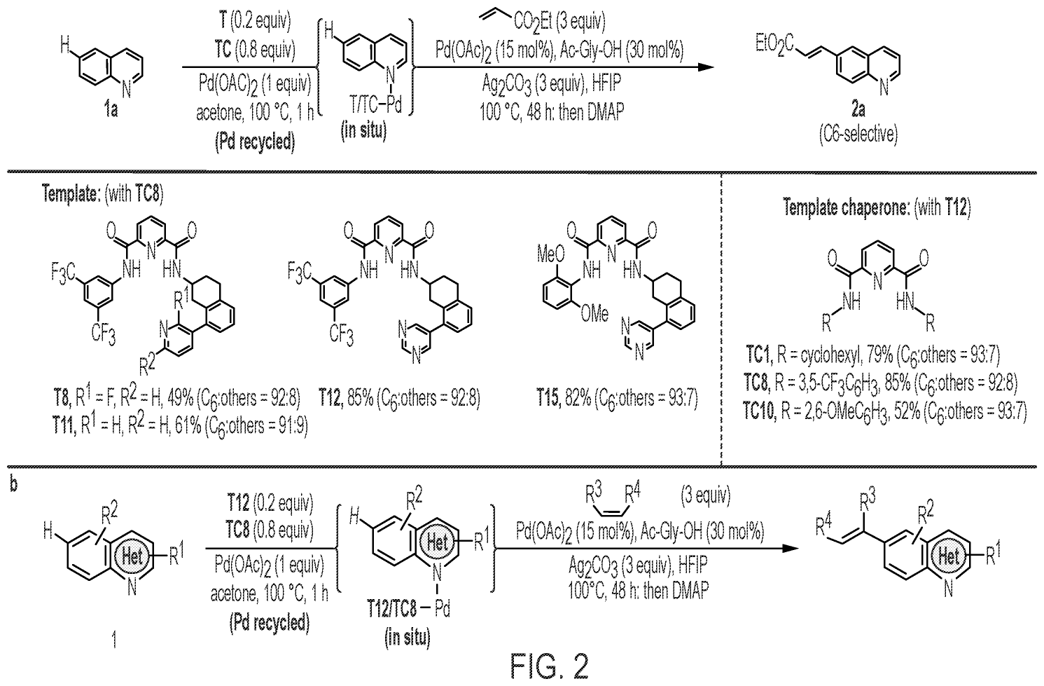

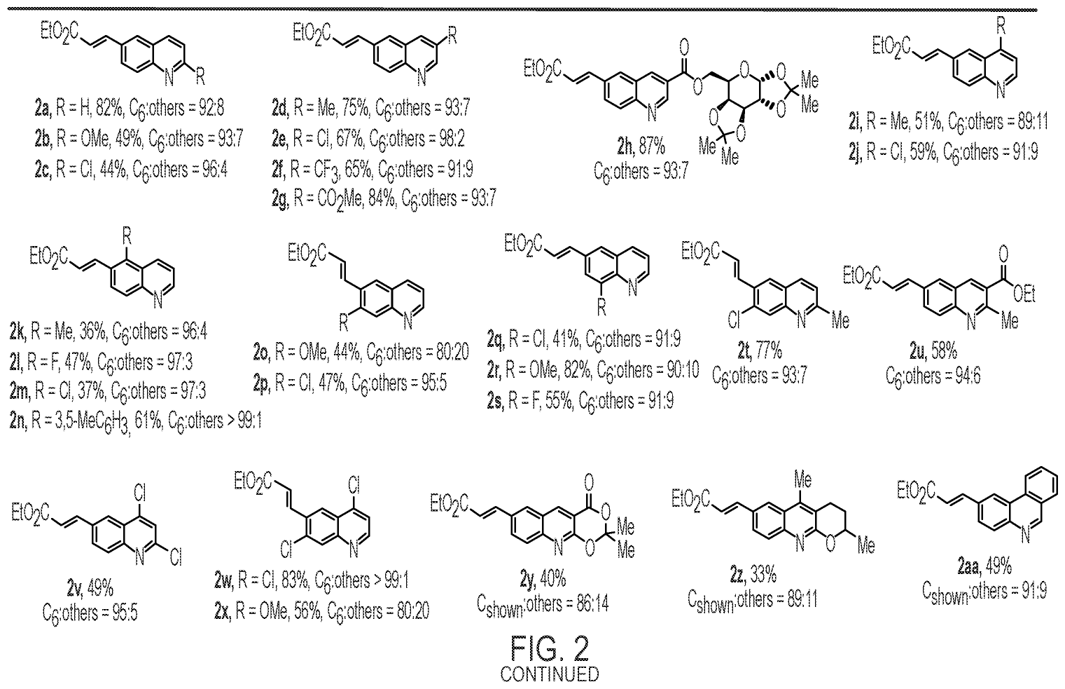

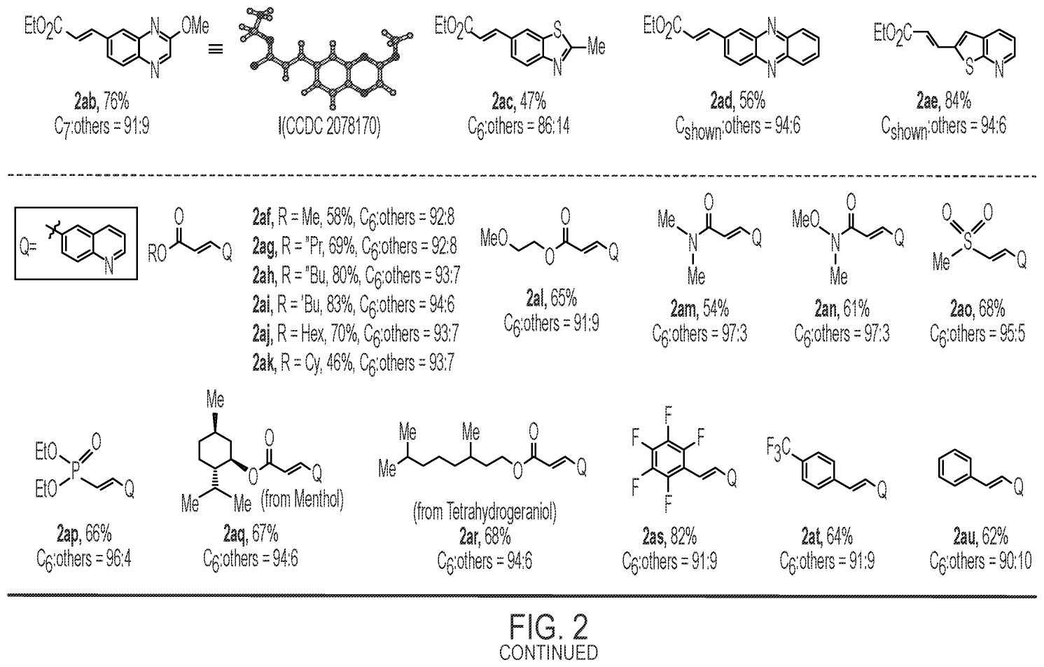

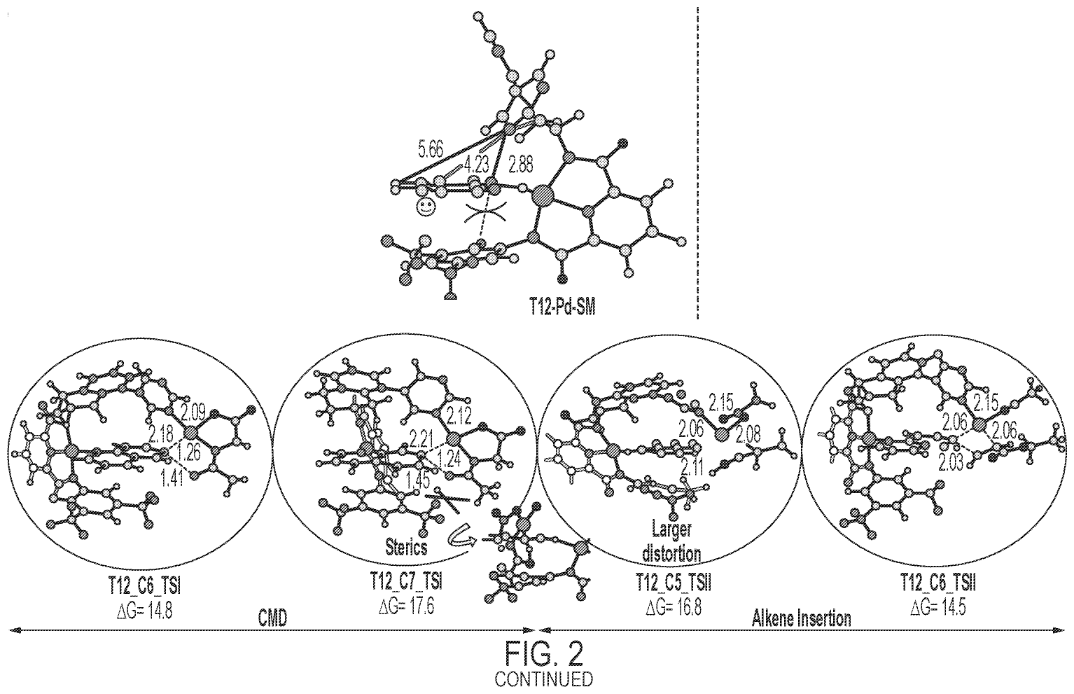

Using quinoline 1a as the model substrate, we initially targeted the development of a selective C6-H olefination reaction. Considering that our previous C5 template is used in stoichiometric amounts, a key objective at the outset of our studies was the catalytic use of our templates. Therefore, we introduced easily synthetized symmetrical template chaperone TC8 (two steps, no chromatography) to mask the quinoline nitrogen and facilitate product turnover from the directing template. An initial hit was found through a systematic screen of linker length in the presence of 2-fluoro-3-phenylpyridyl motif as the directing group (T3, see Supplementary Information, Table S1)26. A rigidified analogue of T3 bearing an alicyclic two-carbon spacer (T8) was next pursued, which to our delight, gave a marked improvement to both yield and C6 selectivity. Further tuning of the directing motif (T9 to T12, Table S1) showed that yields were improved using 3-phenylpyrimidyl-bearing T12 (FIG. 2a), while an assessment of the left-hand portion of the template and template chaperone scaffolds gave no noticeable improvements in reactivity and selectivity. The optimal result with T12 and TC8, both bearing 3,5-ditrifluoromethylphenyl side arms, likely arises from their structural homology, improving the efficacy of substrate/product exchange in the reaction. In all cases, the incorporated palladium within the directing template and template chaperones can be easily recovered as the TC-Pd-MeCN complex, and recycled with no loss in reaction efficacy (see Supplementary Information 2.9).

With optimized template and conditions in hand, we next evaluated the reaction scope with respects to quinoline and related heterocycles (2a to 2aa, FIG. 2b). Various electron-donating and electron-withdrawing groups were compatible in the reaction showing little difference in yield or selectivity (2a to 2s), confirming the power of our proximity-driven directing approach in overriding inherent electronic preferences. A series of multiply-substituted and polycyclic quinolines were also well tolerated (2t to 2aa), and we were pleased to find that the reaction tolerates a variety of aza-arenes; quinoxaline (2ab), benzothiophene (2ac), phenazine (2ad), and thieno[2,3-b]pyridine (2ae) all afforded the desired products in good yields and high selectivities. Next, we examined the scope of coupling partners with unsubstituted quinoline under the standard conditions. A variety of acrylates (2af to 2al), vinylamides (2am and 2an), vinylsulfone (2ao), vinylphosphonate (2ap), styrenes (2as to 2au) and more complex terpenoid-derived acrylate coupling partners (2aq, from menthol; 2ar, from tetrahydrogeraniol) were well tolerated, delivering the corresponding products in good to excellent yields and selectivities.

Density functional theory (DFT) studies were conducted to understand the origin of C6-selectivity in our catalytic templates. Analysis of the concerted metalation deprotonation (CMD) step immediately excluded a C7-selective pathway because of higher energies incurred by repulsive interactions between the template phenyl ring and ligand acetyl groups (FIG. 2c). The analysis also suggested that the initial C—H metalation was likely unselective between C5 and C6, which was corroborated by observing unselective substrate deuterium incorporation (see Supplementary Information 2.14). A larger template distortion required to access the C5 position results in a lower energy barrier for the C6-selective alkene insertion step relative to C5. Therefore, a combination of a more favored C—H activation (disfavoring C7) and alkene insertion steps (disfavoring C5) give rise to the observed C6 selectivity for this template.

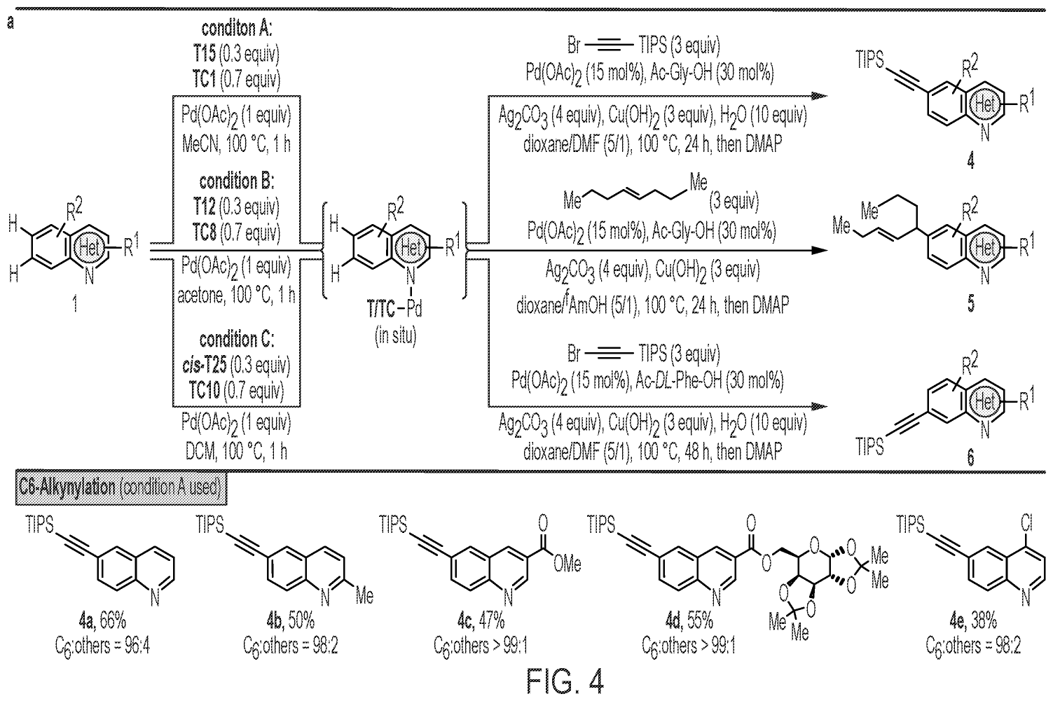

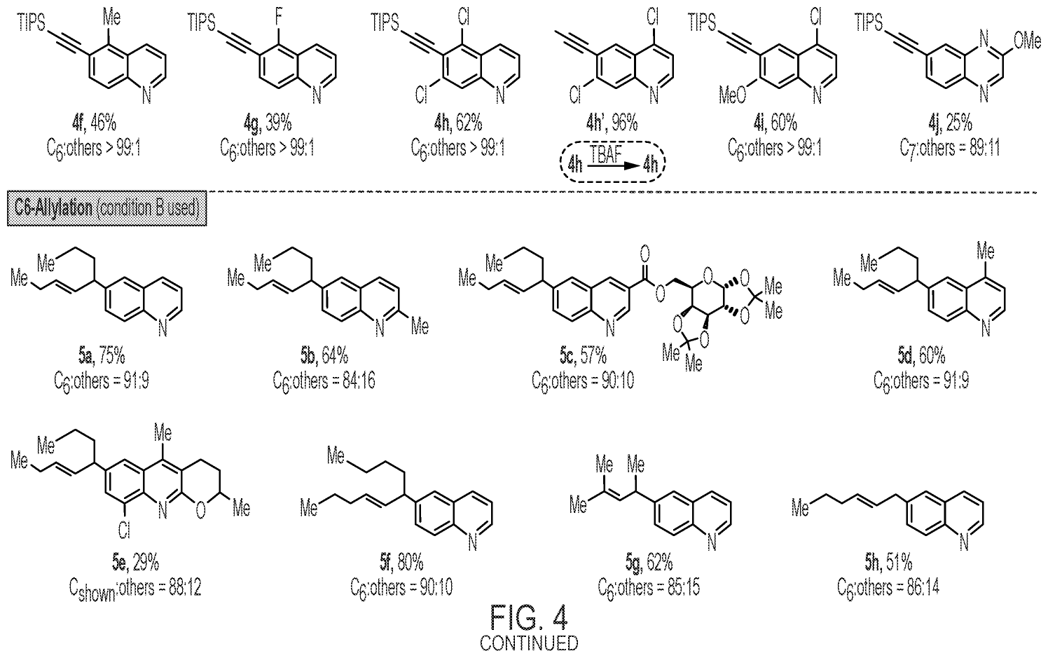

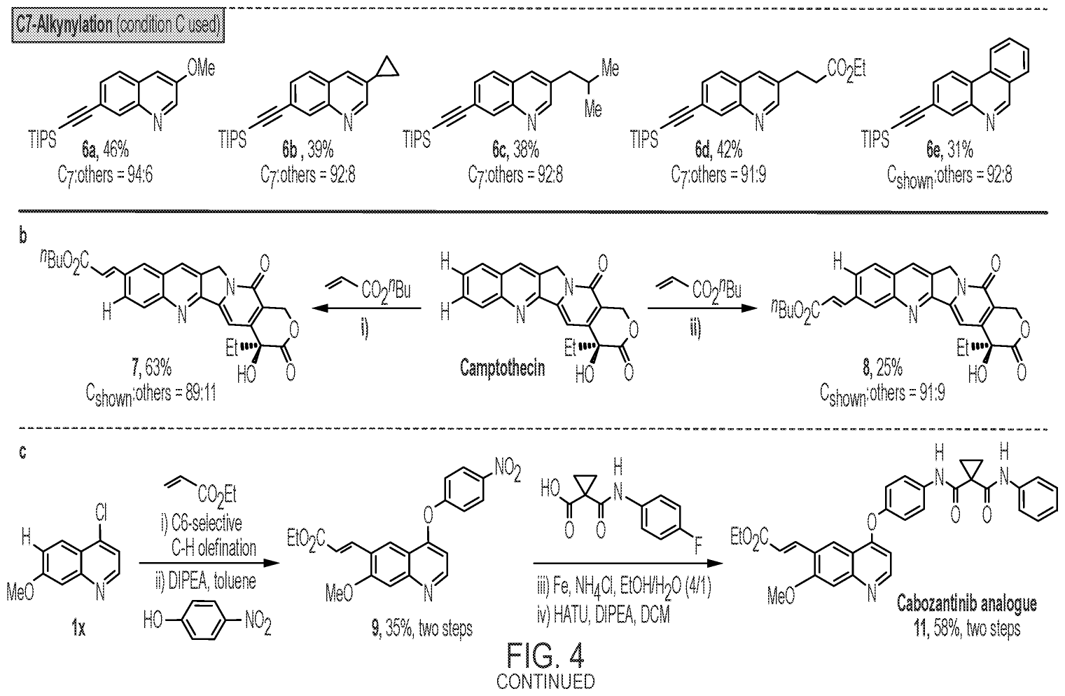

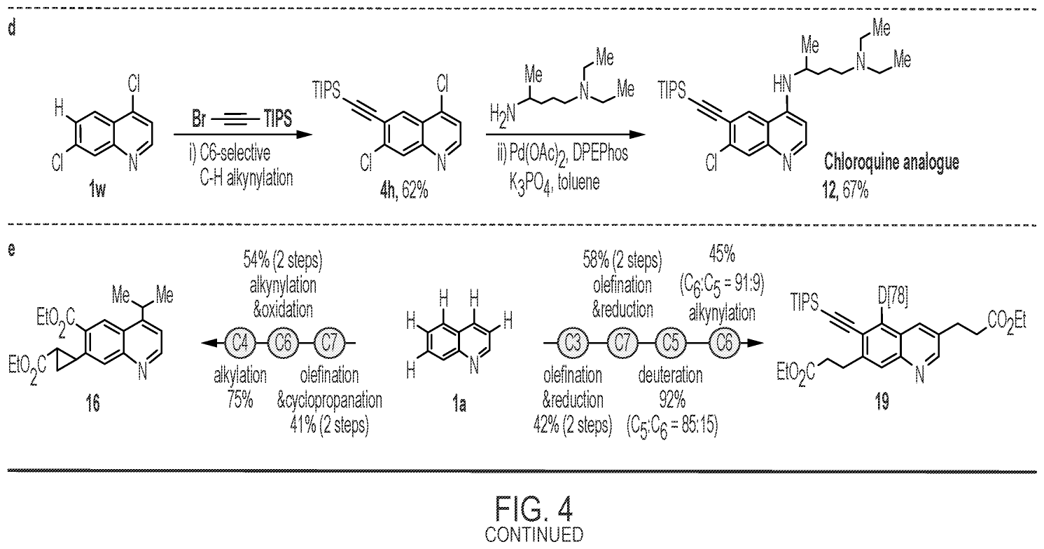

FIG. 2. C6 (and related) selective C—H olefination reactions of quinoline and related heterocycles, a, Selected optimization of directing template and template chaperone scaffolds. Yield and selectivity of 2a are determined by 1H NMR analysis, b, Scope of aza-arenes and olefins. Data are reported as isolated yields. Conditions for 2v: using T8 (0.2 equiv). Conditions for 2aa and 2ab: using T15 (0.2 equiv) and TC10 (0.8 equiv). Conditions for 2at and 2au: using Condition B in FIG. 4a, c, DFT analysis rationalizes the observed C6 selectivity for template T12. Bond lengths are denoted in A.

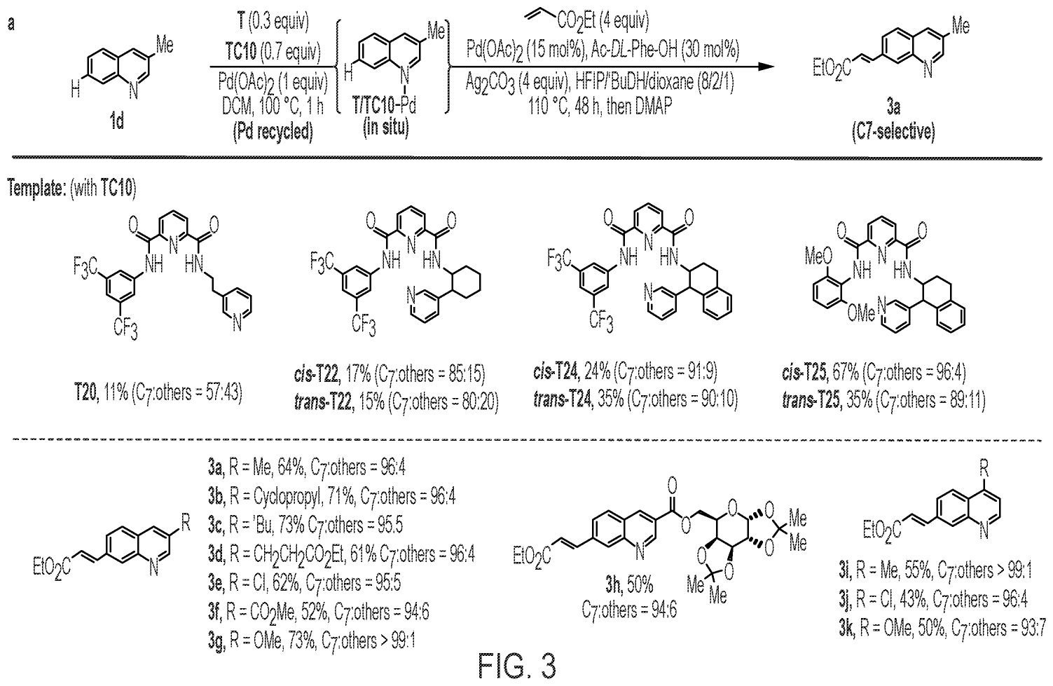

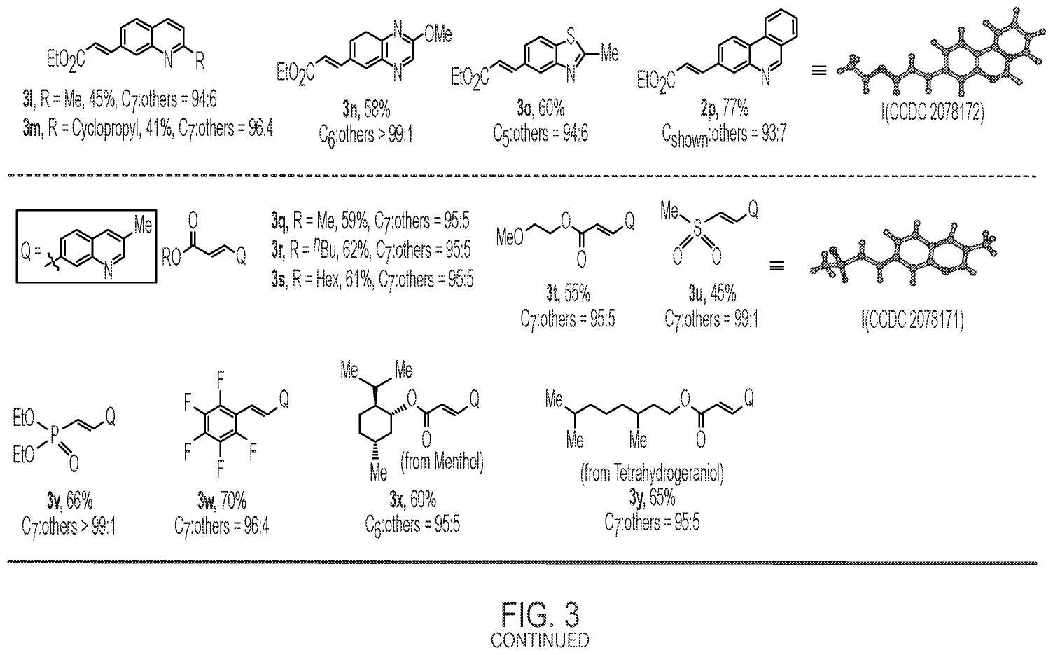

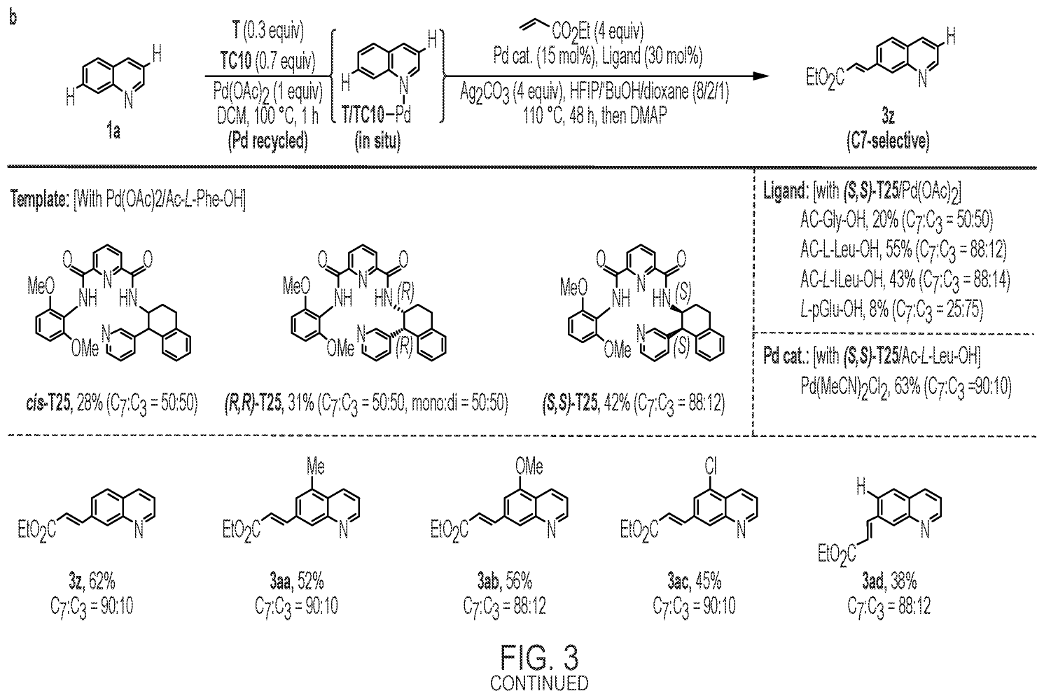

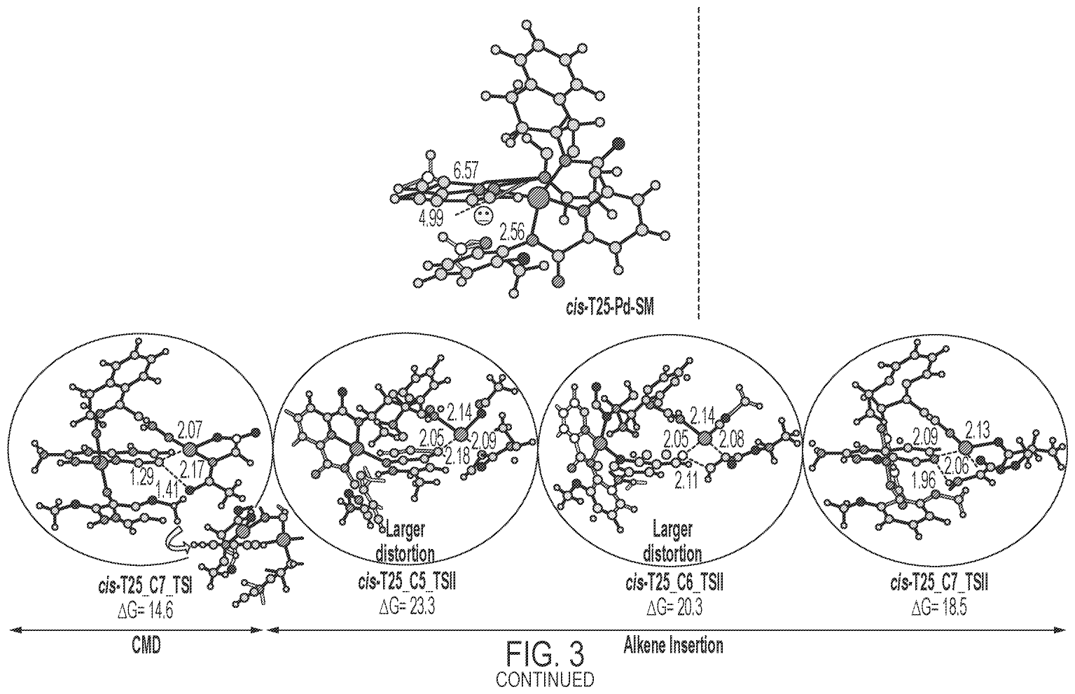

The success of our C6-selective catalytic template prompted us to investigate whether C7-selectivity was also feasible through judicious spatial optimization. Our study commenced with 3-methylquinoline bearing no C—H bond at the C3, wherein an initial hit was found using a template bearing a two-carbon spacer to the directing 3-pyridyl motif (T20, FIG. 3a). Template rigidification (T22, T24, FIG. 3a) improved yield and selectivity, with cis- and trans-T24 notably delivered the product 3a in high selectivity. Systematic tuning of the template's left arm identified that a 2,6-dimethoxyphenyl motif provided markedly improved C7 yield and selectivity, with the best C7-selective template (cis-T25, FIG. 3a) afforded 3a in 67% NMR yield with 96:4 selectivity. Using racemic cis-T25 and Ac-DL-Phe-OH ligand, a range of C3-, C4-, and C2-substituted quinolines smoothly afforded the C7-olefinated products (3a to 3m) in good yield and excellent selectivity (FIG. 3a). Other pharmaceutically important heterocycles (quinoxaline, benzothiophene, and phenanthridine) were also compatible, generating distally-olefinated products 3n to 3p in a site-selective manner. Additionally, a diverse set of olefinic coupling partners were competent using 1d as the substrate, successfully reacting with acrylates (3q to 3t, 3x and 3y), vinyl sulfone (3u), vinyl phosphonate (3v) and styrene (3w) in moderate to good yields with excellent C7-selectivity.

However, subjecting unsubstituted quinoline 1a with racemic cis-T25 under our optimized conditions gave a disappointing 50:50 mixture of products at the C3 and C7 positions in 56% total yield (FIG. 3b) which restricts the order of iterative C—H activation sequence. This outcome affirmed initial analyses indicating the similar regiochemical (distance and geometry) positioning of the C3 and C7-H bonds relative to the anchoring azine nitrogen; an observation supported by DFT calculations (see Supplementary Information, Fig. S12). Further inspired by the use of chiral catalysts to control site-selective modifications in chiral polyol-containing natural products23,24, we wondered if a matched combination of an enantiopure directing template and a chiral catalyst could distinguish between these two highly similar positions. Thus, C7-selectivity was re-evaluated against unsubstituted quinoline 1a in the presence of enantiopure (R,R)- and (S,S)-T25 with Ac-L-Phe-OH as the chiral ligand. Gratifyingly, the use of (S,S)-T25 matches the chiral ligand, providing 3z with high C7 selectivity (C7:C3=88:12). The mismatched combination of (R,R)-T25 with Ac-L-Phe-OH gave mixture products (C7:C3=50:50). Further optimization of (S,S)-T25 with chiral ligands afforded 3z in improved yield (63%) and selectivity (C7:C3=90:10) (FIG. 3b). These results indicate that competing C—H bonds that are positionally (distance and geometrically) similar can be further distinguished through matched chirality-recognition. Under the optimal conditions, C5- and C6-substituted quinolines, which gave no selectivity in the presence of racemic cis-T25 and chiral ligand, provided 3aa to 3ad in moderate yields and high C7 selectivities (FIG. 3b). To rationalize the observed C7-selectivity, DFT analysis was conducted for directing template cis-T25 with 1d (FIG. 3c). Contrasting with the C6-template, cis-T25 gave the lowest energy transition state for all key steps at C7 compared to other positions (see Supplementary Information, Fig. S8). Further inspection revealed that increased template distortion was required to access both C5 and C6 positions, leaving C7 as the sole favorable pathway for this template.

FIG. 3. C7 (and related) selective C—H olefination reactions of quinoline and related heterocycles, a, Selected template optimization for C7-olefination of 1d and scope of heterocyclic substituted substrates, b, Selected condition optimization for C7-olefination of 1a and scope of benzocyclic substituted substrates. Optimization yield and selectivity are determined by 1H NMR analysis. For each entry, data are reported as isolated yields, c, DFT analysis rationalizes the observed C7 selectivity for template cis-T25. Bond lengths are denoted in Å.

The scope of transformation intercepted from this catalytic directed remote C—H palladation was broadened through achieving the site-selective C—H alkynylation and allylation of aza-arenes (FIG. 4a, 4a to 4j; 5a to 5h; 6a to 6e); representing versatile linchpins for further diversification (27, 28).

Uniformly, high site-selectivity was obtained for the template-assisted C6 and C7-selective alkynylation reactions. The corresponding allylation reactions were similarly effective, giving comparatively higher reactivity albeit with slightly reduced C6-selectivity. In all cases, a range of substitutions were tolerated, signaling the robustness of this catalytic template strategy for direct remote functionalization.

FIG. 4. Other transformations and synthetic applications, a, Site-selective C—H alkynylation and allylation of aza-arenes. Conditions for 5f: using trans-5-decene (3 equiv). Conditions for 5g: using trans-4-methyl-2-pentene (3 equiv). Conditions for 5h: using 1-hexene (3 equiv), b, Late-stage remote site-selective C—H modification of camptothecin. Reaction conditions are provided in Supplementary Information 2.10, c, Synthesis of cabozantinib analogue through C6-H olefination, d, Synthesis of chloroquine analogue through C6-H alkynylation, e, Molecular editing of quinoline through iterative C—H activation in different orders. Reaction conditions are provided in Supplementary Information 2.13. Deuterium incorporation is shown in square brackets.

The applicability of this method in a drug discovery context was first exemplified by the divergent late-stage site-selective C—H functionalization of the anticancer natural product camptothecin (FIG. 4b)30. Subjecting camptothecin to our optimized C6-selective template generated novel analogue 7 in 63% yield, while the corresponding C7-selective template generated its regioisomer 8 in 25% yield. Successful C—H editing of key pharmacophores was also demonstrated, providing novel analogues of anticancer agent cabozantinib 1131,32 and antimalarial agent chloroquine 1233 (FIG. 4c,d). Finally, we were eager to address the ultimate challenge of executing sequential site-selective late-stage ‘molecular editing’ in any desired order on a quinoline scaffold; its feasibility demonstrated by successful iterative C—H activations to access products 16 and 19 bearing diverse substitutions (FIG. 4e).

In summary, a unified catalytic remote-directing template strategy allowed for precise differentiation of remote and adjacent C6 and C7-H bonds, as well as similar C3 and C7-H bonds of a pharmaceutically-relevant bicyclic aza-arene scaffold. The modularity of C6/C7 functionalization described herein, combined with previously reported methods, completes the suite of reactions required to edit all C—H bonds with cyclic aza-arene scaffolds in different orders. Notably, the realization of C7-H selective activation over C3-H also established chiral recognition as an effective mean in fine-tuning remote site-selectivity between positionally similar C—H bonds, complementing previously employed distance and geometric parameters in directing template design.

### REFERENCES

1) Szpilman, A. M. & Carreira, E. M. Probing the biology of natural products: molecular editing by diverted total synthesis. Angew. Chem. Int. Ed. 49, 9592-9628 (2010).
2) Das, S., Incarvito, C. D., Crabtree, R. H. & Brudvig, G. W. Molecular recognition in the selective oxygenation of saturated C—H bonds by a dimanganese catalyst. Science 312, 1941-1943 (2006).
3) Wencel-Delord, J. & Glorius, F. C—H bond activation enables the rapid construction and late-stage diversification of functional molecules. Nat. Chem. 5, 369-375 (2013).
4) Nakao, Y., Kanyiva, K. S. & Hiyama, T. A strategy for C—H activation of pyridines: direct C2 selective alkenylation of pyridines by nickel/lewis acid catalysis. J. Am. Chem. Soc. 130, 2448-2449 (2008).
5) Berman, A. M., Lewis, J. C., Bergman, R. G. & Ellman, J. A. Rh(I)-catalyzed direct arylation of pyridines and quinolines. J. Am. Chem. Soc. 130, 14926-14927 (2008).
6) Nakao, Y., Yamada, Y., Kashihara, N. & Hiyama, T. Selective C4 alkylation of pyridine by nickel/lewis acid catalysis. J. Am. Chem. Soc. 132, 13666-13668 (2010).
7) Tsai, C.-C. et al. Bimetallic nickel aluminum mediated para-selective alkenylation of pyridine: direct observation of η²,η¹-pyridine Ni(0)-Al(III) intermediates prior to C—H bond activation. J. Am. Chem. Soc. 132, 11887-11889 (2010).
8) Chen, Q., du Jourdin, X. M. & Knochel, P. Transition-metal-free BF₃-mediated regioselective direct alkylation and arylation of functionalized pyridines using Grignard or organozinc reagents. J. Am. Chem. Soc. 135, 4958-4961 (2013).
9) Yamamoto, S., Saga, Y., Andou, T., Matsunaga, S. & Kanai, M. Cobalt-catalyzed C4 selective alkylation of quinolines. Adv. Synth. Catal. 356, 401-405 (2014).
10) Takagi, J., Sato, K., Hartwig, J. T., Ishiyama, T. & Miyaura, N. Iridium-catalyzed C—H coupling reaction of heteroaromatic compounds with bis(pinacolato)diboron: regioselective synthesis of heteroarylboronates. Tetrahedron Lett. 43, 5649-5651 (2002).
11) Ye, M., Gao, G.-L. & Yu, J.-Q. Ligand-promoted C3 selective C—H olefination of pyridines with Pd catalysts. J. Am. Chem. Soc. 133, 6964-6967 (2011).
12) Kwak, J., Kim, M. & Chang, S. Rh(NHC)-catalyzed direct and selective arylation of quinolines at the 8-position. J. Am. Chem. Soc. 133, 3780-3783 (2011).
13) Konishi, S. et al. Site-selective C—H borylation of quinolines at the C8 position catalyzed by a silica-supported phosphane-iridium system. Chem. Asian. J. 9, 434-438 (2014).
14) Murai, M., Nishinaka, N. & Takai, K. Iridium-catalyzed sequential silylation and borylation of heteroarenes cased on regioselective C—H bond activation. Angew. Chem. Int. Ed. 57, 5843-5847 (2018).
15) Leow, D., Li, G., Mei, T.-S. & Yu, J.-Q. Activation of remote meta-C—H bonds assisted by an end-on template. Nature 486, 518-522 (2012).
16) Kuninobu, Y., Ida, H., Nishi, M. & Kanai, M. A meta-selective C—H borylation directed by a secondary interaction between ligand and substrate. Nat. Chem. 7, 712-717 (2015).
17) H. J. Davis, M. T. Mihai, R. J. Phipps, J. Am. Chem. Soc. 138, 12759-12762 (2016).
18) Hoque, M. E., Bisht, R., Haldar, C. & Chattopadhyay, B. Noncovalent interactions in Ir-catalyzed C—H activation: L-shaped ligand for para-selective borylation of aromatic esters. J. Am. Chem. Soc. 139, 7745-7748 (2017).
19) Zhang, T. et al. A directive Ni catalyst overrides conventional site selectivity in pyridine C—H alkenylation. Nat. Chem. 13, 1207-1213 (2021).
20) Zhang, Z., Tanaka, K. & Yu, J.-Q. Remote site-selective C—H activation directed by a catalytic bifunctional template. Nature 543, 538-542 (2017).
21) Ramakrishna, K. et al. Coordination assisted distal C—H alkylation of fused heterocycles. Angew. Chem. Int. Ed. 58, 13808-13812 (2019).
22) Shi, H. et al. Differentiation and functionalization of remote C—H bonds in adjacent positions. Nat. Chem. 12, 399-404 (2020).
23) Lewis, C. A. & Miller, S. J. Site-selective derivatization and remodeling of erythromycin A by using simple peptide-based chiral catalysts. Angew. Chem. Int. Ed. 45, 5616-5619 (2006).
24) Tay, J.-H. et al. Regiodivergent glycosylations of 6-deoxy-erythronolide B and oleandomycin-derived macrolactones enabled by chiral acid catalysis. J. Am. Chem. Soc. 139, 8570-8578 (2017).
25) Dimakos, V. & Taylor, M. S. Site-selective functionalization of hydroxyl groups in carbohydrate derivatives. Chem. Rev. 118, 11457-11517 (2018).
26) Chu, L. et al. Remote meta-C—H activation using a pyridine-based template: achieving site-selectivity via the recognition of distance and geometry. ACS Cent. Sci. 1, 394-399 (2015).
27) Lerchen, A. et al. Non-directed cross-dehydrogenative (hetero)arylation of allylic C(sp³)-H bonds enabled by C—H activation. Angew. Chem. Int. Ed. 57, 15248-15252 (2018).
28) Fu, L., Zhang, Z., Chen, P., Lin, Z. & Liu, G. Enantioselective copper-catalyzed alkynylation of benzylic C—H bonds via radical relay. J. Am. Chem. Soc. 142, 12493-12500 (2020).
29) Porey, S. et al. Alkyne linchpin strategy for drug: pharmacophore conjugation: experimental and computational realization of a meta-selective inverse sonogashira coupling. J. Am. Chem. Soc. 142, 3762-3774 (2020).
30) Pan, P. et al. Structure-based drug design and identification of H₂O-soluble and low toxic hexacyclic camptothecin derivatives with improved efficacy in cancer and lethal inflammation models in vivo. J. Med. Chem. 61, 8613-8624 (2018).
31) Krajewska, J., Olczyk, T. & Jarzab, B. Cabozantinib for the treatment of progressive metastatic medullary thyroid cancer. Expert Rev. Clin. Pharmacol. 9, 69-79 (2016).
32) Personeni, N., Rimassa, L., Pressiani, T., Smiroldo, V. & Santoro, A. Cabozantinib for the treatment of hepatocellular carcinoma. Expert Rev. Anticancer Ther. 19, 847-855 (2019).
33) Hwang, J. Y. et al. Synthesis and evaluation of 7-substituted 4-aminoquinoline analogues for antimalarial activity. J. Med. Chem. 54, 7084-7093 (2011).

### EXPERIMENTALS

### 1.1 General Information

Compounds of the invention can be made by a variety of methods depicted in the illustrative synthetic reactions described below in the Examples section.

The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, 1991, Volumes 1-15; Rodd's Chemistry of Carbon Compounds, Elsevier Science Publishers, 1989, Volumes 1-5 and Supplementals; and Organic Reactions, Wiley & Sons: New York, 1991, Volumes 1-40. It should be appreciated that the synthetic reaction schemes shown in the Examples section are merely illustrative of some methods by which the compounds of the invention can be synthesized, and various modifications to these synthetic reaction schemes can be made and will be suggested to one skilled in the art having referred to the disclosure contained in this application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein are typically conducted under an inert atmosphere at atmospheric pressure at a reaction temperature range of from about −78° C. to about 150° C., often from about 0° C. to about 125° C., and more often and conveniently at about room (or ambient) temperature, e.g., about 20° C.

Various substituents on the compounds of the invention can be present in the starting compounds, added to any one of the intermediates or added after formation of the final products by known methods of substitution or conversion reactions. If the substituents themselves are reactive, then the substituents can themselves be protected according to the techniques known in the art. A variety of protecting groups are known in the art, and can be employed. Examples of many of the possible groups can be found in “Protective Groups in Organic Synthesis” by Green et al., John Wiley and Sons, 1999. For example, nitro groups can be added by nitration and the nitro group can be converted to other groups, such as amino by reduction, and halogen by diazotization of the amino group and replacement of the diazo group with halogen. Acyl groups can be added by Friedel-Crafts acylation. The acyl groups can then be transformed to the corresponding alkyl groups by various methods, including the Wolff-Kishner reduction and Clemmenson reduction. Amino groups can be alkylated to form mono- and di-alkylamino groups; and mercapto and hydroxy groups can be alkylated to form corresponding ethers. Primary alcohols can be oxidized by oxidizing agents known in the art to form carboxylic acids or aldehydes, and secondary alcohols can be oxidized to form ketones. Thus, substitution or alteration reactions can be employed to provide a variety of substituents throughout the molecule of the starting material, intermediates, or the final product, including isolated products.

All reactions were performed in round-bottom flask, sealed tube or vials. Liquids and solutions were transferred with syringes and pipettes. Unless otherwise stated, all solvents and chemical reagents were obtained from commercial sources and used without further purifications. The analytical thin layer chromatography was performed on 0.25 mm silica gel 60-F254 and spots were visualized by UV light at 254 nM. Column chromatography was performed using E. Merck silica (60, particle size 0.043-0.063 mm), and preparative TLC (pTLC) was performed on Merck silica plates (60F254). ¹H NMR was recorded on Bruker DRX-600 instrument (600 MHz). Chemical shifts were quoted in parts per million (ppm) referenced to 0.0 ppm for tetramethylsilane. ¹³C NMR spectra were recorded on Bruker DRX-600 instrument (150 MHz), and were fully decoupled by broad band proton decoupling. Chemical shifts were reported in ppm referenced to the center line of a triplet at 77.0 ppm of CDCl₃ or the center line of a heptet at 49.0 ppm of CD₃OD. ¹⁹F NMR spectra were recorded on Bruker AMX-400 instrument (376 MHz), and were fully decoupled by broad band proton decoupling. High-resolution mass spectra (HRMS) were recorded on an Agilent Mass spectrometer using ESI-TOF (electrospray ionization-time of flight). Chiral separation and detection were conducted on the Agilent Technologies supercritical fluid chromatography (SFC) system using commercially available chiral columns. The single crystal X-ray diffraction studies were carried out on a Bruker Smart APEX II CCD diffractometer equipped with Cu K_{α} radiation or Bruker D8-Venture 3-circle diffractometer equipped with a Photon 3 detector and Mo K_{α} radiation.

### Abbreviations

Commonly used abbreviations include: acetyl (Ac), azo-bis-isobutyrylnitrile (AlN), atmospheres (Atm), 9-borabicyclo[3.3.1]nonane (9-BBN or BBN), tert-butoxycarbonyl (Boc), di-tert-butyl pyrocarbonate or boc anhydride (BOC₂O), benzyl (Bn), butyl (Bu), Chemical Abstracts Registration Number (CASRN), benzyloxycarbonyl (CBZ or Z), carbonyl diimidazole (CDI), 1,4-diazabicyclo[2.2.2]octane (DABCO), diethylaminosulfur trifluoride (DAST), dibenzylideneacetone (dba), 1,5-diazabicyclo[4.3.0]non-5-ene (DBN), 1,8-diazabicyclo[5.4.0]undec-7-ene (DBU), N,N′-dicyclohexylcarbodiimide (DCC), 1,2-dichloroethane (DCE), dichloromethane (DCM), diethyl azodicarboxylate (DEAD), di-iso-propylazodicarboxylate (DIAD), di-iso-butylaluminumhydride (DIBAL or DIBAL-H), di-iso-propylethylamine (DIPEA), N,N-dimethyl acetamide (DMA), 4-N,N-dimethylaminopyridine (DMAP), N,N-dimethylformamide (DMF), dimethyl sulfoxide (DMSO), 1,1′-bis-(diphenylphosphino)ethane (dppe), 1,1′-bis-(diphenylphosphino)ferrocene (dppf), 1-(3-dimethylaminopropyl)-3-ethylcarbodiimide hydrochloride (EDCI), ethyl (Et), ethyl acetate (EtOAc), ethanol (EtOH), 2-ethoxy-2H-quinoline-1-carboxylic acid ethyl ester (EEDQ), diethyl ether (Et₂O), O-(7-azabenzotriazole-1-yl)-N, N,N′N′-tetramethyluronium hexafluorophosphate acetic acid (HATU), acetic acid (HOAc), 1-N-hydroxybenzotriazole (HOBt), high pressure liquid chromatography (HPLC), iso-propanol (IPA), lithium hexamethyl disilazane (LiHMDS), methanol (MeOH), melting point (mp), MeSO₂— (mesyl or Ms), methyl (Me), acetonitrile (MeCN), m-chloroperbenzoic acid (MCPBA), mass spectrum (ms), methyl t-butyl ether (MTBE), N-bromosuccinimide (NBS), N-carboxyanhydride (NCA), N-chlorosuccinimide (NCS), N-methylmorpholine (NMM), N-methylpyrrolidone (NMP), pyridinium chlorochromate (PCC), pyridinium dichromate (PDC), phenyl (Ph), propyl (Pr), iso-propyl (i-Pr), pounds per square inch (psi), pyridine (pyr), room temperature (rt or RT), tert-butyldimethylsilyl or t-BuMe₂Si (TBDMS), triethylamine (TEA or Et₃N), 2,2,6,6-tetramethylpiperidine 1-oxyl (TEMPO), triflate or CF₃SO₂— (Tf), trifluoroacetic acid (TFA), 1,1′-bis-2,2,6,6-tetramethylheptane-2,6-dione (TMHD), 0-benzotriazol-1-yl-N,N,N′,N′-tetramethyluronium tetrafluoroborate (TBTU), thin layer chromatography (TLC), tetrahydrofuran (THF), trimethylsilyl or Me₃Si (TMS), p-toluenesulfonic acid monohydrate (TsOH or pTsOH), 4-Me-C₆H₄SO₂— or tosyl (Ts), N-urethane-N-carboxyanhydride (UNCA). Conventional nomenclature including the prefixes normal (n), iso (i-), secondary (sec-), tertiary (tert-) and neo have their customary meaning when used with an alkyl moiety. (J. Rigaudy and D. P. Klesney, Nomenclature in Organic Chemistry, IUPAC 1979 Pergamon Press, Oxford.).

### 2.1 Preparation of Templates and Template Chaperones

### Synthesis of S1

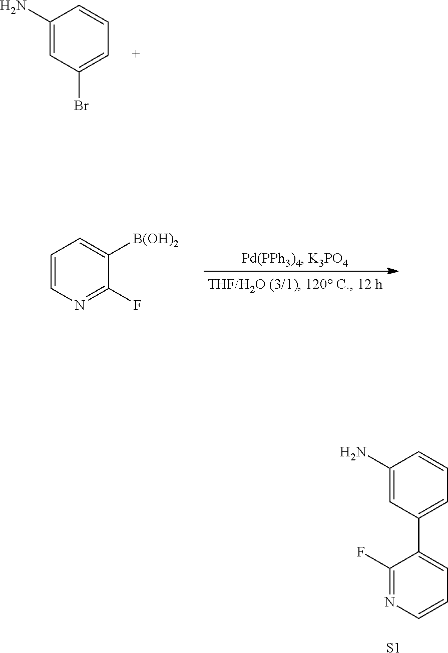

To a solution of K₃PO₄ (1.27 g, 6 mmol) in water (10 mL) was added 3-bromoaniline (516 mg, 3 mmol), 2-fluoro-3-pyridylboronic acid (634 mg, 4.5 mmol), Pd(PPh₃)₄ (347 mg, 0.3 mmol), and THE (30 mL). The Schlenk tube was evacuated and refilled with nitrogen three times, then sealed and put into a preheated oil bath at 120° C. for 12 h. After completion, the reaction mixture was cooled to room temperature. Water was added and the mixture was extracted with EA. The combined organic layers were dried with Na₂SO₄ and concentrated. The residue was purified by silica gel chromatography eluting with hexane/ethyl acetate (EA) (70/30, v/v) to provide the product S1 (441 mg, 78% yield) as pale yellow oil. ¹H NMR (600 MHz, CDCl₃) δ 8.18 (dt, J=4.8, 1.5 Hz, 1H), 7.85 (ddd, J=9.6, 7.4, 2.0 Hz, 1H), 7.26-7.22 (m, 2H), 6.94 (d, J=7.7 Hz, 1H), 6.88 (s, 1H), 6.76-6.70 (m, 1H), 3.78 (s, 2H). ¹³C NMR (151 MHz, CDCl₃) δ 161.21, 159.62, 146.21, 146.11, 140.64, 140.61, 134.95, 134.92, 129.65, 121.72, 121.69. HRMS (ESI-TOF) m/z Calcd for C₁₁H₁₀FN₂⁻ [M+H]⁺ 189.0828, found 189.0828.

### Synthesis of S4a-b

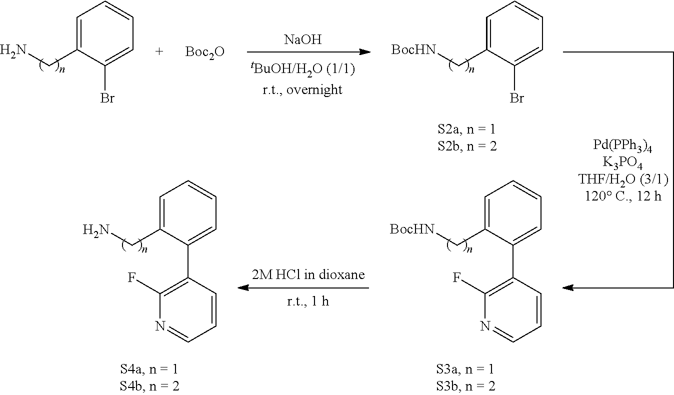

To a solution of NaOH (480 mg, 12 mmol) in water (6 mL) was added Boc₂O (1.57 g, 7.2 mmol), ^{t}BuOH (6 mL) and amine (6 mmol). The resulting mixture was stirred at room temperature for overnight. After completion, the reaction mixture was extracted with EA. The organic layers were collected, dried with Na₂SO₄, and concentrated. The residue was purified by silica gel chromatography eluting with hexane/EA (97/3, v/v) to provide the product S2a (or S2b).

For S2a: (1.1 g, colorless oil, 65% yield)¹H NMR (600 MHz, CDCl₃) δ 7.54 (dd, J=7.9, 1.2 Hz, 1H), 7.38 (d, J=7.7 Hz, 1H), 7.29 (td, J=7.5, 1.3 Hz, 1H), 7.14 (td, J=7.7, 1.8 Hz, 1H), 5.02 (s, 1H), 4.39 (d, J=6.3 Hz, 2H), 1.45 (s, 9H). ¹³C NMR (151 MHz, CDCl₃) δ 155.74, 137.97, 132.74, 129.78, 128.96, 127.66, 123.55, 79.66, 44.90, 28.40. HRMS (ESI-TOF) m/z Calcd for C₈H₉BrNO₂⁺ [M−^{t}Bu+2H]⁺ 229, 9817, found 229.9824.

For S2b: (1.6 g, pale white solid, 89% yield)¹H NMR (600 MHz, CDCl₃) δ 7.54 (d, J=7.6 Hz, 1H), 7.26-7.18 (m, 2H), 7.09 (ddd, J=7.9, 6.8, 2.2 Hz, 1H), 4.60 (s, 1H), 3.39 (t, J=6.9 Hz, 2H), 2.95 (t, J=7.1 Hz, 2H), 1.43 (s, 9H). ¹³C NMR (151 MHz, CDCl₃) δ 155.85, 138.41, 132.91, 131.03, 128.16, 127.54, 124.63, 79.23, 40.26, 36.38, 28.41. HRMS (ESI-TOF) m/z Calcd for C₉H₁₁BrNO₂⁺ [M-^{t}Bu+2H]⁺ 243.9973, found 243.9980.

Following the procedure for synthesis of S1 with 3 mmol of S2a (or S2b), the residue was purified by silica gel chromatography eluting with hexane/EA (85/15, v/v) to provide the product S3a (or S3b).

For S3a: (725 mg, colorless oil, 80% yield)¹H NMR (600 MHz, CDCl₃) δ 8.27-8.23 (m, 1H), 7.72 (ddd, J=9.4, 7.3, 1.9 Hz, 1H), 7.49 (d, J=7.7 Hz, 1H), 7.44-7.41 (m, 1H), 7.37-7.34 (m, 1H), 7.28 (ddd, J=7.1, 4.9, 1.9 Hz, 1H), 7.21 (dd, J=7.6, 1.4 Hz, 1H), 4.77 (s, 1H), 4.19 (s, 2H), 1.42 (s, 9H). ¹³C NMR (151 MHz, CDCl₃) δ 160.35 (d, J=237.8 Hz), 155.67, 147.13 (d, J=15.3 Hz), 141.94 (d, J=4.9 Hz), 137.31, 132.97 (d, J=4.5 Hz), 130.41, 129.12, 128.42, 127.53, 122.83 (d, J=32.3 Hz), 121.59 (d, J=4.7 Hz), 79.55, 42.24, 28.37. HRMS (ESI-TOF) m/z Calcd for C₁₃H₁₂FN₂O₂⁺ [M-Bu+2H]⁺ 247.0883, found 247.0888.

For S3b: (787 mg, pale yellow oil, 83% yield)¹H NMR (600 MHz, CDCl₃) δ 8.25 (ddd, J=4.9, 2.0, 1.1 Hz, 1H), 7.71 (ddd, J=9.4, 7.3, 2.0 Hz, 1H), 7.39 (td, J=7.5, 1.4 Hz, 1H), 7.35 (d, J=7.7 Hz, 1H), 7.31 (td, J=7.4, 1.5 Hz, 1H), 7.28 (ddd, J=7.1, 4.9, 1.8 Hz, 1H), 7.19 (dd, J=7.6, 1.4 Hz, 1H), 4.45 (s, 1H), 3.23 (q, J=6.9 Hz, 2H), 2.69 (t, J=7.2 Hz, 2H), 1.39 (s, 9H). ¹³C NMR (151 MHz, CDCl₃) δ 160.40 (d, J=238.1 Hz), 155.68, 146.95 (d, J=14.6 Hz), 142.15 (d, J=5.5 Hz), 137.36, 133.86 (d, J=3.9 Hz), 130.46, 129.60, 128.94, 126.62, 123.54 (d, J=32.0 Hz), 121.47 (d, J=4.8 Hz), 79.22, 40.89, 33.44, 28.40. HRMS (ESI-TOF) m/z Calcd for C₁₄H₁₄FN₂O₂⁺ [M-^{t}Bu+2H]⁺ 261.1039, found 261.1044.

Adding S3a (or S3b) (2.5 mmol) into a 2 M HCl (14 mL) solution. The reaction mixture was stirred at room temperature for 1 h. The mixture was basified with aqueous NaOH solution and extracted with EA. The combined organic layers were dried with Na₂SO₄ and concentrated to give the crude product S4a (or S4b). This product was used in the next step without further purification.

### Synthesis of S7

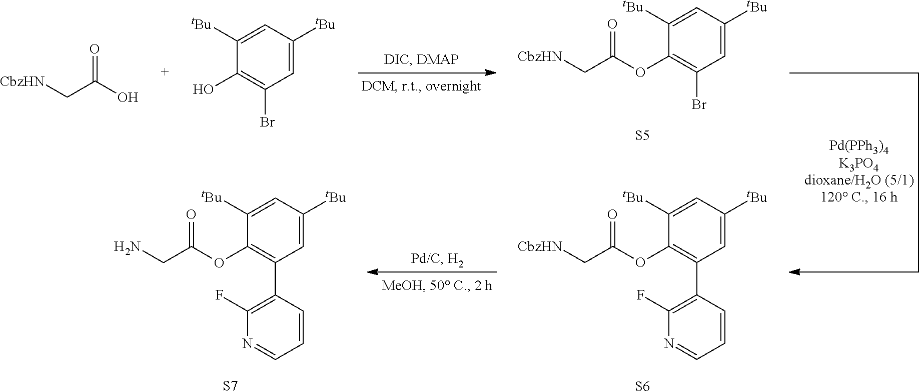

To a solution of Cbz-Gly-OH (2.51 g, 12 mmol), 4-bromo-2,6-di-tert-butyphenol (2.85 g, 10 mmol), and DMAP (183 mg, 1.5 mmol) in DCM (50 mL) at 0° C. was added DIC (2.32 mL, 15 mmol). The solution was warmed to room temperature and stirred for overnight. The insolubles were filtered and discarded. The filtrate was evaporated and purified by silica gel chromatography eluting with hexane/EA (92/8, v/v) to provide the product S5 (3.5 g, 74% yield) as colorless oil. ¹H NMR (600 MHz, CDCl₃) δ 7.45 (d, J=2.3 Hz, 1H), 7.39-7.30 (m, 6H), 5.37 (s, 1H), 5.16 (s, 2H), 4.34 (d, J=5.7 Hz, 2H), 1.32 (s, 9H), 1.30 (s, 9H). ¹³C NMR (151 MHz, CDCl₃) δ 168.05, 156.15, 150.14, 143.91, 143.07, 136.13, 128.56, 128.44, 128.24, 128.15, 123.90, 117.57, 67.23, 43.56, 35.34, 34.85, 31.29, 30.43. HRMS (ESI-TOF) m/z Calcd for C₂₄H₃BrNO₄⁺ [M+H]⁺ 476.1436, found 476.1435.

Following the procedure for synthesis of S1 with dioxane instead of THF as a co-solvent with 5 mmol of S5, the residue was purified by silica gel chromatography eluting with hexane/EA (70/30, v/v) to provide the product S6 (345 mg, 14% yield) as a white solid. ¹H NMR (600 MHz, CDCl₃) δ 8.20 (d, J=4.0 Hz, 1H), 7.72 (t, J=7.6 Hz, 1H), 7.51 (d, J=2.3 Hz, 1H), 7.33 (h, J=6.7, 6.1 Hz, 5H), 7.23-7.19 (m, 1H), 7.18 (s, 1H), 5.08 (s, 2H), 5.03-4.96 (m, 1H), 3.86 (s, 2H), 1.37 (s, 9H), 1.34 (s, 9H). ¹³C NMR (151 MHz, CDCl₃) δ 168.19, 163.33, 160.36 (d, J=236.2 Hz), 159.55, 155.84, 148.99, 147.14 (d, J=14.4 Hz), 144.18, 142.31 (d, J=5.5 Hz), 141.22, 136.09, 128.53, 128.23, 128.12, 126.10, 125.32, 121.32 (d, J=4.9 Hz), 120.97 (d, J=30.2 Hz), 67.14, 43.01, 35.00, 34.83, 31.42, 30.50. HRMS (ESI-TOF) m/z Calcd for C₂₉H₃₄FN₂O₄⁺ [M+H]⁺ 493.2503, found 493.2506.

To a stirred solution of S6 (340 mg, 0.7 mmol) in MeOH (5 mL) was added Pd/C (70 mg). The resulting mixture was stirred at 50° C. for 2 h under hydrogen atmosphere. Upon completion, the reaction mixture was filtered. The filtrate was concentrated to afford the crude amine S7, which was used in the next step without further purification.

### Synthesis of S10

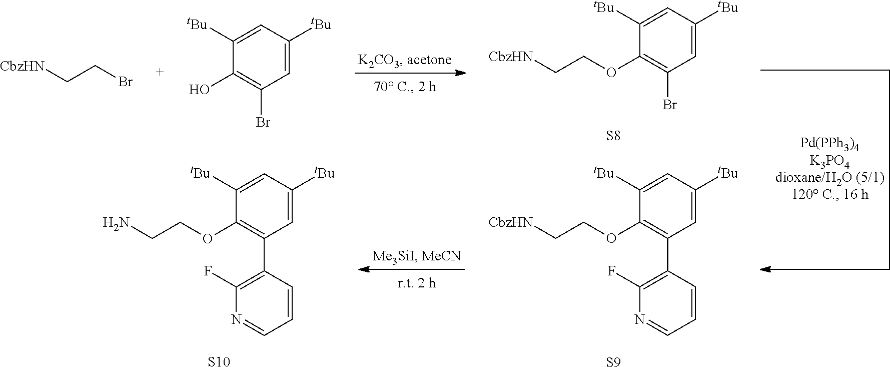

Benzyl 2-bromoethylcarbamate (2.06 g, 8 mmol), 4-bromo-2,6-di-tert-butyphenol (2.28 g, 8 mmol), and K₂CO₃ (2.76 g, 20 mmol) were charged in the flask and acetone (50 mL) was added to this mixture. The resulting mixture was refluxed for 2 h. The insolubles were filtered and discarded. The filtrate was evaporated and purified by silica gel chromatography eluting with hexane/EA (88/12, v/v) to provide the product S8 (3.3 g, 89% yield) as colorless oil. ¹H NMR (600 MHz, CDCl₃) δ 7.40-7.34 (m, 5H), 7.34-7.30 (m, 1H), 7.28 (d, J=2.4 Hz, 1H), 5.35 (s, 1H), 5.15 (s, 2H), 4.13 (t, J=5.1 Hz, 2H), 3.66 (q, J=5.4 Hz, 2H), 1.36 (s, 9H), 1.28 (s, 9H). ¹³C NMR (151 MHz, CDCl₃) δ 156.55, 151.78, 147.66, 144.13, 136.66, 128.95, 128.52, 128.12, 128.09, 123.95, 117.82, 71.09, 66.71, 41.34, 35.80, 34.59, 31.32, 31.08. HRMS (ESI-TOF) m/z Calcd for C₂₄H₃₃BrNO₃⁺ [M+H]⁺ 462.1644, found 462.1642.

Following the procedure for synthesis of S6 with 5 mmol of S8, the residue was purified by silica gel chromatography eluting with hexane/EA (70/30, v/v) to provide the product S9 (287 mg, 12% yield) as pale yellow oil. ¹H NMR (600 MHz, CDCl₃) δ 8.16-8.09 (m, 1H), 7.86 (ddd, J=9.4, 7.4, 2.0 Hz, 1H), 7.41 (d, J=2.5 Hz, 1H), 7.39 (d, J=4.4 Hz, 4H), 7.35-7.33 (m, 1H), 7.18-7.15 (m, 1H), 7.14 (s, 1H), 5.08 (s, 2H), 4.88 (s, 1H), 3.49-3.46 (m, 2H), 3.22 (q, J=5.3 Hz, 2H), 1.41 (s, 9H), 1.32 (s, 9H). ¹³C NMR (151 MHz, CDCl₃) δ 161.28, 159.69, 156.12, 153.19, 146.68 (d, J=14.5 Hz), 146.11, 142.39, 142.07 (d, J=6.1 Hz), 136.60, 128.54, 128.17, 128.14, 126.86-126.33 (m), 125.13, 122.21 (d, J=32.0 Hz), 121.30 (d, J=5.5 Hz), 71.61, 66.67, 40.87, 35.41, 34.60, 31.45, 30.99. HRMS (ESI-TOF) m/z Calcd for C₂₉H₃₆FN₂O₃⁺ [M+H]⁺ 479.2710, found 479.2711.

To a stirred solution of S9 (250 mg, 0.52 mmol) in MeCN (5 mL) was added Me₃SiI (370 μL, 2.6 mmol). The resulting mixture was stirred at room temperature for 2 h. The reaction was quenched with MeOH. The resulting mixture was concentrated to give the crude amine S10, which was used in the next step without further purification.

### Synthesis of S13

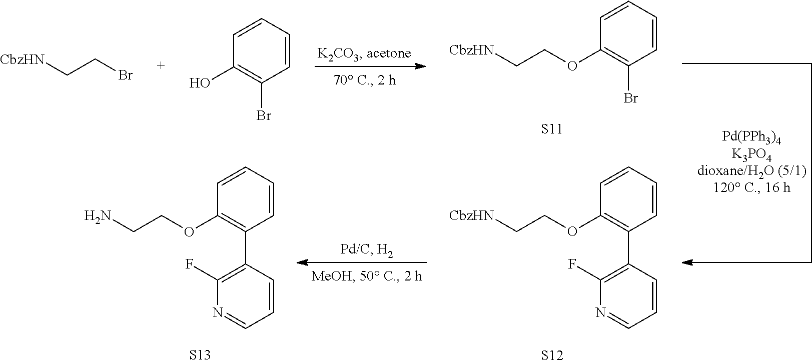

Following the procedure for synthesis of S8 with 5 mmol of 2-bromophenol, the residue was purified by silica gel chromatography eluting with hexane/EA (85/15, v/v) to provide the product Sit (1.3 g, 75% yield) as colorless oil. ¹H NMR (600 MHz, CDCl₃) δ 7.53 (dd, J=7.9, 1.6 Hz, 1H), 7.37-7.33 (m, 4H), 7.31 (ddd, J=8.5, 5.2, 2.5 Hz, 1H), 7.26-7.22 (m, 1H), 6.90-6.87 (m, 1H), 6.87-6.84 (m, 1H), 5.33 (s, 1H), 5.12 (s, 2H), 4.10 (t, J=5.1 Hz, 2H), 3.66 (q, J=5.4 Hz, 2H). ¹³C NMR (151 MHz, CDCl₃) δ 156.44, 154.82, 136.41, 133.40, 128.59, 128.55, 128.17, 128.14, 122.49, 113.65, 112.40, 68.43, 66.87, 40.53. HRMS (ESI-TOF) m/z Calcd for C₁₆H₁₆BrNO₃Na⁺ [M+Na]⁺ 372.0211, found 372.0211.

Following the procedure for synthesis of S6 with 3.7 mmol of S11, the residue was purified by silica gel chromatography eluting with hexane/EA (70/30, v/v) to provide the product S12 (596 mg, 44% yield) as colorless oil. ¹H NMR (600 MHz, CDCl₃) δ 8.19 (d, J=4.4 Hz, 1H), 7.76 (ddd, J=9.3, 7.3, 2.0 Hz, 1H), 7.38 (t, J=8.0 Hz, 1H), 7.35 (d, J=4.4 Hz, 4H), 7.33-7.29 (m, 1H), 7.28 (dd, J=7.5, 1.8 Hz, 1H), 7.22 (t, J=5.8 Hz, 1H), 7.07 (td, J=7.5, 1.0 Hz, 1H), 6.97 (d, J=8.3 Hz, 1H), 5.09 (s, 2H), 5.03 (s, 1H), 4.08 (t, J=5.1 Hz, 2H), 3.52 (q, J=5.4 Hz, 2H). ¹³C NMR (151 MHz, CDCl₃) δ 161.49, 159.90, 155.63, 146.48 (d, J=14.7 Hz), 142.06 (d, J=5.4 Hz), 136.48, 131.20, 130.20, 128.51, 128.10, 128.06, 121.30, 121.28, 121.26, 112.15, 67.52, 66.76, 40.50. HRMS (ESI-TOF) m/z Calcd for C₂₁H₂₀FN₂O₃⁺ [M+H]⁺ 367.1458, found 367.1458.

Following the procedure for synthesis of S7 with Pd/C (40%, w/w) with 1 mmol of S12. The crude product S13 was obtained and used in the next step without further purification.

### Synthesis of S15

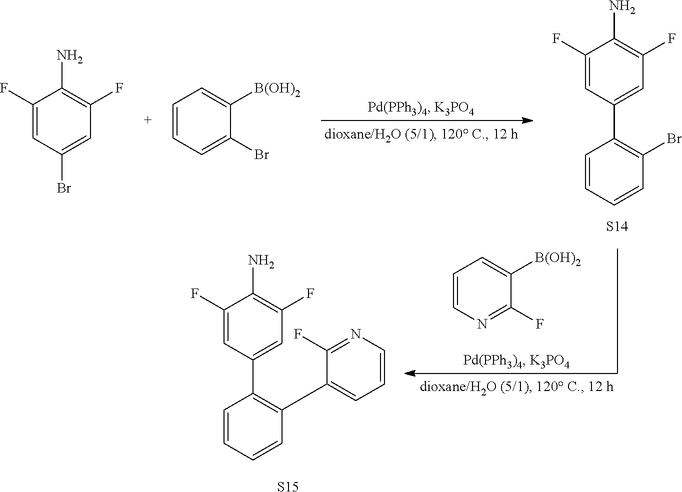

Following the procedure for synthesis of S6 with 3 mmol of 4-bromo-2,6-difluoroanaline, the residue was purified by silica gel chromatography eluting with hexane/EA (95/5, v/v) to provide the product S14 (699 mg, 82% yield) as a brown solid. ¹H NMR (600 MHz, CDCl₃) δ 7.64 (dd, J=8.0, 1.3 Hz, 1H), 7.33 (td, J=7.5, 1.3 Hz, 1H), 7.27 (dd, J=7.6, 1.8 Hz, 1H), 7.20-7.16 (m, 1H), 6.92 (dd, J=7.3, 2.0 Hz, 2H), 3.80 (s, 2H). ¹³C NMR (151 MHz, CDCl₃) δ152.15 (d, J=8.4 Hz), 150.56 (d, J=8.7 Hz), 140.67, 133.28, 131.21, 129.88 (t, J=9.0 Hz), 128.87, 127.49, 123.41 (t, J=16.5 Hz), 122.61, 112.27 (dd, J=16.7, 6.4 Hz). HRMS (ESI-TOF) m/z Calcd for C₁₂H₉BrF₂N⁺ [M+H]⁺ 283.9886, found 283.9894.

Following the procedure for synthesis of S6 with 1 mmol of 514, the residue was purified by silica gel chromatography eluting with hexane/EA (85/15, v/v) to provide the product S15 (177 mg, 59% yield) as a white solid. ¹H NMR (600 MHz, CDCl₃) δ 8.14 (ddd, J=4.9, 2.0, 1.1 Hz, 1H), 7.57 (ddd, J=9.5, 7.4, 2.0 Hz, 1H), 7.47 (td, J=7.5, 1.5 Hz, 1H), 7.43 (td, J=7.5, 1.6 Hz, 1H), 7.41-7.37 (m, 2H), 7.14 (ddd, J=7.3, 4.9, 1.8 Hz, 1H), 6.59 (dd, J=7.4, 2.0 Hz, 2H), 3.68 (s, 2H). ¹³C NMR (151 MHz, CDCl₃) δ 160.99, 159.40, 152.34 (d, J=8.5 Hz), 150.75 (d, J=8.8 Hz), 146.61 (d, J=14.4 Hz), 142.19 (d, J=4.5 Hz), 139.88 (d, J=2.7 Hz), 132.43 (d, J=4.5 Hz), 130.90 (d, J=1.7 Hz), 130.20, 128.93, 127.58, 123.71 (d, J=30.8 Hz), 122.89, 121.27 (d, J=4.5 Hz), 111.89 (dd, J=16.5, 6.2 Hz). HRMS (ESI-TOF) m/z Calcd for C₁₇H₁₂F₃N₂⁺ [M+H]⁺ 301.0953, found 301.0952.

### Synthesis of S18¹² and S21

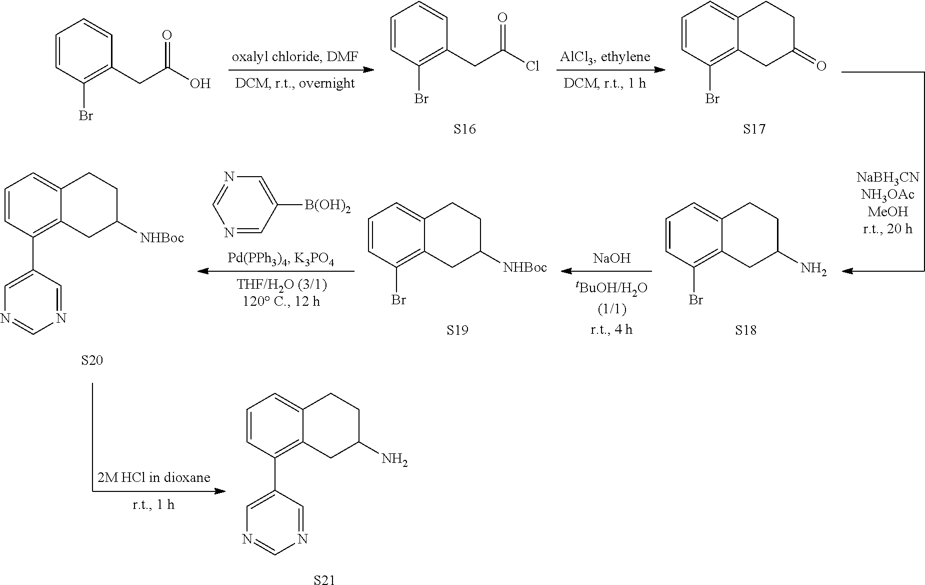

To a stirred solution of 2-bromophenylacetic acid (25 g, 116.3 mmol) in DCM (200 mL) at 0° C. was added DMF (100 L) and oxalyl chloride (10.83 mL, 127.9 mmol). The reaction solution was warmed to room temperature and stirred for overnight. The mixture was concentrated to obtain the crude product S16, which was used in the next step without further purification.

To a stirred suspension of high-quality AlCl₃ (31 g, 232.6 mmol) in DCM (150 mL) at −10° C. was added a solution of S16 (116.3 mmol) in DCM (100 mL) over 30 min. Then, ethylene was bubbled into the reaction mixture at −10° C. for 1 h. After full conversion (GC-MS detection), the ethylene flow was stopped and the reaction mixture continued to be stirred at −10° C. for 15 min. Then the mixture was poured slowly into a beaker containing lots of ice. [Caution: addition of ice into the reaction mixture causes significant exotherms which can lead to explosions] The layers were separated, and the collected organic phase was sequentially washed with water, NaHCO₃ solution, and brine, dried with Na₂SO₄, and concentrated. The residue was dissolved in MeCN (200 mL), washed with pentane to remove polyethylene, and concentrated to give the product S17 (24 g, 92% for two steps) as a pale black solid, which was used in the next step without further purification.

To a solution of S17 (24 g, 106.7 mmol) and NH₄OAc (65.8 g, 853.6 mmol) in MeOH (250 mL) at room temperature was added NaBH₃CN (8 g, 128 mmol). The resulting mixture was stirred for 20 h. The reaction mixture was basified with NaOH solution and evaporated. Then water was added and the solution was extracted with EA. The organic phase was washed with brine, dried with Na₂SO₄, and concentrated to afford the crude product S18 as brown oil, which was used in the next step without further purification.

Following the procedure for synthesis of S2a with 30 mmol of crude S18, the residue was purified by silica gel chromatography eluting with hexane/EA (95/5, v/v) to provide the product S19 (3.3 g, 34% yield for above two steps) as a white solid. ¹H NMR (600 MHz, CDCl₃) δ 7.39 (d, J=7.7 Hz, 1H), 7.05 (d, J=7.0 Hz, 1H), 6.99 (t, J=7.7 Hz, 1H), 4.60 (s, 1H), 3.98 (s, 1H), 3.16 (dd, J=17.2, 5.6 Hz, 1H), 2.88 (q, J=6.7, 5.4 Hz, 2H), 2.52 (dd, J=17.1, 8.5 Hz, 1H), 2.09-2.01 (m, 1H), 1.72-1.64 (m, 1H), 1.47 (s, 9H). ¹³C NMR (151 MHz, CDCl₃) δ 155.24, 134.04, 130.10, 127.94, 127.22, 125.75, 79.43, 46.60, 36.88, 28.78, 28.44, 28.10. HRMS (ESI-TOF) m/z Calcd for C₁₁H₁₃BrNO₂⁺ [M+H]⁺ 270.0130, found 270.0139.

Following the procedure for synthesis of S1 with 4.3 mmol of S19 and 8.6 mmol of 5-pyrimidinylboronic acid, the residue was purified by silica gel chromatography eluting with hexane/EA (85/15, v/v) to provide the product S20 (1.22 g, 87% yield) as pale yellow glue. ¹H NMR (600 MHz, CDCl₃) δ 9.21 (s, 1H), 8.69 (s, 2H), 7.25 (d, J=7.4 Hz, 1H), 7.22 (d, J=7.6 Hz, 1H), 7.04 (d, J=7.3 Hz, 1H), 4.56 (s, 1H), 3.91 (s, 1H), 2.99 (t, J=6.6 Hz, 2H), 2.87 (dd, J=16.3, 5.1 Hz, 1H), 2.47 (dd, J=16.3, 8.8 Hz, 1H), 2.12 (s, 1H), 1.73 (ddt, J=9.8, 7.8, 2.0 Hz, 1H), 1.41 (s, 9H). ¹³C NMR (151 MHz, CDCl₃) δ 157.38, 156.61, 155.11, 136.79, 135.00, 134.80, 132.37, 129.78, 127.78, 126.44, 79.51, 46.46, 34.98, 28.99, 28.39, 28.06. HRMS (ESI-TOF) m/z Calcd for C₁₉H₂₄N₃O₂⁺ [M+H]⁺ 326.1869, found 326.1865.

Following the procedure for synthesis of S4a with 2.2 mmol of S20. The crude product S21 was obtained and used in the next step without further purification.

### Synthesis of S28

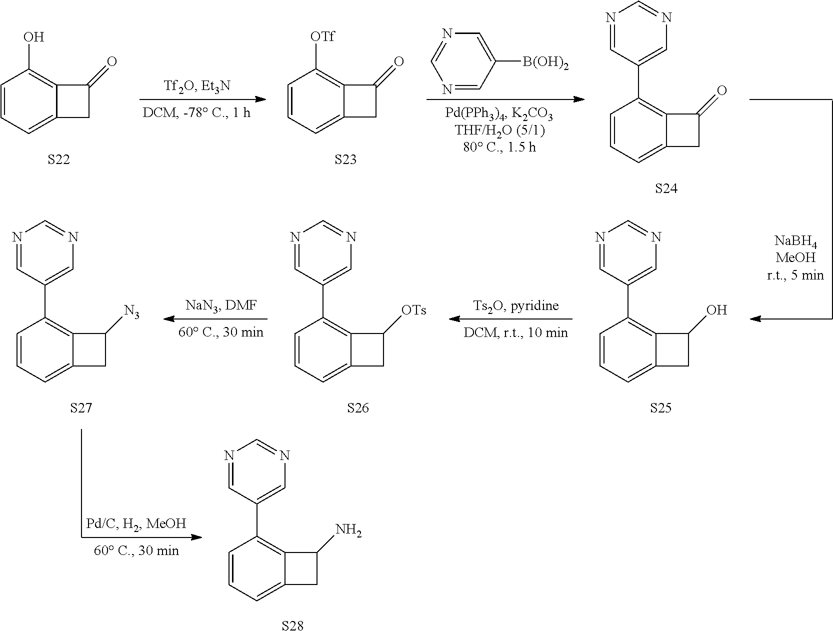

S23 was synthesized according to modified literature procedures.³ To a stirred solution of S22 (1.6 g, 12 mmol) in DCM (100 mL) at −78° C. was sequentially added Et₃N (3.34 mL, 24 mmol) and Tf₂O (3.03 mL, 18 mmol). The reaction solution was stirred at −78° C. for 1 h. NH₄Cl solution was added to quench the reaction. Then water was added and the solution was extracted with DCM. The combined organic layers were dried with Na₂SO₄ and concentrated. The residue was purified by silica gel chromatography eluting with hexane/EA (85/15, v/v) to provide S23 (3.1 g, 97% yield) as colorless oil.

For S22:^{4 1}H NMR (600 MHz, CDCl₃) δ 7.45 (dd, J=8.4, 7.1 Hz, 1H), 7.01 (d, J=7.1 Hz, 1H), 6.79 (d, J=8.5 Hz, 1H), 3.90 (s, 2H). ¹³C NMR (151 MHz, CDCl₃) δ 181.27, 152.58, 150.56, 138.60, 132.10, 116.20, 114.97, 51.16.

For S23: ¹H NMR (600 MHz, CDCl₃) δ 7.68-7.64 (m, 1H), 7.58 (d, J=7.3 Hz, 1H), 7.27-7.25 (m, 1H), 4.10 (s, 2H). ¹³C NMR (151 MHz, CDCl₃) δ 181.69, 152.48, 139.25, 137.71, 137.45, 123.92, 121.31, 118.69 (q, J=320.9 Hz), 53.41. HRMS (ESI-TOF) m/z Calcd for C₉H₆F₃O₄S⁺ [M+H]⁺ 266.9939, found 266.9944.

To a solution of K₂CO₃ (1.7 g, 12.5 mmol) in water (4 mL) was added S23 (565 mg, 2.5 mmol), 5-pyrimidinylboronic acid (620 mg, 5 mmol), Pd(PPh₃)₄ (289 mg, 0.25 mmol), and THE (20 mL). The Schlenk tube was evacuated and refilled with nitrogen for three times. Then sealed and put into a preheated oil bath at 80° C. for 1.5 h. After completion, the reaction mixture was cooled to room temperature. Water was added and the mixture was extracted with EA. The combined organic layers were dried with Na₂SO₄ and concentrated. The residue was purified by silica gel chromatography eluting with hexane/EA (60/40, v/v) to provide the product S24 (299 mg, 61% yield) as a white flocculent solid. ¹H NMR (600 MHz, CDCl₃) δ 9.37 (s, 2H), 9.24 (s, 1H), 7.75 (d, J=7.8 Hz, 1H), 7.68 (t, J=7.6 Hz, 1H), 7.61 (d, J=7.3 Hz, 1H), 4.09 (s, 2H). ¹³C NMR (151 MHz, CDCl₃) δ 187.18, 158.60, 155.44, 152.15, 144.78, 136.00, 129.03, 129.02, 125.49, 124.08, 52.12. HRMS (ESI-TOF) m/z Calcd for C₁₂H₉N₂O⁺ [M+H]⁺ 197.0715, found 197.0713.

To a solution of S24 (98 mg, 0.5 mmol) in MeOH (5 mL) at room temperature was slowly added NaBH₄ (38 mg, 1 mmol). The resulting mixture was stirred for 5 min. The reaction mixture was evaporated. Then water was added and the solution was extracted with EA. The organic phase was washed with brine, dried with Na₂SO₄, and concentrated to afford the crude product S25 (99 mg, quant.) as a white solid. ¹H NMR (600 MHz, CDCl₃) δ 9.21 (s, 2H), 9.14 (s, 1H), 7.52 (d, J=7.9 Hz, 1H), 7.45 (t, J=7.6 Hz, 1H), 7.23 (d, J=7.2 Hz, 1H), 5.45 (ddd, J=8.4, 4.5, 1.9 Hz, 1H), 3.71 (dd, J=14.3, 4.5 Hz, 1H), 3.21-3.13 (m, 2H). ¹³C NMR (151 MHz, CDCl₃) δ 157.39, 155.38, 145.26, 143.47, 131.09, 130.71, 129.48, 124.62, 124.31, 70.85, 42.48. HRMS (ESI-TOF) m/z Calcd for C₁₂H₁₁N₂O⁺ [M+H]⁺ 199.0871, found 199.0874.

To a solution of S25 (99 mg, 0.5 mmol) in DCM (5 mL) at room temperature was added pyridine (402 μL, 5 mmol) and Ts₂O (815 mg, 2.5 mmol). The resulting mixture was stirred for 5 min. Water was added and the mixture was extracted with DCM. The combined organic layers were dried with Na₂SO₄ and concentrated. The residue was purified by silica gel chromatography eluting with DCM/EA (50/50, v/v) to provide the product S26 (176 mg, quant.) as a white solid. ¹H NMR (600 MHz, CDCl₃) δ 9.19 (s, 1H), 8.90 (s, 2H), 7.86 (d, J=8.3 Hz, 2H), 7.51 (t, J=7.6 Hz, 1H), 7.45 (d, J=7.9 Hz, 1H), 7.37 (d, J=8.0 Hz, 2H), 7.21 (d, J=7.1 Hz, 1H), 5.83 (dd, J=4.2, 1.8 Hz, 1H), 3.66 (dd, J=14.8, 4.2 Hz, 1H), 3.44 (d, J=14.8 Hz, 1H), 2.46 (s, 3H). ¹³C NMR (151 MHz, CDCl₃) δ 157.88, 155.25, 145.33, 143.60, 139.12, 133.05, 132.13, 130.52, 130.36, 130.09, 128.04, 126.05, 123.98, 75.00, 40.22, 21.72. HRMS (ESI-TOF) m/z Calcd for C₁₉H₁₇N₂O₃S⁺ [M+H]⁺ 353.0960, found 353.0958.

To a solution of S26 (176 mg, 0.5 mmol) in DMF (5 mL) was added NaN₃ (98 mg, 1.5 mmol). The reaction mixture was stirred at 60° C. for 30 min. Then water was added and the mixture was extracted with EA. The combined organic layers were dried with Na₂SO₄ and concentrated. The residue was purified by silica gel chromatography eluting with DCM/EA (70/30, v/v) to provide the product S27 (89 mg, 80% yield) as colorless oil. ¹H NMR (600 MHz, CDCl₃) δ 9.22 (s, 1H), 9.08 (s, 2H), 7.53-7.48 (m, 2H), 7.24 (d, J=6.9 Hz, 1H), 5.06 (d, J=2.8 Hz, 1H), 3.73 (dd, J=14.5, 4.8 Hz, 1H), 3.43-3.36 (m, 1H). ¹³C NMR (151 MHz, CDCl₃) δ 157.83, 155.19, 143.63, 140.82, 131.22, 130.81, 129.73, 125.68, 124.11, 59.29, 38.21. HRMS (ESI-TOF) m/z Calcd for C₁₂H₁₀N₅⁺ [M+H]⁺ 224.0936, found 224.0942.

Following the procedure for synthesis of S7 with 0.4 mmol of S27 at 60° C. for 30 min. The crude product S28 was obtained and used in the next step without further purification.

### Synthesis of S29

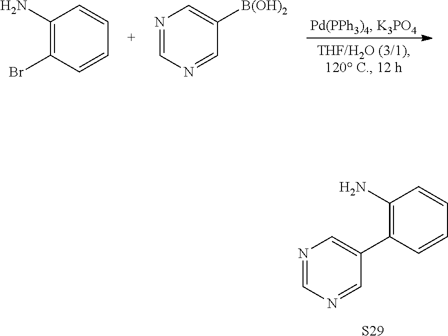 [SMILES: Nc1ccccc1Br ; OB(O)c1cncnc1 ; Nc1ccccc1-c1cncnc1 ; "S29"]

Following the procedure for synthesis of S1, purification by silica gel chromatography eluting with hexane/EA (50/50, v/v) afforded product S29 (328 mg, 64% yield) as a pale yellow solid. ¹H NMR (600 MHz, CDCl₃) δ 9.20 (s, 1H), 8.88 (s, 2H), 7.25 (td, J=7.7, 1.6 Hz, 1H), 7.10 (dd, J=7.6, 1.6 Hz, 1H), 6.89 (td, J=7.5, 1.2 Hz, 1H), 6.81 (dd, J=8.1, 1.2 Hz, 1H), 3.72 (s, 2H). ¹³C NMR (151 MHz, CDCl₃) δ 157.44, 156.97, 143.86, 133.41, 130.56, 130.26, 119.97, 119.36, 116.28. HRMS (ESI-TOF) m/z Calcd for C₁₀H₁₀N₃⁺ [M+H]⁻ 172.0875, found 172.0875.

### Synthesis of S31⁵

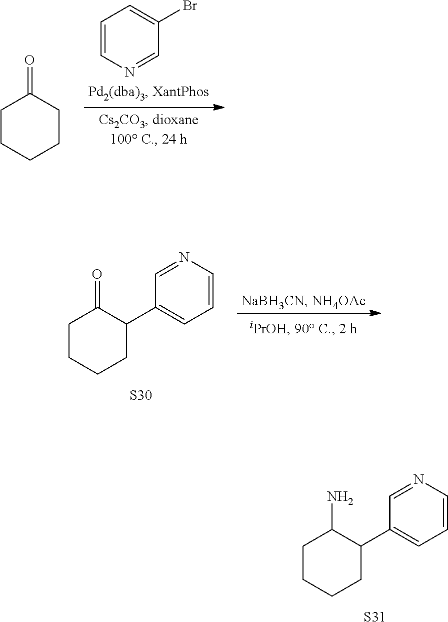 [SMILES: O=C1CCCCC1 ; Brc1cccnc1 ; O=C1CCCCC1c1cccnc1 ; "S30" ; NC1CCCCC1c1cccnc1 ; "S31"]

To a solution of Pd₂(dba)₃ (14 mg, 0.015 mmol), XantPhos (21 mg, 0.036 mmol), and Cs₂CO₃ (2.15 g, 6.6 mmol) in dioxane (3 mL) was added cyclohexanone (622 L, 6 mmol) and 3-bromopyridine (289 μL, 3 mmol). The Schlenk tube was evacuated and refilled with nitrogen for three times. Then sealed and put into a preheated oil bath at 100° C. for 24 h. After completion, the reaction mixture was cooled to room temperature. Water was added and the mixture was extracted with EA. The combined organic layers were dried with Na₂SO₄ and concentrated. The residue was purified by silica gel chromatography eluting with hexane/EA (50/50, v/v) to provide the product S30 (140 mg, 27% yield) as a pale yellow solid. ¹H NMR (600 MHz, CDCl₃) δ 8.50 (dd, J=4.8, 1.7 Hz, 1H), 8.38 (d, J=2.2 Hz, 1H), 7.49 (dt, J=7.8, 2.0 Hz, 1H), 7.29-7.26 (m, 1H), 3.63 (dd, J=12.7, 5.5 Hz, 1H), 2.56 (dddd, J=13.9, 4.6, 3.1, 1.5 Hz, 1H), 2.53-2.46 (m, 1H), 2.32-2.27 (m, 1H), 2.21 (ddq, J=9.2, 6.0, 3.2 Hz, 1H), 2.03 (dddd, J=8.4, 6.7, 4.1, 2.8 Hz, 1H), 1.99-1.94 (m, 1H), 1.85 (dddd, J=16.2, 8.9, 4.2, 1.9 Hz, 2H). ¹³C NMR (151 MHz, CDCl₃) δ 209.18, 149.91, 148.29, 136.29, 134.29, 123.25, 54.93, 42.22, 35.43, 27.80, 25.45. HRMS (ESI-TOF) m/z Calcd for C₁₁H₁₄NO⁺ [M+H]⁺ 176.1075, found 176.1081.

To a solution of S30 (100 mg, 0.57 mmol) and NH₄OAc (439 mg, 5.7 mmol) in ⁱPrOH (5 mL) at room temperature was added NaBH₃CN (71 mg, 1.14 mmol). The sealed tube was put into a preheated oil bath at 90° C. for 2 h. The reaction mixture was basified with NaOH solution and evaporated. Then water was added and the solution was extracted with EA. The organic phase was washed with brine, dried with Na₂SO₄, and concentrated to afford the crude product S31 as brown oil, which was used in the next step without further purification.

### Synthesis of S35⁶

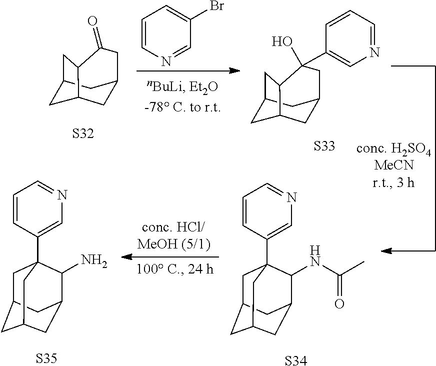

To a solution of 3-bromopyridine (586 μL, 6 mmol) in Et₂O (20 mL) at −78° C. was slowly added ⁿBuLi (6 mmol). S32 (450 mg, 3 mmol) in Et₂O (10 mL) was added after the solution was stirred for 30 min. The reaction mixture was stirred for 2 h (−78° C., 1 h; r.t., 1h). Cooling to 0° C., NH₄Cl saturated solution was added to quench the reaction. Then NaHCO₃ saturated solution was added and the solution was extracted with EA. The organic phase was washed with brine, dried with Na₂SO₄, and concentrated. The residue was purified by silica gel chromatography eluting with hexane/EA (15/85, v/v) to provide the product S33 (289 mg, 42% yield) as a white solid.

For S32: ¹H NMR (600 MHz, CDCl₃) δ 2.76-2.71 (m, 1H), 2.62 (q, J=6.0 Hz, 1H), 2.55 (dd, J=17.9, 2.7 Hz, 1H), 2.44-2.39 (m, 1H), 2.32-2.27 (m, 1H), 2.25 (dt, J=9.6, 4.7 Hz, 1H), 2.00-1.93 (m, 2H), 1.83-1.79 (m, 1H), 1.74 (dtt, J=11.4, 4.1, 2.0 Hz, 1H), 1.71-1.66 (m, 2H), 1.64 (dd, J=11.2, 2.7 Hz, 1H), 1.54 (ddd, J=13.1, 5.2, 2.4 Hz, 1H). ¹³C NMR (151 MHz, CDCl₃) δ 216.79, 51.18, 45.09, 41.43, 38.18, 37.44, 37.31, 37.20, 34.88, 29.58.

For S33: Diastereomer 1: ¹H NMR (600 MHz, CDCl₃) δ 8.75 (d, J=2.4 Hz, 1H), 8.47 (dd, J=4.8, 1.6 Hz, 1H), 7.82 (ddd, J=8.1, 2.4, 1.6 Hz, 1H), 7.28-7.26 (m, 1H), 2.79-2.74 (m, 1H), 2.48 (q, J=6.3 Hz, 1H), 2.41-2.36 (m, 1H), 2.33-2.23 (m, 2H), 2.23-2.16 (m, 2H), 1.97 (s, 1H), 1.84 (ddt, J=13.6, 5.6, 2.8 Hz, 1H), 1.77-1.72 (m, 2H), 1.61 (dd, J=11.0, 3.0 Hz, 1H), 1.47 (dd, J=12.4, 3.1 Hz, 1H), 1.37 (dt, J=13.2, 3.0 Hz, 1H), 1.27 (dt, J=12.8, 2.5 Hz, 1H). ¹³C NMR (151 MHz, CDCl₃) δ 148.10, 147.70, 144.22, 133.73, 123.22, 75.46, 44.96, 42.36, 41.75, 39.75, 36.30, 35.85, 33.79, 32.30, 28.79. HRMS (ESI-TOF) m/z Calcd for C₁₅H₂₀NO⁺ [M+H]⁺ 230.1545, found 230.1548. Diastereomer 2: ¹H NMR (600 MHz, CDCl₃) δ 8.91 (d, J=2.5 Hz, 1H), 8.49 (dd, J=4.7, 1.6 Hz, 1H), 7.95 (dt, J=8.1, 2.0 Hz, 1H), 7.28 (dd, J=8.1, 4.7 Hz, 1H), 2.92-2.85 (m, 1H), 2.80 (dd, J=14.6, 8.9 Hz, 1H), 2.35-2.29 (m, 1H), 2.26 (t, J=4.2 Hz, 1H), 2.22 (ddd, J=13.1, 3.1, 1.5 Hz, 1H), 2.13-2.04 (m, 2H), 1.88 (dddd, J=13.0, 10.5, 5.2, 2.3 Hz, 2H), 1.82-1.76 (m, 2H), 1.54 (dd, J=10.8, 3.1 Hz, 2H), 1.20-1.16 (m, 2H). ¹³C NMR (151 MHz, CDCl₃) δ 148.31, 148.07, 144.12, 134.19, 123.20, 74.09, 45.02, 42.45, 41.79, 40.17, 35.64, 34.72, 34.14, 31.43, 29.13. HRMS (ESI-TOF) m/z Calcd for C₁₅H₂₀NO⁺ [M+H]⁺ 230.1545, found 230.1544.

To 10 ml of MeCN at 0° C. was slowly added conc. H₂SO₄ (3.75 mL) and S33 (114 mg, 1.5 mmol). The reaction mixture was warm to room temperature and stirred for 3 h. Then the mixture was poured into ice and basified with 10% KOH solution. The suspension was filtered and the solid residue was washed with water and hexane. Using rotovap to dry the residue gave product S34 (284 mg, 70% yield) as colorless oil. ¹H NMR (600 MHz, CDCl₃) δ 8.54 (d, J=2.5 Hz, 1H), 8.45 (d, J=3.5 Hz, 1H), 7.70 (dt, J=8.1, 2.0 Hz, 1H), 7.25 (dd, J=8.0, 4.8 Hz, 1H), 5.43 (d, J=9.1 Hz, 1H), 4.49 (d, J=8.9 Hz, 1H), 2.20-2.12 (m, 3H), 2.07-1.99 (m, 4H), 1.91 (dq, J=12.9, 2.8 Hz, 1H), 1.83-1.79 (m, 1H), 1.78-1.74 (m, 2H), 1.74 (s, 3H), 1.73-1.68 (m, 2H). ¹³C NMR (151 MHz, CDCl₃) b 169.01, 147.73, 147.07, 141.88, 133.37, 123.31, 55.29, 45.77, 38.67, 36.70, 36.47, 34.97, 32.75, 30.90, 28.19, 27.60, 23.39. HRMS (ESI-TOF) m/z Calcd for C₁₇H₂₃N₂O⁺ [M+H]⁺ 271.1810, found 271.1813.

To a solution of S34 (100 mg, 0.37 mmol) in MeOH (2 mL) was added 10 mL of conc. HCl. The solution was put into a preheated oil bath at 100° C. for 24 h. The reaction mixture was evaporated and basified with NaOH solution. Then water was added and the solution was extracted with EA. The organic phase was washed with brine, dried with Na₂SO₄, and concentrated to afford the crude product S35 as brown oil, which was used in the next step without further purification.

### Synthesis of S39

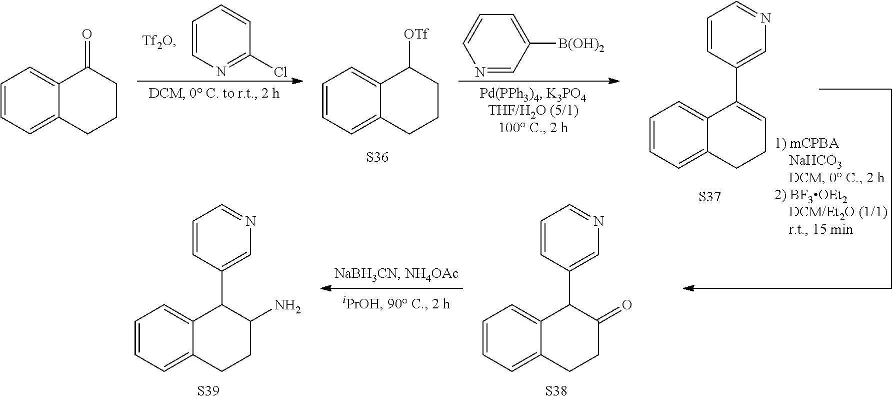

To a solution of α-tetralone (4.55 mL, 34.2 mmol) and 2-chloropyridine (3.5 mL, 37.6 mmol) in DCM (100 mL) at 0° C. was slowly added Tf₂O (6.4 mL, 37.6 mmol) under N₂ protection. The reaction mixture was warm to room temperature and stirred for 2 h. After completion, the mixture was evaporated, and redissolved in hexane. The solution was filtered and the filtrate was concentrated. The residue was purified by silica gel chromatography eluting with hexane to provide the product S36 (4.9 g, 52% yield) as pale yellow oil. ¹H NMR (600 MHz, CDCl₃) δ 7.36-7.32 (m, 1H), 7.27-7.24 (m, 2H), 7.17 (dd, J=5.2, 3.6 Hz, 1H), 6.01 (t, J=4.8 Hz, 1H), 2.87 (t, J=8.2 Hz, 2H), 2.51 (td, J=8.2, 4.8 Hz, 2H). ¹³C NMR (151 MHz, CDCl₃) δ 146.37, 136.22, 129.18, 129.18, 128.67, 127.75, 126.94, 121.23, 117.74, 116.50 (d, J=320.2 Hz), 26.85, 22.32. HRMS (ESI-TOF) m/z Calcd for C₁₁H₁₀F₃O₃S⁺ [M+H]⁺ 279.0303, found 279.0308.

To a solution of K₃PO₄ (8.48 g, 40 mmol) in water (15 mL) was added 3-pyridylboronic acid (4.92 g, 40 mmol), S36 (5.56 g, 20 mmol), Pd(PPh₃)₄ (1.4 g, 1.2 mmol), and THF (60 mL). The Schlenk tube was evacuated and refilled with nitrogen for three times. Then sealed and put into a preheated oil bath at 100° C. for 2 h. After completion, the reaction mixture was cooled to room temperature. Water was added and the mixture was extracted with EA. The combined organic layers were dried with Na₂SO₄ and concentrated. The residue was purified by silica gel chromatography eluting with hexane/EA (70/30, v/v) to provide the product S37 (3.9 g, 94% yield) as pale yellow oil. ¹H NMR (600 MHz, CDCl₃) δ 8.61 (d, J=2.3 Hz, 1H), 8.56 (dd, J=4.9, 1.7 Hz, 1H), 7.63 (dt, J=7.8, 2.0 Hz, 1H), 7.28 (ddd, J=7.8, 4.8, 0.9 Hz, 1H), 7.22-7.18 (m, 1H), 7.17 (td, J=7.3, 1.4 Hz, 1H), 7.11 (td, J=7.5, 1.6 Hz, 1H), 6.91 (d, J=7.7 Hz, 1H), 6.12 (t, J=4.7 Hz, 1H), 2.88-2.83 (m, 2H), 2.42 (td, J=8.0, 4.7 Hz, 2H). ¹³C NMR (151 MHz, CDCl₃) δ 149.67, 148.39, 136.67, 136.61, 136.29, 136.05, 134.29, 129.27, 127.77, 127.41, 126.39, 124.92, 123.02, 28.04, 23.47. HRMS (ESI-TOF) m/z Calcd for C₁₅H₁₄N⁺ [M+H]⁺ 208.1126, found 208.1126.

S38 was synthesized according to modified literature procedures:⁷ To a solution of S37 (1.51 g, 7.3 mmol) in DCM (50 mL) at 0° C. was sequentially added NaHCO₃ (1.84 g, 21.9 mmol) and mCPBA (21.9 mmol). The reaction mixture was stirred at 0° C. for 2 h. Then Na₂S₂O₃ and NaOH solution were sequentially added and the solution was extracted with DCM. The organic phase was washed with brine, dried with Na₂SO₄, and concentrated. The residue was redissolved in 50 mL of DCM/Et₂O (50/50, v/v) and BF₃ Et₂O (18.3 mmol) was added. The solution was stirred at r.t. for 15 min and evaporated in the fume hood. Then NaOH solution was added and the solution was extracted with EA. The organic phase was washed with brine, dried with Na₂SO₄, and concentrated. The residue was purified by silica gel chromatography eluting with hexane/EA (65/35, v/v) to provide the product S38 (260 mg, 16% yield) as pale yellow oil. [Note: S38 is easy to be oxidized in the air and quickly used for next step]

Following the procedure for synthesis of S31 with 1.1 mmol of S38. The crude product S39 was obtained and used in the next step without further purification.

### Synthesis of S40

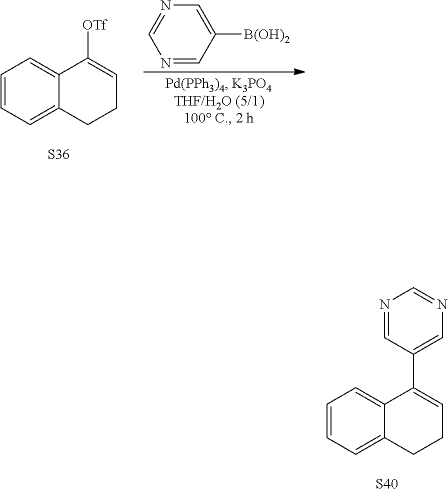 [SMILES: O=S(=O)(OC1=CCCc2ccccc21)C(F)(F)F ; OB(O)c1cncnc1 ; "S36" ; C1=C(c2cncnc2)c2ccccc2CC1 ; "S40"]

Following the procedure for synthesis of S37 with 3 mmol of S36, purification by silica gel chromatography eluting with hexane/EA (70/30, v/v) afforded product S40 (593 mg, 95% yield) as pale yellow oil. ¹H NMR (600 MHz, CDCl₃) δ 9.19 (s, 1H), 8.73 (s, 2H), 7.24-7.20 (m, 2H), 7.15 (td, J=7.6, 2.0 Hz, 1H), 6.89 (d, J=7.6 Hz, 1H), 6.18 (t, J=4.7 Hz, 1H), 2.89 (t, J=8.0 Hz, 2H), 2.49-2.45 (m, 2H). ¹³C NMR (151 MHz, CDCl₃) δ 157.53, 156.46, 136.52, 134.20, 133.64, 133.45, 130.96, 128.05, 127.93, 126.64, 124.51, 27.86, 23.50. HRMS (ESI-TOF) m/z Calcd for C₁₄H₁₃N₂⁺ [M+H]⁺ 209.1079, found 209.1082.

### Optimized Route for Synthesis of Cis-T25⁸

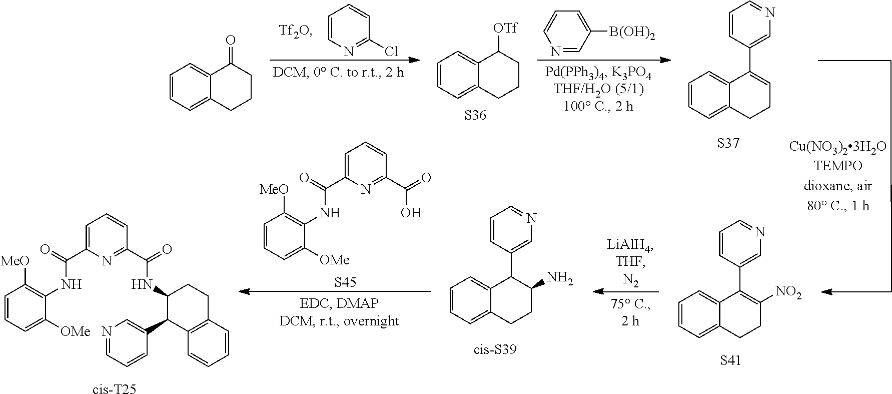

To a solution of S37 (3.1 g, 15 mmol) in dioxane (150 mL) was sequentially added Cu(NO₃)₂·3H₂O (7.26 g, 30 mmol) and TEMPO (937 mg, 6 mmol). The flask was keeping open and the reaction mixture was stirred at 80° C. for 1 h. After filtering off the precipitate, NaOH solution was added into the filtrate and the solution was extracted with EA. The organic phase was washed with brine, dried with Na₂SO₄, and concentrated. The residue was purified by silica gel chromatography eluting with hexane/EA (70/30, v/v) to provide the product S41 (1.7 g, 45% yield) as a yellow solid. ¹H NMR (600 MHz, CDCl₃) δ 8.69 (s, 1H), 8.46 (s, 1H), 7.55 (dt, J=7.8, 1.9 Hz, 1H), 7.40 (dd, J=7.8, 4.8 Hz, 1H), 7.32 (td, J=7.4, 1.3 Hz, 1H), 7.28 (d, J=6.9 Hz, 1H), 7.15 (td, J=7.7, 1.2 Hz, 1H), 6.73-6.70 (m, 1H), 3.14-3.07 (m, 4H). ¹³C NMR (151 MHz, CDCl₃) δ 149.50, 148.77, 146.87, 136.16, 136.04, 135.99, 132.99, 130.49, 128.98, 127.96, 127.20, 123.42, 28.15, 25.81. HRMS (ESI-TOF) m/z Calcd for C₁₅H₁₃N₂O₂⁺ [M+H]⁺ 253.0977, found 253.0983.

To a solution of LAH (24 mmol) in THF (60 mL) at 0° C. was slowly added a solution of S41 (1.5 g, 6 mmol) in THF (40 mL) under N₂ protection. After warming to the room temperature, the flask was put into a preheated oil bath at 75° C. for 2 h. Upon completion, 1 mL of water, 1 mL of NaOH solution, 3 mL water, and Na₂SO₄ were sequentially added. Then, the reaction mixture was filtered. Water was added and the solution was extracted with EA. The organic phase was washed with brine, dried with Na₂SO₄, and concentrated to afford the crude product cis-S39 as brown oil, which was used in the next step without further purification.

To a solution of S45 (1.8 g, 6 mmol), cis-S39 (1.12 g, 5 mmol), and DMAP (61 mg, 0.5 mmol) in DCM (50 mL) at 0° C. was added EDC (1.44 g, 7.5 mmol). The solution was warmed to room temperature and stirred for overnight. The reaction mixture was evaporated and purified by silica gel chromatography eluting with EA to provide the product cis-T25 (509 mg, 17% yield) as a white solid. If necessary, MeOH can be used to further recrystallization.

### Synthesis of S44

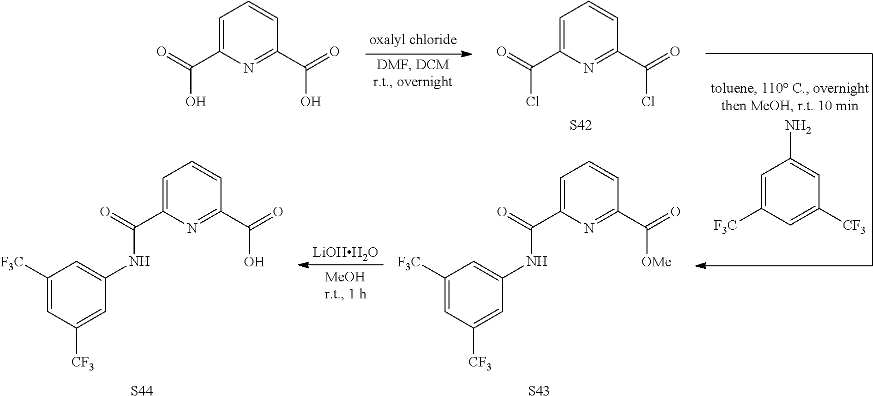

To a stirred solution of 2,6-pyridinedicarboxylic acid (6.68 g, 40 mmol) in DCM (100 mL) at 0° C. was added DMF (40 μL) and oxalyl chloride (7.8 mL, 92 mmol). The reaction solution was warmed to room temperature and stirred for overnight. The mixture was concentrated to obtain the crude product S42, which was then dissolved in toluene (100 mL). 3,5-Bis(trifluoromethyl)aniline (4.4 mL, 28 mmol) was added to the solution at room temperature. The reaction mixture was put into a preheated oil bath at 110° C. for overnight. Upon completion, MeOH (10 mL) was added to quench the reaction. The solvent was removed. The recrystallization from MeOH afforded the product S43 (14.6 g, 93% yield) as a white solid. ¹H NMR (600 MHz, CDCl₃) δ 10.37 (s, 1H), 8.50 (d, J=8.9 Hz, 1H), 8.35 (s, 2H), 8.32 (s, 1H), 8.12 (t, J=7.8 Hz, 1H), 7.67 (s, 1H), 4.08 (s, 3H). ¹³C NMR (151 MHz, CDCl₃) δ 164.64, 161.75, 149.14, 146.65, 139.24, 132.46 (q, J=33.7 Hz), 128.13, 128.08, 125.81, 124.05, 122.24, 119.77 (q, J=4.3 Hz), 118.59-116.89 (m), 53.18. HRMS (ESI-TOF) m/z Calcd for C₁₆H₁₁F₆N₂O₃⁺ [M+H]⁺ 393.0674, found 393.0672.

To a solution of S43 (14.6 g, 37.3 mmol) in MeOH (100 mL) was added LiOH·H₂O (6.3 g, 149.2 mmol). The resulting mixture was stirred at room temperature for 1 h. The mixture was acidified with aqueous HCl solution and extracted with EA. The combined organic layers were dried with Na₂SO₄ and concentrated to give the product S44 (10 g, 71% yield) as a white solid. H NMR (600 MHz, CD₃OD) δ 8.61 (s, 2H), 8.31 (dd, J=7.7, 1.6 Hz, 2H), 8.10 (t, J=7.7 Hz, 1H), 7.69 (s, 1H). ¹³C NMR (151 MHz, CD₃OD) δ 163.35, 152.59, 147.89, 140.27, 137.87, 131.40 (q, J=33.2 Hz), 126.55, 123.87, 122.95, 122.07, 118.94 (dd, J=8.9, 3.4 Hz), 116.28-115.01 (m). HRMS (ESI-TOF) m/z Calcd for C₁₅H₉F₆N₂O₃⁺ [M+H]⁺ 379.0517, found 379.0518.

### Synthesis of S48, T1-7, T18-28, TC11-12

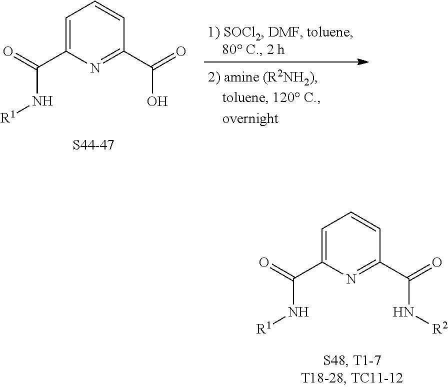

To a solution of S44-47 (1 mmol) in dry toluene (20 mL) was added DMF (3 drops) and thionyl chloride (181 μL, 2.5 mmol) under nitrogen protection. The reaction mixture was stirred at 80° C. for 2 h. After completion, the mixture was concentrated and redissolved in dry toluene (20 mL). Amine (1 mmol) was added, and the reaction solution was stirred at 120° C. for overnight. The mixture was concentrated and purified by silica gel chromatography eluting with hexane/EA to provide the corresponding product S48, T1-7, T18-28, TC11-12.

### Synthesis of T8-12, T16

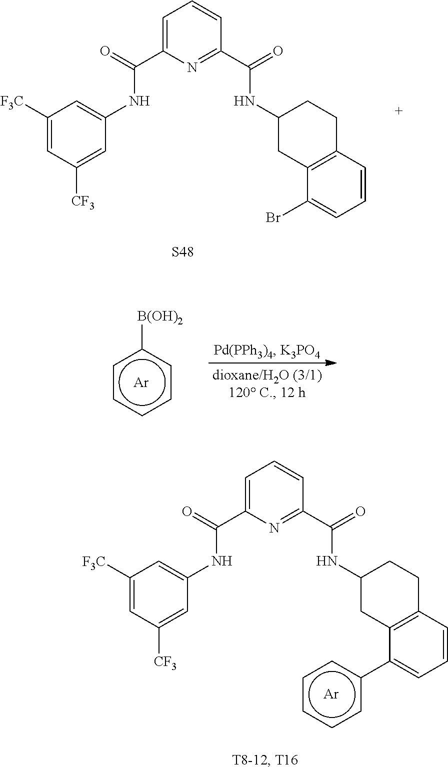

To a solution of K₃PO₄ (127 mg, 0.6 mmol) in water (1 mL) was added S48 (176 mg, 0.3 mmol), arylboronic acid (0.6 mmol), Pd(PPh₃)₄ (35 mg, 0.03 mmol), and dioxane (3 mL). The Schlenk tube was evacuated and refilled with nitrogen for three times. The reaction solution was stirred at 120° C. for 12 h. After completion, the reaction mixture was cooled to room temperature. Water was added and the mixture was extracted with EA. The combined organic layers were dried with Na₂SO₄ and concentrated. The residue was purified by silica gel chromatography eluting with hexane/EA to provide the product T8-12, T16.

### Synthesis of T13-15

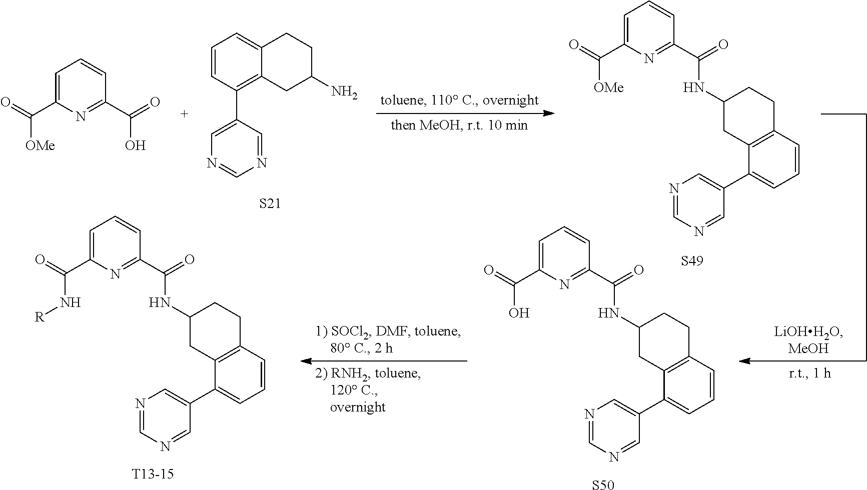

To a solution of 2,6-Pyridinedicarboxylic acid monomethyl ester (592 mg, 2.9 mmol) in toluene (40 mL) was added amine S21 (484 mg, 2 mmol) under nitrogen protection. The reaction mixture was stirred at 110° C. for overnight. Upon completion, MeOH (2 mL) was added to quench the reaction. The solvent was removed. The recrystallization from MeOH afforded the product S49 (621 mg, 80% yield) as colorless oil. ¹H NMR (600 MHz, CDCl₃) δ 9.19 (s, 1H), 8.72 (s, 2H), 8.36 (dd, J=7.8, 1.1 Hz, 1H), 8.21 (dd, J=7.8, 1.1 Hz, 1H), 8.10 (d, J=8.4 Hz, 1H), 8.00 (t, J=7.8 Hz, 1H), 7.31-7.26 (m, 2H), 7.06 (dd, J=7.2, 1.7 Hz, 1H), 4.42-4.36 (m, 1H), 4.01 (s, 3H), 3.12-3.07 (m, 2H), 2.97 (dd, J=16.2, 4.4 Hz, 1H), 2.73 (dd, J=16.2, 10.0 Hz, 1H), 2.29-2.26 (m, 1H), 1.98-1.90 (m, 1H). ¹³C NMR (151 MHz, CDCl₃) δ 164.94, 162.87, 157.41, 156.63, 150.07, 146.52, 138.57, 136.76, 135.02, 134.79, 132.48, 129.79, 127.83, 127.32, 126.51, 125.46, 52.95, 46.08, 34.63, 28.96, 28.80. HRMS (ESI-TOF) m/z Calcd for C₂₂H₂₁N₄O₃⁺ [M+H]⁺ 389.1614, found 389.1615.

To a solution of S49 (608 mg, 1.5 mmol) in MeOH (10 mL) was added LiOH H₂O (252 mg, 6 mmol). The resulting mixture was stirred at room temperature for 1 h. The mixture was acidified with aqueous HCl solution and extracted with EA. The combined organic layers were dried with Na₂SO₄ and concentrated to give the product S50 (477 mg, 85% yield) as colorless oil. ¹H NMR (600 MHz, CDCl₃) δ 9.23 (s, 1H), 8.80 (s, 2H), 8.38 (d, J=7.7 Hz, 1H), 8.30 (d, J=7.6 Hz, 1H), 8.21 (d, J=8.6 Hz, 1H), 8.03 (t, J=7.8 Hz, 1H), 7.28 (t, J=7.5 Hz, 1H), 7.25 (d, J=6.8 Hz, 1H), 7.06 (d, J=6.6 Hz, 1H), 4.41 (t, J=10.1 Hz, 1H), 3.12-3.04 (m, 2H), 2.89 (dd, J=16.0, 5.1 Hz, 1H), 2.77 (dd, J=16.2, 10.4 Hz, 1H), 2.23 (dd, J=12.1, 3.4 Hz, 1H), 1.95-1.88 (m, 1H). ¹³C NMR (151 MHz, CDCl₃) δ 165.38, 162.74, 160.36, 156.61, 156.57, 149.78, 139.12, 136.74, 135.32, 134.25, 132.46, 129.99, 127.76, 127.20, 126.62, 126.07, 46.20, 34.77, 29.06, 29.02. HRMS (ESI-TOF) m/z Calcd for C₂₁H₁₈N₄O₃⁺ [M+H]⁺ 375.1457, found 375.1455.

To a solution of S50 (235 mg, 0.6 mmol) in dry toluene (10 mL) was added DMF (3 drops) and thionyl chloride (109 μL, 1.5 mmol) under nitrogen protection. The reaction mixture was stirred at 80° C. for 2 h. After completion, the mixture was concentrated and redissolved in dry toluene (10 mL). Amine (0.6 mmol) was added and the reaction solution was stirred at 120° C. for overnight. The mixture was concentrated and purified by silica gel chromatography eluting with hexane/EA to provide the corresponding product T13-15.

### Synthesis of T17

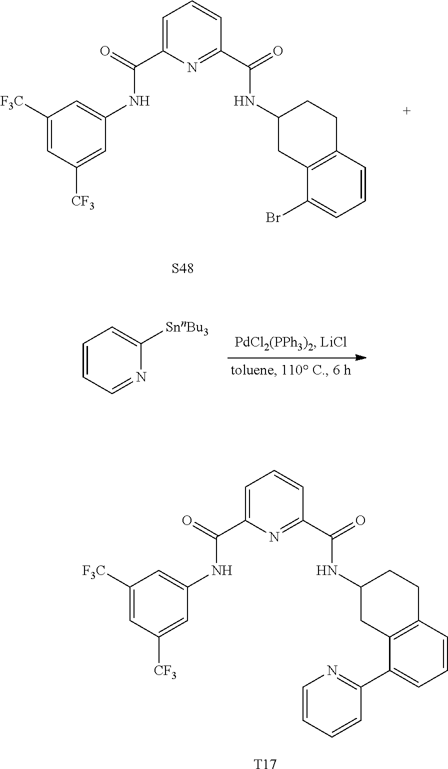

To a solution of S48 (117 mg, 0.2 mmol), PdCl₂(PPh₃)₂ (9.8 mg, 0.014 mmol), LiCl (48 mg, 1.14 mmol) in toluene (10 mL) was added 2-(tributylstannyl)pyridine (96 μL, 0.3 mmol). The Schlenk tube was evacuated and refilled with nitrogen for three times. The reaction solution was stirred at 110° C. for 6 h. KF solution was added and the mixture was stirred for 30 min. After filtration, NaHCO₃ solution was added and the mixture was extracted with EA. The combined organic layers were dried with Na₂SO₄ and concentrated. The residue was purified by silica gel chromatography eluting with hexane/EA to provide the product T17.

### Synthesis of TC1-10

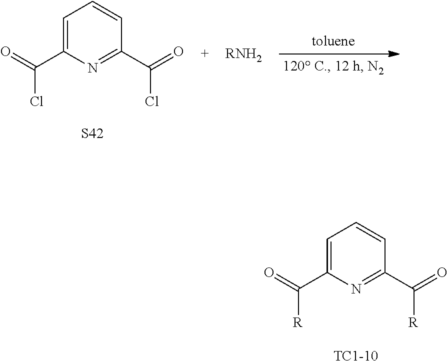

To a stirred solution of 2,6-pyridinedicarbonyl dichloride (255 mg, 1.25 mmol) in toluene (10 mL) was added amine (2.75 mmol). The reaction solution was stirred at 120° C. for 12 h. Upon completion, the reaction mixture was filtered. The residue was washed with toluene and MeOH or purified by silica gel chromatography eluting with hexane/EA to afford the corresponding product TC1-10.

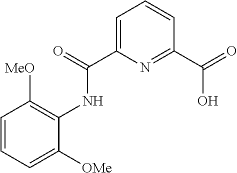 [SMILES: COc1cccc(OC)c1NC(=O)c1cccc(C(=O)O)n1]

6-((2,6-Dimethoxyphenyl)carbamoyl)picolinic acid (S45) Following procedure for synthesis of S44, S45 was obtained as a white solid in 52% yield. ¹H NMR (600 MHz, CD₃OD) δ 8.38 (ddd, J=14.2, 7.7, 1.1 Hz, 2H), 8.21 (t, J=7.8 Hz, 1H), 7.31 (t, J=8.5 Hz, 1H), 6.75 (d, J=8.5 Hz, 2H), 3.83 (s, 6H). ¹³C NMR (151 MHz, CD₃OD) δ 165.67, 162.64, 155.80, 149.41, 146.18, 138.76, 127.87, 126.60, 125.01, 112.75, 103.52, 54.54. HRMS (ESI-TOF) m/z Calcd for C₁₅H₁₅N₂O₅⁺ [M+H]⁺ 303.0981, found 303.0986.

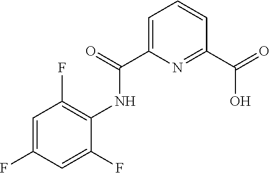 [SMILES: O=C(O)c1cccc(C(=O)Nc2c(F)cc(F)cc2F)n1]

6-((2,4,6-Trifluorophenyl)carbamoyl)picolinic acid (S46) Following procedure for synthesis of S44, S46 was obtained as a white solid in 90% yield. ¹H NMR (600 MHz, CD₃OD) δ 8.41 (ddd, J=8.6, 7.7, 1.1 Hz, 2H), 8.24 (t, J=7.8 Hz, 1H), 7.06-7.01 (m, 2H). ¹³C NMR (151 MHz, CD₃OD) δ 165.53, 162.99, 160.09 (t, J=14.9 Hz), 159.17 (dd, J=15.6, 7.4 Hz), 157.50 (dd, J=15.5, 7.3 Hz), 148.40, 146.35, 139.01, 127.21, 125.30, 99.82 (dd, J=29.6, 26.9 Hz). HRMS (ESI-TOF) m/z Calcd for C₁₃H₈F₃N₂O₃⁺ [M+H]⁺ 297.0487, found 297.0489.

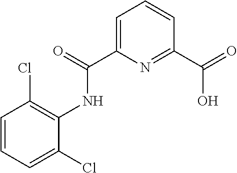 [SMILES: O=C(O)c1cccc(C(=O)Nc2c(Cl)cccc2Cl)n1]

6-((2,6-Dichlorophenyl)carbamoyl)picolinic acid (S47) Following procedure for synthesis of S44, S47 was obtained as a white solid in 85% yield. ¹H NMR (600 MHz, CD₃OD) δ 8.42 (t, J=8.1 Hz, 2H), 8.25 (t, J=7.8 Hz, 1H), 7.54 (d, J=8.2 Hz, 2H), 7.38 (t, J=8.2 Hz, 1H). ¹³C NMR (151 MHz, CD₃OD) δ 165.49, 162.66, 148.54, 146.30, 139.04, 133.83, 131.78, 128.75, 127.87, 127.16, 125.31. HRMS (ESI-TOF) m/z Calcd for C₁₃H₉Cl₂N₂O₃⁻ [M+H]⁺ 310.9990, found 310.9997.

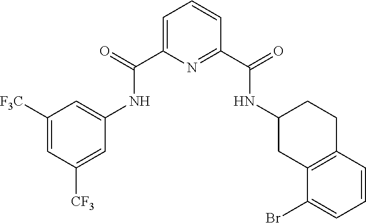 [SMILES: O=C(Nc1cc(C(F)(F)F)cc(C(F)(F)F)c1)c1cccc(C(=O)NC2CCc3cccc(Br)c3C2)n1]

N²-(3,5-Bis(trifluoromethyl)phenyl)-N⁶-(8-bromo-1,2,3,4-tetrahydronaphthalen-2-yl)pyridine-2,6-dicarboxamide (S48) Purification by silica gel chromatography eluting with hexane/EA (75/25, v/v), white solid, 40% yield. ¹H NMR (600 MHz, CDCl₃) δ 9.64 (s, 1H), 8.48 (dd, J=7.8, 1.2 Hz, 1H), 8.44 (dd, J=7.8, 1.2 Hz, 1H), 8.18 (s, 2H), 8.12 (t, J=7.8 Hz, 1H), 7.74 (d, J=8.3 Hz, 1H), 7.68 (s, 1H), 7.42 (d, J=7.7 Hz, 1H), 7.10 (d, J=7.5 Hz, 1H), 7.03 (t, J=7.8 Hz, 1H), 4.61 (qdd, J=8.4, 5.5, 3.1 Hz, 1H), 3.26 (dd, J=17.1, 5.6 Hz, 1H), 2.97 (q, J=7.3, 6.1 Hz, 2H), 2.84 (dd, J=17.1, 7.7 Hz, 1H), 2.20-2.15 (m, 1H), 2.00-1.90 (m, 1H). ¹³C NMR (151 MHz, CDCl₃) δ 162.69, 161.61, 149.21, 147.83, 139.68, 138.63, 137.93, 133.27, 132.57 (q, J=33.6 Hz), 130.42, 128.01, 127.67, 126.24, 125.81, 125.67, 123.95, 122.14, 119.82, 45.61, 36.34, 28.05, 27.63. HRMS (ESI-TOF) m/z Calcd for C₂₅H₁₉BrF₆N₃O₂⁺ [M+H]⁺ 586.0565, found 586.0566.

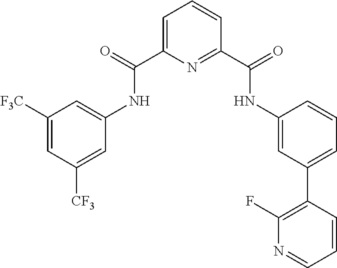 [SMILES: O=C(Nc1cccc(-c2cccnc2F)c1)c1cccc(C(=O)Nc2cc(C(F)(F)F)cc(C(F)(F)F)c2)n1]

N²-(3,5-Bis(trifluoromethyl)phenyl)-N⁶-(3-(2-fluoropyridin-3-yl)phenyl)pyridine-2,6-dicarboxamide (T1) Purification by silica gel chromatography eluting with hexane/EA (60/40, v/v), white solid, 72% yield. ¹H NMR (600 MHz, CDCl₃) δ 9.82 (s, 1H), 9.52 (s, 1H), 8.51 (dd, J=16.6, 7.7 Hz, 2H), 8.31 (s, 2H), 8.21 (d, J=4.8 Hz, 1H), 8.17 (t, J=7.7 Hz, 1H), 7.96 (s, 1H), 7.91-7.85 (m, 1H), 7.72 (d, J=8.0 Hz, 1H), 7.69 (s, 1H), 7.48 (t, J=7.8 Hz, 1H), 7.38 (d, J=7.5 Hz, 1H), 7.28 (s, 1H). ¹³C NMR (151 MHz, CDCl₃) δ 161.69, 161.34, 161.07, 159.48, 149.14, 148.20, 146.77 (d, J=15.9 Hz), 140.70 (d, J=4.3 Hz), 139.97, 138.60, 137.18, 134.99, 132.63 (q, J=34.1 Hz), 129.62, 126.40, 126.12, 125.70 (d, J=2.9 Hz), 123.91 (d, J=4.6 Hz), 123.01, 122.13, 121.95 (d, J=5.1 Hz), 120.96 (d, J=3.6 Hz), 120.61, 120.11 (q, J=4.1 Hz), 118.29 (d, J=2.3 Hz), 78.09. HRMS (ESI-TOF) m/z Calcd for C₂₆H₁₆F₇N₄O₂⁺ [M+H]⁺ 549.1161, found 549.1167.

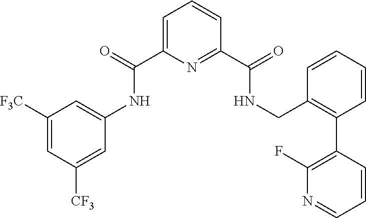 [SMILES: O=C(NCc1ccccc1-c1cccnc1F)c1cccc(C(=O)Nc2cc(C(F)(F)F)cc(C(F)(F)F)c2)n1]

N²-(3,5-Bis(trifluoromethyl)phenyl)-N⁶-(2-(2-fluoropyridin-3-yl)benzyl)pyridine-2,6-dicarboxamide (T2) Purification by silica gel chromatography eluting with hexane/EA (65/35, v/v), white solid, 50% yield. ¹H NMR (600 MHz, CDCl₃) δ 9.90 (s, 1H), 8.54 (s, 2H), 8.46 (dd, J=7.8, 1.2 Hz, 1H), 8.42 (dd, J=7.8, 1.2 Hz, 1H), 8.27 (ddd, J=4.9, 2.0, 1.2 Hz, 1H), 8.24 (q, J=5.0 Hz, 1H), 8.12 (t, J=7.8 Hz, 1H), 7.82 (ddd, J=10.1, 7.3, 2.0 Hz, 1H), 7.67 (s, 1H), 7.65 (dd, J=7.7, 1.4 Hz, 1H), 7.48 (td, J=7.5, 1.6 Hz, 1H), 7.45 (td, J=7.5, 1.6 Hz, 1H), 7.38 (ddd, J=7.2, 4.9, 2.1 Hz, 1H), 7.30 (dd, J=7.5, 1.5 Hz, 1H), 4.94 (s, 1H), 4.14 (s, 1H). ¹³C NMR (151 MHz, CDCl₃) δ 162.60, 161.81, 161.21, 159.68, 148.99, 148.07, 147.40 (d, J=15.4 Hz), 142.23 (d, J=4.8 Hz), 139.55, 138.98, 136.21, 133.44 (d, J=3.7 Hz), 132.38 (q, J=33.5 Hz), 130.62, 130.54, 129.72, 128.52, 125.92, 125.61, 124.11, 123.01, 122.79, 122.43 (d, J=4.1 Hz), 120.64-119.49 (m), 117.89 (d, J=4.5 Hz), 41.75. HRMS (ESI-TOF) m/z Calcd for C₂₇H₁₈F₇N₄O₂⁺ [M+H]⁺ 563.1318, found 563.1320.

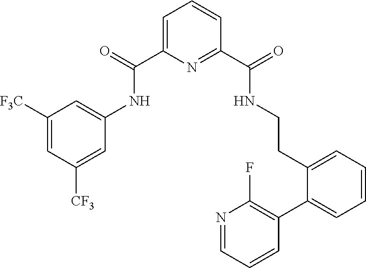 [SMILES: O=C(NCCc1ccccc1-c1cccnc1F)c1cccc(C(=O)Nc2cc(C(F)(F)F)cc(C(F)(F)F)c2)n1]

N²-(3,5-Bis(trifluoromethyl)phenyl)-N⁶-(2-(2-fluoropyridin-3-yl)phenethyl)pyridine-2,6-dicarboxamide (T3) Purification by silica gel chromatography eluting with hexane/EA (65/35, v/v), colorless oil, 71% yield. ¹H NMR (600 MHz, CDCl₃) δ 9.78 (s, 1H), 8.45 (dd, J=7.8, 1.2 Hz, 1H), 8.40 (dd, J=7.8, 1.2 Hz, 1H), 8.27 (s, 2H), 8.17 (ddd, J=4.9, 2.0, 1.0 Hz, 1H), 8.10 (t, J=7.8 Hz, 1H), 7.80 (t, J=5.8 Hz, 1H), 7.71 (ddd, J=9.6, 7.3, 2.0 Hz, 1H), 7.66 (s, 1H), 7.42 (dd, J=7.8, 1.4 Hz, 1H), 7.40-7.35 (m, 1H), 7.32-7.27 (m, 2H), 7.23 (dd, J=7.6, 1.4 Hz, 1H), 3.72 (q, J=6.5 Hz, 2H), 2.86 (t, J=6.9 Hz, 2H). ¹³C NMR (151 MHz, CDCl₃) δ 163.26, 161.99, 160.95, 159.40, 149.04, 148.03, 147.05 (d, J=14.7 Hz), 142.31 (d, J=4.6 Hz), 139.45, 138.83, 137.02, 133.89 (d, J=3.8 Hz), 132.33 (q, J=33.6 Hz), 130.54, 129.35, 129.14, 127.05, 126.02, 125.66, 124.04, 123.54, 123.32, 122.23, 121.95 (d, J=4.4 Hz), 120.50-119.97 (m), 117.95 (d, J=4.0 Hz), 39.61, 32.25. HRMS (ESI-TOF) m/z Calcd for C₂₈H₂₀F₇N₄O₂⁺ [M+H]⁺ 577.1474, found 577.1477.

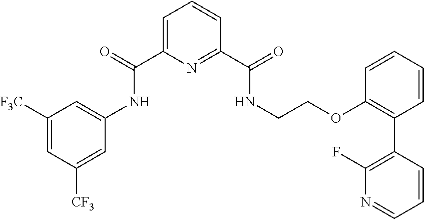 [SMILES: O=C(NCCOc1ccccc1-c1cccnc1F)c1cccc(C(=O)Nc2cc(C(F)(F)F)cc(C(F)(F)F)c2)n1]

N²-(3,5-Bis(trifluoromethyl)phenyl)-N⁶-(2-(2-(2-fluoropyridin-3-yl)phenoxy)ethyl)pyridine-2,6-dicarboxamide (T4) Purification by silica gel chromatography eluting with hexane/EA (70/30, v/v), colorless oil, 56% yield. ¹H NMR (600 MHz, CDCl₃) δ 10.20 (s, 1H), 8.50 (ddd, J=10.2, 7.8, 1.2 Hz, 2H), 8.30 (s, 3H), 8.12 (t, J=7.8 Hz, 1H), 7.99-7.95 (m, 1H), 7.77 (ddd, J=9.5, 7.3, 2.0 Hz, 1H), 7.60 (s, 1H), 7.42 (ddd, J=8.2, 7.5, 1.7 Hz, 1H), 7.30 (dd, J=7.5, 1.7 Hz, 1H), 7.25 (dd, J=4.9, 2.3 Hz, 1H), 7.10 (td, J=7.5, 1.0 Hz, 1H), 6.99 (d, J=7.9 Hz, 1H), 4.30-4.23 (m, 2H), 3.91 (q, J=5.4 Hz, 2H). ¹³C NMR (151 MHz, CDCl₃) δ 163.67, 162.51, 161.31, 159.76, 155.25, 149.23, 148.70, 146.14 (d, J=15.3 Hz), 142.17 (d, J=5.6 Hz), 139.13, 138.99, 131.95 (q, J=33.5 Hz), 130.85, 130.54, 126.29, 126.01, 124.02, 123.30 (d, J=4.5 Hz), 122.22, 122.18, 122.16, 121.56, 121.45, 121.44, 121.39, 121.17, 118.06-117.62 (m), 111.40, 67.25, 39.43. HRMS (ESI-TOF) m/z Calcd for C₂₈H₂₀F₇N₄O₃⁺ [M+H]⁺ 593.1424, found 593.1435.

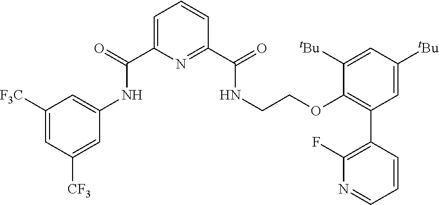 [SMILES: CC(C)(C)c1cc(-c2cccnc2F)c(OCCNC(=O)c2cccc(C(=O)Nc3cc(C(F)(F)F)cc(C(F)(F)F)c3)n2)c(C(C)(C)C)c1]

N²-(3,5-Bis(trifluoromethyl)phenyl)-N⁶-(2-(2,4-di-tert-butyl-6-(2-fluoropyridin-3-yl)phenoxy)ethyl)pyridine-2,6-dicarboxamide (T5) Purification by silica gel chromatography eluting with hexane/EA (70/30, v/v), white solid, 36% yield. ¹H NMR (600 MHz, CDCl₃) δ10.79 (s, 1H), 8.46 (dd, J=7.8, 1.2 Hz, 1H), 8.41 (s, 2H), 8.38 (dd, J=7.8, 1.2 Hz, 1H), 8.12 (t, J=7.8 Hz, 1H), 8.04 (t, J=6.0 Hz, 1H), 7.87 (ddd, J=9.3, 7.3, 2.0 Hz, 1H), 7.67 (dd, J=4.9, 1.9 Hz, 1H), 7.60 (s, 1H), 7.49 (d, J=2.5 Hz, 1H), 7.17 (ddd, J=7.0, 4.9, 1.7 Hz, 1H), 7.11-7.08 (m, 1H), 3.62 (s, 4H), 1.46 (s, 9H), 1.34 (s, 9H). ¹³C NMR (151 MHz, CDCl₃) δ 163.22, 163.03, 161.31, 159.73, 153.05, 149.11, 148.94, 146.94, 145.77 (d, J=14.1 Hz), 142.36, 141.71 (d, J=5.3 Hz), 139.46, 139.11, 132.86-130.97 (m), 127.60 (d, J=4.8 Hz), 126.28, 125.78, 125.76, 125.74, 124.03, 123.19, 122.98, 122.23, 122.08, 122.05, 120.96-119.74 (m), 117.96-117.13 (m), 72.40, 39.42, 35.41, 34.68, 31.45, 31.03. HRMS (ESI-TOF) m/z Calcd for C₃₆H₃₆F₇N₄O₃⁺ [M+H]⁺ 705.2676, found 705.2672.

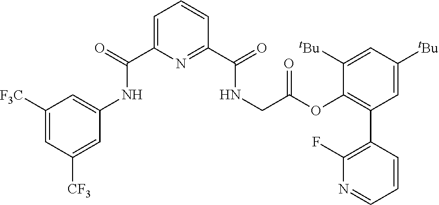 [SMILES: CC(C)(C)c1cc(-c2cccnc2F)c(OC(=O)CNC(=O)c2cccc(C(=O)Nc3cc(C(F)(F)F)cc(C(F)(F)F)c3)n2)c(C(C)(C)C)c1]

2,4-Di-tert-butyl-6-(2-fluoropyridin-3-yl)phenyl(6-((3,5-bis(trifluoromethyl)phenyl)carbamoyl)picolinoyl)glycinate (T6) Purification by silica gel chromatography eluting with hexane/EA (65/35, v/v), white solid, 78% yield. ¹H NMR (600 MHz, CDCl₃) δ 10.25 (s, 1H), 8.51 (dd, J=7.8, 1.2 Hz, 1H), 8.46 (d, J=8.1 Hz, 3H), 8.21 (d, J=3.7 Hz, 1H), 8.17-8.08 (m, 2H), 7.84 (ddd, J=9.5, 7.3, 2.0 Hz, 1H), 7.64 (s, 1H), 7.53 (d, J=2.3 Hz, 1H), 7.34 (t, J=5.6 Hz, 1H), 7.22 (d, J=2.4 Hz, 1H), 4.50 (s, 1H), 3.79 (s, 1H), 1.35 (d, J=4.0 Hz, 18H). ¹³C NMR (151 MHz, CDCl₃) δ 167.61, 163.17, 161.97, 149.47, 148.35 (d, J=8.4 Hz), 146.93 (d, J=14.1 Hz), 144.42, 141.36, 139.57, 139.04, 132.28 (q, J=33.5 Hz), 127.54 (d, J=4.5 Hz), 126.06 (d, J=2.3 Hz), 125.73, 125.61, 124.05, 122.24, 120.60 (dd, J=6.0, 3.0 Hz), 118.17-117.45 (m), 41.59, 34.99, 34.88, 31.40, 30.43. HRMS (ESI-TOF) m/z Calcd for C₃₆H₃₄F₇N₄O₄⁺ [M+H]⁺ 719.2468, found 719.2464.

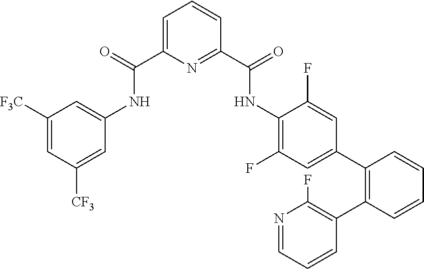 [SMILES: O=C(Nc1cc(C(F)(F)F)cc(C(F)(F)F)c1)c1cccc(C(=O)Nc2c(F)cc(-c3ccccc3-c3cccnc3F)cc2F)n1]

N²-(3,5-Bis(trifluoromethyl)phenyl)-N⁶-(3,5-difluoro-2′-(2-fluoropyridin-3-yl)-[1,1′-biphenyl]-4-yl)pyridine-2,6-dicarboxamide (T7) Purification by silica gel chromatography eluting with hexane/EA (60/40, v/v), white solid, 52% yield. ¹H NMR (600 MHz, CDCl₃) δ 10.22 (s, 1H), 9.43 (s, 1H), 8.54 (ddd, J=15.3, 7.8, 1.1 Hz, 2H), 8.26 (s, 2H), 8.18 (t, J=7.8 Hz, 1H), 8.13 (dt, J=4.1, 1.2 Hz, 1H), 7.73 (ddd, J=9.4, 7.4, 2.0 Hz, 1H), 7.61 (s, 1H), 7.52-7.48 (m, 2H), 7.40-7.37 (m, 1H), 7.36-7.31 (m, 1H), 7.24 (ddd, J=7.4, 4.9, 1.6 Hz, 1H), 6.64 (d, J=8.5 Hz, 2H). ¹³C NMR (151 MHz, CDCl₃) δ 162.04, 162.02, 160.68, 159.09, 158.11 (d, J=6.1 Hz), 156.44 (d, J=5.8 Hz), 148.21, 148.06, 146.85 (d, J=14.0 Hz), 142.25 (d, J=4.9 Hz), 141.90-141.52 (m), 139.68, 138.98-138.82 (m), 138.78, 132.93-131.72 (m), 131.11, 129.95, 129.27, 128.73, 126.52, 126.26, 123.93, 123.55, 123.34, 122.12, 121.82 (d, J=5.0 Hz), 120.66-119.85 (m), 118.02 (dd, J=8.6, 4.0 Hz), 113.06-112.37 (m), 111.96. HRMS (ESI-TOF) m/z Calcd for C₃₂H₁₈F₉N₄O₂⁺ [M+H]⁺ 661.1286, found 661.1291.

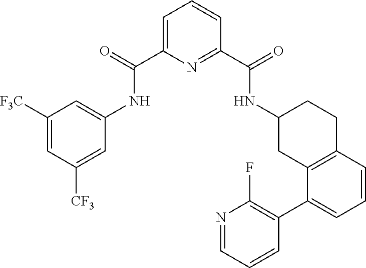 [SMILES: O=C(Nc1cc(C(F)(F)F)cc(C(F)(F)F)c1)c1cccc(C(=O)NC2CCc3cccc(-c4cccnc4F)c3C2)n1]

N²-(3,5-Bis(trifluoromethyl)phenyl)-N⁶-(8-(2-fluoropyridin-3-yl)-1,2,3,4-tetrahydronaphthalen-2-yl)pyridine-2,6-dicarboxamide (T8) Purification by silica gel chromatography eluting with hexane/EA (65/35, v/v), white solid, 57% yield. ¹H NMR (600 MHz, CDCl₃) δ 9.68 (s, 1H), 8.43 (td, J=7.7, 1.2 Hz, 2H), 8.20 (d, J=4.2 Hz, 1H), 8.15 (s, 2H), 8.09 (t, J=7.8 Hz, 1H), 7.79 (d, J=59.2 Hz, 2H), 7.63 (s, 1H), 7.29 (d, J=15.4 Hz, 1H), 7.25 (s, 1H), 7.22 (s, 1H), 7.08 (d, J=8.8 Hz, 1H), 4.56 (h, J=5.9 Hz, 1H), 2.97 (m, 2H), 2.74 (m, 2H), 2.13 (m, 2H). ¹³C NMR (151 MHz, CDCl₃) δ 162.85, 161.87, 149.14, 148.03, 147.06, 146.95, 142.05, 139.48, 138.73, 136.61, 134.26, 132.42, 132.16, 129.58, 127.94, 126.62, 126.06, 125.78, 125.66, 123.97, 123.57, 123.34, 122.17, 120.36, 120.24, 117.97, 45.14, 33.09, 27.83, 26.81. HRMS (ESI-TOF) m/z Calcd for C₃₀H₂₂F₇N₄O₂⁺ [M+H]⁺ 603.1631, found 603.1638.

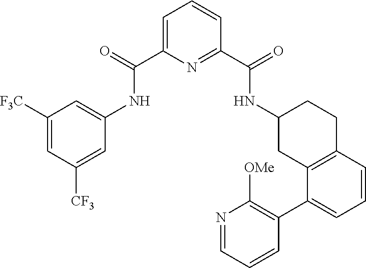 [SMILES: COc1ncccc1-c1cccc2c1CC(NC(=O)c1cccc(C(=O)Nc3cc(C(F)(F)F)cc(C(F)(F)F)c3)n1)CC2]

N²-(3,5-Bis(trifluoromethyl)phenyl)-N⁶-(8-(2-methoxypyridin-3-yl)-1,2,3,4-tetrahydronaphthalen-2-yl)pyridine-2,6-dicarboxamide (T9) Purification by silica gel chromatography eluting with hexane/EA (70/30, v/v), white solid, 56% yield. ¹H NMR (600 MHz, CDCl₃) rotameric mixture, resonances for the minor rotamer are enclosed in parenthesis [ ]: δ [9.51 (s, 0.31H)], 9.39 (s, 1H), 8.47 (dd, J=12.9, 7.8 Hz, 2H), [8.43 (d, J=7.9 Hz, 0.65H)], 8.20 (dd, J=5.0, 2.0 Hz, 1H), [8.18-8.16 (m, 0.34H)], 8.13 (t, J=7.8 Hz, 2H), 8.04 (s, 2H), 7.89 (d, J=7.4 Hz, 1H), [7.69 (s, 0.34H)], 7.61 (s, 1H), [7.57 (d, J=8.4 Hz, 0.31H)], 7.52 (dd, J=7.2, 2.0 Hz, 1H), [7.36 (d, J=6.0 Hz, 0.34H)], [7.25-7.14 (m, 2.62H)], [7.07-7.01 (m, 2.34H)], [6.90 (t, J=6.1 Hz, 0.32H)], [4.57 (s, 1.35H)], [3.93 (s, 1H)], 3.76 (s, 3H), [3.13 (dt, J=15.0, 6.9 Hz, 0.32H)], [3.06 (dd, J=11.9, 4.7 Hz, 0.67H)], 2.98 (t, J=7.6 Hz, 2H), 2.84-2.68 (m, 2H), [2.50 (dd, J=16.6, 7.4 Hz, 0.33H)], 2.38 (dq, J=11.7, 5.9 Hz, 1H), [2.25 (s, 0.32H)], [2.04 (dtd, J=11.0, 8.2, 4.1 Hz, 1.34H)]. ¹³C NMR (151 MHz, CDCl₃) rotameric mixture, resonances for the minor rotamer are enclosed in parenthesis [ ]: δ 162.40, [162.36], 161.69, [161.64], [161.01], 159.91, [149.39], 149.22, 147.76, [147.71], 146.38, [146.31], 139.79, 139.64, [138.99], 138.68, [138.61], 137.58, 136.02, [135.47], 132.33 (q, J=33.5 Hz), 131.90, 129.06, [128.71], [128.06], 127.90, 126.62, [126.31], [126.26], 126.01, 125.72, [125.69], [125.56], 124.21, [124.17], 123.88, 122.07, [120.27], 120.11-119.77 (m), [119.75], 118.05 (dq, J=8.9, 4.6, 3.8 Hz), 117.68, 116.81-116.62 (m), [53.64], 53.34, [45.38], 44.89, [33.31], 32.56, [28.28], [27.31], 26.72, 25.57. HRMS (ESI-TOF) m/z Calcd for C₃₁H₂₅F₆N₄O₃⁺ [M+H]⁺ 615.1831, found 615.1837.

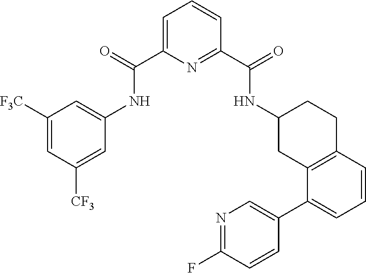 [SMILES: O=C(Nc1cc(C(F)(F)F)cc(C(F)(F)F)c1)c1cccc(C(=O)NC2CCc3cccc(-c4ccc(F)nc4)c3C2)n1]

N²-(3,5-Bis(trifluoromethyl)phenyl)-N⁶-(8-(6-fluoropyridin-3-yl)-1,2,3,4-tetrahydronaphthalen-2-yl)pyridine-2,6-dicarboxamide (T10) Purification by silica gel chromatography eluting with hexane/EA (65/35, v/v), white solid, 85% yield. ¹H NMR (600 MHz, CDCl₃) δ 10.07 (s, 1H), 8.42 (dd, J=7.8, 1.2 Hz, 1H), 8.38 (dd, J=7.8, 1.2 Hz, 1H), 8.25 (s, 2H), 8.08-8.01 (m, 2H), 7.90 (d, J=2.4 Hz, 1H), 7.70 (td, J=8.1, 2.5 Hz, 1H), 7.65 (s, 1H), 7.20 (t, J=7.6 Hz, 1H), 7.12 (d, J=8.1 Hz, 1H), 6.98 (d, J=7.0 Hz, 1H), 6.94 (dd, J=8.4, 2.2 Hz, 1H), 4.44-4.36 (m, 1H), 2.96 (dt, J=17.4, 5.1 Hz, 1H), 2.87 (ddd, J=17.1, 10.4, 6.3 Hz, 1H), 2.70 (dd, J=16.2, 5.0 Hz, 1H), 2.52 (dd, J=16.1, 9.6 Hz, 1H), 2.05-1.98 (m, 1H), 1.86 (dtd, J=12.4, 10.4, 6.0 Hz, 1H). ¹³C NMR (151 MHz, CDCl₃) δ 163.58, 162.74, 161.99, 161.85, 148.85, 148.11, 147.23 (d, J=14.3 Hz), 141.91 (d, J=8.3 Hz), 139.69, 138.85, 137.31, 135.84, 134.74 (d, J=5.0 Hz), 132.41 (q, J=33.6 Hz), 131.71, 129.12, 127.80, 126.41, 126.12, 125.79, 123.97, 122.17, 120.36, 120.06 (q, J=4.1 Hz), 118.53-117.55 (m), 109.43, 109.18, 45.92, 34.71, 28.77, 28.34. HRMS (ESI-TOF) m/z Calcd for C₃₀H₂₂F₇N₄O₂⁺ [M+H]⁺ 603.1631, found 603.1649.

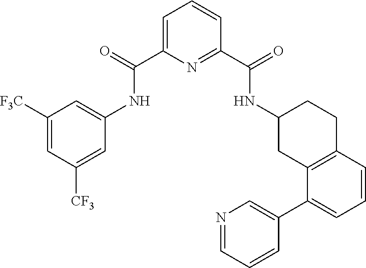 [SMILES: O=C(Nc1cc(C(F)(F)F)cc(C(F)(F)F)c1)c1cccc(C(=O)NC2CCc3cccc(-c4cccnc4)c3C2)n1]

N²-(3,5-Bis(trifluoromethyl)phenyl)-N⁶-(8-(pyridin-3-yl)-1,2,3,4-tetrahydronaphthalen-2-yl)pyridine-2,6-dicarboxamide (T11) Purification by silica gel chromatography eluting with hexane/EA (50/50, v/v), white solid, 45% yield. ¹H NMR (600 MHz, CDCl₃) δ10.05 (s, 1H), 8.52 (dd, J=4.9, 1.7 Hz, 1H), 8.42 (dd, J=7.8, 1.2 Hz, 1H), 8.40-8.37 (m, 2H), 8.22 (s, 2H), 8.07 (t, J=7.8 Hz, 1H), 7.98 (d, J=8.8 Hz, 1H), 7.66 (s, 1H), 7.59 (dt, J=7.8, 1.9 Hz, 1H), 7.31 (dd, J=7.8, 4.9 Hz, 1H), 7.22 (t, J=7.6 Hz, 1H), 7.15 (d, J=7.8 Hz, 1H), 7.01 (d, J=6.7 Hz, 1H), 4.43 (ddt, J=13.8, 9.0, 4.6 Hz, 1H), 3.03-2.89 (m, 2H), 2.79 (dd, J=16.3, 4.9 Hz, 1H), 2.59 (dd, J=16.2, 9.2 Hz, 1H), 2.07 (d, J=12.4 Hz, 1H), 1.93-1.84 (m, 1H). ¹³C NMR (151 MHz, CDCl₃) δ 162.70, 161.92, 149.69, 149.00, 148.22, 148.11, 139.60, 138.87, 138.59, 136.97, 136.58, 135.85, 132.99-131.99 (m), 131.60, 128.94, 127.84, 126.39, 126.13, 125.81, 125.74, 124.00, 123.44, 122.19, 120.39, 120.15 (dd, J=8.8, 4.6 Hz), 118.20-117.69 (m), 45.89, 34.63, 28.73, 28.23. HRMS (ESI-TOF) m/z Calcd for C₃₀H₂₃F₆N₄O₂⁺ [M+H]⁺ 585.1725, found 585.1729.

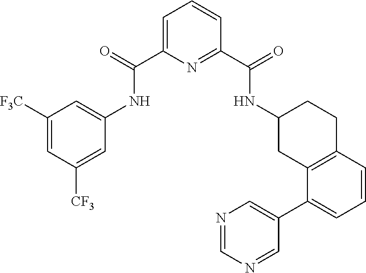 [SMILES: O=C(Nc1cc(C(F)(F)F)cc(C(F)(F)F)c1)c1cccc(C(=O)NC2CCc3cccc(-c4cncnc4)c3C2)n1]

N²-(3,5-Bis(trifluoromethyl)phenyl)-N⁶-(8-(pyrimidin-5-yl)-1,2,3,4-tetrahydronaphthalen-2-yl)pyridine-2,6-dicarboxamide (T12) Purification by silica gel chromatography eluting with hexane/EA (50/50, v/v), white solid, 72% yield. ¹H NMR (600 MHz, CDCl₃) δ 9.85 (s, 1H), 9.16 (s, 1H), 8.65 (s, 2H), 8.42 (dd, J=7.8, 1.7 Hz, 2H), 8.21 (s, 2H), 8.09 (t, J=7.8 Hz, 1H), 7.85 (d, J=8.7 Hz, 1H), 7.67 (s, 1H), 7.28 (t, J=7.6 Hz, 1H), 7.23 (d, J=7.5 Hz, 1H), 7.05 (d, J=7.4 Hz, 1H), 4.52-4.43 (m, 1H), 3.08-2.98 (m, 2H), 2.87 (dd, J=16.2, 5.0 Hz, 1H), 2.65 (dd, J=16.2, 9.1 Hz, 1H), 2.18-2.11 (m, 1H), 1.99-1.92 (m, 1H). ¹³C NMR (151 MHz, CDCl₃) δ 162.64, 161.72, 157.39, 156.61, 148.99, 148.01, 139.71, 138.70, 136.24, 134.78, 134.76, 132.51 (q, J=33.6 Hz), 131.75, 129.82, 127.96, 126.79, 126.25, 125.80, 125.77, 123.97, 122.16, 120.35, 120.17-119.80 (m), 118.32-117.89 (m), 45.75, 34.67, 28.63, 28.16. HRMS (ESI-TOF) m/z Calcd for C₂₉H₂₂F₆N₅O₂⁺ [M+H]⁺ 586.1678, found 586.1677.

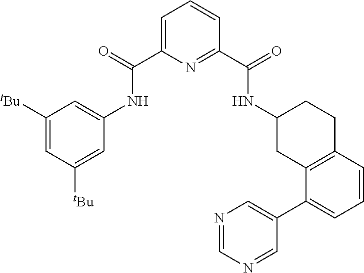 [SMILES: CC(C)(C)c1cc(NC(=O)c2cccc(C(=O)NC3CCc4cccc(-c5cncnc5)c4C3)n2)cc(C(C)(C)C)c1]

N²-(3,5-Di-tert-butylphenyl)-N⁶-(8-(pyrimidin-5-yl)-1,2,3,4-tetrahydronaphthalen-2-yl)pyridine-2,6-dicarboxamide (T13) Purification by silica gel chromatography eluting with hexane/EA (30/70, v/v), white solid, 80% yield. ¹H NMR (600 MHz, CDCl₃) δ 9.30 (s, 1H), 9.18 (s, 1H), 8.71 (s, 2H), 8.44 (dd, J=7.8, 1.2 Hz, 1H), 8.37 (dd, J=7.8, 1.2 Hz, 1H), 8.08 (t, J=7.8 Hz, 1H), 7.67 (d, J=8.4 Hz, 1H), 7.51 (d, J=1.7 Hz, 2H), 7.31-7.27 (m, 3H), 7.07 (dd, J=6.3, 2.6 Hz, 1H), 4.49 (tdd, J=10.0, 8.7, 3.3 Hz, 1H), 3.21-3.09 (m, 2H), 3.06 (dd, J=16.6, 4.7 Hz, 1H), 2.72 (dd, J=16.3, 9.0 Hz, 1H), 2.33-2.26 (m, 1H), 1.98 (dtd, J=12.6, 9.7, 6.3 Hz, 1H), 1.37 (s, 18H). ¹³C NMR (151 MHz, CDCl₃) δ 162.82, 161.23, 157.47, 156.59, 151.97, 149.20, 148.78, 139.38, 136.35, 136.31, 134.96, 134.88, 132.15, 129.83, 128.07, 126.69, 125.43, 125.37, 119.41, 115.21, 45.80, 35.02, 34.62, 31.44, 28.72, 28.29. HRMS (ESI-TOF) m/z Calcd for C₃₅H₄₀N₅O₂⁺ [M+H]⁺ 562.3182, found 562.3181.

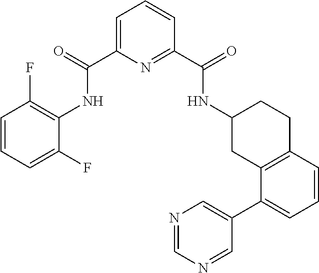 [SMILES: O=C(Nc1c(F)cccc1F)c1cccc(C(=O)NC2CCc3cccc(-c4cncnc4)c3C2)n1]

N²-(2,6-Difluorophenyl)-N⁶-(8-(pyrimidin-5-yl)-1,2,3,4-tetrahydronaphthalen-2-yl)pyridine-2,6-dicarboxamide (T14) Purification by silica gel chromatography eluting with hexane/EA (20/80, v/v), white solid, 51% yield. ¹H NMR (600 MHz, CDCl₃) δ 9.18 (s, 1H), 8.95 (s, 1H), 8.70 (s, 2H), 8.42 (dd, J=7.7, 1.2 Hz, 1H), 8.39 (dd, J=7.8, 1.2 Hz, 1H), 8.08 (t, J=7.8 Hz, 1H), 7.70 (d, J=8.5 Hz, 1H), 7.28 (ddd, J=8.5, 6.1, 2.4 Hz, 1H), 7.25-7.21 (m, 2H), 7.05-7.02 (m, 3H), 4.47 (tq, J=14.4, 5.4, 4.3 Hz, 1H), 3.13-3.06 (m, 2H), 3.00 (dd, J=16.1, 5.0 Hz, 1H), 2.71 (dd, J=16.2, 9.3 Hz, 1H), 2.28-2.24 (m, 1H), 1.96 (ddt, J=12.5, 9.8, 4.9 Hz, 1H). ¹³C NMR (151 MHz, CDCl₃) δ 162.64, 161.59, 158.70 (d, J=5.2 Hz), 157.43, 157.04 (d, J=5.2 Hz), 156.59, 148.99, 147.91, 139.35, 136.62, 134.89, 134.88, 132.10, 129.81, 128.02 (t, J=9.8 Hz), 127.89, 126.61, 125.82, 125.72, 113.47, 112.02 (d, J=4.3 Hz), 111.89 (d, J=4.3 Hz), 45.88, 34.57, 28.76, 28.27. HRMS (ESI-TOF) m/z Calcd for C₂₇H₂₂F₂N₅O₂⁺ [M+H]⁺ 486.1742, found 486.1739.

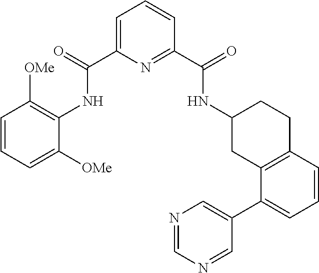 [SMILES: COc1cccc(OC)c1NC(=O)c1cccc(C(=O)NC2CCc3cccc(-c4cncnc4)c3C2)n1]

N²-(2,6-Dimethoxyphenyl)-N⁶-(8-(pyrimidin-5-yl)-1,2,3,4-tetrahydronaphthalen-2-yl)pyridine-2,6-dicarboxamide (T15) Purification by silica gel chromatography eluting with EA, white solid, 50% yield. ¹H NMR (600 MHz, CDCl₃) δ 9.18 (s, 1H), 8.76 (s, 1H), 8.69 (s, 2H), 8.41 (dd, J=7.7, 1.2 Hz, 1H), 8.34 (dd, J=7.8, 1.2 Hz, 1H), 8.03 (t, J=7.8 Hz, 1H), 7.75 (d, J=8.4 Hz, 1H), 7.26-7.20 (m, 3H), 7.02 (dd, J=6.8, 2.0 Hz, 1H), 6.66 (d, J=8.4 Hz, 2H), 4.48-4.42 (m, 1H), 3.84 (s, 6H), 3.09 (qq, J=10.7, 6.0, 5.0 Hz, 2H), 3.01 (dd, J=16.1, 4.9 Hz, 1H), 2.70 (dd, J=16.2, 9.4 Hz, 1H), 2.27 (dd, J=11.4, 4.4 Hz, 1H), 1.96-1.89 (m, 1H). ¹³C NMR (151 MHz, CDCl₃) δ 162.91, 161.61, 157.43, 156.59, 155.59, 148.96, 148.67, 138.99, 136.68, 134.93, 134.88, 132.27, 129.77, 128.20, 127.87, 126.58, 125.52, 125.12, 113.50, 104.48, 56.15, 45.86, 34.57, 28.86, 28.44. HRMS (ESI-TOF) m/z Calcd for C₂₉H₂₈N₅O₄⁺ [M+H]⁺ 510.2141, found 510.2455.

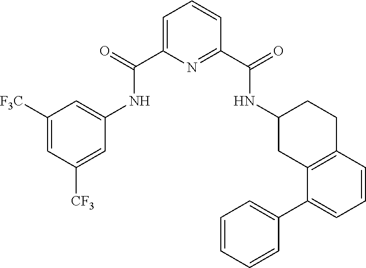 [SMILES: O=C(Nc1cc(C(F)(F)F)cc(C(F)(F)F)c1)c1cccc(C(=O)NC2CCc3cccc(-c4ccccc4)c3C2)n1]

N²-(3,5-Bis(trifluoromethyl)phenyl)-N⁶-(8-phenyl-1,2,3,4-tetrahydronaphthalen-2-yl)pyridine-2,6-dicarboxamide (T16) Purification by silica gel chromatography eluting with hexane/EA (70/30, v/v), white solid, 89% yield. ¹H NMR (600 MHz, CDCl₃) δ 9.52 (s, 1H), 8.42 (ddd, J=7.7, 5.6, 1.1 Hz, 2H), 8.15 (s, 2H), 8.09 (t, J=7.8 Hz, 1H), 7.69 (s, 1H), 7.58 (d, J=8.5 Hz, 1H), 7.37 (t, J=7.3 Hz, 2H), 7.33-7.30 (m, 1H), 7.27 (t, J=1.8 Hz, 1H), 7.26 (s, 1H), 7.24 (t, J=7.6 Hz, 1H), 7.18 (d, J=7.6 Hz, 1H), 7.10 (d, J=7.4 Hz, 1H), 4.52 (qdd, J=8.4, 4.9, 3.3 Hz, 1H), 3.05 (dd, J=14.9, 5.8 Hz, 3H), 2.73 (dd, J=16.5, 8.1 Hz, 1H), 2.23 (ddtd, J=9.5, 7.3, 4.2, 3.7, 1.6 Hz, 1H), 1.98 (dtd, J=12.7, 8.7, 6.4 Hz, 1H). ¹³C NMR (151 MHz, CDCl₃) δ 162.46, 161.68, 149.36, 147.78, 142.74, 141.26, 139.60, 138.61, 135.70, 135.70, 132.56 (q, J=33.6 Hz), 131.25, 129.03, 128.23, 128.12, 127.83, 127.09, 126.24, 125.59, 123.96, 122.16, 119.91 (q, J=4.1 Hz), 118.48-117.90 (m), 45.80, 34.23, 28.57, 27.72. HRMS (ESI-TOF) m/z Calcd for C₃₁H₂₄F₆N₃O₂⁺ [M+H]⁺ 584.1773, found 584.1780.

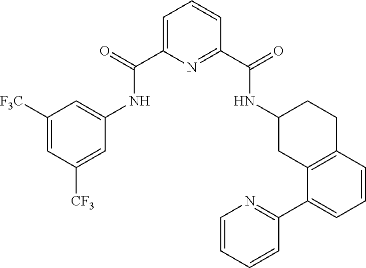 [SMILES: O=C(Nc1cc(C(F)(F)F)cc(C(F)(F)F)c1)c1cccc(C(=O)NC2CCc3cccc(-c4ccccn4)c3C2)n1]

N²-(3,5-Bis(trifluoromethyl)phenyl)-N⁶-(8-(pyridin-2-yl)-1,2,3,4-tetrahydronaphthalen-2-yl)pyridine-2,6-dicarboxamide (T17) Purification by silica gel chromatography eluting with hexane/EA (50/50, v/v), white solid, 35% yield. ¹H NMR (600 MHz, CDCl₃) δ 10.15 (s, 1H), 9.01 (d, J=6.7 Hz, 1H), 8.47-8.43 (m, 2H), 8.41 (d, J=7.7 Hz, 1H), 8.07 (t, J=7.8 Hz, 1H), 7.92 (s, 2H), 7.72 (td, J=7.7, 1.8 Hz, 1H), 7.55 (s, 1H), 7.47 (dd, J=7.8, 1.2 Hz, 1H), 7.28 (d, J=4.8 Hz, 2H), 7.22 (t, J=4.5 Hz, 1H), 7.11 (ddd, J=7.6, 4.9, 1.1 Hz, 1H), 4.58 (h, J=6.2 Hz, 1H), 3.09 (dd, J=15.7, 4.3 Hz, 1H), 2.87 (dt, J=16.0, 5.2 Hz, 1H), 2.76 (ddd, J=16.1, 10.3, 6.0 Hz, 1H), 2.68 (dd, J=15.7, 5.4 Hz, 1H), 2.45 (dt, J=13.8, 5.6 Hz, 1H), 1.83-1.74 (m, 1H). ¹³C NMR (151 MHz, CDCl₃) δ 163.14, 162.31, 159.91, 149.57, 148.64, 148.06, 140.20, 139.37, 139.17, 138.96, 137.34, 133.04, 131.94 (q, J=33.7 Hz), 128.31, 126.98, 126.28, 125.92, 125.80, 125.40, 124.43, 123.99, 122.25, 122.18, 120.24 (q, J=4.2 Hz), 118.01-116.98 (m), 45.77, 30.98, 29.18, 27.62. HRMS (ESI-TOF) m/z Calcd for C₃₀H₂₃F₆N₄O₂⁺ [M+H]⁺ 585.1725, found 585.1728.

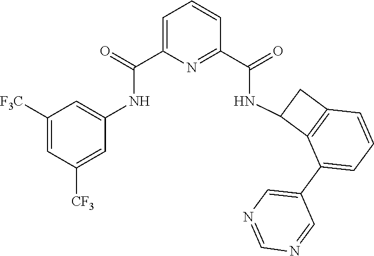 [SMILES: O=C(Nc1cc(C(F)(F)F)cc(C(F)(F)F)c1)c1cccc(C(=O)NC2Cc3cccc(-c4cncnc4)c32)n1]

N²-(3,5-Bis(trifluoromethyl)phenyl)-N⁶-(5-(pyrimidin-5-yl)bicyclo[4.2.0]octa-1 (6),2,4-trien-7-yl)pyridine-2,6-dicarboxamide (T18) Purification by silica gel chromatography eluting with hexane/EA (50/50, v/v), pale yellow solid, 56% yield. ¹H NMR (600 MHz, CDCl₃) δ 9.77 (s, 1H), 9.01 (s, 1H), 8.98 (s, 2H), 8.46 (d, J=9.3 Hz, 1H), 8.41 (dd, J=13.3, 7.5 Hz, 2H), 8.12-8.03 (m, 3H), 7.58 (s, 1H), 7.46-7.40 (m, 2H), 7.18 (d, J=6.9 Hz, 1H), 6.03 (ddd, J=9.5, 5.2, 2.5 Hz, 1H), 3.86 (dd, J=14.6, 5.2 Hz, 1H), 3.33 (dd, J=14.5, 2.5 Hz, 1H). ¹³C NMR (151 MHz, CDCl₃) δ 163.32, 162.01, 157.54, 154.83, 148.93, 148.19, 144.37, 142.46, 139.62, 138.50, 132.29 (q, J=33.6 Hz), 130.68, 130.62, 129.09, 126.56, 125.97, 125.65, 125.37, 124.12, 123.83, 122.03, 120.41 (dd, J=8.7, 4.2 Hz), 120.22, 118.35-117.96 (m), 50.42, 40.42. HRMS (ESI-TOF) m/z Calcd for C₂₇H₁₈F₆N₅O₂⁺ [M+H]⁺ 558.1365, found 558.1369.

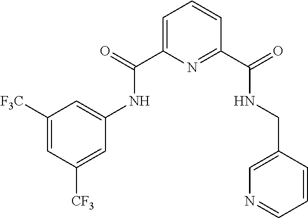 [SMILES: O=C(NCc1cccnc1)c1cccc(C(=O)Nc2cc(C(F)(F)F)cc(C(F)(F)F)c2)n1]

N²-(3,5-Bis(trifluoromethyl)phenyl)-N⁶-(pyridin-3-ylmethyl)pyridine-2,6-dicarboxamide (T19) Purification by silica gel chromatography eluting with hexane/EA (20/80, v/v), white solid, 43% yield. ¹H NMR (600 MHz, CDCl₃) δ 10.41 (s, 1H), 9.03 (t, J=6.4 Hz, 1H), 8.51 (d, J=7.8 Hz, 2H), 8.44 (dd, J=4.9, 1.6 Hz, 1H), 8.22 (d, J=2.2 Hz, 1H), 8.17 (t, J=7.8 Hz, 1H), 8.06 (s, 2H), 7.68 (dt, J=8.0, 2.0 Hz, 1H), 7.57 (s, 1H), 7.20 (dd, J=7.9, 4.8 Hz, 1H), 4.56 (d, J=6.2 Hz, 2H). ¹³C NMR (151 MHz, CDCl₃) δ 163.88, 162.18, 148.88, 148.72, 148.71, 148.33, 139.64, 138.81, 136.40, 134.65, 132.18 (q, J=33.6 Hz), 126.30, 125.96, 125.70, 124.24, 123.89, 122.09, 120.38 (dd, J=7.8, 4.1 Hz), 120.27, 118.03-117.65 (m), 40.77. HRMS (ESI-TOF) m/z Calcd for C₂₁H₁₅F₆N₄O₂⁺ [M+H]⁺ 469.1099, found 469.1093.

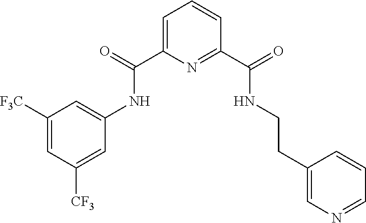 [SMILES: O=C(NCCc1cccnc1)c1cccc(C(=O)Nc2cc(C(F)(F)F)cc(C(F)(F)F)c2)n1]

N²-(3,5-Bis(trifluoromethyl)phenyl)-N⁶-(2-(pyridin-3-yl)ethyl)pyridine-2,6-dicarboxamide (T20) Purification by silica gel chromatography eluting with hexane/EA (20/80, v/v), pale yellow solid, 45% yield. ¹H NMR (600 MHz, CDCl₃) δ 9.86 (s, 1H), 8.49-8.41 (m, 4H), 8.24 (s, 2H), 8.15 (t, J=7.8 Hz, 1H), 8.11 (d, J=6.5 Hz, 1H), 7.65 (s, 1H), 7.58 (dt, J=7.8, 1.9 Hz, 1H), 7.25 (dd, J=7.7, 4.8 Hz, 1H), 3.71 (q, J=6.6 Hz, 2H), 2.96 (t, J=6.7 Hz, 2H). ¹³C NMR (151 MHz, CDCl₃) δ 163.56, 161.98, 150.10, 149.22, 148.20, 148.11, 139.65, 138.79, 136.74, 134.65, 132.35 (q, J=33.6 Hz), 126.13, 125.85, 125.81, 124.00, 123.91, 122.19, 120.74-119.75 (m), 118.23-117.79 (m), 40.56, 32.93. HRMS (ESI-TOF) m/z Calcd for C₂₂H₁₇F₆N₄O₂⁺ [M+H]⁺ 483.1256, found 483.1255.

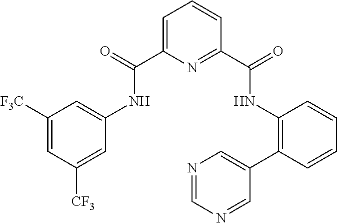 [SMILES: O=C(Nc1cc(C(F)(F)F)cc(C(F)(F)F)c1)c1cccc(C(=O)Nc2ccccc2-c2cncnc2)n1]

N²-(3,5-Bis(trifluoromethyl)phenyl)-N⁶-(2-(pyrimidin-5-yl)phenyl)pyridine-2,6-dicarboxamide (T21) Purification by silica gel chromatography eluting with hexane/EA (50/50, v/v), colorless oil, 49% yield. ¹H NMR (600 MHz, CDCl₃) δ 9.43 (s, 1H), 9.12 (s, 1H), 9.00 (s, 2H), 8.94 (s, 1H), 8.50 (ddd, J=12.2, 7.8, 1.2 Hz, 2H), 8.45 (d, J=8.3 Hz, 1H), 8.34 (s, 2H), 8.17 (t, J=7.8 Hz, 1H), 7.75 (s, 1H), 7.58 (dt, J=8.6, 4.3 Hz, 1H), 7.40 (d, J=4.4 Hz, 2H). ¹³C NMR (151 MHz, CDCl₃) δ 161.80, 161.27, 157.87, 156.95, 149.19, 148.60, 139.89, 138.29, 134.09, 132.96-131.67 (m), 132.25, 130.54, 130.27, 126.65, 126.29, 126.23, 124.04, 123.34, 122.23, 121.44 (dd, J=8.9, 5.0 Hz), 120.42, 118.69-118.21 (m). HRMS (ESI-TOF) m/z Calcd for C₂₅H₁₆F₆N₅O₂⁺ [M+H]⁺ 532.1208, found 532.1207.

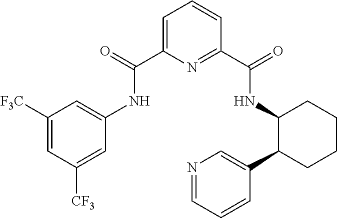 [SMILES: O=C(Nc1cc(C(F)(F)F)cc(C(F)(F)F)c1)c1cccc(C(=O)N[C@H]2CCCC[C@H]2c2cccnc2)n1]

N²-(3,5-Bis(trifluoromethyl)phenyl)-N⁶-cis-(2-(pyridin-3-yl)cyclohexyl)pyridine-2,6-dicarboxamide (cis-T22) Purification by silica gel chromatography eluting with DCM/EA (50/50, v/v), colorless oil, 12% yield. ¹H NMR (600 MHz, CDCl₃) δ 9.94 (s, 1H), 8.35-8.31 (m, 2H), 8.30 (d, J=2.3 Hz, 1H), 8.28 (d, J=7.8 Hz, 3H), 8.02 (t, J=7.8 Hz, 1H), 7.97 (d, J=8.9 Hz, 1H), 7.64 (s, 1H), 7.61 (dt, J=8.0, 2.0 Hz, 1H), 7.13 (dd, J=8.0, 4.8 Hz, 1H), 4.12 (ddt, J=15.3, 11.4, 4.0 Hz, 1H), 2.66 (td, J=11.8, 3.2 Hz, 1H), 2.15 (dd, J=12.6, 3.7 Hz, 1H), 1.86-1.75 (m, 3H), 1.52-1.43 (m, 2H), 1.40 (ddd, J=16.2, 8.1, 3.3 Hz, 1H), 1.34-1.27 (m, 1H). ¹³C NMR (151 MHz, CDCl₃) δ 162.88, 162.37, 149.53, 149.51, 148.24, 147.86, 139.27, 139.24, 138.91, 134.35, 133.15-131.62 (m), 126.03, 125.81, 125.53, 124.00, 123.97, 122.19, 120.56 (q, J=4.8, 4.2 Hz), 120.38, 118.20-117.58 (m), 52.94, 47.26, 34.38, 33.53, 25.81, 25.24. HRMS (ESI-TOF) m/z Calcd for C₂₆H₂₃F₆N₄O₂⁺ [M+H]⁺ 537.1725, found 537.1725.

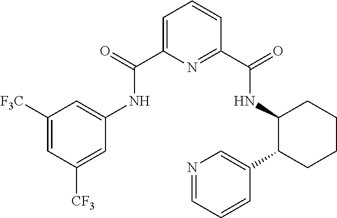 [SMILES: O=C(Nc1cc(C(F)(F)F)cc(C(F)(F)F)c1)c1cccc(C(=O)N[C@H]2CCCC[C@@H]2c2cccnc2)n1]

N²-(3,5-Bis(trifluoromethyl)phenyl)-N⁶-trans-(2-(pyridin-3-yl)cyclohexyl)pyridine-2,6-dicarboxamide (trans-T22) Purification by silica gel chromatography eluting with DCM/EA (50/50, v/v), colorless oil, 10% yield. ¹H NMR (600 MHz, CDCl₃) δ 9.74 (s, 1H), 8.65 (d, J=2.3 Hz, 1H), 8.48 (s, 2H), 8.43 (dd, J=4.8, 1.6 Hz, 1H), 8.40 (dd, J=7.7, 1.2 Hz, 1H), 8.25 (dd, J=7.8, 1.2 Hz, 1H), 8.07 (t, J=7.8 Hz, 1H), 7.71 (d, J=8.6 Hz, 2H), 7.65 (dt, J=8.0, 2.0 Hz, 1H), 7.23 (dd, J=7.9, 4.8 Hz, 1H), 4.56 (dq, J=8.1, 3.9 Hz, 1H), 3.18 (dt, J=11.2, 3.9 Hz, 1H), 2.19-2.13 (m, 1H), 2.08 (dt, J=12.3, 4.1 Hz, 1H), 2.04-1.96 (m, 2H), 1.84 (dddd, J=26.2, 12.7, 7.3, 3.6 Hz, 2H), 1.66-1.62 (m, 1H), 1.59-1.54 (m, 1H). ¹³C NMR (151 MHz, CDCl₃) δ 162.61, 162.02, 149.50, 148.46, 148.21, 148.16, 139.66, 139.00, 137.96, 135.78, 132.60 (q, J=33.6 Hz), 125.95, 125.62, 124.06, 123.68, 122.25, 120.10 (dd, J=8.6, 4.2 Hz), 118.05 (d, J=4.1 Hz), 49.86, 43.29, 30.62, 26.13, 25.10, 21.25. HRMS (ESI-TOF) m/z Calcd for C₂₆H₂₃F₆N₄O₂⁺ [M+H]⁺ 537.1725, found 537.1728.

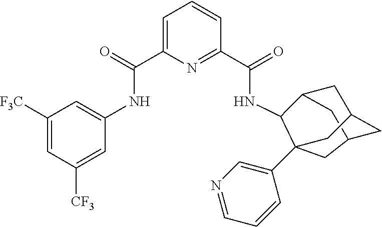 [SMILES: O=C(Nc1cc(C(F)(F)F)cc(C(F)(F)F)c1)c1cccc(C(=O)NC2C3CC4CC(C3)CC2(c2cccnc2)C4)n1]

N²-(3,5-Bis(trifluoromethyl)phenyl)-N-((1R,3S,5R,7S)-1-(pyridin-3-yl)adamantan-2-yl)pyridine-2,6-dicarboxamide (T23) Purification by silica gel chromatography eluting with hexane/EA (15/85, v/v), white solid, 20% yield. ¹H NMR (600 MHz, CDCl₃) δ 9.62 (s, 1H), 8.70 (d, J=1.7 Hz, 1H), 8.41 (dd, J=4.7, 1.6 Hz, 1H), 8.38 (s, 2H), 8.35 (dd, J=7.7, 1.1 Hz, 1H), 8.23 (dd, J=7.8, 1.1 Hz, 1H), 8.04 (t, J=7.8 Hz, 1H), 7.81 (ddd, J=8.2, 2.5, 1.6 Hz, 1H), 7.71-7.66 (m, 2H), 7.24 (dd, J=8.1, 4.7 Hz, 1H), 4.58 (dd, J=8.5, 3.1 Hz, 1H), 2.40 (q, J=3.2 Hz, 1H), 2.38-2.32 (m, 1H), 2.30-2.23 (m, 3H), 2.16 (p, J=3.2 Hz, 1H), 2.12 (dt, J=12.9, 2.7 Hz, 1H), 2.02 (dq, J=12.9, 3.0 Hz, 1H), 1.95-1.88 (m, 3H), 1.85-1.81 (m, 2H). ¹³C NMR (151 MHz, CDCl₃) δ 162.27, 161.75, 149.26, 147.97, 147.87, 146.90, 141.74, 139.67, 138.94, 133.13, 132.67 (q, J=33.6 Hz), 125.85, 125.42, 124.04, 123.62, 122.87, 122.23, 120.02-119.38 (m), 118.16-117.76 (m), 56.61, 44.81, 38.85, 36.56, 36.34, 35.44, 32.68, 31.20, 28.08, 27.70. HRMS (ESI-TOF) m/z Calcd for C₃₀H₂₇F₆N₄O₂⁺ [M+H]⁺ 589.2038, found 589.2037.

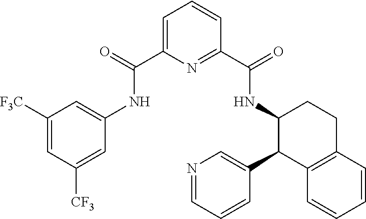 [SMILES: O=C(Nc1cc(C(F)(F)F)cc(C(F)(F)F)c1)c1cccc(C(=O)N[C@H]2CCc3ccccc3[C@H]2c2cccnc2)n1]

N²-(3,5-Bis(trifluoromethyl)phenyl)-N⁶-cis-(1-(pyridin-3-yl)-1,2,3,4-tetrahydronaphthalen-2-yl)pyridine-2,6-dicarboxamide (cis-T24) Purification by silica gel chromatography eluting with DCM/EA (80/20, v/v), colorless oil, 25% yield. ¹H NMR (600 MHz, CDCl₃) δ 9.47 (s, 1H), 8.67 (d, J=2.3 Hz, 1H), 8.46 (ddd, J=13.7, 7.8, 1.2 Hz, 2H), 8.40 (s, 2H), 8.32 (dd, J=4.7, 1.7 Hz, 1H), 8.13 (t, J=7.8 Hz, 1H), 7.72 (s, 1H), 7.26 (s, 1H), 7.26-7.25 (m, 1H), 7.24-7.18 (m, 2H), 7.18-7.13 (m, 2H), 6.97 (d, J=7.6 Hz, 1H), 4.93-4.87 (m, 1H), 4.56 (d, J=5.6 Hz, 1H), 3.18 (ddd, J=17.8, 11.4, 6.5 Hz, 1H), 3.11 (ddd, J=17.4, 6.1, 2.4 Hz, 1H), 1.92 (ddt, J=9.4, 6.1, 3.1 Hz, 1H), 1.81 (ddt, J=18.8, 12.7, 6.1 Hz, 1H). ¹³C NMR (151 MHz, CDCl₃) δ 162.71, 162.25, 151.18, 149.56, 148.69, 148.18, 139.49, 138.82, 138.40, 136.99, 135.86, 135.58, 132.22 (q, J=33.6 Hz), 130.55, 128.99, 127.39, 126.71, 126.32, 126.22, 124.14, 123.64, 122.33, 121.50 (q, J=4.3 Hz), 118.35-118.03 (m), 47.94, 45.73, 28.45, 24.33. HRMS (ESI-TOF) m/z Calcd for C₃₀H₂₃F₆N₄O₂⁺ [M+H]⁺ 585.1725, found 585.1729.

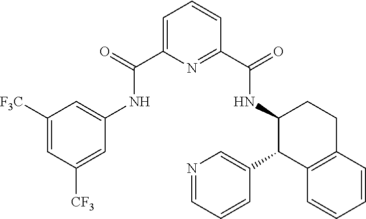 [SMILES: O=C(Nc1cc(C(F)(F)F)cc(C(F)(F)F)c1)c1cccc(C(=O)N[C@H]2CCc3ccccc3[C@@H]2c2cccnc2)n1]

N²-(3,5-Bis(trifluoromethyl)phenyl)-N⁶-trans-(1-(pyridin-3-yl)-1,2,3,4-tetrahydronaphthalen-2-yl)pyridine-2,6-dicarboxamide (trans-T24) Purification by silica gel chromatography eluting with DCM/EA (50/50, v/v), colorless oil, 15% yield. ¹H NMR (600 MHz, CDCl₃) δ 9.43 (s, 1H), 8.53-8.48 (m, 2H), 8.42 (dd, J=7.8, 1.2 Hz, 1H), 8.39 (dd, J=7.8, 1.2 Hz, 1H), 8.24 (s, 2H), 8.11 (t, J=7.8 Hz, 1H), 7.77 (d, J=8.3 Hz, 1H), 7.70 (s, 1H), 7.44 (dt, J=7.9, 2.0 Hz, 1H), 7.29 (d, J=7.2 Hz, 1H), 7.26-7.24 (m, 2H), 7.17 (t, J=7.5 Hz, 1H), 6.94 (d, J=7.7 Hz, 1H), 4.60-4.54 (m, 1H), 4.37 (d, J=6.1 Hz, 1H), 3.17 (dt, J=17.6, 6.3 Hz, 1H), 3.06 (dt, J=17.6, 7.1 Hz, 1H), 2.23 (dddd, J=14.0, 7.9, 6.3, 2.8 Hz, 1H), 2.04 (dq, J=13.4, 6.5 Hz, 1H). ¹³C NMR (151 MHz, CDCl₃) δ 162.76, 161.71, 150.49, 149.18, 148.47, 147.98, 139.69, 139.04, 138.72, 136.28, 135.81, 134.70, 132.54 (q, J=33.5 Hz), 130.80, 129.13, 127.50, 126.88, 126.06, 125.74, 124.02, 123.82, 122.21, 119.93 (dd, J=8.1, 3.8 Hz), 118.40-117.95 (m), 51.81, 48.55, 26.00, 24.49. HRMS (ESI-TOF) m/z Calcd for C₃₀H₂₃F₆N₄O₂⁺ [M+H]⁺ 585.1725, found 585.1730.

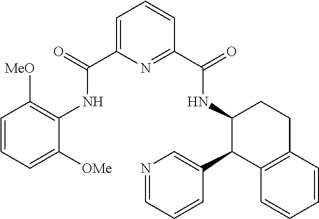 [SMILES: COc1cccc(OC)c1NC(=O)c1cccc(C(=O)N[C@H]2CCc3ccccc3[C@H]2c2cccnc2)n1]

N²-(2,6-Dimethoxyphenyl)-N⁶-cis-(1-(pyridin-3-yl)-1,2,3,4-tetrahydronaphthalen-2-yl)pyridine-2,6-dicarboxamide (cis-T25) Purification by silica gel chromatography eluting with EA, white solid, 17% yield. ¹H NMR (600 MHz, CDCl₃) δ 8.46-8.40 (m, 2H), 8.37 (d, J=7.8 Hz, 1H), 8.30 (s, 1H), 8.06 (t, J=7.8 Hz, 1H), 8.02 (d, J=4.4 Hz, 1H), 7.40 (d, J=9.3 Hz, 1H), 7.36 (d, J=8.2 Hz, 1H), 7.28 (t, J=8.4 Hz, 1H), 7.24 (d, J=7.7 Hz, 1H), 7.20 (t, J=7.4 Hz, 1H), 7.10 (t, J=7.4 Hz, 1H), 7.02 (dd, J=7.9, 4.9 Hz, 1H), 6.92 (d, J=7.7 Hz, 1H), 6.68 (d, J=8.5 Hz, 2H), 4.82 (dddd, J=12.3, 9.2, 5.6, 3.4 Hz, 1H), 4.55 (d, J=5.8 Hz, 1H), 3.83 (s, 6H), 3.22-3.07 (m, 2H), 2.03-1.96 (m, 1H), 1.90 (qd, J=12.1, 6.0 Hz, 1H). ¹³C NMR (151 MHz, CDCl₃) δ 162.80, 161.66, 155.72, 151.72, 149.19, 148.61, 148.21, 139.05, 137.69, 136.77, 135.90, 135.79, 130.63, 128.92, 128.22, 127.14, 126.51, 125.68, 125.01, 122.67, 113.51, 104.41, 56.07, 48.06, 46.02, 28.40, 24.55. HRMS (ESI-TOF) m/z Calcd for C₃₀H₂₉N₄O₄⁺ [M+H]⁺ 509.2189, found 509.2187.

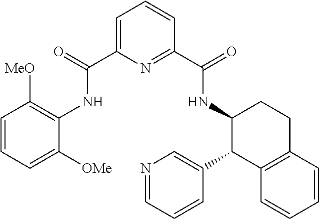 [SMILES: COc1cccc(OC)c1NC(=O)c1cccc(C(=O)N[C@H]2CCc3ccccc3[C@@H]2c2cccnc2)n1]

N²-(2,6-Dimethoxyphenyl)-N⁶-trans-(1-(pyridin-3-yl)-1,2,3,4-tetrahydronaphthalen-2-yl)pyridine-2,6-dicarboxamide (trans-T25) Purification by silica gel chromatography eluting with EA, white solid, 20% yield. ¹H NMR (600 MHz, CDCl₃) δ 8.75 (s, 1H), 8.45 (d, J=6.2 Hz, 2H), 8.40 (d, J=7.7 Hz, 1H), 8.27 (d, J=7.8 Hz, 1H), 8.00 (dt, J=14.6, 8.2 Hz, 2H), 7.48 (dt, J=8.0, 2.0 Hz, 1H), 7.28-7.24 (m, 2H), 7.21-7.17 (m, 2H), 7.10 (t, J=7.4 Hz, 1H), 7.04 (t, J=7.5 Hz, 1H), 6.80 (d, J=7.7 Hz, 1H), 6.67 (d, J=8.5 Hz, 2H), 4.56 (ddt, J=11.1, 8.2, 3.9 Hz, 1H), 4.29 (d, J=6.9 Hz, 1H), 3.86 (s, 6H), 3.14 (dt, J=17.4, 6.8 Hz, 1H), 3.03 (dt, J=17.3, 6.4 Hz, 1H), 2.22 (dtd, J=13.3, 6.6, 2.5 Hz, 1H), 1.98 (dt, J=14.0, 7.1 Hz, 1H). ¹³C NMR (151 MHz, CDCl₃) δ 163.07, 161.36, 155.47, 150.58, 148.89, 148.49, 148.33, 139.21, 139.01, 136.39, 135.87, 135.62, 130.61, 128.93, 128.03, 126.93, 126.49, 125.48, 124.90, 123.59, 113.58, 104.44, 56.17, 51.93, 48.90, 26.71, 25.86. HRMS (ESI-TOF) m/z Calcd for C₃₀H₂₉N₄O₄⁺ [M+H]⁺ 509.2189, found 509.2180.

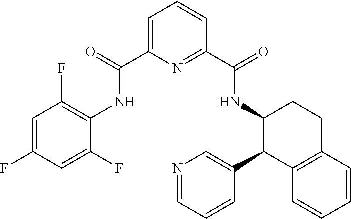 [SMILES: O=C(Nc1c(F)cc(F)cc1F)c1cccc(C(=O)N[C@H]2CCc3ccccc3[C@H]2c2cccnc2)n1]

N²-cis-(1-(Pyridin-3-yl)-1,2,3,4-tetrahydronaphthalen-2-yl)-N⁶-(2,4,6-trifluorophenyl)pyridine-2,6-dicarboxamide (cis-T26) Purification by silica gel chromatography eluting with DCM/EA (80/20, v/v), white solid, 19% yield. ¹H NMR (600 MHz, CDCl₃) δ 8.82 (s, 1H), 8.59 (d, J=2.3 Hz, 1H), 8.41 (ddd, J=11.8, 7.8, 1.2 Hz, 2H), 8.11-8.06 (m, 2H), 7.31 (d, J=9.6 Hz, 1H), 7.25-7.20 (m, 2H), 7.18 (dt, J=7.9, 2.0 Hz, 1H), 7.15-7.09 (m, 2H), 6.94 (d, J=7.7 Hz, 1H), 6.87-6.82 (m, 2H), 4.83 (tdt, J=9.2, 5.8, 3.2 Hz, 1H), 4.52 (d, J=5.8 Hz, 1H), 3.17 (ddd, J=17.8, 11.8, 6.3 Hz, 1H), 3.08 (ddd, J=17.4, 6.0, 2.3 Hz, 1H), 1.96 (ddd, J=11.9, 5.8, 2.7 Hz, 1H), 1.80 (qd, J=12.5, 6.0 Hz, 1H). ¹³C NMR (151 MHz, CDCl₃) δ 162.57, 162.43, 160.41 (t, J=14.9 Hz), 159.62 (dd, J=15.4, 7.2 Hz), 157.95 (dd, J=15.3, 7.4 Hz), 151.45, 149.28, 148.29, 148.21, 139.27, 138.18, 136.91, 135.93, 135.63, 130.60, 128.96, 127.25, 126.59, 126.05, 125.79, 123.40, 101.12-100.23 (m), 47.83, 45.78, 28.48, 24.43. HRMS (ESI-TOF) m/z Calcd for C₂₈H₂₂F₃N₄O₂⁺ [M+H]⁺ 503.1695, found 503.1697.

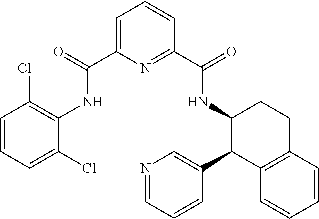 [SMILES: O=C(Nc1c(Cl)cccc1Cl)c1cccc(C(=O)N[C@H]2CCc3ccccc3[C@H]2c2cccnc2)n1]

N²-(2,6-Dichlorophenyl)-N⁶-cis-(1-(pyridin-3-yl)-1,2,3,4-tetrahydronaphthalen-2-yl)pyridine-2,6-dicarboxamide (cis-T27) Purification by silica gel chromatography eluting with DCM/EA (80/20, v/v), white solid, 13% yield. ¹H NMR (600 MHz, CDCl₃) δ 9.03 (s, 1H), 8.50 (d, J=2.3 Hz, 1H), 8.43 (ddd, J=11.9, 7.8, 1.2 Hz, 2H), 8.10 (t, J=7.8 Hz, 1H), 8.00 (dd, J=4.8, 1.7 Hz, 1H), 7.47 (d, J=8.2 Hz, 2H), 7.37 (d, J=9.5 Hz, 1H), 7.28 (t, J=8.1 Hz, 1H), 7.25-7.19 (m, 3H), 7.11 (t, J=7.4 Hz, 1H), 7.07 (dd, J=7.8, 4.7 Hz, 1H), 6.92 (d, J=7.1 Hz, 1H), 4.81 (dddd, J=12.6, 9.2, 5.8, 3.2 Hz, 1H), 4.55 (d, J=5.7 Hz, 1H), 3.18 (ddd, J=17.8, 11.6, 6.3 Hz, 1H), 3.10 (ddd, J=17.4, 6.1, 2.4 Hz, 1H), 1.99 (ddd, J=12.4, 6.1, 3.0 Hz, 1H), 1.89-1.81 (m, 1H). ¹³C NMR (151 MHz, CDCl₃) δ 162.49, 161.55, 151.50, 148.97, 148.33, 148.24, 139.35, 137.90, 136.83, 135.82, 135.76, 134.07, 132.08, 130.63, 128.93, 128.84, 128.49, 127.19, 126.56, 125.88, 125.61, 123.18, 47.96, 45.79, 28.39, 24.49. HRMS (ESI-TOF) m/z Calcd for C₂₈H₂₃Cl₂N₄O₂⁺ [M+H]⁺ 517.1198, found 517.1183.

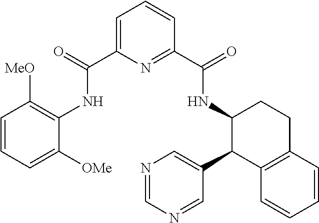 [SMILES: COc1cccc(OC)c1NC(=O)c1cccc(C(=O)N[C@H]2CCc3ccccc3[C@H]2c2cncnc2)n1]

N²-(2,6-Dimethoxyphenyl)-N⁶-cis-(1-(pyrimidin-5-yl)-1,2,3,4-tetrahydronaphthalen-2-yl)pyridine-2,6-dicarboxamide (cis-T28) Purification by silica gel chromatography eluting with DCM/EA (35/65, v/v), colorless oil, 23% yield. ¹H NMR (600 MHz, CDCl₃) δ 8.74 (s, 1H), 8.54 (s, 1H), 8.43 (dd, J=7.7, 1.2 Hz, 1H), 8.38 (s, 2H), 8.36 (dd, J=7.8, 1.2 Hz, 1H), 8.07 (t, J=7.8 Hz, 1H), 7.33 (d, J=8.7 Hz, 1H), 7.26-7.20 (m, 3H), 7.14 (t, J=7.3 Hz, 1H), 6.92 (d, J=7.4 Hz, 1H), 6.66 (d, J=8.5 Hz, 2H), 4.79 (dddd, J=12.0, 8.7, 5.7, 3.1 Hz, 1H), 4.67 (d, J=5.6 Hz, 1H), 3.82 (s, 6H), 3.20-3.12 (m, 2H), 2.03-1.99 (m, 1H), 1.89-1.81 (m, 1H). ¹³C NMR (151 MHz, CDCl₃) δ 163.29, 161.56, 158.15, 157.27, 155.60, 149.50, 148.40, 139.14, 135.67, 134.73, 134.45, 130.44, 129.11, 128.10, 127.59, 126.87, 125.95, 125.05, 113.54, 104.42, 56.09, 48.33, 43.83, 28.15, 24.46. HRMS (ESI-TOF) m/z Calcd for C₂₉H₂₈N₅O₄⁺ [M+H]⁺ 510.2141, found 510.2138.

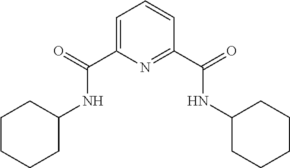 [SMILES: O=C(NC1CCCCC1)c1cccc(C(=O)NC2CCCCC2)n1]

N²,N⁶-Dicyclohexylpyridine-2,6-dicarboxamide (TC1) Recrystallization from MeOH, white solid, 70% yield. H NMR (600 MHz, CDCl₃) δ 8.34 (d, J=7.8 Hz, 2H), 8.01 (t, J=7.8 Hz, 1H), 7.55 (d, J=8.4 Hz, 2H), 4.01 (tdd, J=12.3, 7.2, 4.0 Hz, 2H), 2.05 (dt, J=8.6, 2.4 Hz, 4H), 1.77 (dq, J=12.4, 4.1 Hz, 4H), 1.67 (ddd, J=13.0, 6.3, 2.5 Hz, 2H), 1.47 (ddt, J=11.4, 10.0, 3.5 Hz, 4H), 1.36 (ddd, J=13.6, 10.6, 3.5 Hz, 4H), 1.31-1.25 (m, 2H). ¹³C NMR (151 MHz, CDCl₃) δ 162.47, 149.05, 138.95, 124.88, 48.23, 32.99, 25.56, 24.72. HRMS (ESI-TOF) m/z Calcd for C₁₉H₂₈N₃O₂⁺ [M+H]⁺ 330.2182, found 330.2183.

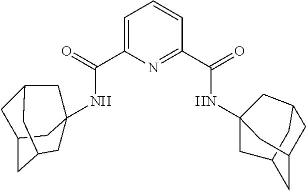 [SMILES: O=C(NC12CC3CC(CC(C3)C1)C2)c1cccc(C(=O)NC23CC4CC(CC(C4)C2)C3)n1]

N²,N⁶-Di((1s,3s)-adamantan-1-yl)pyridine-2,6-dicarboxamide (TC2) Purification by silica gel chromatography eluting with hexane/EA (70/30, v/v), white solid, 95% yield. ¹H NMR (600 MHz, CDCl₃) δ 8.29 (d, J=7.7 Hz, 2H), 7.99 (t, J=7.8 Hz, 1H), 7.49 (s, 2H), 2.17 (d, J=1.8 Hz, 18H), 1.79-1.73 (m, 12H). ¹³C NMR (151 MHz, CDCl₃) δ 162.06, 149.27, 138.99, 124.14, 51.68, 41.48, 36.35, 29.46. HRMS (ESI-TOF) m/z Calcd for C₂₇H₃₆N₃O₂⁺ [M+H]⁺ 434.2808, found 434.2807.

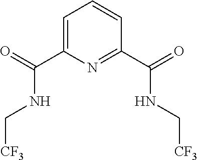 [SMILES: O=C(NCC(F)(F)F)c1cccc(C(=O)NCC(F)(F)F)n1]

N²,N⁶-Bis(2,2,2-trifluoroethyl)pyridine-2,6-dicarboxamide (TC3) Recrystallization from DCM/hexane, white solid, 77% yield. ¹H NMR (600 MHz, CDCl₃) δ 8.45 (d, J=7.8 Hz, 2H), 8.12 (t, J=7.8 Hz, 1H), 7.87 (s, 2H), 4.20 (qd, J=9.0, 6.7 Hz, 4H). ¹³C NMR (151 MHz, CD₃OD) δ 164.38, 147.62, 138.95, 124.06 (q, J=278.3 Hz), 124.85, 39.85 (q, J=35.1 Hz), 39.76 (q, J=35.1 Hz). HRMS (ESI-TOF) m/z Calcd for C₁₁H₁₀F₆N₃O₂⁺ [M+H]⁺ 330.0677, found 330.0682.

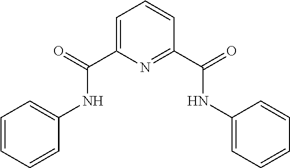 [SMILES: O=C(Nc1ccccc1)c1cccc(C(=O)Nc2ccccc2)n1]

N²,N⁶-Diphenylpyridine-2,6-dicarboxamide (TC4) Recrystallization from MeOH, white solid, 56% yield. ¹H NMR (600 MHz, Acetone-d₆) δ 10.51 (s, 2H), 8.49 (d, J=7.7 Hz, 2H), 8.34 (dd, J=8.1, 7.4 Hz, 1H), 7.98 (dd, J=8.7, 1.1 Hz, 4H), 7.42 (dd, J=8.6, 7.4 Hz, 4H), 7.18 (tt, J=7.4, 1.1 Hz, 2H). ¹³C NMR (151 MHz, Acetone-d₆) δ 160.91, 148.99, 139.29, 137.95, 128.25, 124.77, 123.72, 120.01. HRMS (ESI-TOF) m/z Calcd for C₁₉H₁₆N₃O₂⁺ [M+H]⁺ 318.1243, found 318.1245.

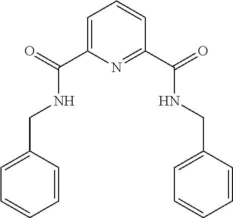 [SMILES: O=C(NCc1ccccc1)c1cccc(C(=O)NCc2ccccc2)n1]

N²,N⁶-Dibenzylpyridine-2,6-dicarboxamide (TC5) Purification by silica gel chromatography eluting with hexane/EA (50/50, v/v), white solid, 52% yield. ¹H NMR (600 MHz, Acetone-d6) δ 9.19 (s, 2H), 8.37 (d, J=7.7 Hz, 2H), 8.24 (dd, J=8.1, 7.4 Hz, 1H), 7.30 (d, J=4.7 Hz, 8H), 7.25-7.20 (m, 2H), 4.59 (d, J=6.5 Hz, 4H). ¹³C NMR (151 MHz, Acetone-d₆) δ 162.84, 148.83, 138.95, 138.79, 127.91, 126.79, 126.45, 124.16, 41.99. HRMS (ESI-TOF) m/z Calcd for C₂₁H₂₀N₃O₂⁺ [M+H]⁺ 346.1556, found 346.1555.

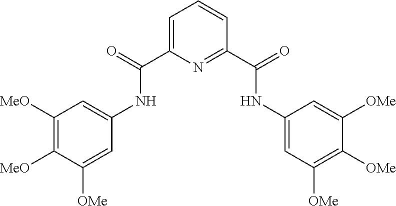 [SMILES: COc1cc(NC(=O)c2cccc(C(=O)Nc3cc(OC)c(OC)c(OC)c3)n2)cc(OC)c1OC]

N²,N⁶-Bis(3,4,5-trimethoxyphenyl)pyridine-2,6-dicarboxamide (TC6) Recrystallization from MeOH, white solid, 80% yield. ¹H NMR (600 MHz, Acetone-d₆) δ 10.42 (s, 2H), 8.47 (d, J=7.7 Hz, 2H), 8.34 (dd, J=8.1, 7.3 Hz, 1H), 7.44 (s, 4H), 3.87 (s, 12H), 3.73 (s, 6H). 13C NMR (151 MHz, Acetone-d₆) δ 160.60, 152.94, 148.91, 139.42, 134.52, 133.87, 124.60, 97.72, 59.29, 54.96. HRMS (ESI-TOF) m/z Calcd for C₂₅H₂₈N₃O₈⁺ [M+H]⁺ 498.1876, found 498.1867.

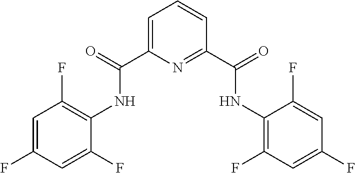 [SMILES: O=C(Nc1c(F)cc(F)cc1F)c1cccc(C(=O)Nc2c(F)cc(F)cc2F)n1]

N²,N⁶-Bis(2,4,6-trifluorophenyl)pyridine-2,6-dicarboxamide (TC7) Recrystallization from toluene, white solid, 85% yield. ¹H NMR (600 MHz, Acetone-d₆) δ 10.20 (s, 2H), 8.51 (d, J=7.8 Hz, 2H), 8.39 (dd, J=8.2, 7.3 Hz, 1H), 7.12 (dd, J=9.0, 7.6 Hz, 4H). ¹³C NMR (151 MHz, Acetone-d₆) δ 161.69, 159.94, 159.36 (dd, J=15.7, 7.4 Hz), 157.70 (dd, J=15.8, 7.6 Hz), 147.79, 139.65, 125.40, 100.90-99.71 (m). HRMS (ESI-TOF) m/z Calcd for C₁₉H₁₀F₆N₃O₂⁺ [M+H]⁺ 426.0677, found 426.0669.

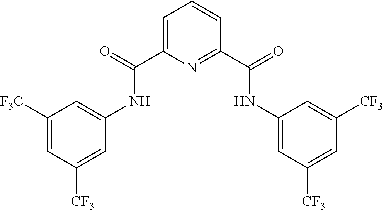 [SMILES: O=C(Nc1cc(C(F)(F)F)cc(C(F)(F)F)c1)c1cccc(C(=O)Nc2cc(C(F)(F)F)cc(C(F)(F)F)c2)n1]

N²,N⁶-Bis(3,5-bis(trifluoromethyl)phenyl)pyridine-2,6-dicarboxamide (TC8) Recrystallization from MeOH, white solid, 95% yield. ¹H NMR (600 MHz, Acetone-d₆) δ 11.13 (s, 2H), 8.73 (s, 4H), 8.57 (d, J=7.7 Hz, 2H), 8.44 (dd, J=8.1, 7.3 Hz, 1H), 7.83 (dt, J=1.6, 0.9 Hz, 2H). ¹³C NMR (151 MHz, Acetone-d₆) δ 161.56, 147.94, 139.98, 139.76, 131.24 (q, J=33.5 Hz), 125.57, 123.97, 122.17, 119.68 (d, J=4.5 Hz), 117.16-115.69 (m). HRMS (ESI-TOF) m/z Calcd for C₂₃H₁₂F₆N₃O₂⁺ [M+H]⁺ 590.0738, found 590.0742.

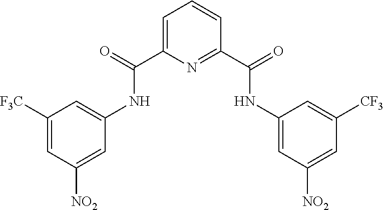 [SMILES: O=C(Nc1cc([N+](=O)[O-])cc(C(F)(F)F)c1)c1cccc(C(=O)Nc2cc([N+](=O)[O-])cc(C(F)(F)F)c2)n1]

N²,N⁶-Bis(3-trifluoromethyl-5-nitrophenyl)pyridine-2,6-dicarboxamide (TC9) Recrystallization from MeOH, pale white solid, 96% yield. ¹H NMR (600 MHz, Acetone-d₆) δ 11.24 (s, 2H), 9.28 (s, 2H), 8.82 (s, 2H), 8.58 (d, J=7.7 Hz, 2H), 8.45 (t, J=7.7 Hz, 1H), 8.30 (s, 2H). ¹³C NMR (151 MHz, Acetone-d₆) δ 161.70, 148.59, 147.84, 140.15, 140.03, 131.88-130.35 (m), 125.76, 121.70 (q, J=4.0 Hz), 117.48, 114.74 (q, J=4.1 Hz). HRMS (ESI-TOF) m/z Calcd for C₂₁H₁₂F₆N₅O⁺ [M+H]⁺ 544.0692, found 544.0691,

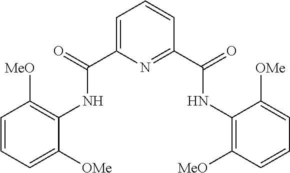 [SMILES: COc1cccc(OC)c1NC(=O)c1cccc(C(=O)Nc2c(OC)cccc2OC)n1]

N²,N⁶-Bis(2,6-dimethoxyphenyl)pyridine-2,6-dicarboxamide (TC10) Purification by silica gel chromatography eluting with hexane/EA (30/70, v/v), pale white solid, 90% yield. ¹H NMR (600 MHz, Acetone-d₆) δ 9.81 (s, 2H), 8.42 (d, J=7.7 Hz, 2H), 8.27 (dd, J=8.1, 7.4 Hz, 1H), 7.25 (t, J=8.4 Hz, 2H), 6.71 (d, J=8.4 Hz, 4H), 3.78 (s, 12H). ¹³C NMR (151 MHz, Acetone-d₆) δ 161.19, 156.24, 149.20, 138.68, 127.52, 124.20, 114.23, 103.81, 54.89. HRMS (ESI-TOF) m/z Calcd for C₂₃H₂₄N₃O₆⁺ [M+H]⁺ 438.1665, found 438.1660.

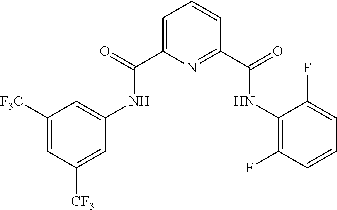 [SMILES: O=C(Nc1cc(C(F)(F)F)cc(C(F)(F)F)c1)c1cccc(C(=O)Nc2c(F)cccc2F)n1]

N²-(3,5-Bis(trifluoromethyl)phenyl)-N⁶-(2,6-difluorophenyl)pyridine-2,6-dicarboxamide (TC11) Recrystallization from MeOH, pale white solid, 82% yield. ¹H NMR (600 MHz, Acetone-d₆) δ 10.93 (s, 1H), 10.07 (s, 1H), 8.57 (dd, J=7.8, 1.2 Hz, 1H), 8.53-8.47 (m, 3H), 8.41 (t, J=7.8 Hz, 1H), 7.82-7.76 (m, 1H), 7.46 (tt, J=8.5, 6.2 Hz, 1H), 7.16 (t, J=8.2 Hz, 2H). ¹³C NMR (151 MHz, Acetone-d₆) δ 161.75, 161.49, 159.09 (d, J=4.9 Hz), 157.43 (d, J=5.0 Hz), 148.06, 147.97, 139.73, 139.68, 131.98-130.00 (m), 128.42 (t, J=10.0 Hz), 125.50 (d, J=17.5 Hz), 123.91, 122.10, 119.88 (q, J=4.3 Hz), 116.62 (dt, J=8.5, 4.2 Hz), 113.67 (t, J=16.8 Hz), 111.36 (dd, J=19.8, 4.4 Hz). HRMS (ESI-TOF) m/z Calcd for C₂₁H₁₂F₈N₃O₂⁺ [M+H]⁺ 490.0802, found 490.0801.

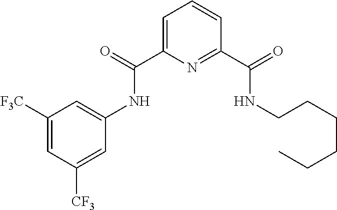 [SMILES: CCCCCCNC(=O)c1cccc(C(=O)Nc2cc(C(F)(F)F)cc(C(F)(F)F)c2)n1]

N²-(3,5-bis(trifluoromethyl)phenyl)-N⁶-hexylpyridine-2,6-dicarboxamide (TC12) Purification by silica gel chromatography eluting with hexane/EA (60/40, v/v), pale white solid, 92% yield. ¹H NMR (600 MHz, CDCl₃) δ 9.93 (s, 1H), 8.50 (d, J=7.7 Hz, 2H), 8.31 (s, 2H), 8.16 (t, J=7.8 Hz, 1H), 7.90 (t, J=6.2 Hz, 1H), 7.70 (s, 1H), 3.53 (q, J=6.8 Hz, 2H), 1.66 (td, J=14.3, 13.8, 6.7 Hz, 2H), 1.38 (t, J=7.5 Hz, 2H), 1.31 (td, J=3.9, 1.8 Hz, 4H), 0.89 (t, J=6.8 Hz, 3H). ¹³C NMR (151 MHz, CDCl₃) δ 163.24, 161.80, 158.10, 149.41, 147.89, 139.67, 138.76, 132.57 (d, J=33.7 Hz), 126.17, 125.56, 123.96, 122.15, 120.21-119.61 (m), 118.05, 39.89, 31.47, 29.74, 26.77, 22.56, 13.97. HRMS (ESI-TOF) m/z Calcd for C₂₁H₂₂F₆N₃O₂⁺ [M+H]⁺ 462.1616, found 462.1620.

### 2.2 Preparation and Determination Absolute Configuration of (S,S)-T25

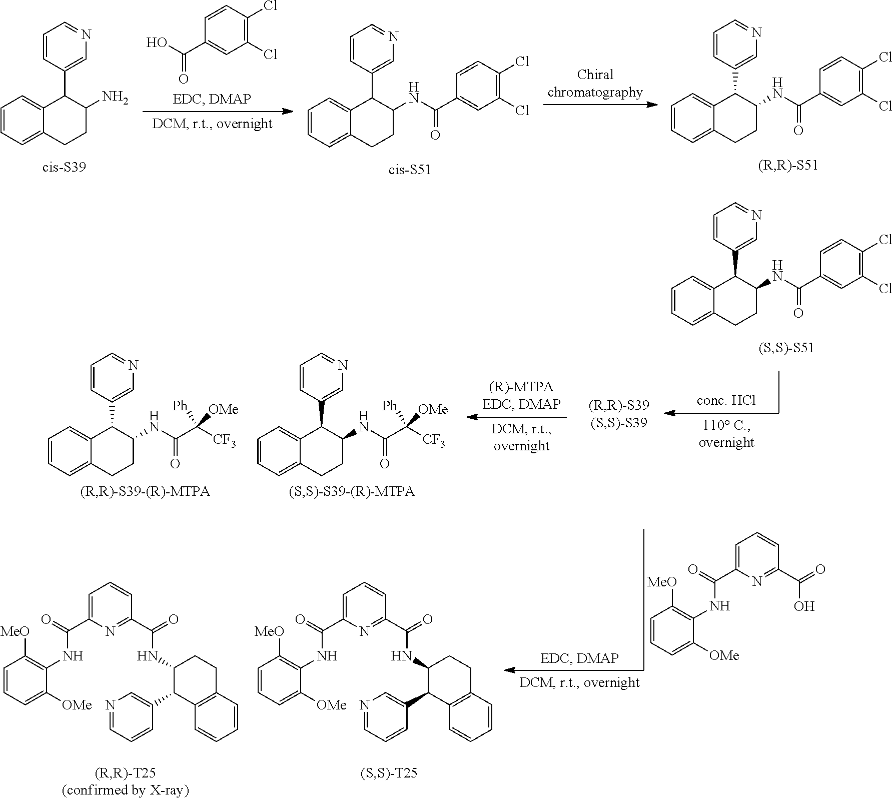

Following the procedure for synthesis of cis-T25 with cis-S39 and 3,4-dichlorobenzoic acid. Compound cis-S51 was obtained as a white solid. ¹H NMR (600 MHz, CDCl₃) δ 8.42 (s, 1H), 8.26 (s, 1H), 7.75 (d, J=2.1 Hz, 1H), 7.47 (d, J=8.3 Hz, 1H), 7.42 (dd, J=8.3, 2.1 Hz, 1H), 7.24-7.22 (m, 2H), 7.21-7.15 (m, 1H), 7.12 (ddd, J=8.4, 6.3, 2.5 Hz, 1H), 6.92 (d, J=7.7 Hz, 1H), 5.72 (d, J=8.0 Hz, 1H), 4.74-4.68 (m, 1H), 4.66 (d, J=5.7 Hz, 1H), 3.13 (qdd, J=14.3, 8.5, 4.9 Hz, 2H), 1.94-1.82 (m, 2H). ¹³C NMR (151 MHz, CDCl₃) δ 164.76, 151.16, 148.21, 137.84, 136.14, 135.87, 135.46, 134.09, 133.24, 130.80, 130.75, 129.22, 128.88, 127.17, 126.62, 125.81, 49.17, 45.31, 28.35, 24.08. HRMS (ESI-TOF) m/z Calcd for C₂₂H₁₉Cl₂N₂O⁺ [M+H]⁺ 397.0874, found 397.0880.

(R,R)—S51 and (S,S)—S51 were separated by chiral chromatography on a chiralpak OD column (30% IPA/hexane, 6.0 mL/min). Retention time: 15 min [(R,R)—S51], 19 min [(S,S)—S51].

(R,R)—S39 and (S,S)—S39 were prepared through treating (R,R)—S51 and (S,S)—S51 with conc. HCl at 110° C. for overnight. The reaction mixture was basified with NaOH solution and extracted with EA. The crude products were used in the next step without further purification.

Following the procedure for synthesis of cis-T25 with (R,R)—S39 [or (S,S)—S39] and Mosher reagent (R)-MTPA. Compounds (R,R)-S39-(R)-MTPA and (S,S)-S39-(R)-MTPA were obtained as colorless oil. For (R,R)-S39-(R)-MTPA, ¹H NMR (600 MHz, CDCl₃) δ 8.50 (dd, J=4.8, 1.7 Hz, 1H), 8.32 (d, J=2.0 Hz, 1H), 7.46 (dd, J=6.5, 2.8 Hz, 2H), 7.42-7.36 (m, 3H), 7.33 (dt, J=7.9, 2.1 Hz, 1H), 7.22 (ddd, J=7.8, 4.8, 0.9 Hz, 1H), 7.21-7.16 (m, 2H), 7.11-7.08 (m, 1H), 6.90 (d, J=7.6 Hz, 1H), 6.53 (d, J=8.6 Hz, 1H), 4.60-4.55 (m, 1H), 4.53 (d, J=5.7 Hz, 1H), 3.24 (q, J=1.5 Hz, 3H), 3.10-3.01 (m, 2H), 1.92-1.86 (m, 1H), 1.84-1.79 (m, 1H). ¹³C NMR (151 MHz, CDCl₃) δ 165.72, 151.43, 148.32, 137.98, 136.75, 136.04, 135.38, 132.26, 130.65, 129.52, 128.86, 128.67, 127.58 (q, J=1.3 Hz), 127.08, 126.56, 123.85 (q, J=221.4 Hz), 123.06, 83.91 (q, J=26.0 Hz), 54.92 (q, J=2.1 Hz), 48.69, 45.76, 28.08, 24.43. ¹⁹F NMR (376 MHz, CDCl₃) δ −70.73. HRMS (ESI-TOF) m/z Calcd for C₂₅H₂₄F₃N₂O₂⁺ [M+H]⁺ 441.1790, found 441.1792. For (S,S)-S39-(R)-MTPA, H NMR (600 MHz, CDCl₃) δ 8.45 (dd, J=4.7, 1.8 Hz, 1H), 8.15 (d, J=2.1 Hz, 1H), 7.50 (td, J=6.3, 5.1, 3.0 Hz, 2H), 7.49-7.43 (m, 3H), 7.22-7.18 (m, 2H), 7.10-7.07 (m, 1H), 7.03 (ddd, J=7.9, 4.7, 0.9 Hz, 1H), 7.00 (dt, J=7.9, 2.0 Hz, 1H), 6.84 (d, J=7.8 Hz, 1H), 6.40 (d, J=8.7 Hz, 1H), 4.63-4.58 (m, 1H), 4.38 (d, J=5.6 Hz, 1H), 3.31 (t, J=1.6 Hz, 3H), 3.11-3.05 (m, 2H), 1.91-1.87 (m, 2H). ¹³C NMR (151 MHz, CDCl₃) δ 165.51, 151.25, 148.16, 137.89, 136.79, 135.73, 135.56, 132.27, 130.50, 129.56, 128.97, 128.71, 127.52 (q, J=1.3 Hz), 127.14, 126.53, 123.63 (q, J=221.0 Hz), 123.05, 83.73, 54.89 (q, J=1.5 Hz), 48.30, 45.80, 27.90, 24.63. ¹⁹F NMR (376 MHz, CDCl₃) δ −71.39. HRMS (ESI-TOF) m/z Calcd for C₂₅H₂₄F₃N₂O₂⁺ [M+H]⁺ 441.1790, found 441.1792.

### Mosher's Method Analysis:

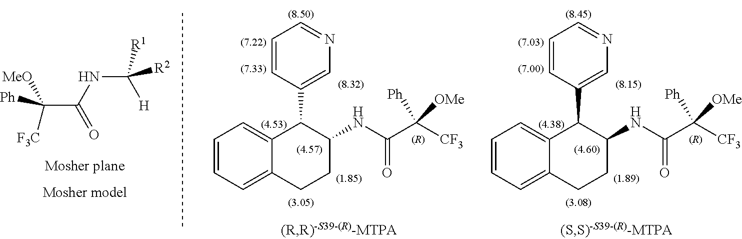

Considering mosher model, the protons in the R² of the molecule are shifted upfield relative to the protons in the R¹, which is the result of anisotropic shielding of phenyl group. In (R,R)-S39-(R)-MTPA, the pyridine portion is trans to the phenyl group. In (S,S)-S39-(R)-MTPA, the pyridine portion is cis to the phenyl group. Therefore, the absolute configurations of all derivatives are confirmed. These configurations are also confirmed by the X-ray of (R,R)-T25.

### 2.3 Condition Optimization

Screening reactions were carried out on a 0.02 mmol scale. Yield and selectivity based on H NMR analysis using 1,3,5-trimethoxybenzene as the internal standard. The yield of single target product is shown in tables.

TABLE S1
Template optimization for C6-selective C—H olefination of quinoline

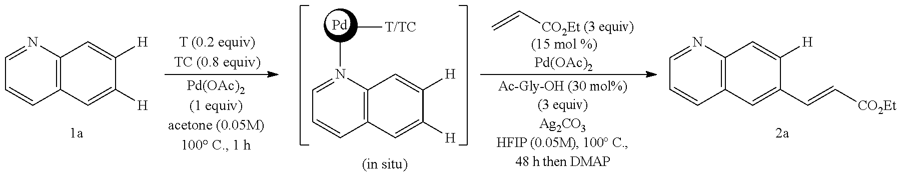

Template: (with TC8)

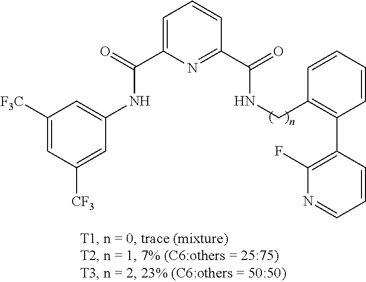

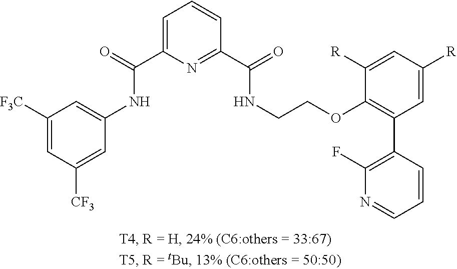

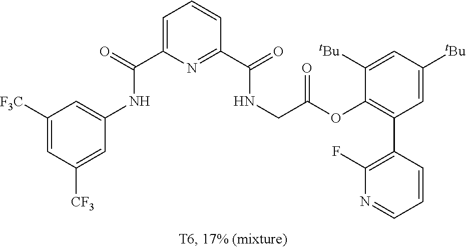 [SMILES: CC(C)(C)c1cc(-c2cccnc2F)c(OC(=O)CNC(=O)c2cccc(C(=O)Nc3cc(C(F)(F)F)cc(C(F)(F)F)c3)n2)c(C(C)(C)C)c1]

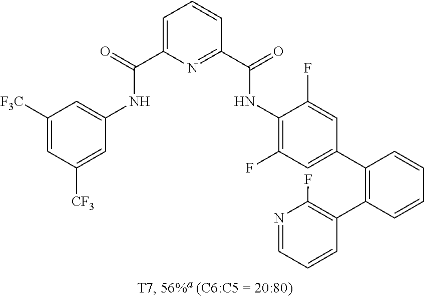 [SMILES: O=C(Nc1cc(C(F)(F)F)cc(C(F)(F)F)c1)c1cccc(C(=O)Nc2c(F)cc(-c3ccccc3-c3cccnc3F)cc2F)n1]

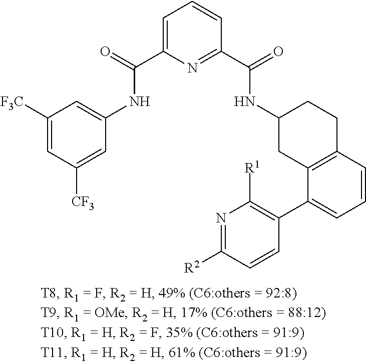

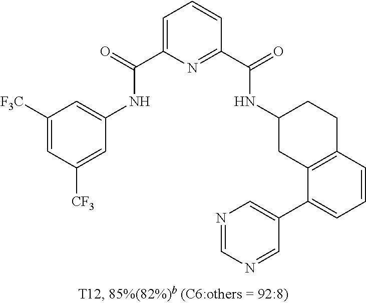 [SMILES: O=C(Nc1cc(C(F)(F)F)cc(C(F)(F)F)c1)c1cccc(C(=O)NC2CCc3cccc(-c4cncnc4)c3C2)n1]

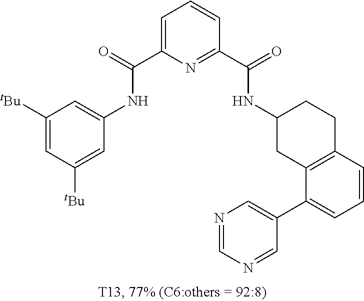 [SMILES: CC(C)(C)c1cc(NC(=O)c2cccc(C(=O)NC3CCc4cccc(-c5cncnc5)c4C3)n2)cc(C(C)(C)C)c1]

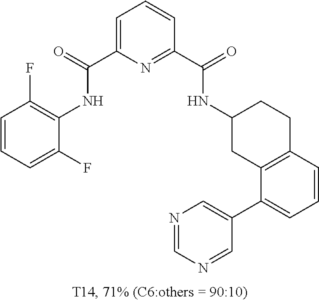 [SMILES: O=C(Nc1c(F)cccc1F)c1cccc(C(=O)NC2CCc3cccc(-c4cncnc4)c3C2)n1]

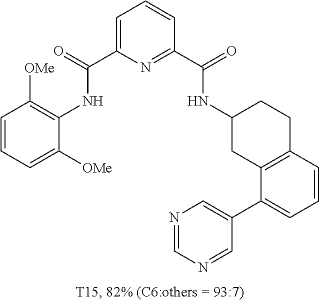 [SMILES: COc1cccc(OC)c1NC(=O)c1cccc(C(=O)NC2CCc3cccc(-c4cncnc4)c3C2)n1]

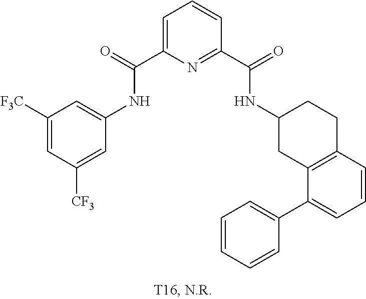 [SMILES: O=C(Nc1cc(C(F)(F)F)cc(C(F)(F)F)c1)c1cccc(C(=O)NC2CCc3cccc(-c4ccccc4)c3C2)n1]

Template chaperone: (with T12)

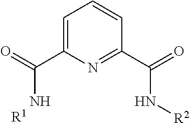

TC1, R¹ = R₂ = cyclohexyl, 79% (C6:others = 93:7)
TC2, R₁ = R₂ = adamantyl, 43% (C6:others = 88:12)
TC3, R₁ = R₂ = CH₂CF₃, 66% (C6:others = 93:7)
TC4, R₁ = R₂ = Ph, N.R.
TC5, R₁ = R₂ = Bn, 16% (C6:others = 75:25)
TC6, R¹ = R₂ = 3,4,5-OMeC₆H₂, 31% (C6:others = 88:12)
TC7, R¹ = R₂ = 2,4,6-FC₆H₂, 51% (C6:others = 94:6)
TC8, R¹ = R₂ = 3,5-CF₃C₆H_{3,} 85% (C6:others = 92:8)
TC9, R¹ = R₂ = 3-CF3, 5-NO₂C₆H₃, 65% (C6:others = (88:12)
TC10, R¹ = R₂ = 2,6-OMeC₆H₃, 52% (C6:others = 93:7)
TC11, R¹ = 3,5-CF₃C₆H₃, R² = 2,6-FC₆H₃, 75% (C6:others = 93:7)
TC12, R¹ = 3,5-CF₃C₆H₃, R² = ⁿBu, 82% (C6:others = 92:8)
^{a}The yield of C5-olefinated product.
^{b}Isolated yield.

TABLE S2
Condition optimization for C6-selective C—H olefination of quinoline

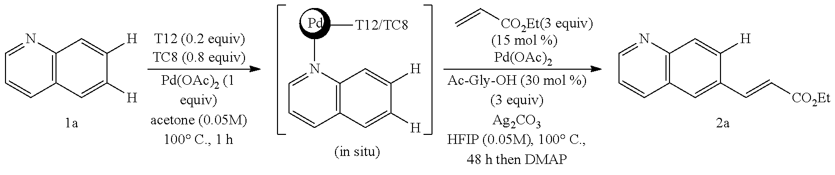

| | | Yield (%), |
|---|---|---|
| | | Selectivity |
| Entry | Deviation from standard conditions | (C6:others) |
| 1 | none | 85, (92:8) |
| 2 | T12 (1 equiv), w/o TC8 | 84, (92:8) |
| 3 | TC8 (1 equiv), w/o T12 | N.R., (−) |
| 4 | Pd(OAc)₂ (0.2 equiv) in the first step, w/o | 27, (83:17) |
| | TC8 | |
| 5 | DCM instead of acetone | 75, (92:8) |
| 6 | MeCN instead of acetone | 80, (92:8) |
| 7 | w/o Pd(OAc)₂ | N.R., (−) |
| 8 | w/o Ac-Gly-OH | 9, (67:33) |
| 9 | w/o Ag₂CO₃ | 48, (90:10) |
| 10 | Pd(MeCN)₂Cl₂ instead of Pd(OAc)₂ | 60, (89:11) |
| 11 | Ac-L-Phe-OH instead of Ac-Gly-OH | 41, (90:10) |
| 12 | TFA-Gly-OH instead of Ac-Gly-OH | 25, (87:13) |
| 13 | Formyl-Gly-OH instead of Ac-Gly-OH | 62, (93:7) |
| 14 | AgOAc instead of Ag₂CO₃ | 70, (91:9) |
| 15 | Cu(OAc₂)₂ instead of Ag₂CO₃ | 27, (80:20) |

TABLE S3
Template optimization for C7-selective C—H olefination of 3-methylquinoline

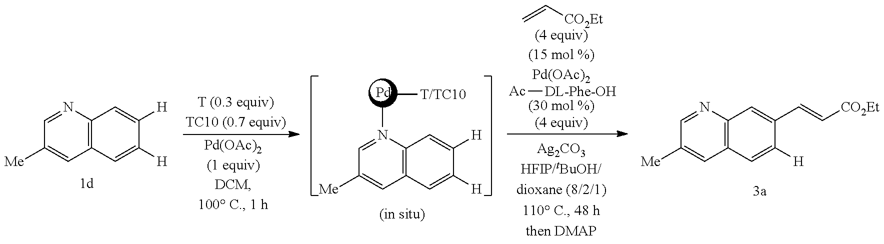

Template (with TC10)

| | |
|---|---|
| 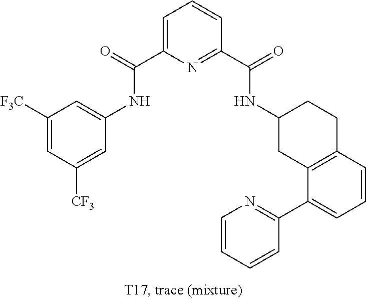 [SMILES: O=C(Nc1cc(C(F)(F)F)cc(C(F)(F)F)c1)c1cccc(C(=O)NC2CCc3cccc(-c4ccccn4)c3C2)n1] | 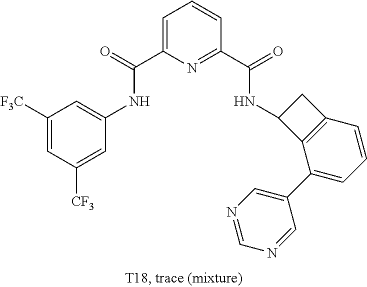 [SMILES: O=C(Nc1cc(C(F)(F)F)cc(C(F)(F)F)c1)c1cccc(C(=O)NC2Cc3cccc(-c4cncnc4)c32)n1] |
| 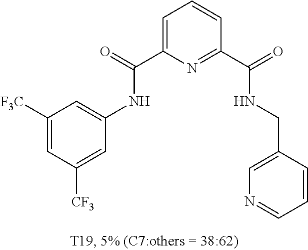 [SMILES: O=C(NCc1cccnc1)c1cccc(C(=O)Nc2cc(C(F)(F)F)cc(C(F)(F)F)c2)n1] | 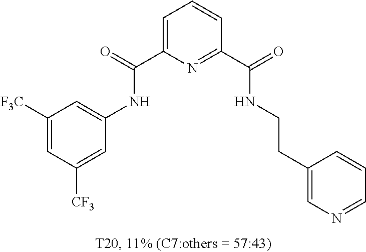 [SMILES: O=C(NCCc1cccnc1)c1cccc(C(=O)Nc2cc(C(F)(F)F)cc(C(F)(F)F)c2)n1] |
| 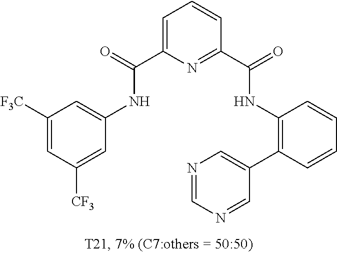 [SMILES: O=C(Nc1cc(C(F)(F)F)cc(C(F)(F)F)c1)c1cccc(C(=O)Nc2ccccc2-c2cncnc2)n1] | 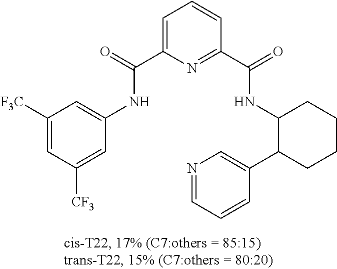 [SMILES: O=C(Nc1cc(C(F)(F)F)cc(C(F)(F)F)c1)c1cccc(C(=O)NC2CCCCC2c2cccnc2)n1] |
| 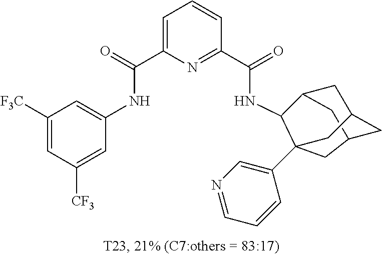 [SMILES: O=C(Nc1cc(C(F)(F)F)cc(C(F)(F)F)c1)c1cccc(C(=O)NC2C3CC4CC(C3)CC2(c2cccnc2)C4)n1] | 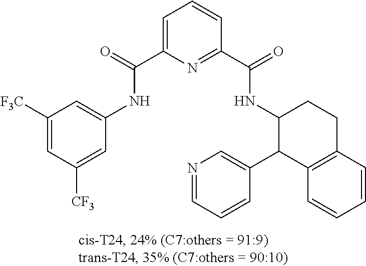 [SMILES: O=C(Nc1cc(C(F)(F)F)cc(C(F)(F)F)c1)c1cccc(C(=O)NC2CCc3ccccc3C2c2cccnc2)n1] |
| 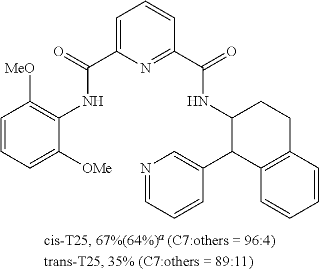 [SMILES: COc1cccc(OC)c1NC(=O)c1cccc(C(=O)NC2CCc3ccccc3C2c2cccnc2)n1] | 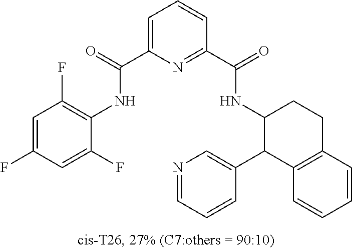 [SMILES: O=C(Nc1c(F)cc(F)cc1F)c1cccc(C(=O)NC2CCc3ccccc3C2c2cccnc2)n1] |
| 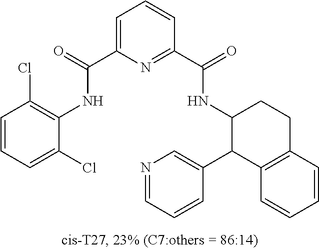 [SMILES: O=C(Nc1c(Cl)cccc1Cl)c1cccc(C(=O)NC2CCc3ccccc3C2c2cccnc2)n1] | 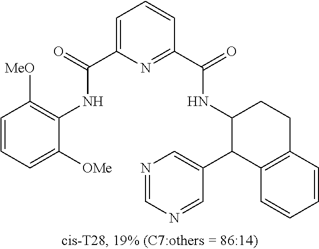 [SMILES: COc1cccc(OC)c1NC(=O)c1cccc(C(=O)NC2CCc3ccccc3C2c2cncnc2)n1] |

^{a}Isolated yield.

TABLE S4
Condition optimization for C7-selective C—H olefination of 3-methylquinoline

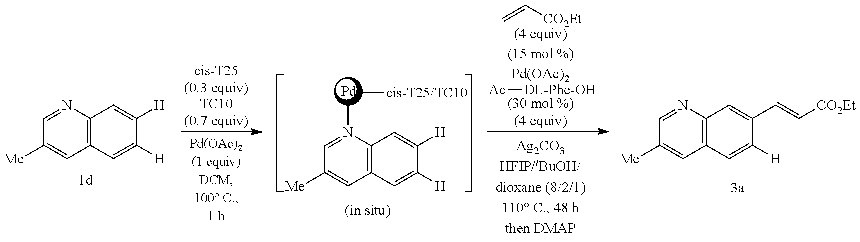

| | | Yield (%), |
|---|---|---|
| | | Selectivity |
| Entry | Deviation from standard condtiions | (C7:others) |
| 1 | none | 67, (96:4) |
| 2 | cis-T25 (1 equiv), w/o TC10 | 60, (96:4) |
| 3 | TC10 (1 equiv), w/o cis-T25 | N.R., (−) |
| 4 | Pd(OAc)₂ (0.3 equiv) in the first step, w/o TC10 | 33, (84:16) |
| 5 | acetone instead of DCM | 53, (96:4) |
| 6 | MeCN instead of DCM | 56, (94:6) |
| 7 | w/o Pd(OAc)₂ | N.R., (−) |
| 8 | w/o Ac-DL-Phe-OH | N.R., (−) |
| 9 | w/o Ag₂CO₃ | 6, (96:4) |
| 10 | Pd(MeCN)₂Cl₂ instead of Pd(OAc)₂ | 39, (94:6) |
| 11 | Ac-Gly-OH instead of Ac-DL-Phe-OH | 30, (89:11) |
| 12 | Ac-L-Phe-OH instead of Ac-DL-Phe-OH | 53, (96:4) |
| 13 | Ac-D-Phe-OH instead of Ac-DL-Phe-OH | 56, (96:4) |
| 14 | AgOAc instead of Ag₂CO₃ | 40, (94:6) |
| 15 | Cu(OAc)₂ instead of Ag₂CO₃ | 5, (−) |
| 16 | HFIP instead of standard solvent | 39, (96:4) |
| 17 | ^{t}BuOH instead of standard solvent | 48, (96:4) |
| 18 | dioxane instead of standard solvent | 10, (96:4) |
| 19 | HFIP/^{t}BuOH (4/1) instead of standard solvent | 32, (96:4) |

TABLE S5
Condition optimization for C7-selective C—H olefination of quinoline

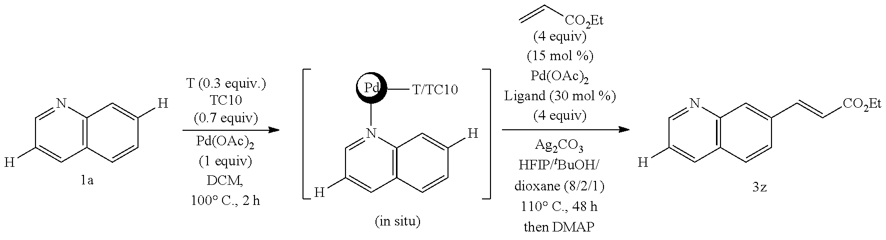

w/Ac-L-Phe-OH

| | |
|---|---|
| 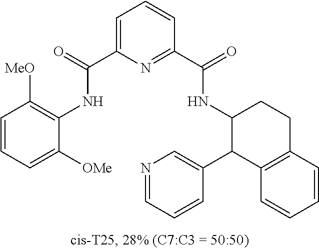 [SMILES: COc1cccc(OC)c1NC(=O)c1cccc(C(=O)NC2CCc3ccccc3C2c2cccnc2)n1] | 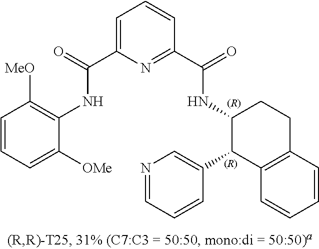 |

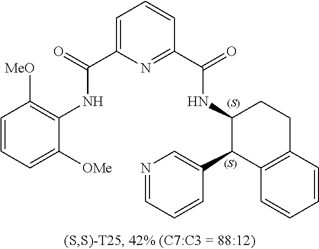

w/(S,S)-T25

| | |
|---|---|
| 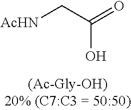 | 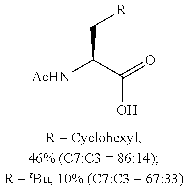 |
| 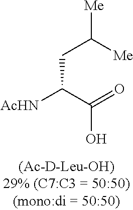 [SMILES: CC(=O)N[C@H](CC(C)C)C(=O)O] | 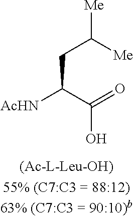 [SMILES: CC(=O)N[C@@H](CC(C)C)C(=O)O] |
| 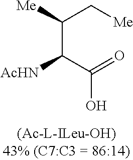 [SMILES: CC[C@H](C)[C@H](NC(C)=O)C(=O)O] | 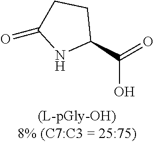 |

^{a}Di-product represents both C3 and C7-olefinated quinoline.
^{b}Using Pd(MeCN)₂Cl₂

TABLE S6
Condition optimization for C6-selective C—H alynylation of quinoline

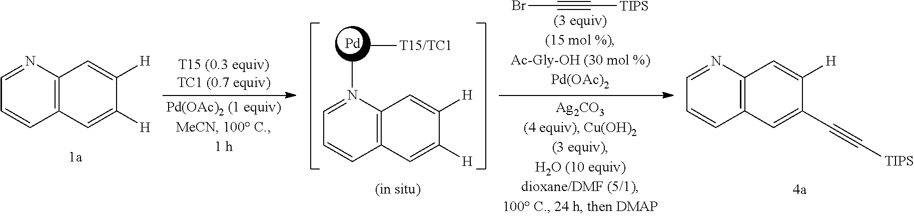

| | | Yield (%), |
|---|---|---|
| | | Selectivity |
| Entry | Deviation from standard conditions | (C6:others) |
| 1 | none | 72(66)^{a}, (96:4) |
| 2 | w/o Pd(OAc)₂ | N.R., (−) |
| 3 | w/o Ac-Gly-OH | 7, (−) |
| 4 | w/o Ag₂CO₃ | 9, (92:8) |
| 5 | w/o Cu(OH)₂ | 23, (90:10) |
| 6 | w/o H₂O | 64, (96:4) |
| 7 | w/o DMF | 67, (96:4) |
| 8 | Pd(MeCN₂Cl₂ instead of Pd(OAc)₂ | 33, (96:4) |
| 9 | Cu(OAc)₂ instead of Cu(OH)₂ | 30, (89:11) |
| 10 | Cu₂O instead of Cu(OH)₂ | 28, (88:12) |
| 11 | HFIP instead of standard solvent | N.R., (−) |

^{a}Isolated yield.

TABLE S7
Condition optimization for C6-selective C—H allylation of quinoline

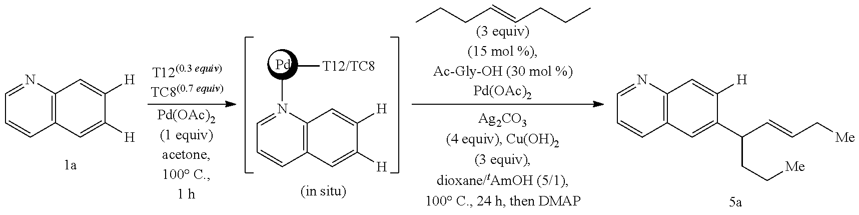

| | | Yield (%), |
|---|---|---|
| | | Selectivity |
| Entry | Deviation from standard conditions | (C6:others) |
| 1 | none | 76(75)^{a}, (91:9) |
| 2 | w/o Pd(OAc)₂ | N.R., (−) |
| 3 | w/o Ac-Gly-OH | N.R., (−) |
| 4 | w/o Ag₂CO₃ | 41, (91:9) |
| 5 | w/o Cu(OH)₂ | 33, (90:10) |
| 6 | w/o ^{t}AmOH | 52, (91:9) |
| 7 | T15/TC10 instead of T12/TC8 | 66, (91:9) |
| 8 | Cu₂O instead of Cu(OH)₂ | 41, (90:10) |
| 9 | dioxane/HFIP (5/1) instead of standard solvent | 39, (85:15) |

^{a}Isolated yield.

### 2.4 General Procedure for C6-Selective C—H Olefination

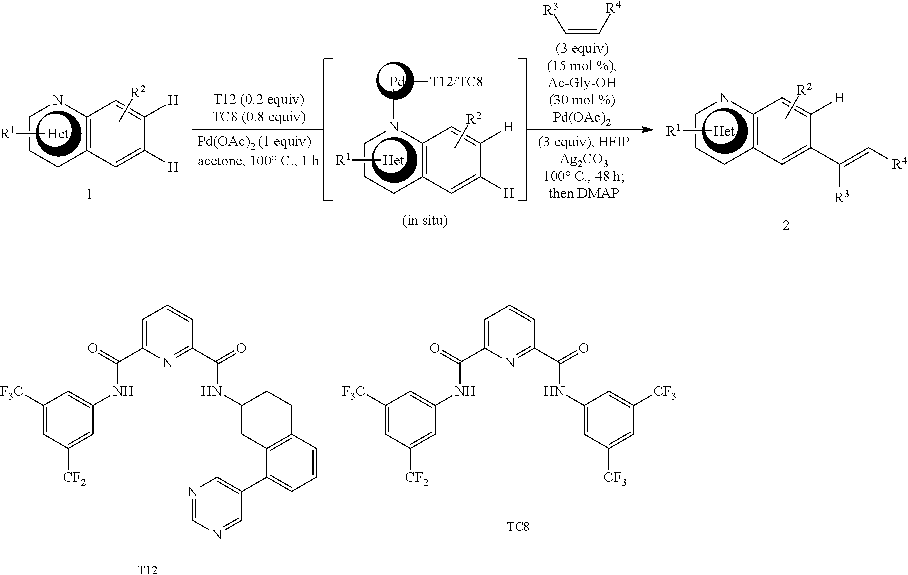

A reaction vial was charged with bicyclic aza-arene (0.1 mmol), 112 (11.7 mg, 0.02 mmol), TC8 (47.1 mg, 0.08 mmol), Pd(OAc)₂ (22.5 mg, 0.1 mmol) and acetone (2 mL). The reaction mixture was stirred at 100° C. for 1 h then concentrated in vacuo. Pd(OAc)₂ (3.4 mg, 0.015 mmol), Ac-Gly-OH (3.5 mg, 0.03 mmol), Ag₂CO₃ (82.7 mg, 0.3 mmol), HFIP (2 ml), and olefin (0.3 mmol) were added in the reaction vial. The vial was capped and allowed to stir at 100° C. for 48 h. After cooling to room temperature, a solution of DMAP (36.7 mg, 0.3 mmol) in toluene (1 mL) and TC8 (23.5 mg, 0.04 mmol) was added. The mixture was stirred at 100° C. for 30 mi. Upon completion, the mixture was passed through a short pad of Celite, washed with DCM, and concentrated. A portion of the sample was passed through a short pad of silica (in the glass dropper) using EA as the eluent to give the product mixture for determining the site-selectivity by H NMR analysis. The rest and the sample for analysis were combined and purified by a preparative TLC to afford the product 2 and TC-Pd-DMAP complex.

### 2.5 General Procedure for C7-Selective C—H Olefination

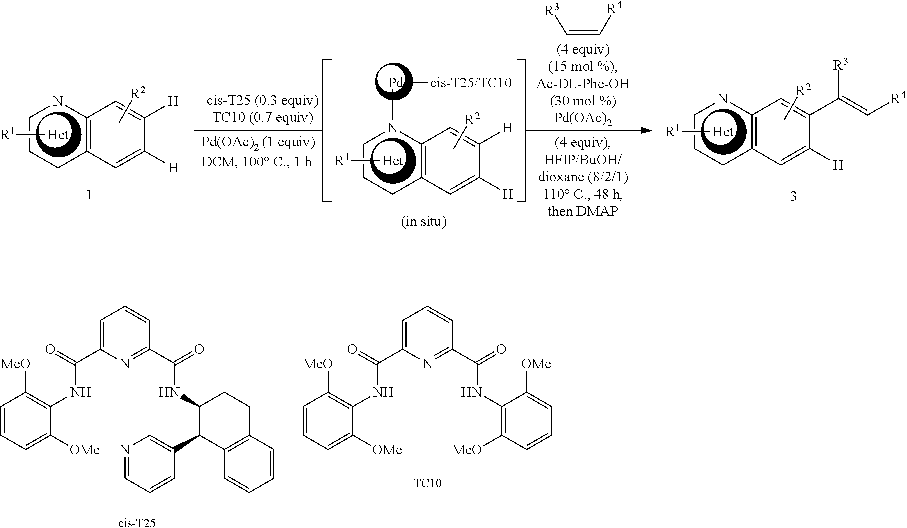

A reaction vial was charged with bicyclic aza-arene (0.05 mmol), cis-T25 (7.6 mg, 0.015 mmol), TC10 (15.3 mg, 0.035 mmol), Pd(OAc)₂ (11.2 mg, 0.05 mmol) and DCM (5 mL). The reaction mixture was stirred at 100° C. for 1 h then concentrated in vacuo. Pd(OAc)₂ (1.7 mg, 0.0075 mmol), Ac-DL-Phe-OH (3.1 mg, 0.015 mmol), Ag₂CO₃ (55.2 mg, 0.2 mmol), HFIP/^{t}BuOH/dioxane (4 ml/1 mL/0.5 mL), and olefin (0.2 mmol) were added in the reaction vial. The vial was capped and allowed to stir at 110° C. for 48 h. After cooling to room temperature, a solution of DMAP (18.3 mg, 0.15 mmol) in toluene (500 μL) was added. The mixture was stirred at 100° C. for 30 min. Upon completion, the mixture was passed through a short pad of Celite, washed with DCM, and concentrated. A portion of the sample was passed through a short pad of silica (in the glass dropper) using hexane/EA (50.50, v/v) as the eluent to give the product mixture for determining the site-selectivity by ¹H NMR analysis. The rest and the sample for analysis were combined and purified by a preparative TLC to afford the product 3. For 3z, 3aa to 3ad, using (S,S)-T25/Pd(MeCN)₂Cl₂/Ac-L-Leu-OH.

### 2.6 General Procedure for C6-Selective C—H Alkynylation

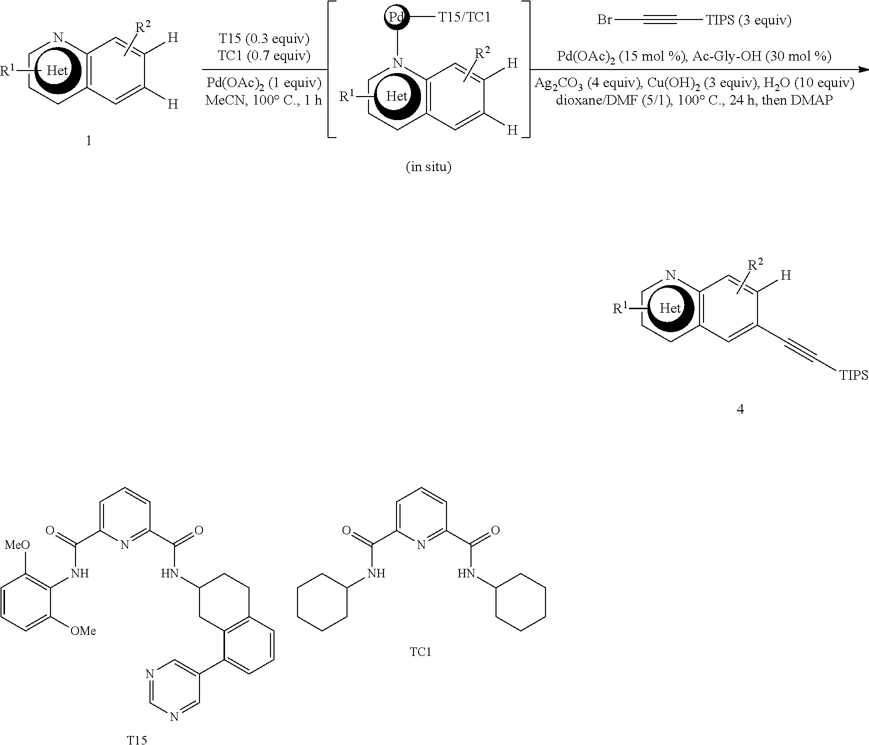

A reaction vial was charged with bicyclic aza-arene (0.1 mmol), 115 (15.2 mg, 0.03 mmol), TC1 (23 mg, 0.07 mmol), Pd(OAc)₂ (22.5 mg, 0.1 mmol) and MeCN (2 mL). The reaction mixture was stirred at 100° C. for 1 h then concentrated in vacuo. Pd(OAc)₂ (3.4 mg, 0.015 mmol), Ac-Gly-OH (3.5 mg, 0.03 mmol), Ag₂CO₃ (110 mg, 0.4 mmol), Cu(OH)₂ (29.2 mg, 0.3 mmol), H₂O (18 μL, 1 mmol), dioxane/DMF (2.5 mL/0.5 mL), and triisopropylsilyl acetylene bromide (78.4 mg, 0.3 mmol) were added in the reaction vial. The vial was capped and allowed to stir at 100° C. for 24 h. After cooling to room temperature, DMAP (73.4 mg, 0.6 mmol) and TFE (1 mL) were added. The mixture was stirred at 100° C. for 30 min. Upon completion, the mixture was passed through a short pad of Celite, washed with DCM, and concentrated. A portion of the sample was passed through a short pad of silica (in the glass dropper) using hexane/EA (50.50, v/v) as the eluent to give the product mixture for determining the site-selectivity by GC-MS and LC-MS analysis. The rest and the sample for analysis were combined and purified by a preparative TLC to afford the product 4.

### 2.7 General Procedure for C6-Selective C—H Allylation

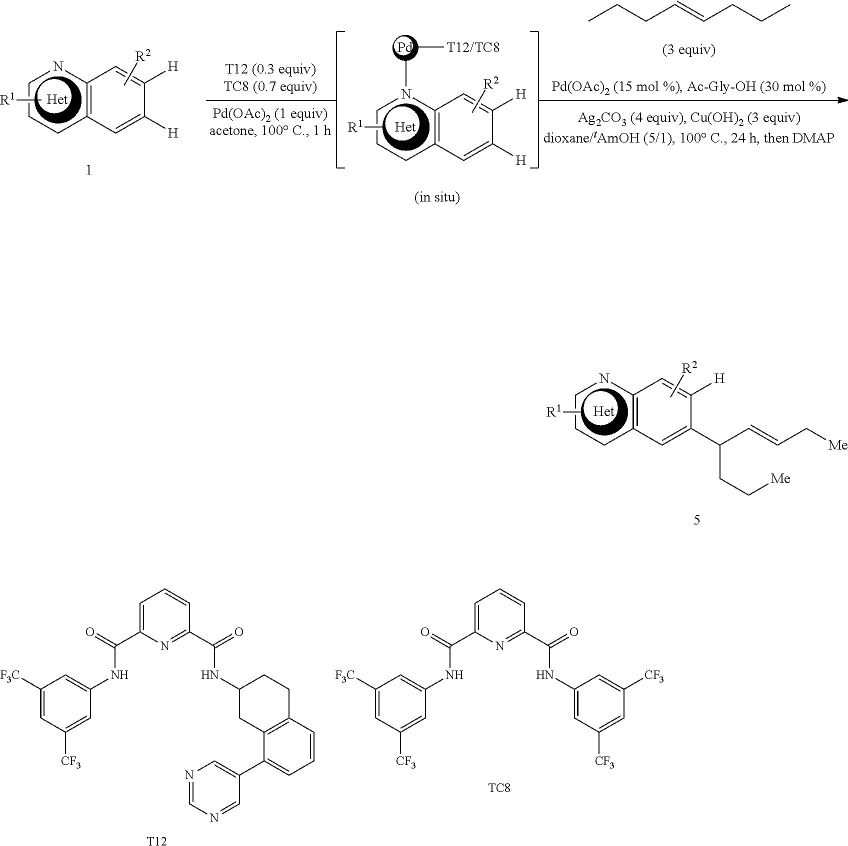

### 2.8 General Procedure for C7-Selective C—H Alkynylation

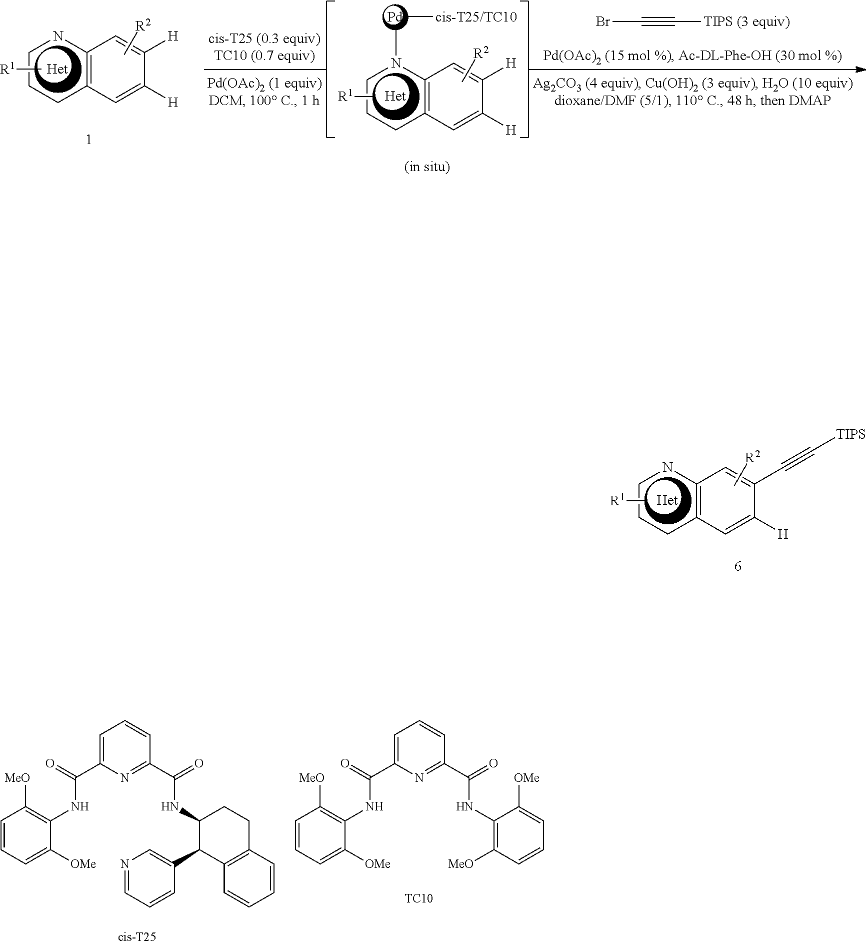

A reaction vial was charged with bicyclic aza-arene (0.05 mmol), cis-T25 (7.6 mg, 0.015 mmol), TC10 (15.3 mg, 0.035 mmol), Pd(OAc)₂ (11.2 mg, 0.05 mmol) and DCM (5 mL). The reaction mixture was stirred at 100° C. for 1 h then concentrated in vacuo. Pd(OAc)₂ (1.7 mg, 0.0075 mmol), Ac-DL-Phe-OH (3.1 mg, 0.015 mmol), Ag₂CO₃ (55.2 mg, 0.2 mmol), Cu(OH)₂ (14.6 mg, 0.15 mmol), H₂O (9 μL, 0.5 mmol), dioxane/DMF (2.5 mL/0.5 mL), and triisopropylsilyl acetylene bromide (39.2 mg, 0.15 mmol) were added in the reaction vial. The vial was capped and allowed to stir at 110° C. for 48 h. After cooling to room temperature, DMAP (36.7 mg, 0.3 mmol) and TFE (500 L) were added. The mixture was stirred at 110° C. for 30 min. Upon completion, the mixture was passed through a short pad of Celite, washed with DCM, and concentrated. A portion of the sample was passed through a short pad of silica (in the glass dropper) using hexane/EA (50:50, v/v) as the eluent to give the product mixture for determining the site-selectivity by GC-MS and LC-MS analysis. The rest and the sample for analysis were combined and purified by a preparative TLC to afford the product 6.

### 2.9 Recycling and Reusing Template-Palladium Complexes^{9,10}

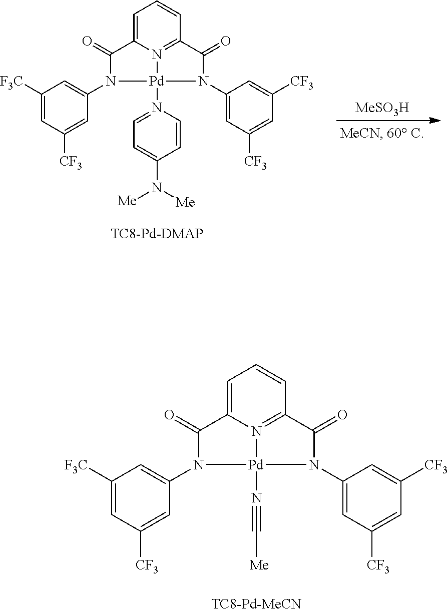

### Pd Recycling

TC8-Pd-DMAP complex was dissolved in MeCN and 1 equiv of MeSO₃H was added. The resulting mixture was heated at 60° C. for 2 h. After removing the solvent, water was added and extracted with DCM. The organic layer was dried with Na₂SO₄ and concentrated. This crude mixture was redissolved in MeCN and additional MeSO₃H (0.5 equiv) was added. The resulting mixture was heated at 60° C. for 30 min. After removing the solvent, water was added and extracted with DCM. The organic layer was dried with Na₂SO₄ and concentrated to give TC8-Pd-MeCN complex in 93% yield.

According to the procedure 2.4, 80% Pd species (TC8-Pd-DMAP) was recycled based on total 1.15 equiv of Pd loadings. Converting TC8-Pd-DMAP to active TC8-Pd-MeCN gave 93% yield. So, the total Pd recycling yield is 74%.Pd reusing

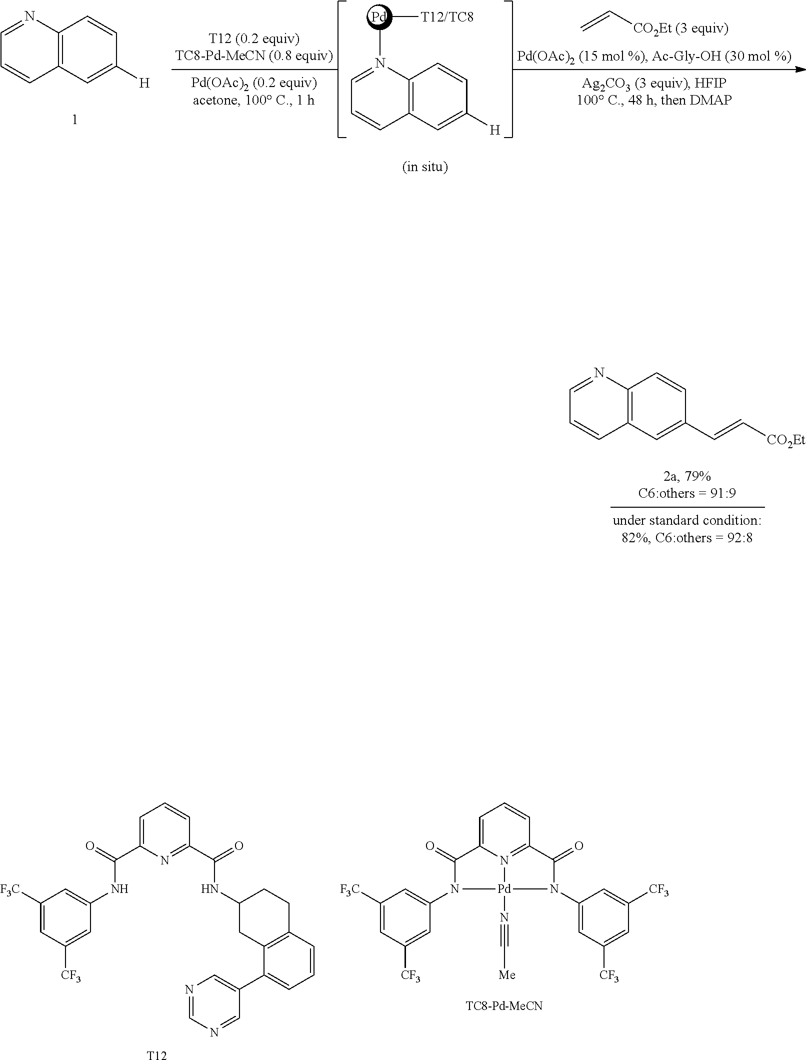

### 2.10 Procedure for Remote Site-Selective C—H Activation of Camptothecin

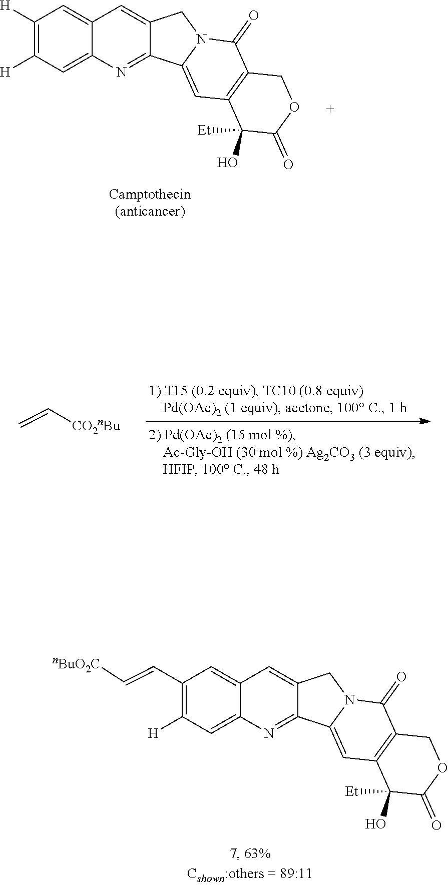

A reaction vial was charged with Camptothecin (35 mg, 0.1 mmol), T15 (10.2 mg, 0.02 mmol), TC10 (35 mg, 0.08 mmol), Pd(OAc)₂ (22.5 mg, 0.1 mmol) and acetone (2 mL). The reaction mixture was stirred at 100° C. for 1 h then concentrated in vacuo. Pd(OAc)₂ (3.4 mg, 0.015 mmol), Ac-Gly-OH (3.5 mg, 0.03 mmol), Ag₂CO₃ (82.7 mg, 0.3 mmol), HFIP (2 ml), and butyl acrylate (42.7 μL, 0.3 mmol) were added in the reaction vial. The vial was capped and allowed to stir at 100° C. for 48 h. After cooling to room temperature, a solution of DMAP (36.7 mg, 0.3 mmol) in toluene (1 mL) was added. The mixture was stirred at 100° C. for 30 min. Upon completion, the mixture was passed through a short pad of Celite, washed with DCM/MeOH (5/1), and concentrated. A portion of the sample was passed through a short pad of silica (in the glass dropper) using DCM/MeOH (10/1) as the eluent to give the product mixture for test the site-selectivity by ¹H NMR analysis. The rest and the sample for analysis were combined and purified by a preparative TLC using EA/acetone (5/1) as eluent to afford the product 7 (30 mg, 63% yield, C_{shown}:others=89.11) as a pale yellow solid. ¹H NMR (600 MHz, CDCl₃) δ 8.38 (s, 1H), 8.22 (d, J=8.8 Hz, 1H), 8.01 (dd, J=8.9, 1.9 Hz, 1H), 7.99 (d, J=1.8 Hz, 1H), 7.86 (d, J=15.9 Hz, 1H), 7.68 (s, 1H), 6.63 (d, J=16.0 Hz, 1H), 5.75 (d, J=16.3 Hz, 1H), 5.33-5.29 (m, 3H), 4.26 (t, J=6.7 Hz, 2H), 1.93 (dd, J=14.3, 7.3 Hz, 1H), 1.88 (dd, J=14.3, 7.3 Hz, 1H), 1.74-1.71 (m, 2H), 1.49-1.45 (m, 2H), 1.05 (t, J=7.4 Hz, 3H), 0.99 (t, J=7.4 Hz, 3H). ¹³C NMR (151 MHz, CDCl₃) δ 173.88, 166.68, 157.60, 153.29, 150.12, 149.71, 146.14, 142.92, 134.12, 131.31, 130.53, 129,37, 129.21, 128.39, 128.17, 120.66, 119.07, 98.40, 72.72, 66.36, 64.77, 50.05, 31.64, 30.77, 19.22, 13.77, 7.83. HRMS (ESI-TOF) m/z Calcd for C₂₇H₂₇N₂O⁺ [M+H]⁺ 475.1869, found 475.1874.

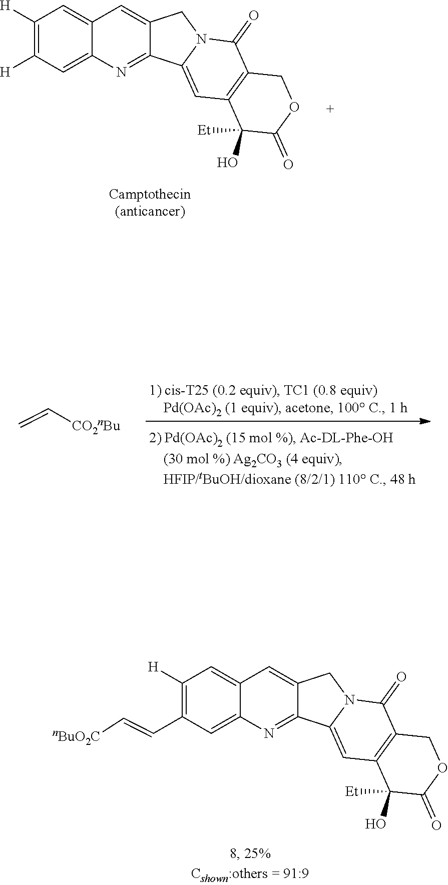

A reaction tube was charged with Camptothecin (35 mg, 0.1 mmol), cis-T25 (15 mg, 0.03 mmol), TC1 (23 mg, 0.07 mmol), Pd(OAc)₂ (22.5 mg, 0.1 mmol) and acetone (5 mL). The reaction mixture was stirred at 100° C. for 1 h then concentrated in vacuo. Pd(OAc)₂ (3.4 mg, 0.015 mmol), Ac-DL-Phe-OH (6.2 mg, 0.03 mmol), Ag₂CO₃ (110 mg, 0.4 mmol), HFIP/^{t}BuOH/dioxane (8 ml/2 mL/1 mL), and butyl acrylate (57, 0.4 mmol) were added in the reaction vial. The vial was capped and allowed to stir at 110° C. for 48 h. After cooling to room temperature, a solution of DMAP (36.7 mg, 0.3 mmol) in toluene (1 mL) was added. The mixture was stirred at 110° C. for 30 min. Upon completion, the mixture was passed through a short pad of Celite, washed with DCM/MeOH (5/1), and concentrated. A portion of the sample was passed through a short pad of silica (in the glass dropper) using DCM/MeOH (10/1) as the eluent to give the product mixture for test the site-selectivity by ¹H NMR analysis. The rest and the sample for analysis were combined and purified by a preparative TLC using DCM/ⁱPrOH (40/1) as eluent to afford the product 8 (11.8 mg, 25% yield, C_{shown}:others=91:9) as a pale yellow solid. ¹H NMR (600 MHz, CDCl₃) δ 8.37 (s, 1H), 8.30 (s, 1H), 7.93 (d, J=8.6 Hz, 1H), 7.88 (d, J=16.0 Hz, 1H), 7.82 (dd, J=8.5, 1.6 Hz, 1H), 7.68 (s, 1H), 6.67 (d, J=16.0 Hz, 1H), 5.75 (d, J=16.2 Hz, 1H), 5.33-5.30 (m, 3H), 4.27 (t, J=6.7 Hz, 2H), 1.94-1.87 (m, 2H), 1.75-1.71 (m, 2H), 1.49-1.45 (m, 2H), 1.05 (t, J=7.4 Hz, 3H), 1.00 (t, J=7.4 Hz, 3H). ¹³C NMR (151 MHz, CDCl₃) δ 173.86, 166.63, 157.60, 153.32, 150.14, 149.07, 146.11, 143.16, 136.73, 130.78, 130.53, 129.40, 128.88, 128.70, 125.98, 121.05, 119.02, 98.29, 72.72, 66.35, 64.79, 50.09, 31.64, 30.77, 19.23, 13.77, 7.83. HRMS (ESI-TOF) m/z Calcd for C₂₇H₂₇N₂O₆⁺ [M+H]⁺ 475.1869, found 475.1869.

### 2.11 Procedure for Synthesis of Cabozantinib Analogue 11

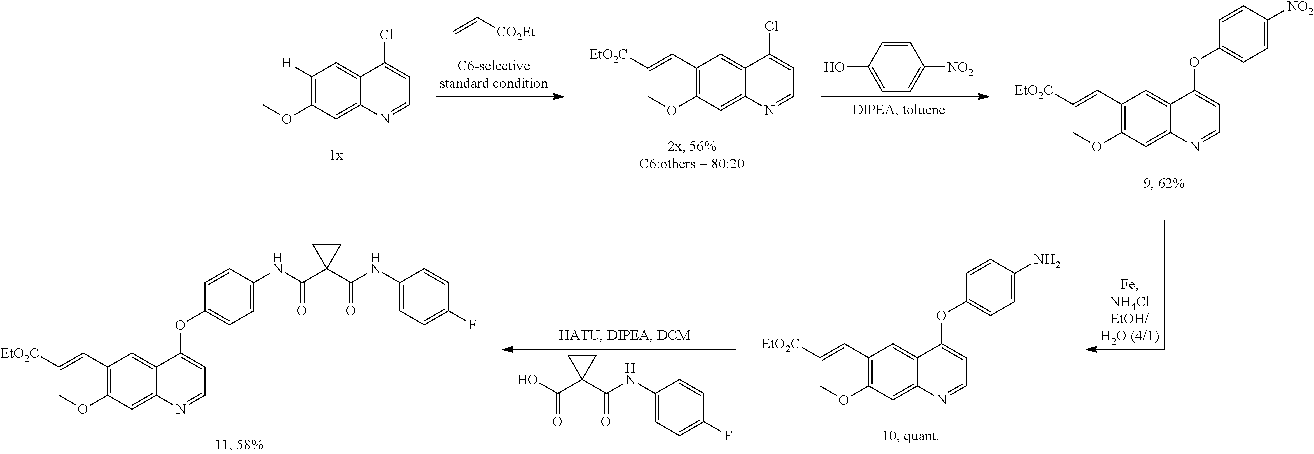

To a solution of 2x (36.7 mg, 0.19 mmol) in dry toluene (3 mL) was added DIPEA (79 μL, 0.48 mmol) and 4-nitrophenol (52.9 mg, 0.38 mmol). The reaction mixture was stirred at 120° C. for overnight. After completion, the mixture was concentrated and purified by a preparative TLC using DCM/EA (20/1) as eluent to afford the product 9 (46.4 mg, 62% yield) as a white solid. ¹H NMR (600 MHz, CDCl₃) δ 8.71 (d, J=5.1 Hz, 1H), 8.38-8.33 (m, 3H), 8.07 (d, J=16.2 Hz, 1H), 7.48 (s, 1H), 7.30 (d, J=9.1 Hz, 2H), 6.72 (d, J=16.2 Hz, 1H), 6.63 (d, J=5.1 Hz, 1H), 4.29 (q, J=7.1 Hz, 2H), 4.06 (s, 3H), 1.35 (t, J=7.1 Hz, 3H). ¹³C NMR (151 MHz, CDCl₃) δ 166.97, 160.02, 160.00, 159.82, 152.37, 152.32, 144.71, 139.19, 126.27, 126.25, 122.42, 121.48, 120.34, 116.12, 107.67, 105.24, 60.64, 55.96, 14.35. HRMS (ESI-TOF) m/z Calcd for C₂₁H₁₉N₂O₆⁺ [M+H]⁺ 395.1243, found 395.1238.

To a solution of 9 (19.7 mg, 0.05 mmol) in EtOH/H₂O (1 mL/0.25 mL) was added Fe dust (14 mg, 0.25 mmol) and NH₄Cl (26.7 mg, 0.5 mmol). The reaction mixture was stirred at 65° C. for 1 h. After completion, water was added and the mixture was extracted with EA. The combined organic layers were dried with Na₂SO₄ and concentrated. The residue was purified by a preparative TLC using EA/DCM (5/1) as eluent to afford the product 10 (18.2 mg, quant. yield) as pale yellow oil. ¹H NMR (600 MHz, CDCl₃) δ 8.55 (d, J=5.3 Hz, 1H), 8.53 (s, 1H), 8.11 (d, J=16.2 Hz, 1H), 7.41 (s, 1H), 6.98 (d, J=8.7 Hz, 2H), 6.77-6.74 (m, 3H), 6.40 (d, J=5.3 Hz, 1H), 4.29 (q, J=7.1 Hz, 2H), 4.03 (s, 3H), 1.35 (t, J=7.1 Hz, 3H). ¹³C NMR (151 MHz, CDCl₃) δ 167.21, 163.05, 159.43, 152.53, 151.87, 145.80, 144.34, 139.65, 125.08, 123.12, 122.17, 120.71, 116.31, 115.85, 107.28, 102.50, 60.49, 55.80, 14.37. HRMS (ESI-TOF) m/z Calcd for C₂₁H₂₁N₂O₄⁺ [M+H]⁺ 365.1501, found 365.1502.

To a solution of 10 (18.2 mg, 0.05 mmol), aliphatic acid (16.7 mg, 0.075 mmol), and DIPEA (16.5 μL, 0.1 mmol) in DCM (3 mL) at 0° C. was added HATU (28.5 mg, 0.075 mmol). The solution was warmed to room temperature and stirred for overnight. The reaction mixture was evaporated and purified by a preparative TLC using EA/DCM (5/1) as eluent to provide the product 11 (16.5 mg, 58% yield) as pale yellow oil. ¹H NMR (600 MHz, CDCl₃) δ 9.48 (s, 1H), 8.90 (s, 1H), 8.57 (d, J=5.3 Hz, 1H), 8.49 (s, 1H), 8.09 (d, J=16.1 Hz, 1H), 7.64 (d, J=8.9 Hz, 2H), 7.48 (dd, J=9.1, 4.8 Hz, 2H), 7.41 (s, 1H), 7.17 (d, J=8.9 Hz, 2H), 7.04 (t, J=8.6 Hz, 2H), 6.75 (d, J=16.1 Hz, 1H), 6.42 (d, J=5.3 Hz, 1H), 4.28 (q, J=7.1 Hz, 2H), 4.02 (s, 3H), 1.75-1.71 (m, 2H), 1.68-1.65 (m, 2H), 1.35 (t, J=7.2 Hz, 3H). ¹³C NMR (151 MHz, CDCl₃) δ 169.27, 168.82, 167.23, 162.18, 160.60, 159.53, 158.98, 152.44, 151.98, 150.50, 139.57, 135.10, 133.10 (d, J=3.0 Hz), 125.35, 122.95, 122.79, 122.74, 122.49, 121.72, 120.87, 115.87, 115.84, 115.72, 107.34, 102.99, 60.58, 55.83, 29.21, 17.61, 14.35. ¹⁹F NMR (376 MHz, CDCl₃) δ −119.55. HRMS (ESI-TOF) m/z Calcd for C₃₂H₂₉FN₃O₆⁺ [M+H]⁺ 570.2040, found 570.2049.

### 2.12 Procedure for Synthesis of Chloroquine Analogue 12

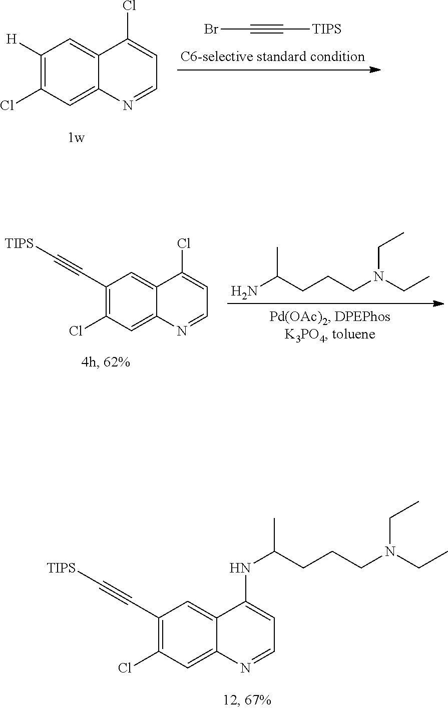

To a solution of 4h (11.3 mg, 0.03 mmol), Pd(OAc)₂ (0.7 mg, 0.003 mmol), DPEPhos (3.2 mg, 0.006 mmol), and K₃PO₄ (12.7 g, 0.06 mmol) in dry toluene (2 mL) was added amine (8.7 μL, 0.045 mmol). The vial was filled with N₂, then sealed and put into a preheated oil bath at 120° C. for 3 h. After completion, the reaction mixture was basified with NaOH solution and the solution was extracted with EA. The organic phase was washed with brine, dried with Na₂SO₄, and concentrated. The residue was purified by a preparative TLC using THF/hexane/Et₃N (10/1/0.005) as eluent to provide the product 12 (10 mg, 67% yield) as colorless oil. ¹H NMR (600 MHz, CDCl₃) δ 8.46 (d, J=5.5 Hz, 1H), 8.11 (s, 1H), 7.98 (s, 1H), 6.38 (d, J=5.6 Hz, 1H), 6.09 (s, 1H), 3.73 (d, J=6.8 Hz, 1H), 2.82 (q, J=7.3 Hz, 4H), 2.70 (d, J=7.7 Hz, 2H), 1.91 (dt, J=13.9, 6.5 Hz, 1H), 1.83-1.74 (m, 3H), 1.34 (d, J=6.4 Hz, 3H), 1.17 (m, 27H). ¹³C NMR (151 MHz, CDCl₃) δ 149.16, 136.50, 128.93, 126.02, 124.96, 123.06, 119.57, 117.34, 103.06, 99.39, 96.88, 62.78, 52.05, 48.37, 46.90, 33.89, 29.96, 20.05, 18.72, 11.38. HRMS (ESI-TOF) m/z Calcd for C₂₉H₄₇ClN₃Si⁺ [M+H]⁺ 500.3228, found 500.3230.

### 2.13 Procedure for Iterative C—H Activation of Quinoline

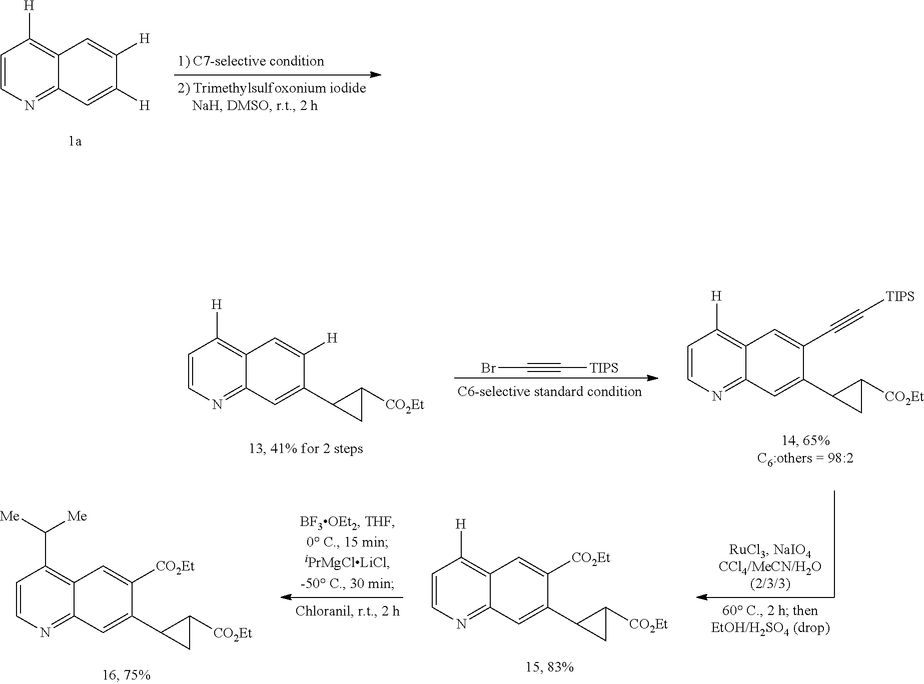

3z was synthesized according to the general procedure 2.5 except using (S,S)-T25/Pd(MeCN)₂Cl₂/Ac-L-Leu-OH.

To a solution of NaH (6 mg, 0.15 mmol) in dry DMSO was added trimethylsulfoxonium iodide (33 mg, 0.15 mmol). The mixture was stirred at room temperature for 0.5 h. 3z (22.7 mg, 0.1 mmol) in dry DMSO was added and the mixture was stirred at room temperature for 2 h. Water was added and the solution was extracted with EA. The organic phase was washed with brine, dried with Na₂SO₄, and concentrated. The residue was purified by a preparative TLC using DCM/EA (8/1, v/v) as eluent to afford the product 13 as a white solid (16 mg, 66%). ¹H NMR (600 MHz, CDCl₃) δ 8.88 (dd, J=4.2, 1.7 Hz, 1H), 8.11 (ddd, J=8.2, 2.0, 0.9 Hz, 1H), 7.79 (dt, J=1.5, 0.7 Hz, 1H), 7.74 (d, J=8.4 Hz, 1H), 7.34 (dd, J=8.2, 4.2 Hz, 1H), 7.32 (dd, J=8.5, 1.8 Hz, 1H), 4.20 (q, J=7.1 Hz, 2H), 2.72 (ddd, J=9.1, 6.4, 4.1 Hz, 1H), 2.06 (ddd, J=8.3, 5.3, 4.1 Hz, 1H), 1.72 (ddd, J=9.2, 5.4, 4.7 Hz, 1H), 1.48 (ddd, J=8.5, 6.4, 4.7 Hz, 1H), 1.30 (t, J=7.1 Hz, 3H). ¹³C NMR (151 MHz, CDCl₃) δ 173.13, 150.78, 148.33, 141.95, 135.73, 127.90, 127.01, 125.76, 125.69, 120.69, 60.86, 26.25, 24.70, 17.37, 14.27. HRMS (ESI-TOF) m/z Calcd for C₁₅H₁₆NO₂⁺ [M+H]⁻ 242.1181, found 242.1191.

14 was synthesized according to the general procedure 2.6. ¹H NMR (600 MHz, CDCl₃) δ 8.85 (dd, J=4.2, 1.7 Hz, 1H), 8.07 (ddd, J=8.5, 1.9, 0.7 Hz, 1H), 7.98 (s, 1H), 7.59 (s, 1H), 7.35 (dd, J=8.2, 4.2 Hz, 1H), 4.17 (q, J=7.2 Hz, 2H), 3.16 (dddd, J=9.2, 6.5, 4.3, 0.7 Hz, 1H), 2.09 (ddd, J=8.5, 5.4, 4.3 Hz, 1H), 1.75-1.72 (m, 1H), 1.50-1.46 (m, 1H), 1.28 (t, J=7.1 Hz, 3H), 1.17 (d, J=3.0 Hz, 21H). ¹³C NMR (151 MHz, CDCl₃) δ 172.78, 151.24, 147.78, 142.92, 135.24, 132.48, 126.53, 124.19, 123.10, 121.30, 104.44, 96.69, 60.70, 24.54, 24.50, 18.71, 17.29, 14.22, 11.33. HRMS (ESI-TOF) m/z Calcd for C₂₆H₃₆NO₂Si⁺ [M+H]⁺ 422.2515, found 422.2514.

A reaction vial was charged with 14 (21 mg, 0.05 mmol), RuCl₃ (1 mg, 0.005 mmol), NaIO₄ (43.2 mg, 0.2 mmol) and CCl₄/MeCN/H₂O (500 μL/750 μL/750 μL). The mixture was stirred at 60° C. for 2 h then concentrated in vacuo. Water was added and the solution was extracted with EA. The organic phase was washed with brine, dried with Na₂SO₄, and concentrated. The residue was re-dissolved in EtOH (2 mL) and conc. H₂SO₄ (1 drop) was added. The mixture was stirred at 90° C. for overnight then concentrated in vacuo. NaOH solution was added and the solution was extracted with EA. The organic phase was washed with brine, dried with Na₂SO₄, and concentrated. The residue was purified by a preparative TLC using hexane/EA (1/1, v/v) as eluent to afford the product 15 as colorless oil (13 mg, 83%). ¹H NMR (600 MHz, CDCl₃) δ 8.96 (dd, J=4.2, 1.7 Hz, 1H), 8.41 (s, 1H), 8.21 (d, J=7.5 Hz, 1H), 7.85 (s, 1H), 7.43 (dd, J=8.2, 4.1 Hz, 1H), 4.48-4.44 (m, 1H), 4.42-4.38 (m, 1H), 4.22 (q, J=7.1 Hz, 2H), 3.28-3.24 (m, 1H), 1.89 (dt, J=7.9, 4.9 Hz, 1H), 1.70 (dd, J=9.3, 4.7 Hz, 1H), 1.53 (ddd, J=8.0, 5.1, 3.5 Hz, 1H), 1.43 (t, J=7.2 Hz, 3H), 1.31 (t, J=7.3 Hz, 3H). ¹³C NMR (151 MHz, CDCl₃) δ 173.41, 167.04, 152.58, 149.24, 140.78, 136.61, 131.51, 130.60, 127.75, 126.20, 121.66, 61.54, 60.68, 25.55, 23.80, 15.91, 14.32, 14.27. HRMS (ESI-TOF) m/z Calcd for C₁₈H₂₀NO₄⁺ [M+H]⁺ 314.1392, found 314.1407.

16 was synthesized according to modified literature procedure:¹¹ A reaction via was charged with 15 (9.4 mg, 0.03 mmol) in dry THF and cooled to 0° C. BF₃·OEt₂ (4 μL, 0.033 mmol) was added and stirred at 0° C. for 0.5 h. Then, ⁱPrMgCl·LiCl (0.036 mmol) was added at −30° C. and stirring at r.t, for 0.5 h. Chloranil (14.8 mg, 0.06 mmol) was added and the mixture continuously stirred at r.t. for 1.5 h. The mixture was quenched with NaOH solution and extracted with EA. The organic phase was washed with brine, dried with Na₂SO₄, and concentrated. The residue was purified by a preparative TLC using hexane/EA (1/1, v/v) as eluent to afford the product 16 as colorless oil (8 mg, 75%). ¹H NMR (600 MHz, CDCl₃) δ 8.88 (d, J=4.6 Hz, 1H), 8.67 (s, 1H), 7.84 (s, 1H), 7.32 (d, J=4.6 Hz, 1H), 4.49-4.46 (m, 1H), 4.42-4.39 (m, 1H), 4.22 (d, J=7.2 Hz, 2H), 3.79-3.76 (m, 1H), 3.24 (dtd, J=6.9, 2.4, 1.0 Hz, 1H), 1.90-1.88 (m, 1H), 1.70-1.67 (m, 1H), 1.54-1.52 (m, 1H), 1.44 (d, J=7.2 Hz, 3H), 1.41 (dd, J=6.8, 1.8 Hz, 6H), 1.31 (t, J=7.1 Hz, 3H). ¹³C NMR (151 MHz, CDCl₃) δ 173.48, 167.57, 155.58, 152.60, 149.52, 139.89, 130.16, 128.64, 127.09, 124.95, 117.55, 61.58, 60.67, 28.40, 25.50, 23.72, 23.05, 15.86, 14.32, 14.28. HRMS (ESI-TOF) m/z Calcd for C₂₁H₂₆NO₄⁺ [M+H]⁺ 356.1862, found 356.1871.

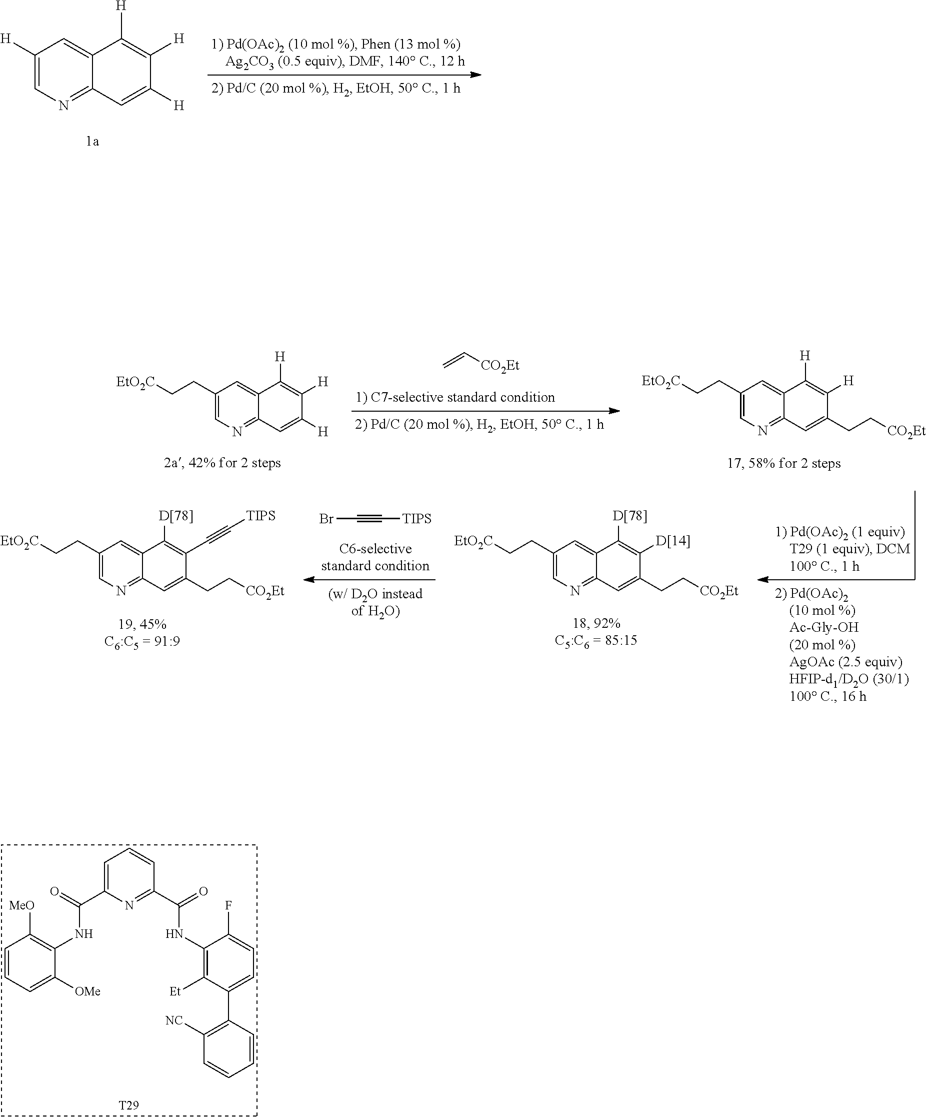

17 was synthesized according to the literature procedure¹² and general procedure 2.5. ¹H NMR (600 MHz, CDCl₃) δ 8.77 (d, J=2.3 Hz, 1H), 7.93 (d, J=1.2 Hz, 1H), 7.88-7.86 (m, 1H), 7.70 (d, J=8.3 Hz, 1H), 7.40 (dd, J=8.3, 1.8 Hz, 1H), 4.16-4.11 (m, 4H), 3.14 (dt, J=17.9, 7.7 Hz, 4H), 2.73 (q, J=7.6 Hz, 4H), 1.27-1.22 (m, 6H). ¹³C NMR (151 MHz, CDCl₃) δ 172.70, 172.36, 151.79, 147.21, 141.68, 134.21, 132.77, 128.00, 127.66, 127.46, 126.59, 60.66, 60.55, 35.52, 35.47, 31.05, 28.22, 14.23, 14.20. HRMS (ESI-TOF) m/z Calcd for C₁₉H₂₄NO₄⁺ [M+H]⁺ 330.1705, found 330.1711.

18 was synthesized according to modified literature procedure:¹³ A reaction vial was charged with 17 (33 mg, 0.1 mmol), T29 (52.4 mg, 0.1 mmol), Pd(OAc)₂ (22.5 mg, 0.1 mmol) and DCM (2 mL). The reaction mixture was stirred at 100° C. for 1 h then concentrated in vacuo. Pd(OAc)₂ (2.2 mg, 0.01 mmol), Ac-Gly-OH (2.3 mg, 0.02 mmol), AgOAc (41.8 mg, 0.25 mmol), HFIP-d₁ (1.2 mL), and D₂O (40 L) were added in the reaction vial. The vial was capped and allowed to stir at 100° C. for 16 h. After cooling to room temperature, a solution of DMAP (36.7 mg, 0.3 mmol) in toluene (1 mL) was added. The mixture was stirred at 100° C. for 30 min. Upon completion, the mixture was concentrated. The residue was purified by a preparative TLC using hexane/EA (2/1, v/v) as eluent to afford the partially deuterated product 18 (30.4 mg, 92% yield, C₅:C₆=85:15). ¹H NMR (600 MHz, CDCl₃) δ 8.77 (d, J=1.9 Hz, 1H), 7.93 (s, 1H), 7.87 (s, 1H), 7.70 (d, J=9.0 Hz, 0.22H), 7.40 (s, 0.86H), 4.16-4.11 (m, 4H), 3.14 (dt, J=17.9, 7.6 Hz, 4H), 2.72 (t, J=7.5 Hz, 4H), 1.23 (q, J=7.2 Hz, 6H). ¹³C NMR (151 MHz, CDCl₃) δ 172.71, 172.37, 151.78, 147.20, 141.69, 134.18, 132.77, 127.89, 127.64, 126.52, 60.66, 60.56, 35.52, 35.47, 31.06, 28.22, 14.22, 14.20. HRMS (ESI-TOF) m/z Calcd for C₁₉H₂₃DNO₄⁺ [M+H]⁺ 331.1768, found 331.1769.

19 was synthesized according to the general procedure 2.6 except using D₂O instead of H₂O. ¹H NMR (600 MHz, CDCl₃) δ 8.75 (d, J=2.4 Hz, 1H), 7.92 (s, 0.22H), 7.87 (d, J=0.9 Hz, 2H), 4.13 (dd, J=8.2, 7.2 Hz, 4H), 3.32-3.29 (m, 2H), 3.11 (d, J=7.6 Hz, 2H), 2.79-2.77 (m, 2H), 2.71 (t, J=7.6 Hz, 2H), 1.25-1.21 (m, 6H), 1.16 (d, J=3.6 Hz, 21H). ¹³C NMR (151 MHz, CDCl₃) δ 172.58, 172.27, 152.44, 146.61, 142.52, 133.61, 133.42, 132.35, 128.27, 126.27, 122.26, 104.51, 96.12, 60.69, 60.44, 35.31, 34.70, 30.23, 28.19, 18.70, 14.24, 14.20, 11.34. HRMS (ESI-TOF) m/z Calcd for C₃₀H₄₃DNO₄Si⁺ [M+H]⁺ 511.3102, found 511.3110.

### 2.14 Isotopic Labelling Studies

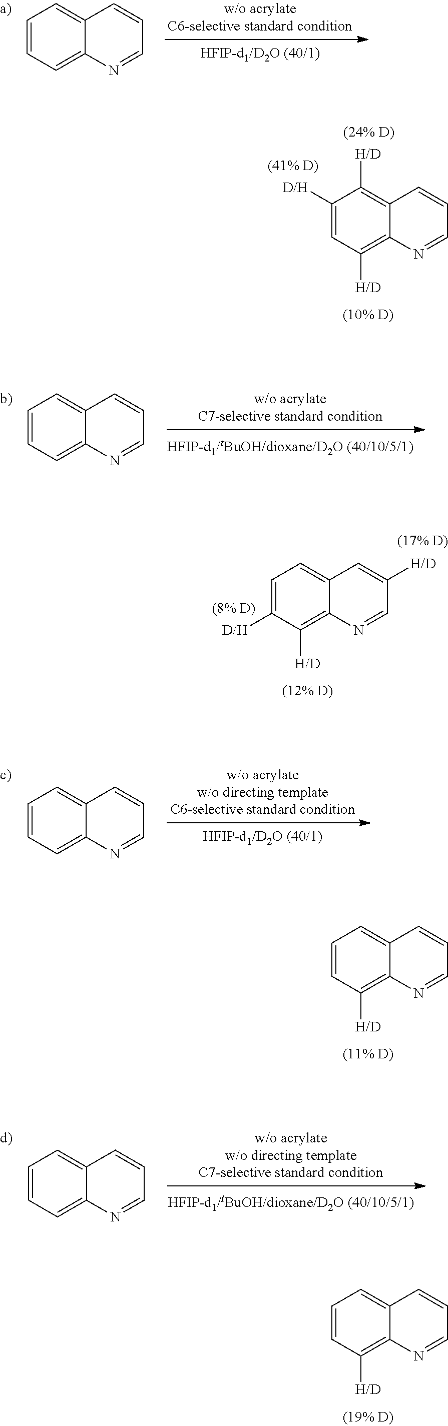

a) A reaction vial was charged with quinoline (2.6 mg, 0.02 mmol), T12 (2.3 mg, 0.004 mmol), TC8 (9.4 mg, 0.016 mmol), Pd(OAc)₂ (4.5 mg, 0.02 mmol) and acetone (1 mL). The reaction mixture was stirred at 100° C. for 1 h then concentrated in vacuo. Pd(OAc)₂ (0.7 mg, 0.003 mmol), Ac-Gly-OH (0.7 mg, 0.006 mmol), Ag₂CO₃ (16 mg, 0.06 mmol), HFIP-d₁ (400 L), and D₂O (10 L) were added in the reaction vial. The vial was capped and allowed to stir at 100° C. for 24 h. After cooling to room temperature, a solution of DMAP (7.3 mg, 0.06 mmol) in toluene (200 μL) was added. The mixture was stirred at 100° C. for 30 min. Upon completion, the mixture was concentrated. The residue was purified by a preparative TLC using hexane/EA (5/1, v/v) as eluent to afford the partially deuterated quinoline.

b) A reaction vial was charged with quinoline (2.6 mg, 0.02 mmol), cis-T25 (3 mg, 0.006 mmol), TC10 (6.1 mg, 0.014 mmol), Pd(OAc)₂ (4.5 mg, 0.02 mmol) and DCM (2 mL). The reaction mixture was stirred at 100° C. for 1 h then concentrated in vacuo. Pd(OAc)₂ (0.7 mg, 0.003 mmol), Ac-DL-Phe-OH (1.2 mg, 0.006 mmol), Ag₂CO₃ (22 mg, 0.08 mmol), and HFIP-d₁/^{t}BuOH/dioxane/D₂O (400 L/100 L/50 μL/10 μL) were added in the reaction vial. The vial was capped and allowed to stir at 110° C. for 24 h. After cooling to room temperature, a solution of DMAP (7.3 mg, 0.06 mmol) in toluene (200 μL) was added. The mixture was stirred at 100° C. for 30 min. Upon completion, the mixture was concentrated. The residue was purified by a preparative TLC using hexane/EA (5/1, v/v) as eluent to afford the partially deuterated quinoline.

c) A reaction vial was charged with quinoline (2.6 mg, 0.02 mmol), TC8 (11.8 mg, 0.02 mmol), Pd(OAc)₂ (4.5 mg, 0.02 mmol) and acetone (1 mL). The reaction mixture was stirred at 100° C. for 1 h then concentrated in vacuo. Pd(OAc)₂ (0.7 mg, 0.003 mmol), Ac-Gly-OH (0.7 mg, 0.006 mmol), Ag₂CO₃ (16 mg, 0.06 mmol), HFIP-d₁ (400 μL), and D₂O (10 L) were added in the reaction vial. The vial was capped and allowed to stir at 100° C. for 24 h. After cooling to room temperature, a solution of DMAP (7.3 mg, 0.06 mmol) in toluene (200 L) was added. The mixture was stirred at 100° C. for 30 min. Upon completion, the mixture was concentrated. The residue was purified by a preparative TLC using hexane/EA (5/1, v/v) as eluent to afford the partially deuterated quinoline.

d) A reaction vial was charged with quinoline (2.6 mg, 0.02 mmol), TC10 (8.7 mg, 0.02 mmol), Pd(OAc)₂ (4.5 mg, 0.02 mmol) and DCM (2 mL). The reaction mixture was stirred at 100° C. for 1 h then concentrated in vacuo. Pd(OAc)₂ (0.7 mg, 0.003 mmol), Ac-DL-Phe-OH (1.2 mg, 0.006 mmol), Ag₂CO₃ (22 mg, 0.08 mmol), and HFIP-d₁/^{t}BuOH/dioxane/D₂O (400 μL/100 μL/50 μL/10 μL) were added in the reaction vial. The vial was capped and allowed to stir at 110° C. for 24 h. After cooling to room temperature, a solution of DMAP (7.3 mg, 0.06 mmol) in toluene (200 μL) was added. The mixture was stirred at 100° C. for 30 min. Upon completion, the mixture was concentrated. The residue was purified by a preparative TLC using hexane/EA (5/1, v/v) as eluent to afford the partially deuterated quinoline.

### 2.15 Kinetic Isotope Effect (KIE) Studies

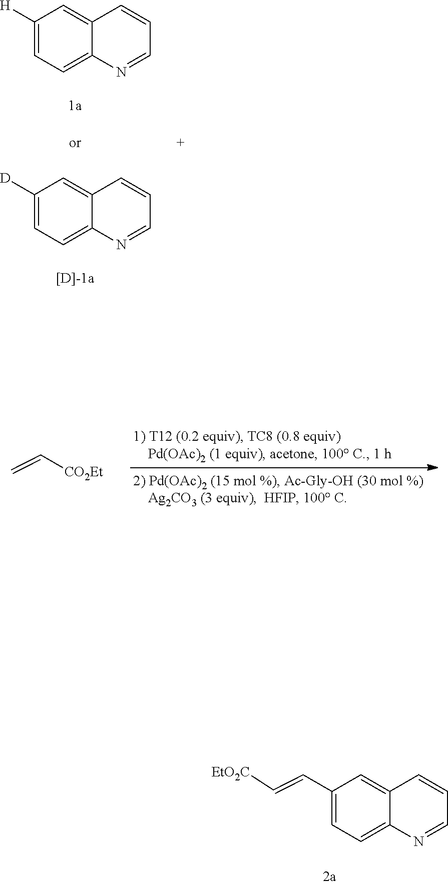

To 5 sets of vials were charged with 1a (2.6 mg, 0.02 mmol) or [D]-1a¹⁴ (2.6 mg, 0.02 mmol), T12 (2.3 mg, 0.004 mmol), TC8 (9.4 mg, 0.016 mmol), Pd(OAc)₂ (4.5 mg, 0.02 mmol) and acetone (0.5 mL). The reaction mixture was stirred at 100° C. for 1 h then concentrated in vacuo. Pd(OAc)₂ (0.7 mg, 0.003 mmol), Ac-Gly-OH (0.7 mg, 0.006 mmol), Ag₂CO₃ (16 mg, 0.06 mmol), HFIP (400 μL), and ethyl acrylate (6.5 μL, 0.06 mmol) were added in the reaction vials. The vials were capped and allowed to stir at 100° C. The reactions were monitored and removed 1 set of reactions after 20 min. The vials were quickly cooled to room temperature using dry ice. A solution of DMAP (7.3 mg, 0.06 mmol) in toluene (0.2 mL) was added. The mixture was stirred at 100° C. for 30 min. Upon completion, the mixture was passed through a short pad of silica (in the glass dropper) using hexane/EA (1/1, v/v) as the eluent to give the product mixture for ¹H NMR analysis.

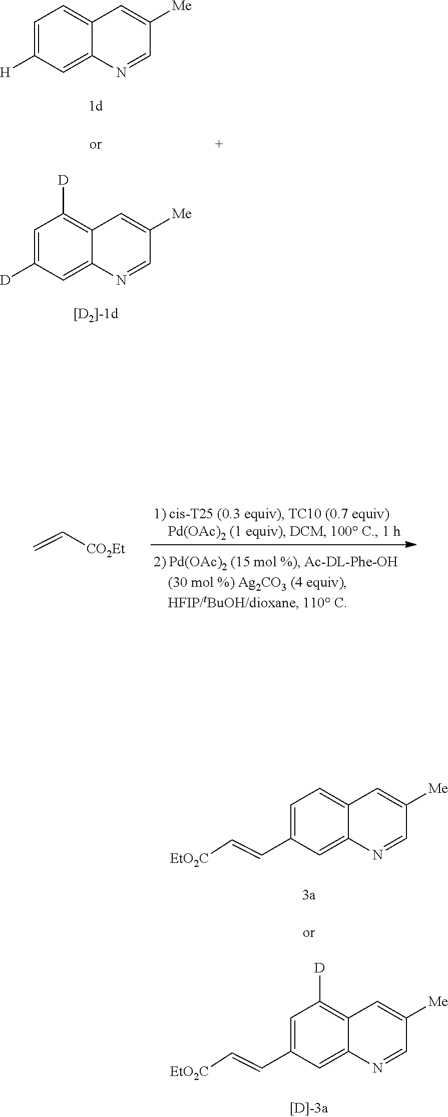

To 5 sets of vials were charged with 1d (2.8 mg, 0.02 mmol) or [D2]-1d¹⁵ (2.9 mg, 0.02 mmol), cis-T25 (3 mg, 0.006 mmol), TC10 (6 mg, 0.014 mmol), Pd(OAc)₂ (4.5 mg, 0.02 mmol) and DCM (1 mL). The reaction mixture was stirred at 100° C. for 1 h then concentrated in vacuo. Pd(OAc)₂ (0.7 mg, 0.003 mmol), Ac-DL-Phe-OH (1.2 mg, 0.006 mmol), Ag₂CO₃ (11 mg, 0.08 mmol), HFIP/^{t}BuOH/dioxane (1.6 mL/400 μL/200 μL), and ethyl acrylate (8.6 μL, 0.08 mmol) were added in the reaction vials. The vials were capped and allowed to stir at 110° C. The reactions were monitored and removed 1 set of reactions after 20 min. The vials were quickly cooled to room temperature using dry ice. A solution of DMAP (7.3 mg, 0.06 mmol) in toluene (0.2 mL) was added. The mixture was stirred at 110° C. for 30 min. Upon completion, the mixture was passed through a short pad of silica (in the glass dropper) using hexane/EA (1/1, v/v) as the eluent to give the product mixture for ¹H NMR analysis.

### 2.16 Spectroscopic Data of Compounds

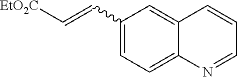 [SMILES: CCOC(=O)C=Cc1ccc2ncccc2c1]

Ethyl (E)-3-(quinolin-6-yl)acrylate (2a) The general procedure 2.4 was followed and purification by preparative TLC (hexane/EA=3/1) furnished compound 2a as a white solid (18.6 mg, 82%, C6:others=92:8). ¹H NMR (600 MHz, CDCl₃) δ 8.93 (dd, J=4.2, 1.6 Hz, 1H), 8.18 (d, J=8.2 Hz, 1H), 8.11 (d, J=9.3 Hz, 1H), 7.91 (dd, J=4.6, 2.6 Hz, 2H), 7.85 (d, J=16.0 Hz, 1H), 7.44 (dd, J=8.3, 4.3 Hz, 11H), 6.58 (d, J=16.0 Hz, 1H), 4.30 (q, J=7.2 Hz, 2H), 1.37 (t, J=7.1 Hz, 3H). ¹³C NMR (151 MHz, CDCl₃) δ 166.80, 151.34, 149.04, 143.61, 136.48, 132.73, 130.27, 129.23, 128.27, 127.28, 121.87, 119.65, 60.69, 14.34. HRMS (ESI-TOF) m/z Calcd for C₁₄H₁₄NO₂⁺ [M+H]⁺ 228.1025, found 228.1028.

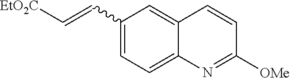 [SMILES: CCOC(=O)C=Cc1ccc2nc(OC)ccc2c1]

Ethyl (E)-3-(2-methoxyquinolin-6-yl)acrylate (2b) The general procedure 2.4 was followed and purification by preparative TLC (hexane/EA=10/1) furnished compound 2b as a white solid (12.6 mg, 49%, C6:others=93:7). ¹H NMR (600 MHz, CDCl₃) δ 7.97 (d, J=8.8 Hz, 1H), 7.84-7.79 (m, 4H), 6.92 (d, J=8.8 Hz, 1H), 6.51 (d, J=16.0 Hz, 1H), 4.29 (q, J=7.1 Hz, 2H), 4.08 (s, 3H), 1.36 (t, J=7.1 Hz, 3H). ¹³C NMR (151 MHz, CDCl₃) δ 167.05, 163.26, 147.82, 144.10, 138.90, 130.25, 128.79, 127.98, 127.61, 124.97, 118.04, 113.90, 60.53, 53.60, 14.36. HRMS (ESI-TOF) m/z Calcd for C₁₅H₁₆NO₃⁺ [M+H]⁺ 258.1130, found 258.1132.

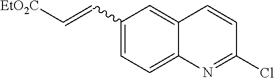 [SMILES: CCOC(=O)C=Cc1ccc2nc(Cl)ccc2c1]

Ethyl (E)-3-(2-chloroquinolin-6-yl)acrylate (2c) The general procedure 2.4 was followed and purification by preparative TLC (hexane/EA=10/1) furnished compound 2c as a white solid (11.5 mg, 44%, C6:others=96:4). ¹H NMR (600 MHz, CDCl₃) δ 8.12 (d, J=8.6 Hz, 1H), 8.02 (d, J=8.8 Hz, 1H), 7.92 (dd, J=8.8, 1.9 Hz, 1H), 7.90 (s, 1H), 7.82 (d, J=16.0 Hz, 1H), 7.43 (d, J=8.6 Hz, 1H), 6.57 (d, J=16.0 Hz, 1H), 4.30 (q, J=7.3 Hz, 2H), 1.36 (t, J=7.1 Hz, 3H). ¹³C NMR (151 MHz, CDCl₃) δ 166.65, 151.76, 148.58, 143.13, 139.11, 133.19, 129.35, 128.65, 128.51, 126.86, 123.21, 120.11, 60.78, 14.34. HRMS (ESI-TOF) m/z Calcd for C₁₄H₁₃ClNO₂⁺ [M+H]⁺ 262.0635, found 262.0634.

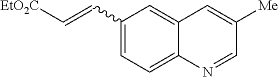 [SMILES: CCOC(=O)C=Cc1ccc2ncc(C)cc2c1]

Ethyl (E)-3-(3-methylquinolin-6-yl)acrylate (2d) The general procedure 2.4 was followed and purification by preparative TLC (hexane/EA=3/1) furnished compound 2d as a white solid (18 mg, 75%, C6:others=93:7). ¹H NMR (600 MHz, CDCl₃) δ 8.78 (d, J=2.1 Hz, 1H), 8.05 (d, J=9.4 Hz, 1H), 7.92 (s, 1H), 7.85-7.82 (m, 3H), 6.56 (d, J=16.0 Hz, 1H), 4.30 (q, J=7.1 Hz, 2H), 2.53 (s, 3H), 1.36 (t, J=7.1 Hz, 3H). ¹³C NMR (151 MHz, CDCl₃) δ 166.87, 153.36, 147.33, 143.85, 135.06, 132.69, 131.41, 129.97, 128.71, 128.12, 126.26, 119.34, 60.66, 18.78, 14.35. HRMS (ESI-TOF) m/z Calcd for C₁₅H₁₆NO₂⁺ [M+H]⁺ 242.1181, found 242.1182,

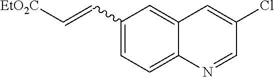 [SMILES: CCOC(=O)C=Cc1ccc2ncc(Cl)cc2c1]

Ethyl (E)-3-(3-chloroquinolin-6-yl)acrylate (2e) The general procedure 2.4 was followed and purification by preparative TLC (hexane/EA=10/1) furnished compound 2e as a white solid (17.5 mg, 67%, C6:others=98:2). ¹H NMR (600 MHz, CDCl₃) δ 8.82 (d, J=2.4 Hz, 1H), 8.14 (d, J=2.7 Hz, 1H), 8.08 (d, J=8.8 Hz, 1H), 7.89 (dd, J=8.8, 2.0 Hz, 1H), 7.84-7.80 (m, 2H), 6.58 (d, J=16.0 Hz, 1H), 4.31 (q, J=7.1 Hz, 2H), 1.37 (t, J=7.2 Hz, 3H). ¹³C NMR (151 MHz, CDCl₃) δ 166.58, 150.51, 146.90, 143.08, 134.22, 133.86, 130.23, 129.31, 128.49, 128.10, 127.45, 120.39, 60.79, 14.33. HRMS (ESI-TOF) m/z Calcd for C₄H₃ClNO₂⁺ [M+H]⁺ 262.0635, found 262.0632.

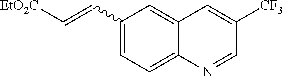 [SMILES: CCOC(=O)C=Cc1ccc2ncc(C(F)(F)F)cc2c1]

Ethyl (E)-3-(3-(trifluoromethyl)quinolin-6-yl)acrylate (2f) The general procedure 2.4 was followed and purification by preparative TLC (hexane/EA=10/1) furnished compound 2f as a white solid (19.2 mg, 65%, C6:others=91:9). ¹H NMR (600 MHz, CDCl₃) δ 9.11 (d, J=2.0 Hz, 1H), 8.46 (s, 1H), 8.19 (d, J=8.8 Hz, 1H), 8.04 (dd, J=8.8, 2.0 Hz, 1H), 8.01 (s, 1H), 7.85 (d, J=16.0 Hz, 1H), 6.62 (d, J=16.0 Hz, 1H), 4.32 (q, J=7.1 Hz, 2H), 1.37 (t, J=7.1 Hz, 3H). ¹³C NMR (151 MHz, CDCl₃) δ 166.49, 150.01, 146.88 (q, J=3.5 Hz), 142.71, 134.33-134.02 (m), 130.42, 129.55, 129.51, 126.40, 124.42 (q, J=32.9 Hz), 122.58, 120.86, 60.87, 14.33. ¹⁹F NMR (376 MHz, CDCl₃) δ −64.62. HRMS (ESI-TOF) m/z Calcd for C₁₅H₁₃F₃NO₂⁺ [M+H]⁺ 296.0898, found 296.0898.

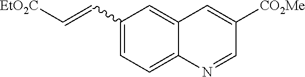 [SMILES: CCOC(=O)C=Cc1ccc2ncc(C(=O)OC)cc2c1]

Methyl (E)-6-(3-ethoxy-3-oxoprop-1-en-1-yl)quinoline-3-carboxylate (2g) The general procedure 2.4 was followed and purification by preparative TLC (hexane/EA=3/1) furnished compound 2g as a white solid (23.9 mg, 84%, C6:others=93:7). ¹H NMR (600 MHz, CDCl₃) δ 9.45 (d, J=2.1 Hz, 1H), 8.85 (d, J=1.3 Hz, 1H), 8.16 (d, J=9.5 Hz, 1H), 8.02-8.00 (m, 2H), 7.85 (d, J=16.0 Hz, 1H), 6.60 (d, J=16.0 Hz, 1H), 4.31 (q, J=7.1 Hz, 2H), 4.04 (s, 3H), 1.37 (t, J=7.1 Hz, 3H). ¹³C NMR (151 MHz, CDCl₃) δ 166.57, 165.56, 150.89, 150.52, 142.94, 138.98, 133.67, 130.29, 130.09, 129.59, 126.95, 123.77, 120.44, 60.81, 52.63, 14.33. HRMS (ESI-TOF) m/z Calcd for C₁₆H₁₆NO₄⁺ [M+H]⁺ 286.1079, found 286.1078.

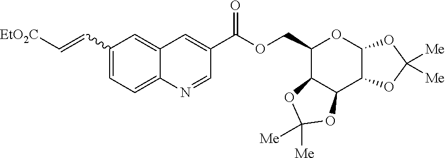 [SMILES: CCOC(=O)C=Cc1ccc2ncc(C(=O)OC[C@H]3O[C@@H]4OC(C)(C)O[C@@H]4[C@H]4OC(C)(C)O[C@H]43)cc2c1]

((3aR,5R,5aS,8aS,8bR)-2,2,7,7-Tetramethyltetrahydro-5H-bis([1,3]dioxolo)[4,5-b:4′,5′-d]pyran-5-yl)methyl 6-((E)-3-ethoxy-3-oxoprop-1-en-1-yl)quinoline-3-carboxylate (2h) The general procedure 2.4 was followed and purification by preparative TLC (hexane/EA=3/1) furnished compound 2h as a white solid (44.6 mg, 87%, C6:others=93:7). ¹H NMR (600 MHz, CDCl₃) δ 9.46 (d, J=2.1 Hz, 1H), 8.85 (d, J=1.9 Hz, 1H), 8.16 (d, J=9.4 Hz, 1H), 8.02-8.00 (m, 2H), 7.85 (d, J=16.0 Hz, 1H), 6.60 (d, J=16.0 Hz, 1H), 5.59 (d, J=5.0 Hz, 1H), 4.68 (dd, J=7.9, 2.5 Hz, 1H), 4.63-4.54 (m, 2H), 4.38-4.35 (m, 2H), 4.31 (q, J=7.1 Hz, 2H), 4.25 (ddd, J=7.8, 4.5, 1.9 Hz, 1H), 1.52 (d, J=21.7 Hz, 6H), 1.39-1.34 (m, 9H). ¹³C NMR (151 MHz, CDCl₃) δ 166.58, 164.96, 150.97, 150.56, 142.96, 139.09, 133.64, 130.28, 130.15, 129.60, 126.92, 123.70, 120.42, 109.84, 108.89, 96.35, 71.16, 70.79, 70.51, 66.13, 64.64, 60.80, 26.07, 26.00, 24.97, 24.51, 14.34. HRMS (ESI-TOF) m/z Calcd for C₂₇H₃₂NO₉⁺ [M+H]⁺ 514.2077, found 514.2077.

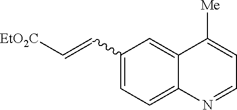 [SMILES: CCOC(=O)C=Cc1ccc2nccc(C)c2c1]

Ethyl (E)-3-(4-methylquinolin-6-yl)acrylate (2i) The general procedure 2.4 was followed and purification by preparative TLC (hexane/EA=3/1) furnished compound 2i as a white solid (12.3 mg, 51%, C6:others=89:11). ¹H NMR (600 MHz, CDCl₃) δ 8.78 (d, J=4.3 Hz, 1H), 8.10-8.06 (m, 2H), 7.91-7.86 (m, 2H), 7.26 (d, J=4.5 Hz, 1H), 6.58 (d, J=16.0 Hz, 1H), 4.31 (q, J=7.1 Hz, 2H), 2.72 (s, 3H), 1.37 (t, J=7.2 Hz, 3H). ¹³C NMR (151 MHz, CDCl₃) δ 166.83, 151.08, 148.83, 144.85, 144.02, 132.37, 130.81, 128.30, 126.82, 125.62, 122.63, 119.39, 60.67, 18.63, 14.36. HRMS (ESI-TOF) m/z Calcd for C₁₅H₁₆NO₂⁺ [M+H]⁻ 242.1181, found 242.1181.

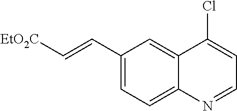 [SMILES: CCOC(=O)/C=C/c1ccc2nccc(Cl)c2c1]

Ethyl (E)-3-(4-chloroquinolin-6-yl)acrylate (2j) The general procedure 2.4 was followed and purification by preparative TLC (hexane/EA=5/1) furnished compound 2j as a white solid (15.4 mg, 59%, C6:others=91:9). ¹H NMR (600 MHz, CDCl₃) δ 8.79 (d, J=4.7 Hz, 1H), 8.32 (d, J=1.9 Hz, 1H), 8.12 (d, J=8.8 Hz, 1H), 7.95 (dd, J=8.8, 2.0 Hz, 1H), 7.89 (d, J=16.0 Hz, 1H), 7.52 (d, J=4.7 Hz, 1H), 6.62 (d, J=16.0 Hz, 1H), 4.31 (q, J=7.1 Hz, 2H), 1.37 (t, J=7.2 Hz, 3H). ¹³C NMR (151 MHz, CDCl₃) δ 166.58, 150.71, 149.82, 143.26, 143.03, 133.78, 130.68, 128.25, 126.64, 125.35, 121.98, 120.49, 60.79, 14.34. HRMS (ESI-TOF) m/z Calcd for C₁₄H₁₃ClNO₂⁺ [M+H]⁺ 262.0635, found 262.0637.

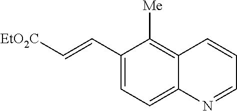 [SMILES: CCOC(=O)/C=C/c1ccc2ncccc2c1C]

Ethyl (E)-3-(5-methylquinolin-6-yl)acrylate (2k) The general procedure 2.4 was followed and purification by preparative TLC (hexane/EA=3/1) furnished compound 2k as a pale yellow solid (8.7 mg, 36%, C6:others=96:4). ¹H NMR (600 MHz, CDCl₃) δ 8.92 (dd, J=4.2, 1.6 Hz, 1H), 8.44 (d, J=8.5 Hz, 1H), 8.26 (d, J=15.8 Hz, 1H), 7.96 (d, J=8.6 Hz, 1H), 7.87 (d, J=8.9 Hz, 1H), 7.46 (dd, J=8.6, 4.2 Hz, 1H), 6.49 (d, J=15.8 Hz, 1H), 4.30 (t, J=7.1 Hz, 2H), 2.77 (s, 3H), 1.37 (t, J=7.2 Hz, 3H). ¹³C NMR (151 MHz, CDCl₃) δ 166.94, 150.60, 149.00, 141.91, 134.66, 133.09, 130.83, 128.13, 127.95, 127.43, 121.38, 120.94, 60.69, 14.35, 14.20. HRMS (ESI-TOF) m/z Calcd for C₁₅H₁₆NO₂⁺ [M+H]⁺ 242.1181, found 242.1183.

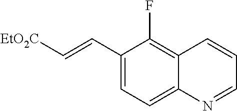 [SMILES: CCOC(=O)/C=C/c1ccc2ncccc2c1F]

Ethyl (E)-3-(5-fluoroquinolin-6-yl)acrylate (21) The general procedure 2.4 was followed and purification by preparative TLC (hexane/EA=3/1) furnished compound 21 as a white solid (11.5 mg, 47%, C6:others=97:3). ¹H NMR (600 MHz, CDCl₃) δ 8.97 (dd, J=4.2, 1.7 Hz, 1H), 8.48 (ddd, J=8.5, 1.8, 0.8 Hz, 1H), 8.06 (d, J=16.2 Hz, 1H), 7.91 (d, J=9.0 Hz, 1H), 7.85 (dd, J=8.9, 7.6 Hz, 1H), 7.50 (dd, J=8.5, 4.2 Hz, 1H), 6.64 (d, J=16.2 Hz, 1H), 4.31 (q, J=7.1 Hz, 2H), 1.37 (t, J=7.1 Hz, 3H). ¹³C NMR (151 MHz, CDCl₃) δ 166.59, 156.27 (d, J=264.1 Hz), 151.95, 135.83 (d, J=4.5 Hz), 129.92 (d, J=5.8 Hz), 127.00 (d, J=4.2 Hz), 125.81 (d, J=4.6 Hz), 121.87 (d, J=3.2 Hz), 121.33 (d, J=5.6 Hz), 119.18 (d, J=12.5 Hz), 117.54 (d, J=10.6 Hz), 112.49 (d, J=7.4 Hz), 60.80, 14.33. ¹⁹F NMR (376 MHz, CDCl₃) δ −125.88. HRMS (ESI-TOF) m/z Calcd for C₁₄H₁₃FNO₂⁺ [M+H]⁺ 246.0930, found 246.0932.

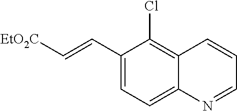 [SMILES: CCOC(=O)/C=C/c1ccc2ncccc2c1Cl]

Ethyl (E)-3-(5-chloroquinolin-6-yl)acrylate (2m) The general procedure 2.4 was followed and purification by preparative TLC (hexane/EA=3/1) furnished compound 2m as a white solid (9.6 mg, 37%, C6:others=97:3). ¹H NMR (600 MHz, CDCl₃) δ 8.97 (dd, J=4.2, 1.7 Hz, 1H), 8.68 (ddd, J=8.6, 1.7, 0.9 Hz, 1H), 8.35 (d, J=15.9 Hz, 1H), 8.03 (d, J=8.9 Hz, 1H), 7.93 (d, J=8.9 Hz, 1H), 7.55 (dd, J=8.6, 4.2 Hz, 1H), 6.58 (d, J=16.0 Hz, 1H), 4.34-4.31 (m, 2H), 1.38 (t, J=7.1 Hz, 3H). ¹³C NMR (151 MHz, CDCl₃) δ 166.39, 151.61, 149.32, 139.97, 133.69, 132.48, 130.44, 128.90, 127.01, 126.89, 122.50, 122.29, 60.88, 14.33. HRMS (ESI-TOF) m/z Calcd for C₁₄H₁₃ClNO₂⁺ [M+H]⁺ 262.0635, found 262.0638.

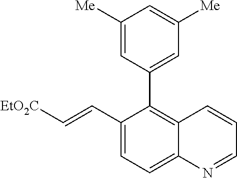 [SMILES: CCOC(=O)/C=C/c1ccc2ncccc2c1-c1cc(C)cc(C)c1]

Ethyl (E)-3-(5-(3,5-dimethylphenyl)quinolin-6-yl)acrylate (2n) The general procedure 2.4 was followed and purification by preparative TLC (hexane/EA=3/1) furnished compound 2n as a white solid (20.2 mg, 61%, C6:others>99:1). ¹H NMR (600 MHz, CDCl₃) δ 8.82 (dd, J=4.1, 1.7 Hz, 1H), 8.03 (d, J=9.0 Hz, 1H), 7.95 (d, J=9.0 Hz, 1H), 7.81 (ddd, J=8.5, 1.7, 0.8 Hz, 1H), 7.58 (d, J=15.9 Hz, 1H), 7.24 (dd, J=8.5, 4.1 Hz, 1H), 7.04 (tt, J=1.6, 0.7 Hz, 1H), 6.80 (dp, J=1.2, 0.6 Hz, 2H), 6.42 (d, J=16.0 Hz, 1H), 4.13 (q, J=7.1 Hz, 2H), 2.32 (s, 6H), 1.21 (t, J=7.1 Hz, 3H). ¹³C NMR (151 MHz, CDCl₃) δ 166.83, 150.81, 148.81, 142.79, 142.02, 137.99, 136.04, 135.80, 130.34, 129.86, 129.23, 128.44, 128.18, 126.50, 121.46, 119.65, 60.44, 21.37, 14.26. HRMS (ESI-TOF) m/z Calcd for C₂₂H₂₂NO₂⁺ [M+H]⁺ 332.1651, found 332.1656.

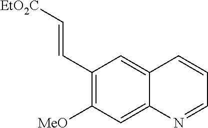 [SMILES: CCOC(=O)/C=C/c1cc2cccnc2cc1OC]

Ethyl (E)-3-(7-methoxyquinolin-6-yl)acrylate (20) The general procedure 2.4 was followed and purification by preparative TLC (hexane/EA=1/1) furnished compound 2o as colorless oil (11.3 mg, 44%, C6:others=80:20). ¹H NMR (600 MHz, CDCl₃) δ 8.84 (d, J=2.9 Hz, 1H), 8.10-8.04 (m, 2H), 7.94 (s, 1H), 7.45 (s, 1H), 7.29 (dd, J=8.2, 4.3 Hz, 1H), 6.71 (d, J=16.1 Hz, 1H), 4.30 (q, J=7.1 Hz, 2H), 4.03 (s, 3H), 1.36 (t, J=7.1 Hz, 3H). ¹³C NMR (151 MHz, CDCl₃) δ 167.11, 151.51, 139.51, 136.09, 128.76, 126.10, 123.14, 121.11, 119.66, 107.61, 60.58, 55.83, 14.37. HRMS (ESI-TOF) m/z Calcd for C₁₅H₁₆NO₃⁺ [M+H]⁻ 258.1130, found 258.1135.

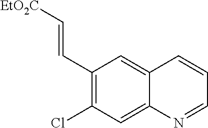 [SMILES: CCOC(=O)/C=C/c1cc2cccnc2cc1Cl]

Ethyl (E)-3-(7-chloroquinolin-6-yl)acrylate (2p) The general procedure 2.4 was followed and purification by preparative TLC (hexane/EA=5/1) furnished compound 2p as a white solid (12.3 mg, 47%, C6:others=95:5). ¹H NMR (600 MHz, CDCl₃) δ 8.93 (dd, J=4.2, 1.7 Hz, 1H), 8.22-8.14 (m, 3H), 8.08 (s, 1H), 7.43 (dd, J=8.3, 4.2 Hz, 1H), 6.57 (d, J=15.9 Hz, 1H), 4.32 (q, J=7.1 Hz, 2H), 1.37 (t, J=7.2 Hz, 3H). ¹³C NMR (151 MHz, CDCl₃) δ 166.19, 152.21, 148.73, 140.10, 136.19, 135.30, 131.96, 129.96, 127.13, 126.81, 122.25, 121.94, 60.86, 14.32. HRMS (ESI-TOF) m/z Calcd for C₁₄H₃ClNO₂⁺ [M+H]⁺ 262.0635, found 262.0635.

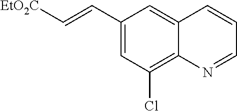 [SMILES: CCOC(=O)/C=C/c1cc(Cl)c2ncccc2c1]

Ethyl (E)-3-(8-chloroquinolin-6-yl)acrylate (2q) The general procedure 2.4 was followed and purification by preparative TLC (hexane/EA=5/1) furnished compound 2q as a white solid (10.7 mg, 41%, C6:others=91:9). ¹H NMR (600 MHz, CDCl₃) δ 9.06 (dd, J=4.2, 1.7 Hz, 1H), 8.21 (dd, J=8.3, 1.7 Hz, 1H), 8.05 (d, J=1.9 Hz, 1H), 7.84 (d, J=1.9 Hz, 1H), 7.78 (d, J=16.0 Hz, 1H), 7.52 (dd, J=8.2, 4.2 Hz, 1H), 6.57 (d, J=16.0 Hz, 1H), 4.30 (q, J=7.1 Hz, 2H), 1.37 (t, J=7.1 Hz, 3H). ¹³C NMR (151 MHz, CDCl₃) δ 166.43, 151.80, 145.10, 142.27, 136.95, 134.53, 133.01, 129.42, 128.08, 127.24, 122.68, 120.61, 60.82, 14.30. HRMS (ESI-TOF) m/z Calcd for C₁₄H₃ClNO₂⁺ [M+H]⁺ 262.0635, found 262.0639.

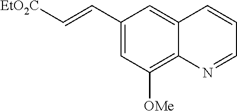 [SMILES: CCOC(=O)/C=C/c1cc(OC)c2ncccc2c1]

Ethyl (E)-3-(8-methoxyquinolin-6-yl)acrylate (2r) The general procedure 2.4 was followed and purification by preparative TLC (hexane/EA=1/1) furnished compound 2r as a white solid (21 mg, 82%, C6:others=90:10). ¹H NMR (600 MHz, CDCl₃) δ 8.94 (dd, J=4.2, 1.7 Hz, 1H), 8.14 (dd, J=8.3, 1.7 Hz, 1H), 7.81 (d, J=15.9 Hz, 1H), 7.51 (d, J=1.7 Hz, 11H), 7.46 (dd, J=8.2, 4.2 Hz, 1H), 7.20 (d, J=1.7 Hz, 1H), 6.55 (d, J=16.0 Hz, 1H), 4.31 (q, J=7.1 Hz, 2H), 4.13 (s, 3H), 1.37 (t, J=7.1 Hz, 3H). ¹³C NMR (151 MHz, CDCl₃) δ 166.76, 155.79, 150.24, 144.13, 141.32, 136.40, 132.95, 129.19, 122.46, 121.92, 119.33, 104.61, 60.70, 56.09, 14.35. HRMS (ESI-TOF) m/z Calcd for C₁₅H₁₆NO₃⁺ [M+H]⁺ 258.1130, found 258.1130.

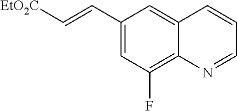 [SMILES: CCOC(=O)/C=C/c1cc(F)c2ncccc2c1]

Ethyl (E)-3-(8-fluoroquinolin-6-yl)acrylate (2s) The general procedure 2.4 was followed and purification by preparative TLC (hexane/EA=3/1) furnished compound 2s as a white solid (13.5 mg, 55%, C6:others=91:9). ¹H NMR (600 MHz, CDCl₃) δ 8.99 (dd, J=4.2, 1.6 Hz, 1H), 8.20 (dt, J=8.4, 1.6 Hz, 1H), 7.80 (d, J=15.9 Hz, 1H), 7.71 (s, 1H), 7.61 (dd, J=11.3, 1.8 Hz, 1H), 7.52 (dd, J=8.3, 4.2 Hz, 1H), 6.53 (d, J=16.0 Hz, 1H), 4.30 (q, J=7.1 Hz, 2H), 1.37 (t, J=7.1 Hz, 3H). ¹³C NMR (151 MHz, CDCl₃) δ 166.48, 158.31 (d, J=258.4 Hz), 151.39 (d, J=2.1 Hz), 142.74 (d, J=2.8 Hz), 139.39 (d, J=12.6 Hz), 136.22 (d, J=3.4 Hz), 133.11 (d, J=7.8 Hz), 129.78 (d, J=3.0 Hz), 125.01 (d, J=4.0 Hz), 122.86, 120.50, 110.93 (d, J=19.9 Hz), 60.83, 14.32. ¹⁹F NMR (376 MHz, CDCl₃) δ −126.92. HRMS (ESI-TOF) m/z Calcd for C₁₄H₃FNO₂⁺ [M+H]⁺ 246.0930, found 246.0937

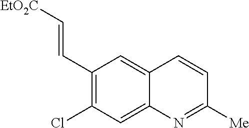 [SMILES: CCOC(=O)/C=C/c1cc2ccc(C)nc2cc1Cl]

Ethyl (E)-3-(7-chloro-2-methylquinolin-6-yl)acrylate (2t) The general procedure 2.4 was followed and purification by preparative TLC (hexane/EA=5/1) furnished compound 2t as a white solid (21.2 mg, 77%, C6:others=93:7). ¹H NMR (600 MHz, CDCl₃) δ 8.19 (d, J=16.0 Hz, 1H), 8.08 (s, 1H), 8.04-8.02 (m, 2H), 7.31 (d, J=8.4 Hz, 1H), 6.55 (d, J=15.9 Hz, 1H), 4.31 (q, J=7.1 Hz, 2H), 2.74 (s, 3H), 1.37 (t, J=7.1 Hz, 3H). ¹³C NMR (151 MHz, CDCl₃) δ 166.32, 161.35, 148.45, 140.27, 136.18, 135.26, 130.93, 129.22, 126.84, 125.04, 122.94, 121.64, 60.79, 25.53, 14.33. HRMS (ESI-TOF) m/z Calcd for C₁₅H₁₅ClNO₂⁺ [M+H]⁺ 276.0791, found 276.0793.

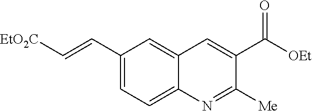 [SMILES: CCOC(=O)/C=C/c1ccc2nc(C)c(C(=O)OCC)cc2c1]

Ethyl (E)-3-(7-chloroquinolin-6-yl)acrylate (2u) The general procedure 2.4 was followed and purification by preparative TLC (hexane/EA=5/1) furnished compound 2u as a white solid (18.2 mg, 58%, C6:others=94:6). ¹H NMR (600 MHz, CDCl₃) δ 8.73 (s, 1H), 8.03 (d, J=8.7 Hz, 1H), 7.97-7.94 (m, 2H), 7.83 (d, J=16.0 Hz, 1H), 6.57 (d, J=16.0 Hz, 1H), 4.46 (q, J=7.1 Hz, 2H), 4.30 (q, J=7.2 Hz, 2H), 3.00 (s, 3H), 1.47 (t, J=7.2 Hz, 3H), 1.37 (t, J=7.1 Hz, 3H). ¹³C NMR (151 MHz, CDCl₃) δ 166.72, 166.26, 159.69, 149.32, 143.25, 140.03, 132.71, 129.65, 129.39, 129.36, 125.80, 124.76, 119.74, 61.57, 60.73, 25.76, 14.34, 14.32. HRMS (ESI-TOF) m/z Calcd for C₁₈H₂₀NO₄⁺[ M+H]⁺ 314.1392, found 314.1393.

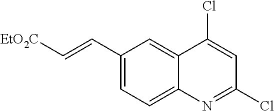 [SMILES: CCOC(=O)/C=C/c1ccc2nc(Cl)cc(Cl)c2c1]

Ethyl (E)-3-(2,4-dichloroquinolin-6-yl)acrylate (2v) The general procedure 2.4 was followed except using T8 (0.2 equiv) and purification by preparative TLC (hexane/EA=20/1) furnished compound 2v as a white solid (14.4 mg, 49%, C6:others=95:5). ¹H NMR (600 MHz, CDCl₃) δ 8.28 (d, J=1.9 Hz, 1H), 8.03 (d, J=8.8 Hz, 1H), 7.96 (dd, J=8.8, 1.9 Hz, 1H), 7.86 (d, J=16.0 Hz, 1H), 7.55 (s, 1H), 6.61 (d, J=16.0 Hz, 1H), 4.31 (q, J=7.1 Hz, 2H), 1.37 (t, J=7.1 Hz, 3H). ¹³C NMR (151 MHz, CDCl₃) δ 166.44, 150.87, 148.81, 144.62, 142.81, 134.10, 129.77, 129.51, 125.37, 125.07, 122.76, 120.89, 60.86, 14.33. HRMS (ESI-TOF) m/z Calcd for C₁₄H₁₂Cl₂NO₂⁺ [M+H]⁺ 296.0245, found 296.0250.

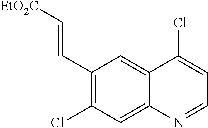 [SMILES: CCOC(=O)/C=C/c1cc2c(Cl)ccnc2cc1Cl]

Ethyl (E)-3-(4,7-dichloroquinolin-6-yl)acrylate (2w) The general procedure 2.4 was followed and purification by preparative TLC (hexane/EA=10/1) furnished compound 2w as a white solid (24.5 mg, 83%, C6:others>99:1). ¹H NMR (600 MHz, CDCl₃) δ 8.78 (d, J=4.7 Hz, 1H), 8.46 (s, 1H), 8.19 (t, J=8.0 Hz, 2H), 7.50 (d, J=4.7 Hz, 1H), 6.64 (d, J=15.9 Hz, 1H), 4.33 (q, J=7.1 Hz, 2H), 1.38 (t, J=7.1 Hz, 3H). ¹³C NMR (151 MHz, CDCl₃) δ 166.05, 151.72, 149.43, 142.83, 139.69, 136.45, 132.97, 130.27, 125.24, 123.57, 123.09, 121.98, 60.96, 14.32. HRMS (ESI-TOF) m/z Calcd for C₁₄H₁₂Cl₂NO₂⁺ [M+H]⁺ 296.0245, found 296.0248.

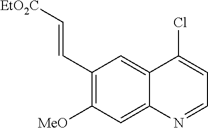 [SMILES: CCOC(=O)/C=C/c1cc2c(Cl)ccnc2cc1OC]

Ethyl (E)-3-(4-chloro-7-methoxyquinolin-6-yl)acrylate (2x) The general procedure 2.4 was followed and purification by preparative TLC (hexane/EA=1/1) furnished compound 2x as a pale yellow solid (16.3 mg, 56%, C6:others=80:20). ¹H NMR (600 MHz, CDCl₃)¹H NMR (600 MHz, Chloroform-d) δ 8.69 (d, J=4.7 Hz, 1H), 8.33 (s, 1H), 8.08 (d, J=16.1 Hz, 1H), 7.45 (s, 1H), 7.36 (d, J=4.7 Hz, 1H), 6.76 (d, J=16.1 Hz, 1H), 4.31 (q, J=7.1 Hz, 2H), 4.04 (s, 3H), 1.37 (t, J=7.2 Hz, 3H). ¹³C NMR (151 MHz, CDCl₃) δ 166.93, 159.55, 151.18, 151.04, 142.69, 139.07, 127.03, 125.00, 121.88, 121.34, 119.82, 107.91, 60.68, 56.00, 14.36. HRMS (ESI-TOF) m/z Calcd for C₁₅H₁₅ClNO₃⁺ [M+H]⁺ 292.0740, found 292.0742.

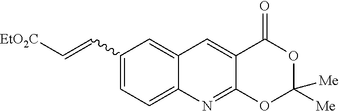 [SMILES: CCOC(=O)C=Cc1ccc2nc3c(cc2c1)C(=O)OC(C)(C)O3]

Ethyl (E)-3-(2,2-dimethyl-4-oxo-4H-[1,3]dioxino[4,5-b]quinolin-7-yl)acrylate (2y) The general procedure 2.4 was followed and purification by preparative TLC (hexane/EA=3/1) furnished compound 2y as a white solid (13.0 mg, 40%, C_{shown}:others=86:14). ¹H NMR (600 MHz, CDCl₃) δ 8.93 (s, 1H), 8.02-7.98 (m, 2H), 7.94 (d, J=8.6 Hz, 1H), 7.82 (d, J=16.0 Hz, 1H), 6.56 (d, J=16.0 Hz, 1H), 4.31 (q, J=7.1 Hz, 2H), 1.86 (s, 6H), 1.37 (t, J=7.1 Hz, 3H). ¹³C NMR (151 MHz, CDCl₃) δ 166.53, 160.27, 158.75, 150.85, 142.66, 142.62, 132.59, 131.41, 130.17, 128.81, 125.58, 120.15, 109.51, 107.07, 60.82, 26.65, 14.33. HRMS (ESI-TOF) m/z Calcd for C₁₈H₁₈NO₅⁺ [M+H]⁺ 328.1185, found 328.1191.

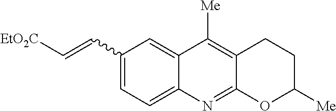 [SMILES: CCOC(=O)C=Cc1ccc2nc3c(c(C)c2c1)CCC(C)O3]

Ethyl (E)-3-(2,5-dimethyl-3,4-dihydro-2H-pyrano[2,3-b]quinolin-7-yl)acrylate (2z) The general procedure 2.4 was followed and purification by preparative TLC (hexane/EA=3/1) furnished compound 2z as a white solid (10.3 mg, 33%, C_{shown}:others=89:11). ¹H NMR (600 MHz, CDCl₃) δ 7.97 (d, J=1.8 Hz, 1H), 7.84 (d, J=15.9 Hz, 1H), 7.80 (d, J=8.7 Hz, 1H), 7.77 (dd, J=8.8, 1.8 Hz, 1H), 6.50 (d, J=15.9 Hz, 1H), 4.41 (ddt, J=12.6, 6.3, 4.2 Hz, 1H), 4.29 (q, J=7.2 Hz, 2H), 3.03-2.98 (m, 1H), 2.91-2.85 (m, 1H), 2.57 (d, J=0.9 Hz, 3H), 2.18-2.14 (m, 1H), 1.85-1.79 (m, 1H), 1.53 (d, J=6.3 Hz, 3H), 1.36 (t, J=7.1 Hz, 3H). ¹³C NMR (151 MHz, CDCl₃) δ 167.15, 160.89, 147.03, 144.72, 144.69, 129.94, 128.77, 126.67, 125.42, 125.04, 117.66, 117.05, 73.48, 60.51, 28.81, 23.53, 21.36, 14.38, 13.93. HRMS (ESI-TOF) m/z Calcd for C₁₉H₂₂NO₃⁺ [M+H]⁺ 312.1600, found 312.1599.

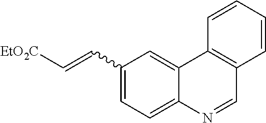 [SMILES: CCOC(=O)C=Cc1ccc2ncc3ccccc3c2c1]

Ethyl (E)-3-(phenanthridin-2-yl)acrylate (2aa) The general procedure 2.4 was followed except using T15 (0.2 equiv) and TC10 (0.8 equiv) and purification by preparative TLC (hexane/EA=3/1) furnished compound 2aa as a pale yellow solid (13.6 mg, 49%, C_{shown}:others=91:9). ¹H NMR (600 MHz, CDCl₃) δ 9.30 (s, 1H), 8.67 (s, 1H), 8.63 (d, J=8.3 Hz, 1H), 8.18 (d, J=8.5 Hz, 1H), 8.07 (d, J=7.9 Hz, 1H), 7.97-7.89 (m, 3H), 7.75 (t, J=7.5 Hz, 1H), 6.64 (dd, J=15.9, 1.1 Hz, 1H), 4.32 (q, J=7.1 Hz, 2H), 1.39 (t, J=7.2 Hz, 3H). ¹³C NMR (151 MHz, CDCl₃) δ 166.87, 154.53, 145.50, 144.14, 133.10, 132.36, 131.43, 130.84, 128.99, 128.00, 126.86, 126.63, 124.30, 123.47, 121.81, 119.40, 60.68, 14.37. HRMS (ESI-TOF) m/z Calcd for C₁₈H₁₆NO₂⁺ [M+H]⁺ 278.1181, found 278.1185.

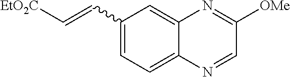 [SMILES: CCOC(=O)C=Cc1ccc2ncc(OC)nc2c1]

Ethyl (E)-3-(3-methoxyquinoxalin-6-yl)acrylate (2ab) The general procedure 2.4 was followed except using T15 (0.2 equiv) and TC10 (0.8 equiv) and purification by preparative TLC (hexane/EA=10/1) furnished compound 2ab as a white solid (19.6 mg, 76%, C7:others=91:9). ¹H NMR (600 MHz, CDCl₃) δ 8.46 (s, 1H), 8.00 (d, J=8.5 Hz, 1H), 7.96 (d, J=1.9 Hz, 1H), 7.83 (d, J=15.9 Hz, 1H), 7.73 (dd, J=8.6, 1.9 Hz, 1H), 6.61 (d, J=16.0 Hz, 1H), 4.30 (q, J=7.1 Hz, 2H), 4.11 (s, 3H), 1.37 (t, J=7.1 Hz, 3H). ¹³C NMR (151 MHz, CDCl₃) δ 166.67, 158.19, 143.48, 140.58, 140.36, 139.72, 136.19, 129.55, 127.72, 124.98, 120.43, 60.74, 53.87, 14.33. HRMS (ESI-TOF) m/z Calcd for C₁₄H₁₅N₂O₃⁺ [M+H]⁺ 259.1083, found 259.1089.

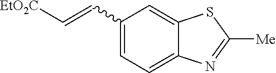 [SMILES: CCOC(=O)C=Cc1ccc2nc(C)sc2c1]

Ethyl (E)-3-(2-methylbenzo[d]thiazol-6-yl)acrylate (2ac) The general procedure 2.4 was followed and purification by preparative TLC (hexane/EA=10/1) furnished compound 2ac as a white solid (11.6 mg, 47%, C6:others=86:14). ¹H NMR (600 MHz, CDCl₃) δ 7.96 (d, J=1.7 Hz, 1H), 7.93 (d, J=8.5 Hz, 1H), 7.77 (d, J=15.9 Hz, 1H), 7.63 (dd, J=8.5, 1.7 Hz, 1H), 6.48 (d, J=15.9 Hz, 1H), 4.28 (t, J=7.1 Hz, 2H), 2.85 (s, 3H), 1.35 (t, J=7.1 Hz, 3H). ¹³C NMR (151 MHz, CDCl₃) δ 168.96, 166.89, 154.62, 143.98, 136.43, 131.32, 125.54, 122.69, 121.62, 118.50, 60.59, 20.32, 14.34. HRMS (ESI-TOF) m/z Calcd for C₁₃H₁₄NO₂S⁺ [M+H]⁺ 248.0745, found 248.0747.

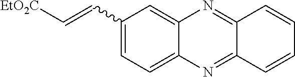 [SMILES: CCOC(=O)C=Cc1ccc2nc3ccccc3nc2c1]

Ethyl (E)-3-(phenazin-2-yl)acrylate (2ad) The general procedure 2.4 was followed and purification by preparative TLC (hexane/EA=3/1) furnished compound 2ad as a white solid (15.6 mg, 56%, C_{shown}:others=94:6). ¹H NMR (600 MHz, CDCl₃) δ 8.25 (d, J=1.5 Hz, 1H), 8.20-8.15 (m, 3H), 7.96 (dd, J=9.1, 1.9 Hz, 1H), 7.87 (dd, J=16.0, 0.6 Hz, 1H), 7.80 (dt, J=6.6, 3.3 Hz, 2H), 6.62 (d, J=16.0 Hz, 1H), 4.26 (t, J=7.1 Hz, 2H), 1.32 (t, J=7.1 Hz, 3H). ¹³C NMR (151 MHz, CDCl₃) δ 166.83, 150.81, 148.81, 142.79, 142.02, 137.99, 136.04, 135.80, 130.34, 129.86, 129.23, 128.44, 128.18, 126.50, 121.46, 119.65, 60.44, 21.37, 14.26. HRMS (ESI-TOF) m/z Calcd for C₇H₁₅N₂O₂⁺ [M+H]⁺ 279.1134, found 279.1135.

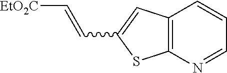 [SMILES: CCOC(=O)C=Cc1cc2cccnc2s1]

Ethyl (E)-3-(thieno[2,3-b]pyridin-2-yl)acrylate (2ae) The general procedure 2.4 was followed and purification by preparative TLC (hexane/EA=3/1) furnished compound 2ae as a white solid (19.6 mg, 84%, C6:others=94:6). ¹H NMR (600 MHz, CDCl₃) δ 8.59 (dd, J=4.6, 1.6 Hz, 1H), 8.04 (dd, J=8.0, 1.6 Hz, 1H), 7.86 (dd, J=15.7, 0.7 Hz, 1H), 7.41 (s, 1H), 7.32 (dd, J=8.0, 4.6 Hz, 1H), 6.39 (d, J=15.6 Hz, 1H), 4.31 (q, J=7.1 Hz, 2H), 1.37 (t, J=7.1 Hz, 3H). ¹³C NMR (151 MHz, CDCl₃) δ 166.27, 161.89, 148.12, 139.81, 137.40, 133.19, 131.56, 125.68, 120.99, 120.13, 60.84, 14.30. HRMS (ESI-TOF) m/z Calcd for C₁₂H₁₂NO₂S⁺ [M+H]⁺ 234.0589, found 234.0593.

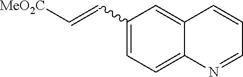 [SMILES: COC(=O)C=Cc1ccc2ncccc2c1]

Methyl (E)-3-(quinolin-6-yl)acrylate (2af) The general procedure 2.4 was followed and purification by preparative TLC (hexane/EA=3/1) furnished compound 2af as a white solid (12.4 mg, 58%, C6:others=92:8). ¹H NMR (600 MHz, CDCl₃) δ 8.93 (dd, J=4.2, 1.7 Hz, 1H), 8.19-8.17 (m, 1H), 8.11 (d, J=9.3 Hz, 1H), 7.92-7.90 (m, 2H), 7.86 (d, J=16.0 Hz, 1H), 7.44 (dd, J=8.3, 4.2 Hz, 1H), 6.58 (d, J=16.0 Hz, 1H), 3.85 (s, 3H). ¹³C NMR (151 MHz, CDCl₃) δ 167.23, 151.39, 149.08, 143.91, 136.47, 132.63, 130.32, 129.31, 128.25, 127.25, 121.88, 119.14, 51.86. HRMS (ESI-TOF) m/z Calcd for C₃H₁₂NO₂⁺ [M+H]⁺ 214.0868, found 214.0871,

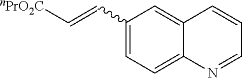

Propyl (E)-3-(quinolin-6-yl)acrylate (2ag) The general procedure 2.4 was followed and purification by preparative TLC (hexane/EA=3/1) furnished compound 2ag as a pale yellow solid (16.6 mg, 69%, C6:others=92:8). ¹H NMR (600 MHz, CDCl₃) δ 8.93 (dd, J=4.2, 1.7 Hz, 1H), 8.17 (dd, J=8.4, 1.2 Hz, 1H), 8.10 (d, J=9.3 Hz, 1H), 7.93-7.89 (m, 2H), 7.85 (d, J=16.2 Hz, 1H), 7.44 (dd, J=8.2, 4.2 Hz, 1H), 6.59 (d, J=16.0 Hz, 1H), 4.21 (t, J=6.7 Hz, 2H), 1.76 (q, J=6.9 Hz, 2H), 1.02 (t, J=7.4 Hz, 3H). ¹³C NMR (151 MHz, CDCl₃) δ 166.88, 151.34, 149.06, 143.59, 136.45, 132.73, 130.28, 129.22, 128.27, 127.28, 121.86, 119.65, 66.33, 22.11, 10.48. HRMS (ESI-TOF) m/z Calcd for C₁₅H₁₆NO₂⁺ [M+H]⁻ 242.1181, found 242.1185.

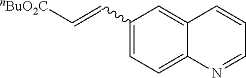

Butyl (E)-3-(quinolin-6-yl)acrylate (2ah) The general procedure 2.4 was followed and purification by preparative TLC (hexane/EA=3/1) furnished compound 2ah as colorless oil (20.4 mg, 80%, C6:others=93:7). ¹H NMR (600 MHz, CDCl₃) δ 8.93 (dd, J=4.2, 1.7 Hz, 1H), 8.17 (dd, J=8.3, 1.3 Hz, 1H), 8.10 (d, J=9.3 Hz, 1H), 7.91 (dq, J=4.5, 2.0 Hz, 2H), 7.85 (d, J=16.0 Hz, 1H), 7.44 (dd, J=8.3, 4.2 Hz, 1H), 6.58 (d, J=16.0 Hz, 1H), 4.25 (t, J=6.7 Hz, 2H), 1.74-1.70 (m, 2H), 1.46 (dt, J=14.8, 7.4 Hz, 2H), 0.98 (t, J=7.4 Hz, 3H). ¹³C NMR (151 MHz, CDCl₃) δ 166.89, 151.34, 149.06, 143.57, 136.45, 132.74, 130.28, 129.22, 128.27, 127.28, 121.86, 119.67, 64.62, 30.79, 19.22, 13.77. HRMS (ESI-TOF) m/z Calcd for C₁₆H₁₈NO₂⁺ [M+H]⁺ 256.1338, found 256.1343.

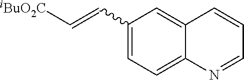

Isobutyl (E)-3-(quinolin-6-yl)acrylate (2ai) The general procedure 2.4 was followed and purification by preparative TLC (hexane/EA=3/1) furnished compound 2ai as colorless oil (21.2 mg, 83%, C6:others=94:6). ¹H NMR (600 MHz, CDCl₃) δ 8.93 (dd, J=4.2, 1.7 Hz, 1H), 8.19-8.16 (m, 1H), 8.10 (d, J=9.5 Hz, 1H), 7.93-7.89 (m, 2H), 7.85 (d, J=16.0 Hz, 1H), 7.44 (dd, J=8.3, 4.2 Hz, 1H), 6.60 (d, J=16.0 Hz, 1H), 4.03 (d, J=6.7 Hz, 2H), 2.04 (dq, J=13.4, 6.7 Hz, 1H), 1.01 (d, J=6.8 Hz, 6H). ¹³C NMR (151 MHz, CDCl₃) δ 166.86, 151.34, 149.05, 143.59, 136.44, 132.73, 130.28, 129.23, 128.26, 127.29, 121.86, 119.65, 70.83, 27.86, 19.18. HRMS (ESI-TOF) m/z Calcd for C₁₆H₁₈NO₂⁺ [M+H]⁺ 256.1338, found 265.1342.

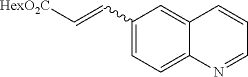

Hexyl (E)-3-(quinolin-6-yl)acrylate (2aj) The general procedure 2.4 was followed and purification by preparative TLC (hexane/EA=3/1) furnished compound 2aj as pale yellow oil (19.8 mg, 70%, C6:others=93:7). ¹H NMR (600 MHz, CDCl₃) δ 8.93 (dd, J=4.2, 1.7 Hz, 1H), 8.17 (dd, J=8.8, 1.2 Hz, 1H), 8.10 (d, J=9.3 Hz, 1H), 7.91 (dq, J=3.9, 2.0 Hz, 2H), 7.85 (d, J=16.0 Hz, 1H), 7.44 (dd, J=8.3, 4.2 Hz, 1H), 6.59 (d, J=16.0 Hz, 1H), 4.24 (t, J=6.7 Hz, 2H), 1.74-1.71 (m, 2H), 1.45-1.39 (m, 2H), 1.34 (ddd, J=7.2, 4.5, 3.2 Hz, 4H), 0.93-0.90 (m, 3H). ¹³C NMR (151 MHz, CDCl₃) δ 166.89, 151.34, 149.06, 143.57, 136.45, 132.74, 130.28, 129.22, 128.27, 127.29, 121.86, 119.68, 64.92, 31.48, 28.71, 25.67, 22.57, 14.03. HRMS (ESI-TOF) m/z Calcd for C₁₈H₂₂NO₂⁺ [M+H]⁺ 284.1651, found 284.2643.

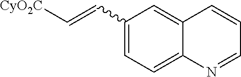

Cyclohexyl (E)-3-(quinolin-6-yl)acrylate (2ak) The general procedure 2.4 was followed and purification by preparative TLC (hexane/EA=3/1) furnished compound 2ak as colorless oil (12.9 mg, 46%, C6:others=93:7). ¹H NMR (600 MHz, CDCl₃) δ 8.93 (dd, J=4.2, 1.7 Hz, 1H), 8.17 (ddd, J=8.3, 1.8, 0.8 Hz, 1H), 8.10 (d, J=9.3 Hz, 1H), 7.91 (dq, J=4.6, 2.0 Hz, 2H), 7.83 (d, J=15.9 Hz, 1H), 7.44 (dd, J=8.3, 4.2 Hz, 1H), 6.58 (d, J=16.0 Hz, 1H), 4.93 (tt, J=9.2, 3.9 Hz, 1H), 1.97-1.92 (m, 2H), 1.79 (dp, J=13.4, 4.3 Hz, 2H), 1.59 (dq, J=130.2, 4.9, 4.5 Hz, 1H), 1.55-1.48 (m, 2H), 1.46-1.40 (m, 2H), 1.32 (ddt, J=13.8, 10.3, 3.6 Hz, 1H). ¹³C NMR (151 MHz, CDCl₃) δ 166.23, 151.29, 149.03, 143.27, 136.44, 132.83, 130.25, 129.14, 128.27, 127.31, 121.84, 120.28, 72.98, 31.75, 25.44, 23.82. HRMS (ESI-TOF) m/z Calcd for C₁₈H₂₀NO₂⁺ [M+H]⁺ 282.1494, found 282.1498.

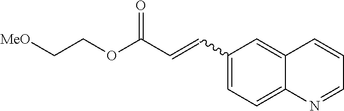 [SMILES: COCCOC(=O)C=Cc1ccc2ncccc2c1]

2-Methoxyethyl (E)-3-(quinolin-6-yl)acrylate (2a1) The general procedure 2.4 was followed and purification by preparative TLC (hexane/EA=1/1) furnished compound 2al as pale yellow oil (16.7 mg, 65%, C6:others=91:9). ¹H NMR (600 MHz, CDCl₃) δ 8.93 (dd, J=4.2, 1.7 Hz, 1H), 8.18 (dd, J=8.5, 1.0 Hz, 1H), 8.10 (d, J=8.5 Hz, 1H), 7.92-7.85 (m, 3H), 7.44 (dd, J=8.3, 4.2 Hz, 1H), 6.64 (d, J=16.0 Hz, 1H), 4.42-4.39 (m, 2H), 3.71-3.68 (m, 2H), 3.44 (s, 3H). ¹³C NMR (151 MHz, CDCl₃) δ 166.75, 151.40, 149.09, 144.15, 136.49, 132.63, 130.32, 129.37, 128.25, 127.26, 121.87, 119.19, 70.57, 63.74, 59.08. HRMS (ESI-TOF) m/z Calcd for C₁₅H₁₆NO₃⁺ [M+H]⁺ 258.1130, found 258.1135.

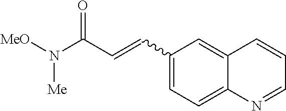 [SMILES: CON(C)C(=O)C=Cc1ccc2ncccc2c1]

(E)-N,N-Dimethyl-3-(quinolin-6-yl)acrylamide (2am) The general procedure 2.4 was followed and purification by preparative TLC (EA) furnished compound 2am as a white solid (12.2 mg, 54%, C6:others=97:3). ¹H NMR (600 MHz, CDCl₃) δ 8.93-8.90 (m, 1H), 8.17 (d, J=8.2 Hz, 1H), 8.09 (d, J=8.8 Hz, 1H), 7.94 (dd, J=8.8, 2.0 Hz, 1H), 7.89 (s, 1H), 7.84 (d, J=15.4 Hz, 1H), 7.43 (dd, J=8.3, 4.2 Hz, 1H), 7.04 (d, J=15.4 Hz, 1H), 3.23 (s, 3H), 3.10 (s, 3H). ¹³C NMR (151 MHz, CDCl₃) δ 166.42, 151.03, 148.81, 141.47, 136.38, 133.58, 130.07, 128.79, 128.37, 127.23, 121.77, 118.72, 37.49, 36.02. HRMS (ESI-TOF) m/z Calcd for C₁₄H₁₅N₂O⁺ [M+H]⁺ 227.1184, found 227.1184.

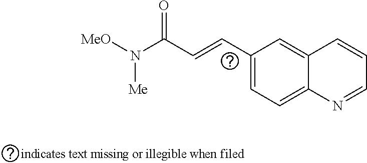 [SMILES: CON(C)C(=O)/C=C/c1ccc2ncccc2c1]

(E)-N-Methoxy-N-methyl-3-(quinolin-6-yl)acrylamide (2an) The general procedure 2.4 was followed and purification by preparative TLC (EA) furnished compound 2an as a white solid (14.8 mg, 61%, C6:others=97:3). ¹H NMR (600 MHz, CDCl₃) δ 8.92 (dd, J=4.2, 1.7 Hz, 1H), 8.19 (dd, J=8.3, 1.2 Hz, 1H), 8.11 (d, J=9.4 Hz, 1H), 7.98 (dd, J=8.8, 2.0 Hz, 1H), 7.93 (d, J=1.9 Hz, 1H), 7.90 (d, J=15.8 Hz, 1H), 7.44 (dd, J=8.3, 4.2 Hz, 1H), 7.18 (d, J=15.8 Hz, 1H), 3.81 (s, 3H), 3.35 (s, 3H). ¹³C NMR (151 MHz, CDCl₃) δ 166.69, 151.14, 148.94, 142.51, 136.44, 133.41, 130.10, 129.18, 128.34, 127.39, 121.79, 117.11, 62.01, 32.57. HRMS (ESI-TOF) m/z Calcd for C₁₄H₁₅N₂O₂⁺ [M+H]⁺ 243.1134, found 243.1140.

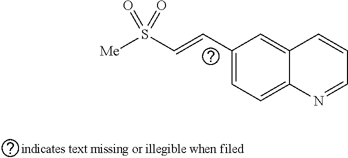 [SMILES: CS(=O)(=O)/C=C/c1ccc2ncccc2c1]

(E)-6-(2-(Methylsulfonyl)vinyl)quinoline (2ao) The general procedure 2.4 was followed and purification by preparative TLC (EA/acetone=10/1) furnished compound 2ao as colorless oil (15.8 mg, 68%, C6:others=95:5). ¹H NMR (600 MHz, CDCl₃) δ 8.98 (dd, J=4.2, 1.7 Hz, 1H), 8.21 (dd, J=8.1, 1.3 Hz, 1H), 8.15 (d, J=8.8 Hz, 1H), 7.96 (d, J=2.0 Hz, 1H), 7.86 (dd, J=8.8, 2.1 Hz, 1H), 7.81 (d, J=15.4 Hz, 1H), 7.48 (dd, J=8.2, 4.2 Hz, 1H), 7.07 (d, J=15.4 Hz, 1H), 3.09 (s, 3H). ¹³C NMR (151 MHz, CDCl₃) δ 152.04, 149.37, 143.11, 136.64, 130.77, 130.56, 130.29, 128.19, 127.41, 127.04, 122.20, 43.32. HRMS (ESI-TOF) m/z Calcd for C₁₂H₁₂NO₂S⁺ [M+H]⁺ 234.0589, found 234.0595.

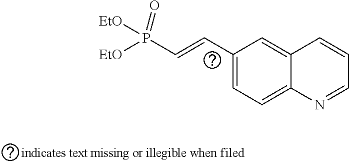 [SMILES: CCOP(=O)(/C=C/c1ccc2ncccc2c1)OCC]

Diethyl (E)-(2-(quinolin-6-yl)vinyl)phosphonate (2ap) The general procedure 2.4 was followed and purification by preparative TLC (EA/acetone=1/1) furnished compound 2ap as colorless oil (19.3 mg, 66%, C6:others=96:4). ¹H NMR (600 MHz, CDCl₃) δ 8.94 (dd, J=4.2, 1.7 Hz, 1H), 8.18 (dd, J=8.4, 1.2 Hz, 1H), 8.11 (d, J=8.6 Hz, 1H), 7.90 (dd, J=11.0, 2.3 Hz, 2H), 7.68 (dd, J=22.4, 17.5 Hz, 1H), 7.44 (dd, J=8.3, 4.2 Hz, 1H), 6.42 (t, J=17.2 Hz, 1H), 4.21-4.14 (m, 4H), 1.38 (t, J=7.1 Hz, 6H). ¹³C NMR (151 MHz, CDCl₃) δ 151.39, 149.03, 147.73, 147.68, 136.54, 133.13, 132.97, 130.26, 128.90, 128.89, 128.22, 128.21, 126.94, 121.89, 116.29, 115.02, 62.01, 61.98, 16.47, 16.43. HRMS (ESI-TOF) m/z Calcd for C₁₅H₁₉NO₃P⁺ [M+H]⁺ 292.1103, found 292.1107.

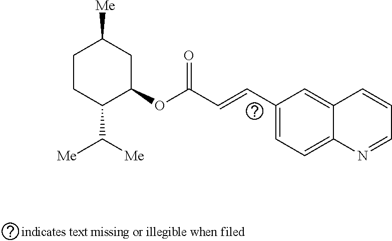 [SMILES: CC(C)[C@@H]1CC[C@@H](C)C[C@H]1OC(=O)/C=C/c1ccc2ncccc2c1]

(1R,2S,5R)-2-Isopropyl-5-methylcyclohexyl (E)-3-(quinolin-6-yl)acrylate (2aq) The general procedure 2.4 was followed and purification by preparative TLC (hexane/EA=5/1) furnished compound 2aq as colorless oil (22.6 mg, 67%, C6:others=94:6). ¹H NMR (600 MHz, CDCl₃) δ 8.93 (dd, J=4.2, 1.7 Hz, 1H), 8.19-8.16 (m, 1H), 8.10 (d, J=9.3 Hz, 1H), 7.92 (td, J=4.5, 1.9 Hz, 2H), 7.84 (d, J=15.8 Hz, 1H), 7.44 (dd, J=8.3, 4.2 Hz, 1H), 6.58 (d, J=16.0 Hz, 1H), 4.86 (td, J=10.9, 4.5 Hz, 1H), 2.09 (dtd, J=12.1, 4.2, 3.7, 1.7 Hz, 1H), 1.95 (pd, J=7.0, 2.7 Hz, 1H), 1.72 (dt, J=12.7, 3.0 Hz, 2H), 1.59-1.51 (m, 1H), 1.50-1.45 (m, 1H), 1.15-1.04 (m, 2H), 0.93 (dd, J=6.8, 2.4 Hz, 7H), 0.81 (d, J=6.9 Hz, 3H). ¹³C NMR (151 MHz, CDCl₃) δ 166.37, 151.30, 149.03, 143.38, 136.44, 132.82, 130.24, 129.16, 128.27, 127.32, 121.84, 120.12, 74.51, 47.23, 41.04, 34.31, 31.45, 26.39, 23.55, 22.06, 20.79, 16.46. HRMS (ESI-TOF) m/z Calcd for C₂₂H₂₈NO₂⁺ [M+H]⁻ 338.2120, found 338.2120.

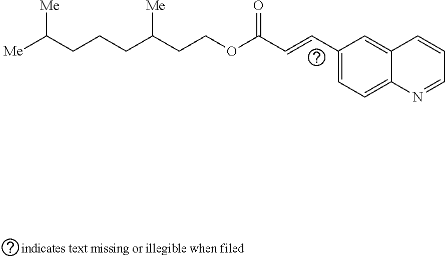 [SMILES: CC(C)CCCC(C)CCOC(=O)/C=C/c1ccc2ncccc2c1]

3,7-Dimethyloctyl (E)-3-(quinolin-6-yl)acrylate (2ar) The general procedure 2.4 was followed and purification by preparative TLC (hexane/EA=5/1) furnished compound 2ar as colorless oil (23.0 mg, 68%, C6:others=94:6). H NMR (600 MHz, CDCl₃) δ 8.93 (dd, J=4.2, 1.7 Hz, 1H), 8.17 (dd, J=8.4, 1.4 Hz, 1H), 8.10 (d, J=9.3 Hz, 1H), 7.92-7.90 (m, 2H), 7.84 (d, J=16.0 Hz, 1H), 7.44 (dd, J=8.2, 4.2 Hz, 1H), 6.58 (d, J=15.9 Hz, 1H), 4.31-4.25 (m, 2H), 1.79-1.74 (m, 1H), 1.65-1.59 (m, 1H), 1.56-1.50 (m, 2H), 1.36-1.31 (m, 2H), 1.30-1.25 (m, 1H), 1.20-1.14 (m, 3H), 0.95 (d, J=6.7 Hz, 3H), 0.88 (dd, J=6.6, 1.4 Hz, 6H). ¹³C NMR (151 MHz, CDCl₃) δ 166.88, 151.34, 149.05, 143.56, 136.45, 132.74, 130.28, 129.22, 128.26, 127.28, 121.86, 119.69, 63.36, 39.23, 37.17, 35.63, 29.92, 27.97, 24.64, 22.71, 22.62, 19.58. HRMS (ESI-TOF) m/z Calcd for C₂₂H₃₀NO₂⁺ [M+H]⁻ 340.2277, found 340.2276.

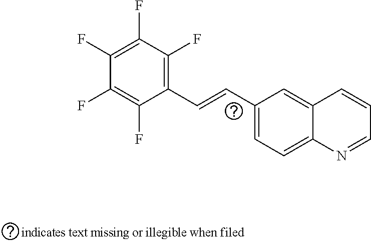 [SMILES: Fc1c(F)c(F)c(/C=C/c2ccc3ncccc3c2)c(F)c1F]

(E)-6-(2-(Perfluorophenyl)vinyl)quinoline (2as) The general procedure 2.4 was followed and purification by preparative TLC (hexane/EA=5/1) furnished compound 2as as a pale yellow solid (26.3 mg, 82%, C6:others=91:9). ¹H NMR (600 MHz, CDCl₃) δ 8.91 (dd, J=4.2, 1.7 Hz, 1H), 8.17 (dd, J=8.7, 1.1 Hz, 1H), 8.12 (d, J=8.8 Hz, 1H), 7.97 (dd, J=8.9, 2.0 Hz, 1H), 7.86 (d, J=2.0 Hz, 1H), 7.60 (d, J=16.8 Hz, 1H), 7.43 (dd, J=8.2, 4.2 Hz, 1H), 7.13 (d, J=16.7 Hz, 1H). ¹³C NMR (151 MHz, CDCl₃) δ 150.86, 148.57, 136.21, 134.67, 130.19, 128.44, 127.36, 126.72, 121.80, 114.11 (d, J=3.9 Hz). ¹⁹F NMR (376 MHz, CDCl₃) δ −145.09 (dd, J=21.4, 8.6 Hz, 2F), −158.42 (t, J=21.0 Hz, 1F), −165.29 (td, J=21.0, 7.3 Hz, 2F). HRMS (ESI-TOF) m/z Calcd for C₁₇H₉F₅N⁺ [M+H]⁺ 322.0655, found 322.0662.

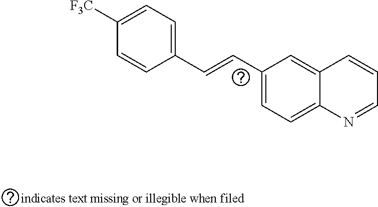 [SMILES: FC(F)(F)c1ccc(/C=C/c2ccc3ncccc3c2)cc1]

(E)-6-(4-(Trifluoromethyl)styryl)quinoline (2at) The general procedure 2.7 was followed and purification by preparative TLC (hexane/EA=3/1) furnished compound 2at as a white solid (19.1 mg, 64%, C6:others=91:9). ¹H NMR (600 MHz, CDCl₃) δ 8.83 (dd, J=4.2, 1.7 Hz, 1H), 8.09 (d, J=8.2 Hz, 1H), 8.04 (d, J=8.8 Hz, 1H), 7.92 (dd, J=8.8, 2.0 Hz, 1H), 7.80 (d, J=2.0 Hz, 1H), 7.63-7.53 (m, 4H), 7.35 (dd, J=8.3, 4.2 Hz, 1H), 7.29 (d, J=16.3 Hz, 1H), 7.21 (d, J=16.2 Hz, 1H). ¹³C NMR (151 MHz, CDCl₃) δ 150.52, 148.34, 140.50, 136.05, 134.89, 130.38, 130.04, 129.63 (q, J=3.8 Hz), 128.59, 128.55, 127.13, 126.88, 126.74, 126.65, 125.75 (q, J=3.8 Hz), 124.18 (q, J=272.0 Hz), 121.70. ¹⁹F NMR (376 MHz, CDCl₃) δ −65.15. HRMS (ESI-TOF) m/z Calcd for C₁₈H₁₃F₃N⁺ [M+H]⁺ 300.1000, found 300.1010.

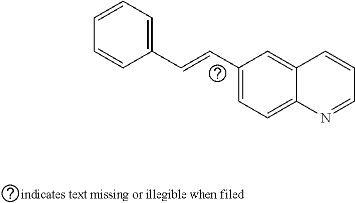 [SMILES: C(=C\c1ccc2ncccc2c1)/c1ccccc1]

(E)-6-Styrylquinoline (2au) The general procedure 2.7 was followed and purification by preparative TLC (hexane/EA=5/1) furnished compound 2au as a white solid (14.3 mg, 62%, C6:others=90:10). ¹H NMR (600 MHz, CDCl₃) δ 8.79 (dd, J=4.2, 1.7 Hz, 1H), 8.06 (d, J=7.8 Hz, 1H), 8.01 (d, J=8.8 Hz, 1H), 7.90 (dd, J=8.8, 2.0 Hz, 1H), 7.74 (d, J=2.0 Hz, 1H), 7.49 (dd, J=8.2, 1.3 Hz, 2H), 7.33-7.30 (m, 3H), 7.23-7.21 (m, 1H), 7.19 (d, J=6.6 Hz, 2H). ¹³C NMR (151 MHz, CDCl₃) δ 150.15, 148.12, 137.03, 135.95, 135.61, 130.23, 129.82, 128.81, 128.61, 128.03, 127.87, 127.27, 126.68, 125.94, 121.55. HRMS (ESI-TOF) m/z Calcd for C₁₇H₁₄N⁺ [M+H]⁺ 232.1126, found 232.1134.

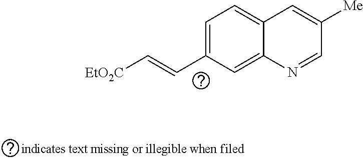 [SMILES: CCOC(=O)/C=C/c1ccc2cc(C)cnc2c1]

Ethyl (E)-3-(3-methylquinolin-7-yl)acrylate (3a) The general procedure 2.5 was followed and purification by preparative TLC (DCM/EA=10/1) furnished compound 3a as a white solid (7.7 mg, 64%, C7:others=96:4). ¹H NMR (600 MHz, CDCl₃) δ 8.79 (d, J=2.2 Hz, 1H), 8.16 (s, 1H), 7.90 (s, 1H), 7.86 (d, J=16.0 Hz, 1H), 7.74 (d, J=8.5 Hz, 1H), 7.69 (dd, J=8.5, 1.7 Hz, 1H), 6.58 (d, J=16.0 Hz, 1H), 4.30 (q, J=7.1 Hz, 2H), 2.53 (s, 3H), 1.37 (t, J=7.1 Hz, 3H). ¹³C NMR (151 MHz, CDCl₃) δ 166.86, 153.27, 146.55, 144.09, 134.61, 134.43, 131.66, 130.41, 129.10, 127.84, 124.57, 119.51, 60.67, 18.87, 14.35. HRMS (ESI-TOF) m/z Calcd for C₁₅H₁₆NO₂⁺ [M+H]⁺ 242.1181, found 242.1185.

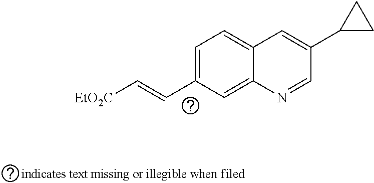 [SMILES: CCOC(=O)/C=C/c1ccc2cc(C3CC3)cnc2c1]

Ethyl (E)-3-(3-cyclopropylquinolin-7-yl)acrylate (3b) The general procedure 2.5 was followed and purification by preparative TLC (hexane/EA=10/1) furnished compound 3b as a white solid (9.5 mg, 71%, C7:others=96:4). ¹H NMR (600 MHz, CDCl₃) δ 8.77 (s, 1H), 8.15 (s, 1H), 7.86 (d, J=16.0 Hz, 1H), 7.72 (d, J=8.5 Hz, 1H), 7.68 (dd, J=8.4, 1.8 Hz, 2H), 6.58 (d, J=16.0 Hz, 1H), 4.30 (q, J=7.2 Hz, 2H), 2.08 (td, J=8.4, 4.2 Hz, 1H), 1.36 (t, J=7.2 Hz, 3H), 1.16-1.11 (m, 2H), 0.89-0.86 (m, 2H). ¹³C NMR (151 MHz, CDCl₃) δ 166.87, 151.48, 146.67, 144.10, 138.02, 134.43, 130.40, 129.09, 127.85, 124.61, 119.40, 60.66, 14.35, 13.53, 9.54. HRMS (ESI-TOF) m/z Calcd for C₁₇H₁₈NO₂⁺ [M+H]⁻ 268.1338, found 268.1342.

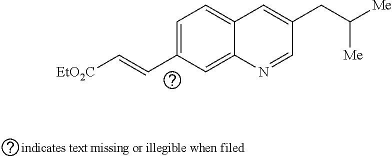 [SMILES: CCOC(=O)/C=C/c1ccc2cc(CC(C)C)cnc2c1]

Ethyl (E)-3-(3-isobutylquinolin-7-yl)acrylate (3c) The general procedure 2.5 was followed and purification by preparative TLC (hexane/EA=10/1) furnished compound 3c as a white solid (10.3 mg, 73%, C7:others=95:5). ¹H NMR (600 MHz, CDCl₃) δ 8.77 (d, J=2.2 Hz, 1H), 8.17 (s, 1H), 7.90-7.85 (m, 2H), 7.77 (d, J=8.5 Hz, 1H), 7.71 (dd, J=8.5, 1.7 Hz, 1H), 6.59 (d, J=16.0 Hz, 1H), 4.30 (q, J=7.1 Hz, 2H), 2.67 (d, J=7.2 Hz, 2H), 2.00 (dq, J=13.6, 6.7 Hz, 1H), 1.37 (t, J=7.1 Hz, 3H), 0.97 (d, J=6.6 Hz, 6H). ¹³C NMR (151 MHz, CDCl₃) δ 166.86, 153.34, 146.80, 144.10, 135.35, 134.69, 134.66, 130.38, 129.07, 128.04, 124.50, 119.52, 60.67, 42.60, 30.15, 22.27, 14.36. HRMS (ESI-TOF) m/z Calcd for C₁₈H₂₂NO₂⁺ [M+H]⁻ 284.1651, found 284.1659.

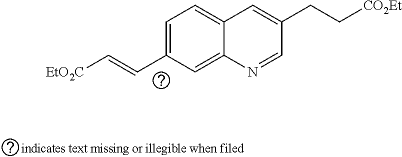 [SMILES: CCOC(=O)/C=C/c1ccc2cc(CCC(=O)OCC)cnc2c1]

Ethyl (E)-3-(3-(3-ethoxy-3-oxopropyl)quinolin-7-yl)acrylate (3d) The general procedure 2.5 was followed and purification by preparative TLC (hexane/EA=3/1) furnished compound 3d as a white solid (10 mg, 61%, C7:others=96:4). ¹H NMR (600 MHz, CDCl₃) δ 8.83 (d, J=2.2 Hz, 1H), 8.17 (s, 1H), 7.95 (s, 1H), 7.86 (d, J=16.0 Hz, 1H), 7.77 (d, J=8.5 Hz, 1H), 7.71 (dd, J=8.5, 1.5 Hz, 1H), 6.59 (d, J=16.0 Hz, 1H), 4.30 (q, J=7.1 Hz, 2H), 4.14 (q, J=7.1 Hz, 2H), 3.15 (t, J=7.5 Hz, 2H), 2.74 (t, J=7.5 Hz, 2H), 1.37 (t, J=7.0 Hz, 3H), 1.23 (t, J=7.1 Hz, 3H). ¹³C NMR (151 MHz, CDCl₃) δ 172.23, 166.80, 152.57, 147.02, 143.95, 135.04, 134.34, 134.21, 130.36, 128.97, 128.11, 124.72, 119.76, 60.70, 35.24, 28.26, 14.35, 14.20. HRMS (ESI-TOF) m/z Calcd for C₁₉H₂₂NO₄⁺ [M+H]⁻ 328.1549, found 328.1555.

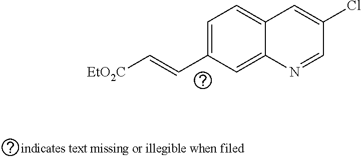 [SMILES: CCOC(=O)/C=C/c1ccc2cc(Cl)cnc2c1]

Ethyl (E)-3-(3-chloroquinolin-7-yl)acrylate (3e) The general procedure 2.5 was followed and purification by preparative TLC (DCM/EA=10:1) furnished compound 3e as a white solid (8.1 mg, 62%, C7:others=95:5). ¹H NMR (600 MHz, CDCl₃) δ 8.85 (d, J=2.4 Hz, 1H), 8.18 (s, 1H), 8.13 (dd, J=2.4, 0.8 Hz, 1H), 7.85 (d, J=16.0 Hz, 1H), 7.76 (s, 2H), 6.61 (d, J=16.0 Hz, 1H), 4.30 (t, J=7.1 Hz, 2H), 1.37 (t, J=7.1 Hz, 3H). ¹³C NMR (151 MHz, CDCl₃) δ 166.61, 150.54, 146.30, 143.40, 135.80, 133.72, 130.34, 129.42, 129.23, 127.66, 125.75, 120.49, 60.79, 14.33. HRMS (ESI-TOF) m/z Calcd for C₁₄H₃ClNO₂⁺ [M+H]⁺ 262.0635, found 262.0636.

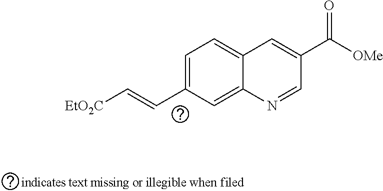 [SMILES: CCOC(=O)/C=C/c1ccc2cc(C(=O)OC)cnc2c1]

Methyl (E)-7-(3-ethoxy-3-oxoprop-1-en-1-yl)quinoline-3-carboxylate (3f) The general procedure 2.5 was followed and purification by preparative TLC (DCM/EA=5/1) furnished compound 3f as a white solid (7.4 mg, 52%, C7:others=94:6). ¹H NMR (600 MHz, CDCl₃) δ 9.47 (d, J=2.1 Hz, 1H), 8.82 (d, J=1.9 Hz, 1H), 8.25 (s, 1H), 7.94 (d, J=8.5 Hz, 1H), 7.88 (d, J=16.0 Hz, 1H), 7.79 (dd, J=8.5, 1.7 Hz, 1H), 6.65 (d, J=16.0 Hz, 1H), 4.32 (q, J=7.1 Hz, 2H), 4.03 (s, 3H), 1.37 (t, J=7.1 Hz, 3H). ¹³C NMR (151 MHz, CDCl₃) δ 166.49, 165.63, 150.85, 149.97, 143.26, 138.31, 137.86, 130.22, 129.71, 127.68, 125.48, 123.60, 121.32, 60.87, 52.62, 14.33. HRMS (ESI-TOF) m/z Calcd for C₁₆H₁₆NO₄⁺ [M+H]⁺ 286.1079, found 286.1084.

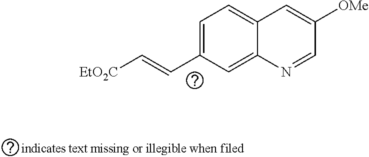 [SMILES: CCOC(=O)/C=C/c1ccc2cc(OC)cnc2c1]

Ethyl (E)-3-(3-methoxyquinolin-7-yl)acrylate (3g) The general procedure 2.5 was followed and purification by preparative TLC (DCM/EA=5/1) furnished compound 3g as a white solid (9.4 mg, 73%, C7:others>99:1). ¹H NMR (600 MHz, CDCl₃) δ 8.69 (d, J=2.8 Hz, 1H), 8.14 (s, 1H), 7.85 (d, J=16.0 Hz, 1H), 7.71 (q, J=8.5 Hz, 2H), 7.36 (d, J=2.9 Hz, 1H), 6.56 (d, J=16.1 Hz, 1H), 4.30 (q, J=7.1 Hz, 2H), 3.97 (s, 3H), 1.36 (t, J=7.1 Hz, 3H). ¹³C NMR (151 MHz, CDCl₃) δ 166.95, 153.84, 145.49, 144.15, 143.36, 132.92, 130.37, 130.02, 127.33, 125.19, 118.82, 111.94, 60.61, 55.59, 14.36. HRMS (ESI-TOF) m/z Calcd for C₁₅H₁₆NO₃⁺ [M+H]⁺ 258.1130, found 258.1128.

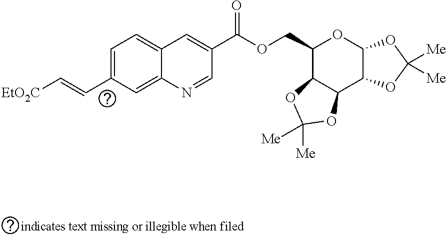 [SMILES: CCOC(=O)/C=C/c1ccc2cc(C(=O)OC[C@H]3O[C@@H]4OC(C)(C)O[C@@H]4[C@H]4OC(C)(C)O[C@H]43)cnc2c1]

((3aR,5R,5aS,8aS,8bR)-2,2,7,7-tetramethyltetrahydro-5H-bis([1,3]dioxolo)[4,5-b:4′,5′-d]pyran-5-yl)methyl 7-((E)-3-ethoxy-3-oxoprop-1-en-1-yl)quinoline-3-carboxylate (3h) The general procedure 2.5 was followed and purification by preparative TLC (DCM/EA=5/1) furnished compound 3h as a white solid (12.8 mg, 50%, C7:others=94:6). ¹H NMR (600 MHz, CDCl₃) δ 9.48 (d, J=2.1 Hz, 1H), 8.83 (d, J=1.8 Hz, 1H), 8.24 (s, 1H), 7.94 (d, J=8.5 Hz, 1H), 7.88 (d, J=16.0 Hz, 1H), 7.79 (dd, J=8.5, 1.7 Hz, 1H), 6.65 (d, J=16.0 Hz, 1H), 5.58 (d, J=4.9 Hz, 1H), 4.68 (dd, J=7.9, 2.5 Hz, 1H), 4.62 (dd, J=11.6, 4.5 Hz, 1H), 4.55 (dd, J=11.5, 7.8 Hz, 1H), 4.38-4.34 (m, 2H), 4.32 (q, J=7.1 Hz, 2H), 4.25 (ddd, J=7.8, 4.4, 1.9 Hz, 1H), 1.52 (d, J=20.1 Hz, 6H), 1.39-1.36 (m, 6H), 1.34 (s, 3H). ¹³C NMR (151 MHz, CDCl₃) δ 166.49, 165.02, 150.93, 150.01, 143.28, 138.43, 137.87, 130.22, 129.76, 127.67, 125.44, 123.52, 121.31, 109.84, 108.89, 96.35, 71.16, 70.78, 70.51, 66.13, 64.62, 60.86, 26.07, 26.00, 24.97, 24.50, 14.33. HRMS (ESI-TOF) m/z Calcd for C₂₇H₃₂NO₉⁺ [M+H]⁺ 514.2077, found 514.2077.

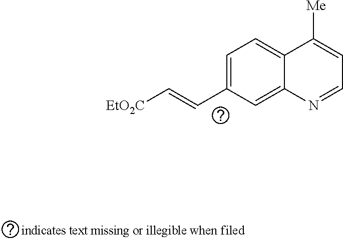 [SMILES: CCOC(=O)/C=C/c1ccc2c(C)ccnc2c1]

Ethyl (E)-3-(4-methylquinolin-7-yl)acrylate (3i) The general procedure 2.5 was followed and purification by preparative TLC (DCM/EA=5/1) furnished compound 3i as a white solid (6.6 mg, 55%, C7:others>99:1). ¹H NMR (600 MHz, CDCl₃) δ 8.80 (d, J=4.3 Hz, 1H), 8.20 (d, J=1.8 Hz, 1H), 8.01 (d, J=8.7 Hz, 1H), 7.88 (d, J=16.0 Hz, 1H), 7.75 (dd, J=8.7, 1.9 Hz, 1H), 7.25 (s, 1H), 6.62 (d, J=16.0 Hz, 1H), 4.31 (q, J=7.0 Hz, 2H), 2.72 (s, 3H), 1.37 (t, J=7.1 Hz, 3H). ¹³C NMR (151 MHz, CDCl₃) δ 166.79, 151.03, 148.08, 144.24, 143.88, 135.20, 131.03, 129.23, 124.64, 124.27, 122.70, 119.95, 60.71, 18.62, 14.35. HRMS (ESI-TOF) m/z Calcd for C₁₅H₁₆NO₂⁺ [M+H]⁺ 242.1181, found 242.1187.

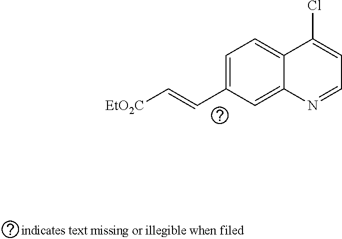 [SMILES: CCOC(=O)/C=C/c1ccc2c(Cl)ccnc2c1]

Ethyl (E)-3-(4-chloroquinolin-7-yl)acrylate (3j) The general procedure 2.5 was followed and purification by preparative TLC (DCM/EA=5/1) furnished compound 3j as a white solid (5.6 mg, 43%, C7:others=94:6). ¹H NMR (600 MHz, CDCl₃) δ 8.81 (d, J=4.7 Hz, 1H), 8.25 (d, J=8.7 Hz, 1H), 8.21 (d, J=1.7 Hz, 1H), 7.88 (d, J=16.0 Hz, 1H), 7.82 (dd, J=8.8, 1.8 Hz, 1H), 7.51 (d, J=4.6 Hz, 1H), 6.64 (d, J=16.0 Hz, 1H), 4.31 (q, J=7.1 Hz, 2H), 1.37 (t, J=7.1 Hz, 3H). ¹³C NMR (151 MHz, CDCl₃) δ 166.55, 150.75, 149.28, 143.17, 142.56, 136.53, 130.59, 127.33, 125.59, 124.96, 121.93, 120.97, 60.83, 14.33. HRMS (ESI-TOF) m/z Calcd for C₁₄H₁₃ClNO₂⁺ [M+H]⁺ 262.0635, found 262.0639.

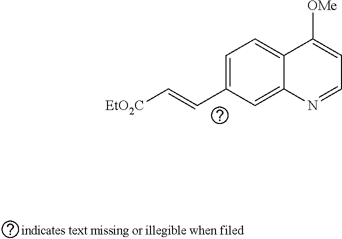 [SMILES: CCOC(=O)/C=C/c1ccc2c(OC)ccnc2c1]

Ethyl (E)-3-(4-methoxyquinolin-7-yl)acrylate (3k) The general procedure 2.5 was followed and purification by preparative TLC (DCM/EA=1/1) furnished compound 3k as a white solid (6.4 mg, 50%, C7:others=93:7). ¹H NMR (600 MHz, CDCl₃) δ 8.78 (d, J=5.2 Hz, 1H), 8.20 (d, J=8.6 Hz, 1H), 8.12 (d, J=1.7 Hz, 1H), 7.86 (d, J=16.0 Hz, 1H), 7.68 (dd, J=8.7, 1.7 Hz, 1H), 6.76 (d, J=5.2 Hz, 1H), 6.60 (d, J=16.0 Hz, 1H), 4.30 (q, J=7.1 Hz, 2H), 4.06 (s, 3H), 1.37 (t, J=7.1 Hz, 3H). ¹³C NMR (151 MHz, CDCl₃) δ 166.79, 162.15, 152.25, 149.27, 143.99, 135.81, 129.97, 123.61, 122.65, 122.25, 120.01, 100.90, 60.69, 55.82, 14.35. HRMS (ESI-TOF) m/z Calcd for C₁₅H₁₆NO₃⁺ [M+H]⁺ 258.1130, found 258.1139.

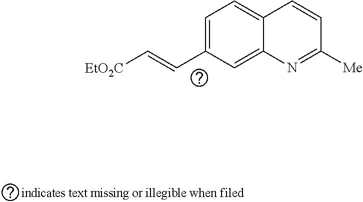 [SMILES: CCOC(=O)/C=C/c1ccc2ccc(C)nc2c1]

Ethyl (E)-3-(2-methylquinolin-7-yl)acrylate (31) The general procedure 2.5 was followed and purification by preparative TLC (hexane/EA=5/1) furnished compound 31 as a white solid (5.4 mg, 45%, C7:others=94:6). ¹H NMR (600 MHz, CDCl₃) δ 8.05 (d, J=0.9 Hz, 1H), 7.96 (d, J=8.3 Hz, 1H), 7.79 (dd, J=16.0, 0.6 Hz, 1H), 7.70 (d, J=8.4 Hz, 1H), 7.59 (dd, J=8.4, 1.7 Hz, 1H), 7.24 (d, J=8.3 Hz, 1H), 6.53 (d, J=16.0 Hz, 1H), 4.23 (q, J=7.2 Hz, 2H), 2.69 (s, 3H), 1.30 (t, J=7.1 Hz, 3H). ¹³C NMR (151 MHz, CDCl₃) δ 166.86, 160.01, 147.94, 144.15, 135.82, 135.50, 129.76, 128.17, 127.48, 123.80, 122.90, 119.78, 60.68, 25.41, 14.36. HRMS (ESI-TOF) m/z Calcd for C₁₅H₁₆NO₂⁺ [M+H]⁺ 242.1181, found 242.1191.

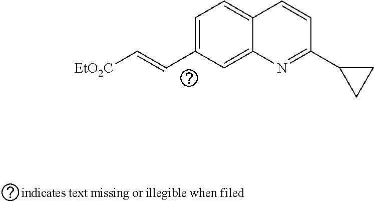 [SMILES: CCOC(=O)/C=C/c1ccc2ccc(C3CC3)nc2c1]

Ethyl (E)-3-(2-cyclopropylquinolin-7-yl)acrylate (3m) The general procedure 2.5 was followed and purification by preparative TLC (hexane/EA=10/1) furnished compound 3m as a white solid (5.5 mg, 41%, C7:others=96:4). ¹H NMR (600 MHz, CDCl₃) δ 8.00 (d, J=1.0 Hz, 1H), 7.90 (dd, J=8.6, 0.8 Hz, 1H), 7.77 (d, J=16.0 Hz, 1H), 7.66 (d, J=8.5 Hz, 1H), 7.53 (dd, J=8.4, 1.7 Hz, 1H), 7.14 (d, J=8.5 Hz, 1H), 6.52 (d, J=16.0 Hz, 1H), 4.22 (q, J=7.1 Hz, 2H), 2.18-2.14 (m, 1H), 1.29 (t, J=7.2 Hz, 3H), 1.12-1.10 (m, 1H), 1.05-1.03 (m, 1H). ¹³C NMR (151 MHz, CDCl₃) δ 166.91, 164.45, 148.06, 144.30, 135.38, 135.34, 129.81, 128.11, 127.73, 123.29, 120.55, 119.54, 60.63, 18.09, 14.35, 10.54. HRMS (ESI-TOF) m/z Calcd for C₁₇H₁₈NO₂⁺ [M+H]⁺ 268.1338, found 268.1349.

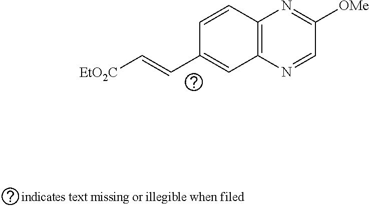 [SMILES: CCOC(=O)/C=C/c1ccc2nc(OC)cnc2c1]

Ethyl (E)-3-(2-methoxyquinoxalin-6-yl)acrylate (3n) The general procedure 2.5 was followed and purification by preparative TLC (DCM/EA=20/1) furnished compound 3n as a white solid (7.5 mg, 58%, C6:others>99:1). ¹H NMR (600 MHz, CDCl₃) δ 8.49 (s, 1H), 8.12 (d, J=1.8 Hz, 1H), 7.87-7.83 (m, 3H), 6.56 (d, J=16.0 Hz, 1H), 4.30 (q, J=7.2 Hz, 2H), 4.12 (s, 3H), 1.36 (t, J=7.1 Hz, 3H). ¹³C NMR (151 MHz, CDCl₃) δ 166.81, 158.22, 143.51, 141.63, 140.49, 138.79, 132.85, 129.62, 128.47, 127.83, 119.35, 60.68, 53.95, 14.35. HRMS (ESI-TOF) m/z Calcd for C₁₄H₁₅N₂O₃⁺ [M+H]⁺ 259.1083, found 259.1088.

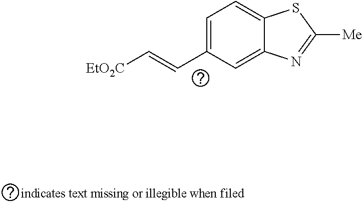 [SMILES: CCOC(=O)/C=C/c1ccc2sc(C)nc2c1]

Ethyl (E)-3-(2-methylbenzo[d]thiazol-5-yl)acrylate (3o) The general procedure 2.5 was followed and purification by preparative TLC (DCM/EA=20/1) furnished compound 3o as a white solid (7.4 mg, 60%, C5:others=94:6). ¹H NMR (600 MHz, CDCl₃) δ 8.07 (d, J=1.7 Hz, 1H), 7.84-7.78 (m, 2H), 7.53 (dd, J=8.3, 1.7 Hz, 1H), 6.51 (d, J=16.0 Hz, 1H), 4.29 (q, J=7.2 Hz, 2H), 2.85 (s, 3H), 1.36 (t, J=7.1 Hz, 3H). ¹³C NMR (151 MHz, CDCl₃) δ 168.26, 166.95, 153.90, 144.33, 137.55, 132.71, 123.99, 122.21, 121.76, 118.54, 60.61, 20.25, 14.36. HRMS (ESI-TOF) m/z Calcd for C₁₃H₁₄NO₂S⁺ [M+H]⁺ 248.0745, found 248.0747.

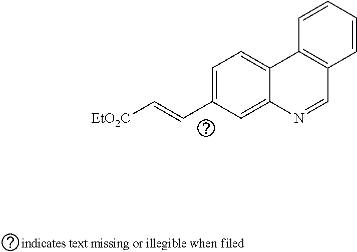 [SMILES: CCOC(=O)/C=C/c1ccc2c(c1)ncc1ccccc12]

Ethyl (E)-3-(phenanthridin-3-yl)acrylate (3p) The general procedure 2.5 was followed and purification by preparative TLC (DCM/EA=5/1) furnished compound 3p as a white solid (10.6 mg, 77%, C_{shown}:others=93:7). ¹H NMR (600 MHz, CDCl₃) δ 9.31 (s, 1H), 8.59 (dd, J=17.9, 8.4 Hz, 2H), 8.31 (d, J=1.8 Hz, 1H), 8.07 (d, J=7.8 Hz, 1H), 7.93-7.85 (m, 3H), 7.76 (t, J=7.8 Hz, 1H), 6.65 (d, J=16.0 Hz, 1H), 4.32 (q, J=7.1 Hz, 2H), 1.38 (t, J=7.1 Hz, 3H). ¹³C NMR (151 MHz, CDCl₃) δ 166.88, 154.42, 144.59, 143.87, 134.81, 132.15, 131.36, 130.63, 128.93, 128.14, 126.68, 125.56, 125.43, 122.97, 122.12, 119.57, 60.68, 14.37. HRMS (ESI-TOF) m/z Calcd for C₁₈H₁₆NO₂⁺ [M+H]⁺ 278.1181, found 278.1185.

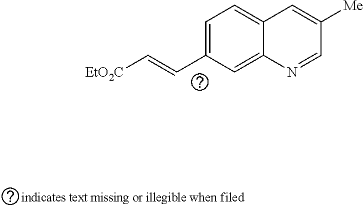 [SMILES: CCOC(=O)/C=C/c1ccc2cc(C)cnc2c1]

Methyl (E)-3-(3-methylquinolin-7-yl)acrylate (3q) The general procedure 2.5 was followed and purification by preparative TLC (DCM/EA=10/1) furnished compound 3q as a white solid (6.7 mg, 59%, C7:others=95:5). ¹H NMR (600 MHz, CDCl₃) δ 8.80 (d, J=2.2 Hz, 1H), 8.16 (s, 1H), 7.91 (s, 1H), 7.88 (d, J=16.0 Hz, 1H), 7.75 (d, J=8.5 Hz, 1H), 7.69 (dd, J=8.5, 1.8 Hz, 1H), 6.59 (d, J=16.0 Hz, 1H), 3.84 (s, 3H), 2.54 (s, 3H). ¹³C NMR (151 MHz, CDCl₃) δ 167.29, 153.30, 146.53, 144.39, 134.52, 134.44, 131.72, 130.46, 129.14, 127.87, 124.57, 119.02, 51.84, 18.87. HRMS (ESI-TOF) m/z Calcd for C₁₄H₁₄NO₂⁺ [M+H]⁺ 228.1025, found 228.1027.

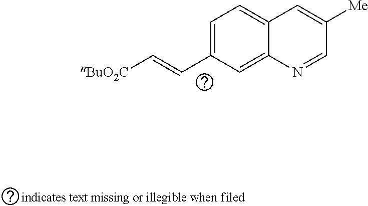

Butyl (E)-3-(3-methylquinolin-7-yl)acrylate (3r) The general procedure 2.5 was followed and purification by preparative TLC (DCM/EA=10/1) furnished compound 3r as a white solid (8.4 mg, 62%, C7:others=95:5). ¹H NMR (600 MHz, CDCl₃) δ 8.79 (d, J=2.2 Hz, 1H), 8.16 (s, 1H), 7.90 (s, 1H), 7.86 (d, J=16.0 Hz, 1H), 7.74 (d, J=8.5 Hz, 1H), 7.70 (dd, J=8.5, 1.8 Hz, 1H), 6.59 (d, J=16.0 Hz, 1H), 4.25 (t, J=6.7 Hz, 2H), 2.53 (s, 3H), 1.74-1.69 (m, 2H), 1.49-1.44 (m, 2H), 0.98 (t, J=7.4 Hz, 3H). ¹³C NMR (151 MHz, CDCl₃) δ 166.96, 153.27, 146.55, 144.07, 134.63, 134.43, 131.66, 130.42, 129.10, 127.83, 124.57, 119.53, 64.60, 30.79, 19.22, 18.87, 13.77. HRMS (ESI-TOF) m/z Calcd for C₁₇H₂₀NO₂⁺ [M+H]⁻ 270.1494, found 270.1497.

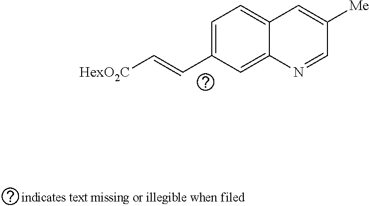

Hexyl (E)-3-(3-methylquinolin-7-yl)acrylate (3s) The general procedure 2.5 was followed and purification by preparative TLC (DCM) furnished compound 3s as a white solid (9.0 mg, 61%, C7:others=95:5). ¹H NMR (600 MHz, CDCl₃) δ 8.79 (d, J=2.2 Hz, 1H), 8.16 (s, 1H), 7.90 (s, 1H), 7.86 (d, J=16.0 Hz, 1H), 7.74 (d, J=8.5 Hz, 1H), 7.70 (dd, J=8.5, 1.7 Hz, 1H), 6.59 (d, J=16.0 Hz, 1H), 4.23 (t, J=6.8 Hz, 2H), 2.53 (s, 3H), 1.75-1.70 (m, 2H), 1.45-1.39 (m, 2H), 1.36-1.32 (m, 4H), 0.93-0.90 (m, 3H). ¹³C NMR (151 MHz, CDCl₃) δ 166.96, 153.27, 146.55, 144.06, 134.63, 134.43, 131.66, 130.43, 129.10, 127.83, 124.57, 119.54, 64.90, 31.48, 28.71, 25.66, 22.57, 18.87, 14.03. HRMS (ESI-TOF) m/z Calcd for C₁₉H₂₄NO₂⁺ [M+H]⁺ 298.1807, found 298.1811.

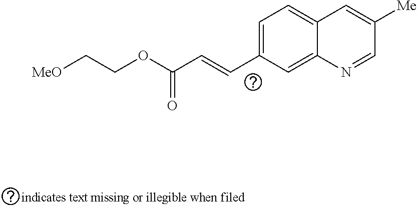 [SMILES: COCCOC(=O)/C=C/c1ccc2cc(C)cnc2c1]

2-Methoxyethyl (E)-3-(3-methylquinolin-7-yl)acrylate (3t) The general procedure 2.5 was followed and purification by preparative TLC (DCM/EA=2/1) furnished compound 3t as a white solid (7.5 mg, 55%, C7:others=95:5). ¹H NMR (600 MHz, CDCl₃) δ 8.79 (d, J=2.2 Hz, 1H), 8.16 (s, 1H), 7.92-7.88 (m, 2H), 7.74 (d, J=8.5 Hz, 1H), 7.69 (dd, J=8.5, 1.8 Hz, 1H), 6.64 (d, J=16.0 Hz, 1H), 4.42-4.39 (m, 2H), 3.71-3.68 (m, 2H), 3.44 (s, 3H), 2.53 (s, 3H). ¹³C NMR (151 MHz, CDCl₃) δ 166.82, 153.29, 146.54, 144.67, 134.51, 134.43, 131.72, 130.61, 129.16, 127.88, 124.52, 119.02, 70.59, 63.72, 59.09, 18.87. HRMS (ESI-TOF) m/z Calcd for C₁₆H₁₈NO₃⁺ [M+H]⁺ 272.1287, found 272.1294.

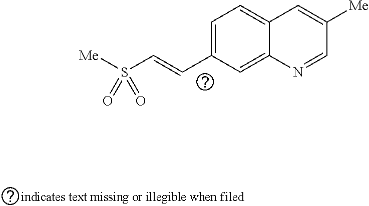 [SMILES: Cc1cnc2cc(/C=C/S(C)(=O)=O)ccc2c1]

(E)-3-Methyl-7-(2-(methylsulfonyl)vinyl)quinoline (3u) The general procedure 2.5 was followed and purification by preparative TLC (EA/acetone=10/1) furnished compound 3u as a white solid (5.5 mg, 45%, C7:others>99:1). ¹H NMR (600 MHz, CDCl₃) δ 8.83 (d, J=2.3 Hz, 1H), 8.20 (s, 1H), 7.93 (s, 1H), 7.83-7.77 (m, 2H), 7.64 (dd, J=8.4, 1.9 Hz, 1H), 7.06 (d, J=15.4 Hz, 1H), 3.08 (s, 3H), 2.55 (s, 3H). ¹³C NMR (151 MHz, CDCl₃) δ 153.67, 146.34, 143.64, 134.45, 132.44, 132.08, 131.44, 129.67, 128.30, 127.24, 124.56, 43.33, 18.92. HRMS (ESI-TOF) m/z Calcd for C₁₃H₁₄NO₂S⁺ [M+H]⁺ 248.0745, found 248.0750.

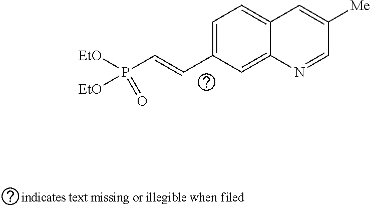 [SMILES: CCOP(=O)(/C=C/c1ccc2cc(C)cnc2c1)OCC]

Diethyl (E)-(2-(3-methylquinolin-7-yl)vinyl)phosphonate (3v) The general procedure 2.5 was followed and purification by preparative TLC (EA/acetone=1/1) furnished compound 3v as a white solid (10.0 mg, 66%, C7:others>99:1). ¹H NMR (600 MHz, CDCl₃) δ 8.80 (d, J=2.2 Hz, 1H), 8.14 (s, 1H), 7.91 (s, 1H), 7.75 (d, J=8.5 Hz, 1H), 7.72-7.65 (m, 2H), 6.42 (t, J=17.3 Hz, 1H), 4.20-4.14 (m, 4H), 2.54 (s, 3H), 1.38 (t, J=7.0 Hz, 6H). ¹³C NMR (151 MHz, CDCl₃) δ 153.32, 148.16, 148.12, 146.52, 146.50, 135.03, 134.88, 134.43, 131.69, 129.94, 129.08, 127.83, 124.36, 116.10, 114.84, 62.01, 61.97, 18.87, 16.47, 16.42. HRMS (ESI-TOF) m/z Calcd for C₁₆H₂₁NO₃P⁺ [M+H]⁺ 306.1259, found 306.1258.

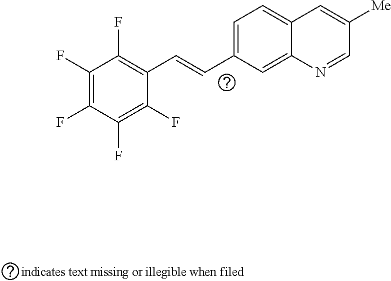 [SMILES: Cc1cnc2cc(/C=C/c3c(F)c(F)c(F)c(F)c3F)ccc2c1]

(E)-3-methyl-7-(2-(perfluorophenyl)vinyl)quinoline (3w) The general procedure 2.5 was followed and purification by preparative TLC (DCM/EA=10/1) furnished compound 3w as a white solid (11.7 mg, 70%, C7:others=96:4). ¹H NMR (600 MHz, CDCl₃) δ 8.79 (d, J=2.2 Hz, 1H), 8.14 (s, 1H), 7.91 (s, 1H), 7.77-7.73 (m, 2H), 7.62 (d, J=16.7 Hz, 1H), 7.16 (d, J=16.7 Hz, 1H), 2.53 (s, 3H). ¹³C NMR (151 MHz, CDCl₃) δ 153.19, 146.76, 136.72 (d, J=3.1 Hz), 136.63, 136.63, 134.43, 131.13, 128.46, 128.41, 127.74, 124.18, 114.03 (d, J=2.8 Hz), 18.84. ¹⁹F NMR (376 MHz, CDCl₃) δ −145.02 (dd, J=22.1, 8.3 Hz, 2F), −158.61 (t, J=21.5 Hz, 1F), −165.39 (td, J=21.8, 7.9 Hz, 2F). HRMS (ESI-TOF) m/z Calcd for C₁₈H₁₁F₅N⁺ [M+H]⁺ 336.0812, found 336.0815.

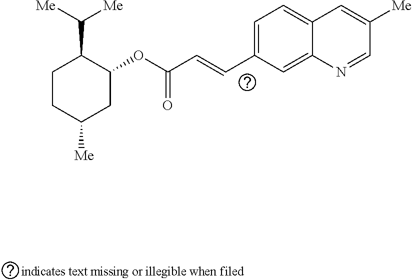 [SMILES: Cc1cnc2cc(/C=C/C(=O)O[C@@H]3C[C@H](C)CC[C@H]3C(C)C)ccc2c1]

(1R,2S,5R)-2-Isopropyl-5-methylcyclohexyl (E)-3-(3-methylquinolin-7-yl)acrylate (3x) The general procedure 2.5 was followed and purification by preparative TLC (DCM/EA=10/1) furnished compound 3x as a white solid (10.5 mg, 60%, C7:others=95:5). ¹H NMR (600 MHz, CDCl₃) δ 8.79 (d, J=2.2 Hz, 1H), 8.16 (s, 1H), 7.90 (s, 1H), 7.85 (d, J=16.0 Hz, 1H), 7.74 (d, J=8.5 Hz, 1H), 7.70 (dd, J=8.5, 1.7 Hz, 1H), 6.58 (d, J=16.0 Hz, 1H), 4.88-4.83 (m, 1H), 2.53 (d, J=1.0 Hz, 3H), 2.11-2.07 (m, 1H), 1.95 (ddd, J=11.3, 7.0, 3.5 Hz, 1H), 1.72 (dt, J=14.4, 2.9 Hz, 2H), 1.56-1.53 (m, 1H), 1.50-1.47 (m, 1H), 1.10 (dd, J=23.4, 11.3 Hz, 2H), 0.93 (dd, J=6.8, 3.6 Hz, 7H), 0.81 (d, J=6.9 Hz, 3H). ¹³C NMR (151 MHz, CDCl₃) δ 166.44, 153.24, 146.56, 143.88, 134.71, 134.43, 131.62, 130.43, 129.06, 127.80, 124.55, 119.97, 74.47, 47.22, 41.04, 34.32, 31.46, 26.41, 23.59, 22.06, 20.78, 18.87, 16.49. HRMS (ESI-TOF) m/z Calcd for C₂₃H₃₀NO₂⁺ [M+H]⁺ 352.2277, found 352.2281.

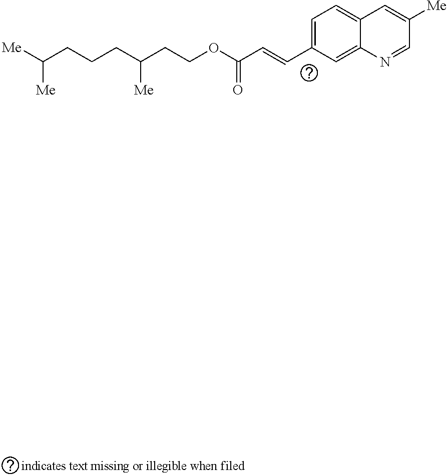 [SMILES: Cc1cnc2cc(/C=C/C(=O)OCCC(C)CCCC(C)C)ccc2c1]

3,7-Dimethyloctyl (E)-3-(3-methylquinolin-7-yl)acrylate (3y) The general procedure 2.5 was followed and purification by preparative TLC (DCM) furnished compound 3y as a white solid (11.5 mg, 65%, C7:others=95:5). ¹H NMR (600 MHz, CDCl₃) δ 8.79 (d, J=2.2 Hz, 1H), 8.16 (s, 1H), 7.90 (s, 1H), 7.86 (d, J=16.0 Hz, 1H), 7.74 (d, J=8.5 Hz, 1H), 7.70 (dd, J=8.5, 1.7 Hz, 1H), 6.59 (d, J=16.0 Hz, 1H), 4.31-4.22 (m, 2H), 2.53 (s, 3H), 1.80-1.74 (m, 1H), 1.62 (dd, J=12.3, 6.9 Hz, 1H), 1.56-1.49 (m, 2H), 1.33 (ddt, J=9.4, 5.1, 2.8 Hz, 2H), 1.29-1.25 (m, 1H), 1.20-1.14 (m, 3H), 0.95 (d, J=6.6 Hz, 3H), 0.87 (dd, J=6.6, 1.2 Hz, 6H). ¹³C NMR (151 MHz, CDCl₃) δ 166.96, 153.27, 146.55, 144.06, 134.63, 134.43, 131.66, 130.43, 129.10, 127.83, 124.57, 119.55, 63.35, 39.23, 37.18, 35.64, 29.94, 27.97, 24.65, 22.71, 22.62, 19.59, 18.87. HRMS (ESI-TOF) m/z Calcd for C₂₃H₃₂NO₂⁺ [M+H]⁺ 354.2433, found 354.2430.

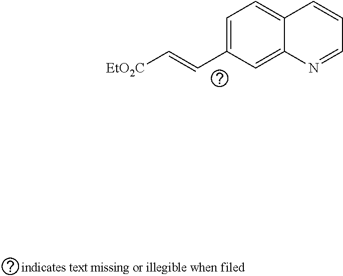 [SMILES: CCOC(=O)/C=C/c1ccc2cccnc2c1]

Ethyl (E)-3-(quinolin-7-yl)acrylate (3z) The general procedure 2.5 was followed except using (S,S)-T25/Pd(MeCN)₂Cl₂/Ac-L-Leu-OH and purification by preparative TLC (DCM/EA=5/1) furnished compound 3z as a white solid (7 mg, 62%, C7:C3=90:10). ¹H NMR (600 MHz, CDCl₃) δ 8.95 (dd, J=4.2, 1.7 Hz, 1H), 8.20 (s, 1H), 8.15 (d, J=7.4 Hz, 1H), 7.88 (d, J=16.0 Hz, 1H), 7.83 (d, J=8.5 Hz, 1H), 7.74 (dd, J=8.5, 1.7 Hz, 1H), 7.43 (dd, J=8.2, 4.2 Hz, 1H), 6.62 (d, J=16.0 Hz, 1H), 4.31 (q, J=7.2 Hz, 2H), 1.37 (t, J=7.2 Hz, 3H). ¹³C NMR (151 MHz, CDCl₃) δ 166.75, 151.27, 148.35, 143.88, 135.76, 135.59, 130.59, 129.20, 128.50, 124.54, 121.93, 120.10, 60.73, 14.35. HRMS (ESI-TOF) m/z Calcd for C₁₄H₁₄NO₂⁺ [M+H]⁺ 228.1025, found 228.1026.

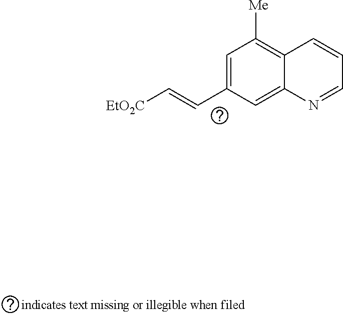 [SMILES: CCOC(=O)/C=C/c1cc(C)c2cccnc2c1]

Ethyl (E)-3-(5-methylquinolin-7-yl)acrylate (3aa) The general procedure 2.5 was followed except using (S,S)-T25/Pd(MeCN)₂Cl₂/Ac-L-Leu-OH and purification by preparative TLC (DCM/EA=10/1) furnished compound 3aa as a white solid (6.3 mg, 52%, C7:C3=90:10). ¹H NMR (600 MHz, CDCl₃) δ 8.94 (dd, J=4.2, 1.6 Hz, 1H), 8.31 (d, J=8.5 Hz, 1H), 8.06 (s, 1H), 7.84 (d, J=16.0 Hz, 1H), 7.57 (s, 1H), 7.45 (dd, J=8.5, 4.1 Hz, 1H), 6.60 (d, J=16.0 Hz, 1H), 4.30 (q, J=7.1 Hz, 2H), 2.71 (s, 3H), 1.37 (t, J=7.1 Hz, 3H). ¹³C NMR (151 MHz, CDCl₃) δ 166.83, 150.79, 148.65, 144.04, 135.41, 135.09, 132.32, 129.07, 128.72, 124.80, 121.56, 119.81, 60.68, 18.77, 14.35. HRMS (ESI-TOF) m/z Calcd for C₁₅H₁₆NO₂⁺ [M+H]⁺ 242.1181, found 242.1187.

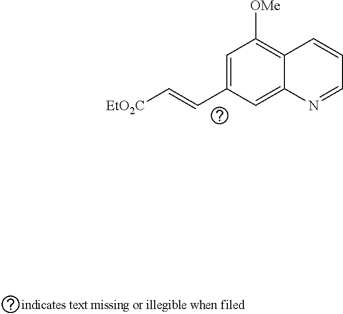 [SMILES: CCOC(=O)/C=C/c1cc(OC)c2cccnc2c1]

Ethyl (E)-3-(5-methoxyquinolin-7-yl)acrylate (3ab) The general procedure 2.5 was followed except using (S,S)-T25/Pd(MeCN)₂Cl₂/Ac-L-Leu-OH and purification by preparative TLC (hexane/EA=5/1) furnished compound 3ab as a white solid (7.2 mg, 56%, C7:C3=88:12). ¹H NMR (600 MHz, CDCl₃) δ 8.92 (dd, J=4.2, 1.8 Hz, 1H), 8.54 (ddd, J=8.4, 1.7, 0.8 Hz, 1H), 7.86-7.80 (m, 2H), 7.40 (dd, J=8.4, 4.2 Hz, 1H), 7.00 (d, J=1.4 Hz, 1H), 6.58 (d, J=16.0 Hz, 1H), 4.31 (q, J=7.1 Hz, 2H), 4.05 (s, 3H), 1.37 (t, J=7.1 Hz, 3H). ¹³C NMR (151 MHz, CDCl₃) δ 166.77, 155.55, 151.48, 149.06, 144.49, 135.52, 130.73, 123.79, 122.16, 121.13, 119.62, 101.46, 60.72, 55.83, 14.36. HRMS (ESI-TOF) m/z Calcd for C₁₅H₁₆NO₃⁺ [M+H]⁺ 258.1130, found 258.1142.

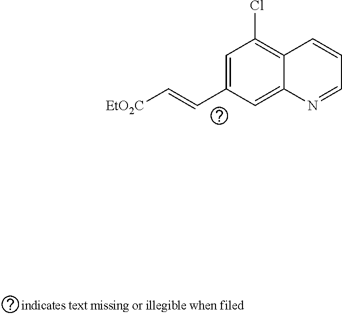 [SMILES: CCOC(=O)/C=C/c1cc(Cl)c2cccnc2c1]

Ethyl (E)-3-(5-chloroquinolin-7-yl)acrylate (3ac) The general procedure 2.5 was followed except using (S,S)-T25/Pd(MeCN)₂Cl₂/Ac-L-Leu-OH and purification by preparative TLC (hexane/EA=5/1) furnished compound 3ac as a white solid (5.9 mg, 45%, C7:C3=90:10). ¹H NMR (600 MHz, CDCl₃) δ 8.99 (dd, J=4.2, 1.7 Hz, 1H), 8.56 (ddd, J=8.5, 1.7, 0.9 Hz, 1H), 8.13 (s, 1H), 7.84-7.77 (m, 2H), 7.54 (dd, J=8.5, 4.2 Hz, 1H), 6.60 (d, J=16.0 Hz, 1H), 4.31 (q, J=7.2 Hz, 2H), 1.37 (t, J=7.2 Hz, 3H). ¹³C NMR (151 MHz, CDCl₃) δ 166.41, 151.83, 148.89, 142.55, 135.61, 132.79, 132.29, 129.66, 127.19, 124.45, 122.65, 120.99, 60.86, 14.32. HRMS (ESI-TOF) m/z Calcd for C₁₄H₃ClNO₂⁺ [M+H]⁺ 262.0635, found 262.0651.

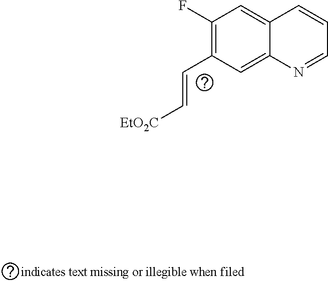 [SMILES: CCOC(=O)/C=C/c1cc2ncccc2cc1F]

Ethyl (E)-3-(6-fluoroquinolin-7-yl)acrylate (3ad) The general procedure 2.5 was followed except using (S,S)-T25/Pd(MeCN)₂Cl₂/Ac-L-Leu-OH and purification by preparative TLC (DCM/EA=10/1) furnished compound 3ad as a white solid (4.7 mg, 38%, C7:C3=88:12). ¹H NMR (600 MHz, CDCl₃) δ 8.91 (d, J=3.0 Hz, 1H), 8.30 (d, J=7.1 Hz, 1H), 8.10 (d, J=8.2 Hz, 1H), 7.93 (d, J=16.2 Hz, 1H), 7.48 (d, J=10.8 Hz, 1H), 7.43 (dd, J=8.3, 4.2 Hz, 1H), 6.76 (d, J=16.2 Hz, 1H), 4.31 (q, J=7.1 Hz, 2H), 1.37 (t, J=7.2 Hz, 3H). ¹³C NMR (151 MHz, CDCl₃) δ 166.60, 158.80 (d, J=254.9 Hz), 150.59 (d, J=2.9 Hz), 145.13, 137.09 (d, J=2.6 Hz), 135.01 (d, J=5.5 Hz), 131.40 (d, J=4.9 Hz), 129.58 (d, J=10.6 Hz), 126.59 (d, J=16.0 Hz), 123.04 (d, J=7.1 Hz), 122.47, 111.71 (d, J=23.0 Hz), 60.85, 14.32. ¹⁹F NMR (376 MHz, CDCl₃) δ −119.18. HRMS (ESI-TOF) m/z Calcd for C₁₄H₁₃FNO₂⁺ [M+H]⁺ 246.0930, found 246.0935.

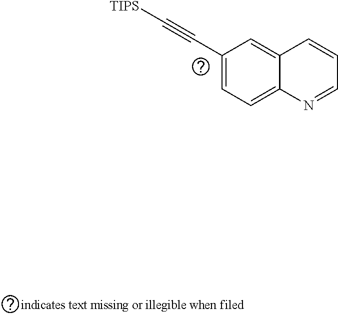

6-((Triisopropylsilyl)ethynyl)quinoline (4a) The general procedure 2.6 was followed and purification by preparative TLC (hexane/EA=3/1) furnished compound 4a as colorless oil (20.4 mg, 66%, C6:others=96:4). ¹H NMR (600 MHz, CDCl₃) δ 8.90 (dd, J=4.2, 1.7 Hz, 1H), 8.10 (dd, J=8.4, 1.7 Hz, 1H), 8.03 (d, J=8.7 Hz, 1H), 7.96 (d, J=1.9 Hz, 1H), 7.76 (dd, J=8.7, 1.8 Hz, 1H), 7.41 (dd, J=8.3, 4.2 Hz, 1H), 1.16 (m, 21H). ¹³C NMR (151 MHz, CDCl₃) δ 150.92, 147.70, 135.70, 132.60, 131.53, 129.45, 127.92, 121.84, 121.71, 106.52, 92.32, 18.70, 11.33. HRMS (ESI-TOF) m/z Calcd for C₂₀H₂₈NSi⁺ [M+H]⁺ 310.1991, found 310.1994.

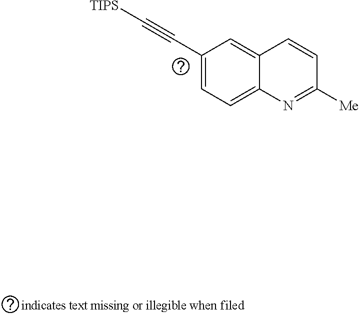

2-Methyl-6-((triisopropylsilyl)ethynyl)quinoline (4b) The general procedure 2.6 was followed and purification by preparative TLC (hexane/EA=3/1) furnished compound 4b as a pale yellow solid (16.1 mg, 50%, C6:others=98:2). ¹H NMR (600 MHz, CDCl₃) δ 7.99 (d, J=8.4 Hz, 1H), 7.94-7.90 (m, 2H), 7.72 (dd, J=8.6, 1.9 Hz, 1H), 7.29 (d, J=8.4 Hz, 1H), 2.74 (s, 3H), 1.16 (m, 21H). ¹³C NMR (151 MHz, CDCl₃) δ 159.76, 147.32, 135.80, 132.63, 131.30, 128.62, 126.11, 122,65, 120,88, 106,72, 91.67, 25.45, 18.70, 11.34. HRMS (ESI-TOF) m/z Calcd for C₂₁H₃₀NSi⁺ [M+H]⁺ 324.2148, found 324.2154.

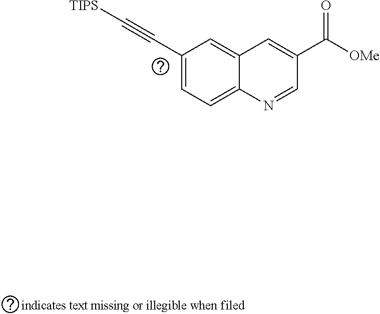

Methyl 6-((triisopropylsilyl)ethynyl)quinoline-3-carboxylate (4c) The general procedure 2.6 was followed and purification by preparative TLC (hexane/EA=3/1) furnished compound 4c as a pale yellow solid (17.2 mg, 47%, C6:others>99:1). ¹H NMR (600 MHz, CDCl₃) δ 9.42 (d, J=2.1 Hz, 1H), 8.79 (d, J=1.7 Hz, 1H), 8.08 (d, J=8.7 Hz, 1H), 8.06 (d, J=1.8 Hz, 1H), 7.86 (dd, J=8.7, 1.8 Hz, 1H), 4.02 (s, 3H), 1.16 (m, 21H). ¹³C NMR (151 MHz, CDCl₃) δ 165.63, 150.47, 149.18, 138.33, 134.90, 132.58, 129.46, 126.59, 123.62, 122.88, 105.87, 93.52, 52.59, 18.68, 11.31. HRMS (ESI-TOF) m/z Calcd for C₂₂H₃₀NO₂Si⁺ [M+H]⁺ 368.2046, found 368.2043.

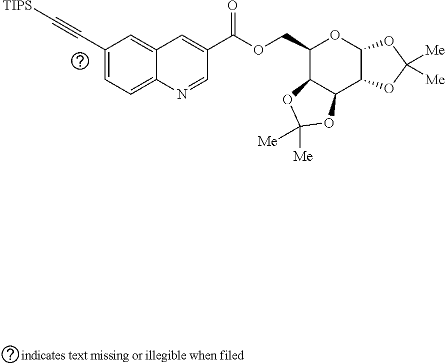

((3aR,5R,5aS,8aS,8bR)-2,2,7,7-tetramethyltetrahydro-5H-bis([1,3]dioxolo)[4,5-b:4′,5′-d]pyran-5-yl)methyl 6-((triisopropylsilyl)ethynyl)quinoline-3-carboxylate (4d) The general procedure 2.6 was followed and purification by preparative TLC (hexane/EA=3/1) furnished compound 4d as pale yellow oil (32.7 mg, 55%, C6:others>99:1). ¹H NMR (600 MHz, CDCl₃) δ 9.43 (d, J=2.1 Hz, 1H), 8.79 (d, J=1.9 Hz, 1H), 8.09-8.05 (m, 2H), 7.86 (dd, J=8.7, 1.8 Hz, 1H), 5.58 (d, J=4.9 Hz, 1H), 4.68 (dd, J=7.8, 2.5 Hz, 1H), 4.61 (dd, J=11.5, 4.7 Hz, 1H), 4.55 (dd, J=11.5, 7.7 Hz, 1H), 4.38-4.35 (m, 2H), 4.24 (ddd, J=7.0, 4.7, 1.9 Hz, 1H), 1.53 (s, 3H), 1.50 (s, 3H), 1.37 (s, 3H), 1.34 (s, 3H), 1.17 (m, 21H). ¹³C NMR (151 MHz, CDCl₃) δ 165.01, 150.55, 149.21, 138.44, 134.93, 132.62, 129.45, 126.57, 123.56, 122.86, 109.82, 108.88, 105.88, 96.34, 93.51, 71.14, 70.78, 70.51, 66.09, 64.52, 29.71, 26.07, 26.00, 24.97, 24.51, 18.69, 11.31. HRMS (ESI-TOF) m/z Calcd for C₃₃H₄₆NO₇Si⁺ [M+H]⁺ 596.3044, found 596.3060.

4-Chloro-6-((triisopropylsilyl)ethynyl)quinoline (4e) The general procedure 2.6 was followed and purification by preparative TLC (hexane/EA=3/1) furnished compound 4e as colorless oil (13.0 mg, 38%, C6:others=98:2). ¹H NMR (600 MHz, CDCl₃) δ 8.76 (d, J=4.7 Hz, 1H), 8.33 (d, J=1.8 Hz, 1H), 8.04 (d, J=8.7 Hz, 1H), 7.80 (dd, J=8.7, 1.8 Hz, 1H), 7.50 (d, J=4.7 Hz, 1H), 1.17 (m, 21H). ¹³C NMR (151 MHz, CDCl₃) δ 150.30, 148.49, 142.24, 133.61, 129.85, 127.72, 126.32, 123.03, 121.85, 106.19, 93.55, 18.69, 11.32. HRMS (ESI-TOF) m/z Calcd for C₂₀H₂₇ClNSi⁺ [M+H]⁺ 344.1601, found 344.1609.

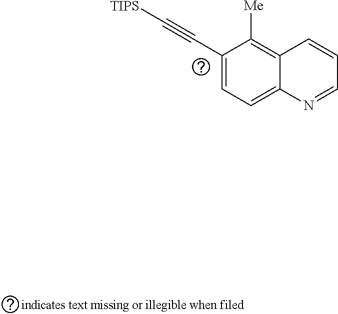

5-Methyl-6-((triisopropylsilyl)ethynyl)quinoline (4f) The general procedure 2.6 was followed and purification by preparative TLC (hexane/EA=15/1) furnished compound 4f as a white solid (14.8 mg, 46%, C6:others>99:1). ¹H NMR (600 MHz, CDCl₃) δ 8.82 (dd, J=4.1, 1.6 Hz, 1H), 8.29 (ddd, J=8.6, 1.7, 0.9 Hz, 1H), 7.81 (d, J=8.8 Hz, 1H), 7.67 (d, J=8.7 Hz, 1H), 7.37 (dd, J=8.5, 4.2 Hz, 1H), 2.78 (s, 3H), 1.10 (d, J=2.9 Hz, 21H). ¹³C NMR (151 MHz, CDCl₃) δ 150.29, 148.00, 137.84, 132.77, 132.72, 127.48, 127.38, 121.26, 121.24, 106.05, 96.31, 18.73, 16.57, 11.36. HRMS (ESI-TOF) m/z Calcd for C₂₁H₃₀NSi⁺ [M+H]⁺ 324.2148, found 324.2160.

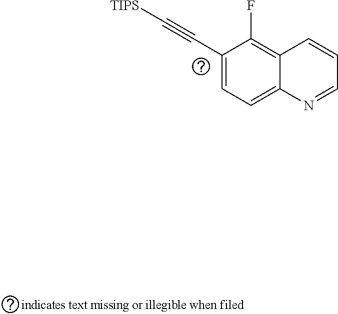

5-Fluoro-6-((triisopropylsilyl)ethynyl)quinoline (4g) The general procedure 2.6 was followed and purification by preparative TLC (hexane/EA=15/1) furnished compound 4g as colorless oil (12.8 mg, 39%, C6:others>99:1). ¹H NMR (600 MHz, CDCl₃) δ 8.87 (dd, J=4.2, 1.7 Hz, 1H), 8.34 (ddd, J=8.4, 1.8, 0.9 Hz, 1H), 7.77 (d, J=8.8 Hz, 1H), 7.63 (dd, J=8.8, 7.4 Hz, 1H), 7.40 (dd, J=8.5, 4.2 Hz, 1H), 1.10 (d, J=3.4 Hz, 16H). ¹³C NMR (151 MHz, CDCl₃) δ 158.97 (d, J=261.6 Hz), 151.56, 148.44 (d, J=3.3 Hz), 132.35, 129.19 (d, J=4.5 Hz), 125.05 (d, J=4.4 Hz), 121.70, 118.90 (d, J=15.9 Hz), 107.30 (d, J=14.8 Hz), 99.47, 98.75 (d, J=4.8 Hz), 18.65, 11.27. ¹⁹F NMR (376 MHz, CDCl₃) δ −120.11. HRMS (ESI-TOF) m/z Calcd for C₂₀H₂₇FNSi⁺ [M+H]⁺ 328.1897, found 328.1906.

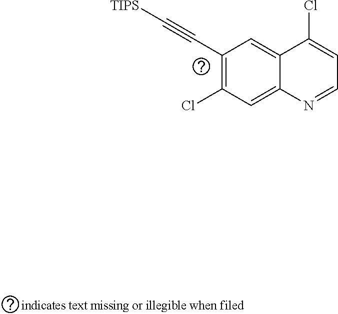

4,7-Dichloro-6-((triisopropylsilyl)ethynyl)quinoline (4h) The general procedure 2.6 was followed and purification by preparative TLC (hexane/EA=3/1) furnished compound 4h as a pale yellow solid (23.4 mg, 62%, C6:others>99:1). ¹H NMR (600 MHz, CDCl₃) δ 8.76 (d, J=4.7 Hz, 1H), 8.37 (s, 1H), 8.17 (s, 1H), 7.48 (d, J=4.7 Hz, 1H), 1.18 (m, 21H). ¹³C NMR (151 MHz, CDCl₃) δ 151.34, 148.47, 142.08, 137.78, 129.46, 124.95, 123.38, 121.87, 102.32, 99.28, 18.68, 11.31. HRMS (ESI-TOF) m/z Calcd for C₂₀H₂₆Cl₂NSi⁺ [M+H]⁺ 378.1212, found 378.1212.

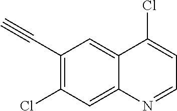

4,7-Dichloro-6-ethynylquinoline (4h′) Treating 4 h with TBAF (2 equiv) in THF for 5 min furnished compound 4h′ (colorless oil, 96%). ¹H NMR (600 MHz, CDCl₃) δ 8.72 (d, J=4.7 Hz, 1H), 8.38 (s, 1H), 8.12 (s, 1H), 7.44 (d, J=4.7 Hz, 1H), 3.44 (s, 1H). 13C NMR (151 MHz, CDCl₃) δ 151.75, 148.73, 142.23, 137.36, 130.34, 129.67, 124.94, 122,06, 122.01, 83.87, 79,65. HRMS (ESI-TOF) m/z Calcd for C₁₁H₆C₁₂N⁺ [M+H]⁺ 221.9877, found 221.9885.

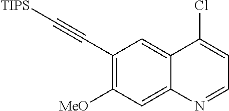

4-Chloro-7-methoxy-6-((triisopropylsilyl)ethynyl)quinoline (4i) The general procedure 2.6 was followed and purification by preparative TLC (hexane/EA=2/1) furnished compound 4i as a pale yellow solid (22.4 mg, 60%, C6:others>99:1). ¹H NMR (600 MHz, CDCl₃) δ 8.66 (d, J=4.8 Hz, 1H), 8.28 (s, 1H), 7.39 (s, 1H), 7.34 (d, J=4.8 Hz, 1H), 4.00 (s, 3H), 1.18 (m, 21H). ¹³C NMR (151 MHz, CDCl₃) δ 161.13, 150.64, 150.33, 141.90, 129.43, 121.18, 119.70, 116.62, 107.28, 101.88, 98.02, 56.12, 18.68, 11.36. HRMS (ESI-TOF) m/z Calcd for C₂₁H₂₉ClNOSi⁺ [M+H]⁺ 374.1707, found 374.1719.

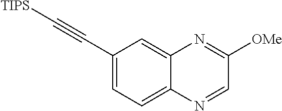

2-Methoxy-7-((triisopropylsilyl)ethynyl)quinoxaline (4j) The general procedure 2.6 was followed and purification by preparative TLC (hexane/EA=20/1) furnished compound 4j as colorless oil (8.5 mg, 25%, C7:others=89:11). ¹H NMR (600 MHz, CDCl₃) δ 8.43 (s, 1H), 7.98 (d, J=1.7 Hz, 1H), 7.92 (d, J=8.4 Hz, 1H), 7.61 (dd, J=8.5, 1.8 Hz, 1H), 4.09 (s, 3H), 1.16 (m, 21H). ¹³C NMR (151 MHz, CDCl₃) δ 158,09, 140.14, 139.86, 138.58, 130.82, 129.82, 128.85, 125.38, 106.30, 93.50, 53.80, 18.68, 11.31. HRMS (ESI-TOF) m/z Calcd for C₂₀H₂₉N₂OSi⁺ [M+H]⁺ 341.2049, found 341.2049.

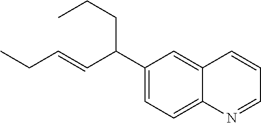 [SMILES: CC/C=C/C(CCC)c1ccc2ncccc2c1]

(E)-6-(Oct-5-en-4-yl)quinoline (5a) The general procedure 2.7 was followed and purification by preparative TLC (DCM/EA=10/1) furnished compound 5a as colorless oil (18.0 mg, 75%, C6:others=91:9). ¹H NMR (600 MHz, CDCl₃) δ 8.85 (dd, J=4.2, 1.8 Hz, 1H), 8.10 (dd, J=8.7, 1.5 Hz, 1H), 8.04 (d, J=8.5 Hz, 1H), 7.61-7.55 (m, 2H), 7.36 (dd, J=8.3, 4.2 Hz, 1H), 5.60 (dd, J=15.3, 7.5 Hz, 1H), 5.53 (dt, J=15.3, 6.1 Hz, 1H), 3.40 (q, J=7.4 Hz, 1H), 2.03 (p, J=7.1 Hz, 2H), 1.75 (q, J=7.6 Hz, 2H), 1.34 (dt, J=15.0, 7.0 Hz, 1H), 1.24 (dt, J=13.7, 7.1 Hz, 1H), 0.97 (t, J=7.5 Hz, 3H), 0.91 (t, J=7.4 Hz, 3H). ¹³C NMR (151 MHz, CDCl₃) δ 149.67, 147.27, 143.99, 135.72, 132.50, 132.40, 130.05, 129.35, 128.35, 125.32, 121.00, 48.46, 38.17, 25.62, 20.72, 14.02, 13.83. HRMS (ESI-TOF) m/z Calcd for C₁₇H₂₂N⁺ [M+H]⁺ 240.1752, found 240.1758.

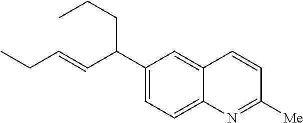 [SMILES: CC/C=C/C(CCC)c1ccc2nc(C)ccc2c1]

(E)-2-Methyl-6-(oct-5-en-4-yl)quinoline (5b) The general procedure 2.7 was followed and purification by preparative TLC (DCM/EA=10/1) furnished compound 5b as colorless oil (16.2 mg, 64%, C6:others=84:16). ¹H NMR (600 MHz, CDCl₃) δ 7.99 (d, J=8.3 Hz, 1H), 7.94 (d, J=8.6 Hz, 1H), 7.55-7.51 (m, 2H), 7.25 (d, J=8.3 Hz, 1H), 5.59 (dd, J=15.3, 7.5 Hz, 1H), 5.52 (dt, J=15.3, 6.1 Hz, 1H), 3.37 (q, J=7.5 Hz, 1H), 2.72 (s, 3H), 2.02 (q, J=7.1 Hz, 2H), 1.75-1.71 (m, 2H), 1.33 (dd, J=14.3, 7.1 Hz, 1H), 1.23 (dd, J=14.3, 7.1 Hz, 1H), 0.97 (t, J=7.5 Hz, 3H), 0.90 (t, J=7.4 Hz, 3H). ¹³C NMR (151 MHz, CDCl₃) δ 158.13, 146.80, 143.01, 135.89, 132.65, 132.22, 129.94, 128.51, 126.50, 125.09, 121.88, 48.38, 38.22, 25.62, 25.27, 20.72, 14.03, 13.85. HRMS (ESI-TOF) m/z Calcd for C₁₈H₂₄N⁺ [M+H]⁺ 254.1909, found 254.1915.

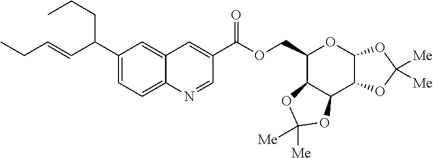 [SMILES: CC/C=C/C(CCC)c1ccc2ncc(C(=O)OC[C@H]3O[C@@H]4OC(C)(C)O[C@@H]4[C@H]4OC(C)(C)O[C@H]43)cc2c1]

((3aR,5R,5aS,8aS,8bR)-2,2,7,7-Tetramethyltetrahydro-5H-bis([1,3]dioxolo)[4,5-b:4′,5′-d]pyran-5-yl)methyl 6-((E)-oct-5-en-4-yl)quinoline-3-carboxylate (5c) The general procedure 2.7 was followed and purification by preparative TLC (DCM/EA=5/1) furnished compound 5c as colorless oil (29.9 mg, 57%, C6:others=90:10). ¹H NMR (600 MHz, CDCl₃) δ 9.40 (d, J=2.2 Hz, 1H), 8.81 (d, J=2.1 Hz, 1H), 8.08 (d, J=8.5 Hz, 1H), 7.72-7.67 (m, 2H), 5.61-5.52 (m, 3H), 4.68 (dd, J=7.9, 2.5 Hz, 1H), 4.62 (dd, J=11.5, 4.6 Hz, 1H), 4.53 (dd, J=11.5, 7.7 Hz, 1H), 4.38-4.36 (m, 2H), 4.25 (ddd, J=6.9, 4.6, 1.9 Hz, 1H), 3.43 (q, J=7.3 Hz, 1H), 2.07-2.01 (m, 2H), 1.76 (q, J=7.6 Hz, 2H), 1.53 (s, 3H), 1.50 (s, 3H), 1.37 (s, 3H), 1.34 (m, 4H), 1.26-1.23 (m, 1H), 0.98 (t, J=7.4 Hz, 3H), 0.92 (t, J=7.4 Hz, 3H). ¹³C NMR (151 MHz, CDCl₃) δ 165.41, 149,38, 148.89, 145,05, 138.59, 132,79, 132,62, 132,14, 129,33, 126,90, 126.64, 122.80, 109.80, 108.88, 96.35, 71.17, 70.78, 70.53, 66.16, 64.37, 48.34, 38.08, 26.07, 26.00, 25.61, 24.98, 24.51, 20.69, 14.00, 13.80. HRMS (ESI-TOF) m/z Calcd for C₃₀H₄₀NO₇⁺ [M+H]⁺ 526.2805, found 526.2806.

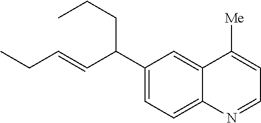 [SMILES: CC/C=C/C(CCC)c1ccc2nccc(C)c2c1]

(E)-4-Methyl-6-(oct-5-en-4-yl)quinoline (5d) The general procedure 2.7 was followed and purification by preparative TLC (DCM/EA=10/1) furnished compound 5d as colorless oil (15.2 mg, 60%, C6:others=91:9). ¹H NMR (600 MHz, CDCl₃) δ 8.71 (d, J=4.4 Hz, 1H), 8.03 (d, J=8.6 Hz, 1H), 7.73 (d, J=2.0 Hz, 1H), 7.57 (dd, J=8.7, 2.0 Hz, 1H), 7.20 (d, J=4.0 Hz, 1H), 5.61 (dd, J=15.3, 7.5 Hz, 1H), 5.54 (dt, J=15.4, 6.1 Hz, 1H), 3.42 (q, J=7.5 Hz, 1H), 2.70 (s, 3H), 2.04 (p, J=7.1 Hz, 2H), 1.77-1.73 (m, 2H), 1.37-1.31 (m, 1H), 1.26-1.21 (m, 1H), 0.98 (t, J=7.5 Hz, 3H), 0.92 (t, J=7.4 Hz, 3H). ¹³C NMR (151 MHz, CDCl₃) δ 149.41, 146.90, 143.84, 143.73, 132.65, 132.35, 129.95, 129.44, 128.26, 121.85, 121.52, 48.81, 38.32, 25.63, 20.77, 18.73, 14.04, 13.86. HRMS (ESI-TOF) m/z Calcd for C₁₈H₂₄N⁺ [M+H]⁺ 254.1909, found 254.1911.

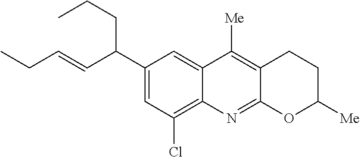 [SMILES: CC/C=C/C(CCC)c1cc(Cl)c2nc3c(c(C)c2c1)CCC(C)O3]

(E)-9-Chloro-2,5-dimethyl-7-(oct-5-en-4-yl)-3,4-dihydro-2H-pyrano[2,3-b]quinoline (5e) The general procedure 2.7 was followed and purification by preparative TLC (hexane/DCM=1/1) furnished compound 5e as a white solid (10.3 mg, 29%, C6:others=88:12). ¹H NMR (600 MHz, CDCl₃) δ 7.56 (d, J=5.4 Hz, 2H), 5.58-5.49 (m, 2H), 4.41-4.36 (m, 1H), 3.31 (q, J=7.2 Hz, 1H), 3.01-2.97 (m, 1H), 2.87 (td, J=11.6, 5.8 Hz, 1H), 2.55 (s, 3H), 2.16-2.12 (m, 1H), 2.04 (p, J=7.3 Hz, 2H), 1.81 (dd, J=12.0, 5.7 Hz, 1H), 1.71 (dd, J=11.5, 4.8 Hz, 2H), 1.53 (d, J=6.3 Hz, 3H), 1.33 (t, J=7.2 Hz, 1H), 1.25-1.21 (m, 1H), 0.98 (t, J=7.4 Hz, 3H), 0.91 (t, J=7.4 Hz, 3H). ¹³C NMR (151 MHz, CDCl₃) δ 160.04, 144.43, 141.04, 140.87, 132.44, 132.40, 131.43, 129.01, 126.13, 120.29, 117.06, 73.33, 48.54, 38.26, 28.87, 25.60, 23.66, 21.32, 20.72, 14.29, 14.01, 13.84. HRMS (ESI-TOF) m/z Calcd for C₂₂H₂₉ClNO⁺ [M+H]⁺ 358.1938, found 358.1937.

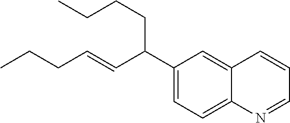 [SMILES: CCC/C=C/C(CCCC)c1ccc2ncccc2c1]

(E)-6-(Dec-6-en-5-yl)quinoline (5f) The general procedure 2.7 was followed except using trans-5-decene (3 equiv) and purification by preparative TLC (DCM/EA=10/1) furnished compound 5f as colorless oil (21.4 mg, 80%, C6:others=90:10). ¹H NMR (600 MHz, CDCl₃) δ 8.85 (dd, J=4.2, 1.8 Hz, 1H), 8.10 (dd, J=8.4, 1.0 Hz, 1H), 8.04 (d, J=8.5 Hz, 1H), 7.60-7.57 (m, 2H), 7.36 (dd, J=8.2, 4.2 Hz, 1H), 5.60 (ddt, J=15.3, 7.7, 1.3 Hz, 1H), 5.48 (dtd, J=15.1, 6.7, 1.0 Hz, 1H), 3.38 (q, J=7.5 Hz, 1H), 2.00 (q, J=7.0 Hz, 2H), 1.79-1.75 (m, 2H), 1.38 (q, J=7.3 Hz, 2H), 1.34-1.29 (m, 3H), 1.22-1.17 (m, 1H), 0.87 (q, J=7.2 Hz, 6H). ¹³C NMR (151 MHz, CDCl₃) δ 149.67, 147.27, 144.06, 135.74, 133.73, 130.70, 130.06, 129.33, 128.36, 125.30, 121.00, 48.78, 35.68, 34.68, 29.84, 22.65, 22.59, 14.04, 13.65. HRMS (ESI-TOF) m/z Calcd for C₁₉H₂₆5N⁺ [M+H]⁺ 268,2065, found 268.2066.

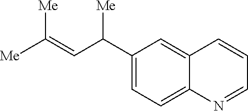 [SMILES: CC(C)=CC(C)c1ccc2ncccc2c1]

6-(4-Methylpent-3-en-2-yl)quinoline (5g) The general procedure 2.7 was followed except using trans-4-methyl-2-pentene (3 equiv), and purification by preparative TLC (hexane/EA=10/1) furnished compound 5g as colorless oil (13.1 mg, 62%, C6:others=85:15). ¹H NMR (600 MHz, CDCl₃) δ 8.78 (dd, J=4.2, 1.7 Hz, 1H), 8.03 (d, J=7.8 Hz, 1H), 7.96 (d, J=8.5 Hz, 1H), 7.57-7.53 (m, 2H), 7.30-7.28 (m, 1H), 5.27 (dp, J=9.2, 1.4 Hz, 1H), 3.78 (dq, J=9.3, 6.9 Hz, 1H), 1.67 (d, J=1.4 Hz, 3H), 1.64 (d, J=1.4 Hz, 3H), 1.33 (d, J=6.9 Hz, 311). ¹³C NMR (151 MHz, CDCl₃) δ 149.64, 147.18, 145.63, 135.75, 131.47, 129.91, 129.51, 129.36, 128.36, 124.23, 121.01, 38.12, 25.84, 22.36, 18.08. HRMS (ESI-TOF) m/z Calcd for C₁₅H₁₈N [M+H]⁺ 212.1439, found 212.1450.

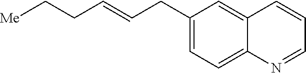 [SMILES: CCC/C=C/Cc1ccc2ncccc2c1]

(E)-6-(Hex-2-en-1-yl)quinoline (5h) The general procedure 2.7 was followed except using 1-hexene (3 equiv) and purification by preparative TLC (hexane/EA=10/1) furnished compound 5h as colorless oil (10.8 mg, 51%, C6:others=86:14). ¹H NMR (600 MHz, CDCl₃) δ 8.88 (dd, J=4.2, 1.7 Hz, 1H), 8.12-8.10 (m, 1H), 8.05 (d, J=8.5 Hz, 1H), 7.61-7.59 (m, 2H), 7.40-7.38 (m, 1H), 5.69-5.64 (m, 1H), 5.63-5.58 (m, 1H), 3.54 (d, J=6.4 Hz, 2H), 2.09-2.04 (m, 2H), 1.46-1.42 (m, 2H), 0.94 (t, J=7.4 Hz, 3H). ¹³C NMR (151 MHz, CDCl₃) δ 166.80, 151.34, 149.04, 143.61, 136.48, 132.73, 130.27, 129.23, 128.27, 127.28, 121.87, 119.65, 60.69, 14.34. HRMS (ESI-TOF) m/z Calcd for C₁₅H₁₈N⁺ [M+H]⁺ 212.1439, found 212.1445.

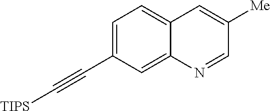

3-Methyl-7-((triisopropylsilyl)ethynyl)quinoline (6a) The general procedure 2.8 was followed and purification by preparative TLC (hexane/EA=20/1) furnished compound 6a as pale yellow oil (7.4 mg, 46%, C7:others=94:6). ¹H NMR (600 MHz, CDCl₃) δ 8.77 (d, J=2.2 Hz, 1H), 8.20 (s, 1H), 7.87 (s, 1H), 7.66 (d, J=8.4 Hz, 1H), 7.54 (dd, J=8.4, 1.6 Hz, 1H), 2.52 (s, 3H), 1.16 (m, 21H). ¹³C NMR (151 MHz, CDCl₃) δ 153.14, 146.09, 134.40, 133.04, 131.07, 129.54, 127.85, 127.04, 123.57, 106.77, 92.53, 18.83, 18.68, 11.33. HRMS (ESI-TOF) m/z Calcd for C₂₁H₃₀NSi⁺ [M+H]⁺ 324.2148, found 324.2144.

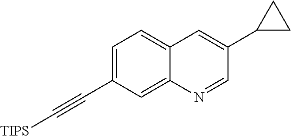

3-Cyclopropyl-7-((triisopropylsilyl)ethynyl)quinoline (6b) The general procedure 2.8 was followed and purification by preparative TLC (hexane/EA=20/1) furnished compound 6b as colorless oil (6.8 mg, 39%, C7:others=92:8). ¹H NMR (600 MHz, CDCl₃) δ 8.75 (d, J=2.2 Hz, 1H), 8.19 (s, 1H), 7.66 (d, J=2.3 Hz, 1H), 7.64 (d, J=8.4 Hz, 1H), 7.53 (dd, J=8.4, 1.6 Hz, 1H), 2.07 (td, J=8.5, 4.2 Hz, 1H), 1.16 (m, 21H), 1.12 (dt, J=6.6, 1.6 Hz, 2H), 0.86 (dd, J=5.1, 1.5 Hz, 2H). ¹³C NMR (151 MHz, CDCl₃) δ 151.36, 146.24, 137.35, 133.01, 130.40, 129.58, 127.82, 127.04, 123.36, 106.45, 92.49, 18.69, 13.46, 11.33, 9.39. HRMS (ESI-TOF) m/z Calcd for C₂₃H₃₂NSi⁺ [M+H]⁺ 350.2304, found 350.2306.

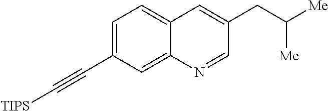

3-Isobutyl-7-((triisopropylsilyl)ethynyl)quinoline (6c) The general procedure 2.8 was followed and purification by preparative TLC (hexane/EA=20/1) furnished compound 6c as colorless oil (6.9 mg, 38%, C7:others=92:8). ¹H NMR (600 MHz, CDCl₃) δ 8.74 (d, J=2.2 Hz, 1H), 8.21 (s, 1H), 7.83 (s, 1H), 7.68 (d, J=8.3 Hz, 1H), 7.55 (dd, J=8.4, 1.6 Hz, 1H), 2.66 (d, J=7.2 Hz, 2H), 2.00 (dt, J=13.5, 7.0 Hz, 1H), 1.16 (m, 21H), 0.96 (d, J=6.6 Hz, 6H). ¹³C NMR (151 MHz, CDCl₃) δ 153.20, 146.36, 134.75, 134.62, 133.00, 129.47, 127.81, 127.23, 123.66, 106.80, 92.58, 42.58, 30.12, 22.28, 18.68, 11.33. HRMS (ESI-TOF) m/z Calcd for C₂₄H₃₆NSi⁺ [M+H]⁺ 366.2617, found 366.2621.

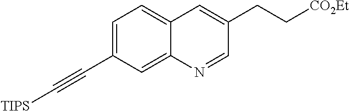

Ethyl 3-(7-((triisopropylsilyl)ethynyl)quinolin-3-yl)propanoate (6d) The general procedure 2.8 was followed and purification by preparative TLC (hexane/EA=5/1) furnished compound 6d as colorless oil (8.6 mg, 42%, C7:others=91:9). ¹H NMR (600 MHz, CDCl₃) δ 8.80 (d, J=2.2 Hz, 1H), 8.20 (s, 1H), 7.92 (s, 1H), 7.68 (d, J=8.4 Hz, 1H), 7.56 (dd, J=8.4, 1.6 Hz, 1H), 4.13 (q, J=7.1 Hz, 2H), 3.14 (t, J=7.6 Hz, 2H), 2.74 (t, J=7.6 Hz, 2H), 1.22 (t, J=7.2 Hz, 3H), 1.16 (m, 21H). ¹³C NMR (151 MHz, CDCl₃) δ 172.28, 152,42, 146,56, 134.15, 133,74, 133,01, 129,69, 127,72, 127,30, 124,04, 106.65, 92.89, 60.71, 35.26, 28.26, 18.68, 14.19, 11.33. HRMS (ESI-TOF) m/z Calcd for C₂₅H₃₆NO₂Si⁺ [M+H]⁺ 410.2515, found 410.2528.

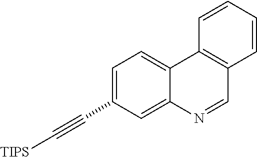

3-((Triisopropylsilyl)ethynyl)phenanthridine (6e) The general procedure 2.8 was followed and purification by preparative TLC (hexane/EA=20/1) furnished compound 6e as colorless oil (5.6 mg, 31%, C_{shown}:others=92:8). ¹H NMR (600 MHz, CDCl₃) δ 9.29 (s, 1H), 8.59 (d, J=8.2 Hz, 1H), 8.50 (d, J=8.5 Hz, 1H), 8.32 (d, J=1.7 Hz, 1H), 8.06 (d, J=8.5 Hz, 1H), 7.88 (t, J=7.7 Hz, 1H), 7.76-7.71 (m, 2H), 1.18 (m, 21H). ¹³C NMR (151 MHz, CDCl₃) δ 154.26, 144.14, 133.89, 132.19, 131.24, 130.15, 128.89, 127.88, 126.53, 124.03, 123.86, 122.22, 122.08, 106.61, 92.64, 18.70, 11.35. HRMS (ESI-TOF) m z Calcd for C₂₄H₃₀NSi⁺ [M+H]⁺ 360.2148, found 360.2148.

### Experimental Section References

1) Ellingboe, J. W. et al. Antihyperglycemic activity of novel naphthalenyl 3H-1,2,3,5-Oxathiadiazole 2-Oxides. J. Med. Chem. 36, 2485-2493 (1993).
2) Aguilar Izquierdo, N., Carrascal Riera, M., Castro Palomino Laria, J. C. & Erra Sola, M. WO 2010081692A1 (2010).
3) Xu, T. & Dong, G. Rhodium-catalyzed regioselective carboacylation of olefins, a C—C bond activation approach for accessing fused-ring systems. Angew. Chem. Int. Ed. 51, 7567-7571 (2012).
4) Chen, P.-H., Savage, N. A. & Dong, G. Concise synthesis of functionalized benzocyclobutenones. Tetrahedron 70, 4135-4146 (2014).
5) Willis, M. C., Brace, G. N. & Holmes, I. P. Palladium-catalyzed tandem alkenyl and aryl C—N bond formation: a cascade N-annulation route to 1-functionalized indoles. Angew. Chem. Int. Ed 44, 403-406 (2005).
6) Lenoir, D., Glaser, R., Mison, P. & Schleyer, P. V. R. Synthesis of 1,2- and 2,4-disubstituted adamantanes. The protoadamantane route. J. Org. Chem. 36, 1821-1826 (1971).
7) Jolliffe, J. D., Armstrong, R. J. & Smith, M. D. Catalytic enantioselective synthesis of atropisomeric biaryls by a cation-directed O-alkylation. Nat. Chem. 9, 558-562 (2017).
8) Clark, A. H., McCorvy, J. D., Watts, V. J. & Nichols, D. E. Assessment of dopamine D1 receptor affinity and efficacy of three tetracyclic conformationally-restricted analogs of SKF38393. Bioorg. Med. Chem. 19, 5420-5431 (2011).
9) Beves, J. E. et al. Toward metal complexes that can directionally walk along tracks: controlled stepping of a molecular biped with a palladium(II) foot. J. Am. Chem. Soc. 136, 2094-2100 (2014).
10) Zhao, Y. & Swager, T. M. Simultaneous chirality sensing of multiple amines by ¹⁹F NMR. J. Am. Chem. Soc. 137, 3221-3224 (2015).
11) Chen, Q., du Jourdin, X. M. & Knochel, P. Transition-metal-free BF₃-mediated regioselective direct alkylation and arylation of functionalized pyridines using Grignard or organozinc reagents. J. Am. Chem. Soc. 135, 4958-4961 (2013).
12) Ye, M., Gao, G.-L. & Yu, J.-Q. Ligand-promoted C-3 selective C—H olefination of pyridines with Pd catalysts. J. Am. Chem. Soc. 133, 6964-6967 (2011).
13) Zhang, Z., Tanaka, K. & Yu, J.-Q. Remote site-selective C—H activation directed by a catalytic bifunctional template. Nature 543, 538-542 (2017).
14) Wang, X. et al. General and practical potassium methoxide/disilane-mediated dehalogenative deuteration of (hetero)arylhalides. J. Am. Chem. Soc. 140, 10970-10974 (2018).
15) Monrad, R. N. & Madsen, R. Ruthenium-catalysed synthesis of 2- and 3-substituted quinolines from anilines and 1,3-diols. Org. Biomol. Chem. 9, 610-615 (2011).

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilized for realizing the invention in diverse forms thereof.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

What is claimed is:

1. A palladium-coordinating template compound for directing C6 selective functionalization of polycyclic aza-arenes having a hydrogen atom disposed on the 6-position thereof, comprising the structure of Formula I 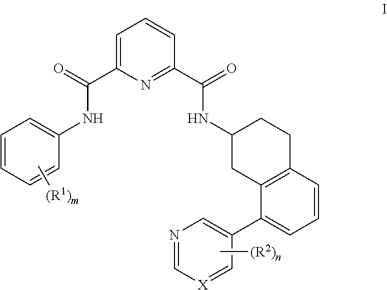
wherein:
each R¹ and R² is independently selected from halo, trifluoromethyl, nitro, optionally substituted (C₁-C₁₂)alkyl, and optionally substituted (C₁-C₁₂)alkoxy;
m is 0, 1, 2, 3, 4 or 5;
n is 0, 1, 2, or 3; and
X is CH, CR², or N.

2. The palladium-coordinating template compound of claim 1, wherein X is N, m is 2, both R¹ are —CF₃, and n is 0.

3. The palladium-coordinating template compound of claim 2, having the structure of Formula II:

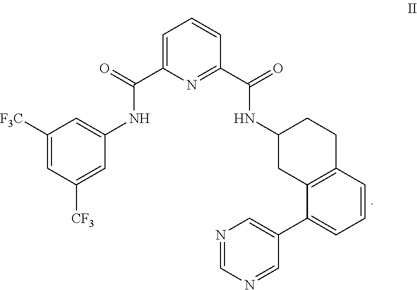 [SMILES: O=C(Nc1cc(C(F)(F)F)cc(C(F)(F)F)c1)c1cccc(C(=O)NC2CCc3cccc(-c4cncnc4)c3C2)n1]

4. The palladium-coordinating template compound of claim 1, wherein X is N, m is 2, both R¹ are —OMe, and n is 0.

5. The palladium-coordinating template compound of claim 4, having the structure of Formula III:

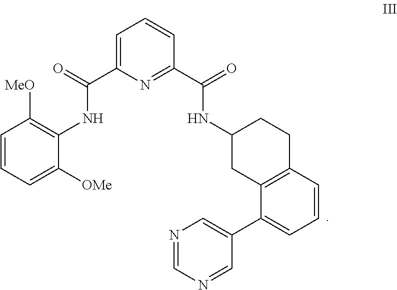 [SMILES: COc1cccc(OC)c1NC(=O)c1cccc(C(=O)NC2CCc3cccc(-c4cncnc4)c3C2)n1]

6. A palladium-coordinating template chaperone compound for catalytic conversion of Formula I after C6 selective functionalization of polycyclic aza-arenes having a hydrogen atom disposed on the 6-position thereof, comprising the structure of Formula IV 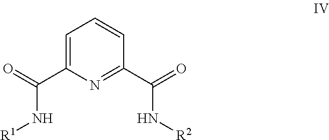
wherein:
each R¹ and R² is independently selected from (C₁-C₁₂)alkyl, Bn, and phenyl, each of which is optionally mono, di-, tri-, tetra-, or penta-substituted with one or more substituents including, but not limited to, halo, trifluoromethyl, nitro, (C₁-C₁₂)alkyl, and C₁-C₁₂)alkoxy.

7. The palladium-coordinating template chaperone compound of claim 6, wherein both R¹ and R² are optionally substituted phenyl.

8. The palladium-coordinating template chaperone compound of claim 7, having the structure of Formula V:

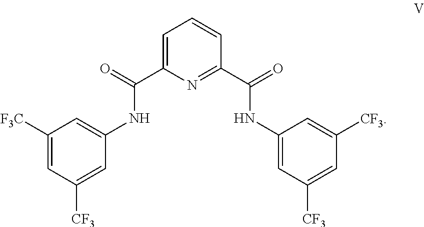 [SMILES: O=C(Nc1cc(C(F)(F)F)cc(C(F)(F)F)c1)c1cccc(C(=O)Nc2cc(C(F)(F)F)cc(C(F)(F)F)c2)n1]

9. A template palladium complex of Formula VI for directing C6 selective olefination or allylation of polycyclic aza-arenes having a hydrogen atom disposed on the 6-position thereof, comprising the template chaperone compound of Formula V of claim 8, an atom of Pd(II), and a molecule of acetonitrile (L):

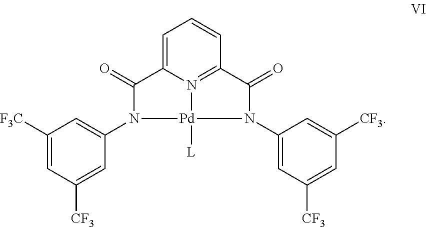

10. The palladium-coordinating template chaperone compound of claim 6, wherein both R¹ and R² are optionally substituted cyclohexyl.

11. The palladium-coordinating template chaperone compound of claim 10, having the structure of Formula VII:

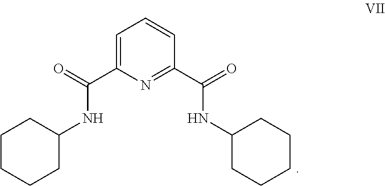 [SMILES: O=C(NC1CCCCC1)c1cccc(C(=O)NC2CCCCC2)n1]

12. A template palladium complex of Formula VIII for directing C6 selective olefination or allylation of polycyclic aza-arenes having a hydrogen atom disposed on the 6-position thereof, comprising the template chaperone compound of Formula VII of claim 11, an atom of Pd(II), and a molecule of acetonitrile(L):

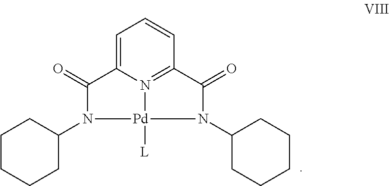

13. A method of directing diverse C6 selective functionalization of a polycyclic aza-arene having a hydrogen atom disposed on the 6-position thereof, including, but not limited to, olefination, allylation, alkynylation, arylation, iodination and cyanation, comprising mixing a polycyclic aza-arene with the palladium-coordinating template compound of Formula I of claim 1 with the template chaperone compound of Formula II.

14. The method of claim 13, wherein the selective functionalization is olefination.

15. The method of claim 13, wherein the selective functionalization is allylation.

16. The method of claim 13, wherein the selective functionalization is alkynylation.

17. The method of claim 13, wherein the selective functionalization is arylation.

18. The method of claim 13, wherein the selective functionalization is iodination.

19. The method of claim 13, wherein the selective functionalization is cyanation.

20. The method of claim 14, comprising i) mixing the polycyclic aza-arene and the template compound of Formula I with the template chaperone compound of Formula II and a Pd catalyst; and ii) addition of an acrylate, additional Pd catalyst, an N-acylamino acid and an Ag salt.

21. The method of claim 20, wherein the Pd catalyst is Pd(OAc)₂.

22. The method of claim 20, wherein the N-acylamino acid is Ac-Gly-OH.

23. The method of claim 20, wherein the Ag salt is Ag₂CO₃.

24. The method of claim 15, comprising i) mixing the polycyclic aza-arene with the template compound of Formula I, the template chaperone compound of Formula II, and a Pd catalyst; and ii) addition of additional Pd catalyst, an N-acylamino acid, an olefin, an Ag salt, and a Cu salt.

25. The method of claim 24, wherein the Pd catalyst is Pd(OAc)₂.

26. The method of claim 24, wherein the N-acylamino acid is Ac-Gly-OH.

27. The method of claim 24, wherein the Ag salt is Ag₂CO₃.

28. The method of claim 24, wherein the Cu salt is Cu(OH)₂.

29. The method of claim 24, wherein the olefin is (E)-4-octene.

30. The method of claim 24, wherein the Pd catalyst is Pd(II)(OAc)₂, the N-acylamino acid is Ac-Gly-OH, the olefin is (E)-4-octene, the Ag salt is Ag₂CO₃, and the Cu salt is Cu(OH)₂.

31. A template palladium complex of Formula IX for directing C6 selective functionalization of polycyclic aza-arenes having a hydrogen atom disposed on the 6-position thereof, comprising the template compound of Formula I of claim 1, an atom of Pd(II), and a second molecule of the template compound of Formula I (L) 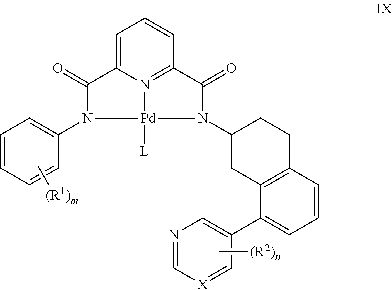
wherein:
each R¹ and R² is independently selected from halo, trifluoromethyl, nitro, optionally substituted (C₁-C₁₂)alkyl, and optionally substituted (C₁-C₁₂)alkoxy;
m is 0, 1, 2, 3, 4 or 5;
n is 0, 1, 2, or 3; and
X is CH, CR², or N.

32. A template palladium complex of Formula X for directing C6 selective functionalization of polycyclic aza-arenes having a hydrogen atom disposed on the 6-position thereof, comprising the template compound of Formula II of claim 3, an atom of Pd(II), and a second molecule of the template compound of Formula II (L):

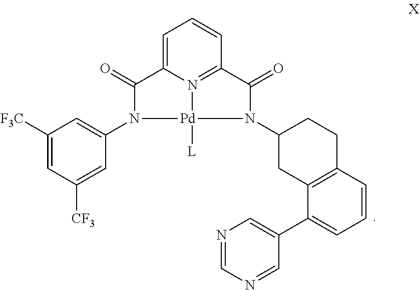

33. A template palladium complex of Formula XI for directing C6 selective functionalization of polycyclic aza-arenes having a hydrogen atom disposed on the 6-position thereof, comprising the template compound of Formula III of claim 5, an atom of Pd(II), and a second molecule of the template compound of Formula III (L)

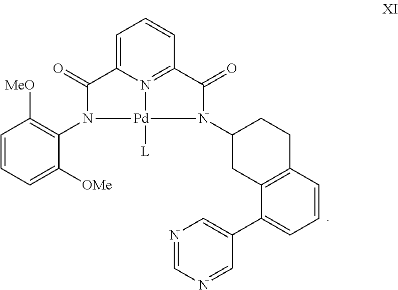

34. A method for directing C6 selective alkynylation of polycyclic aza-arenes having a hydrogen atom disposed on the 6-position thereof, comprising i) mixing the polycyclic aza-arene with the template compound of Formula I, the template chaperone compound of Formula II, and a Pd catalyst; and ii) addition of an alkynyl functional group, additional Pd catalyst, an N-acylamino acid, an Ag salt and a Cu salt.

35. The method of claim 34, wherein the Pd catalyst is Pd(OAc)₂.

36. The method of claim 34, wherein the N-acylamino acid is Ac-Gly-OH.

37. The method of claim 34, wherein the Ag salt is Ag₂CO₃.

38. The method of claim 34, wherein the Cu salt is Cu(OH)₂.

39. The method of claim 34, wherein the alkynyl functional group is triisopropylsilyl acetylene bromide.

40. The method of claim 34, wherein the Pd catalyst is Pd(II)(OAc)₂, the N-acylamino acid is Ac-Gly-OH, the alkynyl functional group is triisopropylsilyl acetylene bromide, the Ag salt is Ag₂CO₃, and the Cu salt is Cu(OH)₂.

41. A palladium-coordinating template compound for directing C7 selective functionalization of polycyclic aza-arenes having a hydrogen atom disposed on the 7-position thereof, having the structure of Formula XII 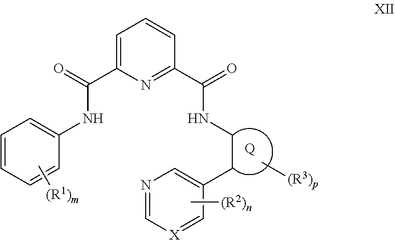
wherein:
each R¹, R², and R³ is independently selected from halo, trifluoromethyl, nitro, (C₁-C₁₂)alkyl, and (C₁-C₁₂)alkoxy;
X is CH, CR², or N;
m is 0, 1, 2, 3, 4 or 5;
n is 0, 1, 2, or 3;
p is 0, 1, 2, 3, or 4; and
Q is selected from the following:

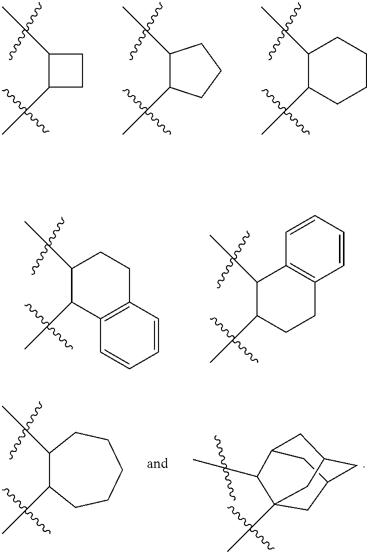

42. The palladium-coordinating template compound for directing C7 selective functionalization of polycyclic aza-arenes of claim 41 having the structure of Formula XIII:

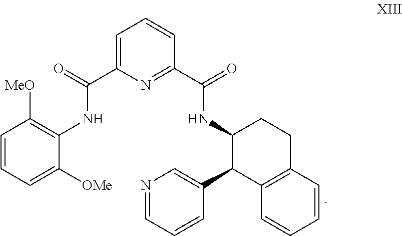 [SMILES: COc1cccc(OC)c1NC(=O)c1cccc(C(=O)N[C@H]2CCc3ccccc3[C@H]2c2cccnc2)n1]

43. A method of directing diverse C7 selective functionalization of C2, C3, and C4-substituted polycyclic aza-arenes having a hydrogen atom disposed on the 7-position thereof, including, but not limited to, olefination and alkynylation, comprising mixing the polycyclic aza-arene with the palladium-coordinating template compound of Formula XII of claim 41 with the template chaperone compound of Formula IV.

44. The method of claim 43, wherein the functionalization is olefination.

45. The method of claim 44, further comprising addition of a Pd catalyst, an N-acylamino acid, an olefin, and an Ag salt.

46. The method of claim 45, wherein the Pd catalyst is Pd(OAc)₂.

47. The method of claim 45, wherein the N-acylamino acid is Ac-DL-Phe-OH.

48. The method of claim 45, wherein the Ag salt is Ag₂CO₃.

49. A method for directing C7 selective olefination of C2, C3, and C4-substituted polycyclic aza-arenes having a hydrogen atom disposed on the 7-position thereof, comprising i) mixing the polycyclic aza-arene, the template compound of Formula XIII of claim 42, the template chaperone compound of Formula IV, and Pd(II)(OAc)₂; and ii) addition of Pd(II)(OAc)₂, Ac-DL-Phe-OH, an olefin, and Ag₂CO₃.

50. The method of claim 43, wherein the functionalization is alkynylation.

51. The method of claim 50, further comprising addition of a Pd catalyst, an N-acylamino acid, an alkynyl functional group, an Ag salt, and a Cu salt.

52. The method of claim 51, wherein the Pd catalyst is Pd(OAc)₂.

53. The method of claim 51, wherein the N-acylamino acid is Ac-DL-Phe-OH.

54. The method of claim 51, wherein the Ag salt is Ag₂CO₃.

55. The method of claim 51, wherein the Cu salt is Cu(OH)₂.

56. The method of claim 51, wherein the alkynyl functional group is triisopropylsilyl acetylene bromide.

57. A method for directing C7 selective alkynylation of C2, C3, and C4-substituted polycyclic aza-arenes having a hydrogen atom disposed on the 7-position thereof, comprising i) mixing the polycyclic aza-arene, the template compound of Formula XII of claim 41, the template chaperone compound of Formula II, and Pd(II)(OAc)₂; and ii) addition of Pd(II)(OAc)₂, Ac-DL-Phe-OH, triisopropylsilyl acetylene bromide, Ag₂CO₃, and Cu(OH)₂.

58. A palladium-coordinating enantiopure template compound for directing C7 selective functionalization of non-, C5, C6 or C8-substituted polycyclic aza-arenes having the structure of Formula XIVa or XIVb:

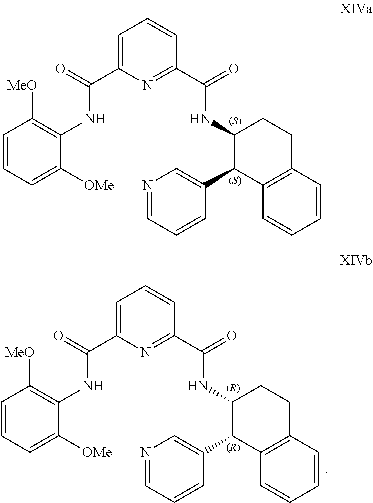

59. A method of olefination of the C7 position of non-, C5, C6 or C8-substituted polycyclic aza-arene having a hydrogen atom disposed on the 7-position thereof, comprising i) mixing the polycyclic aza-arene with the template compound of Formula XIVa or XIVb of claim 58, the template chaperone compound of Formula IV, and a Pd catalyst and ii) mixing the product of step i) with additional Pd catalyst, an N-acylamino acid, an olefin functional group, and an Ag salt.

60. The method of claim 59, wherein the Pd catalyst is Pd(OAc)₂.

61. The method of claim 59, wherein the N-acylamino acid is Ac-L-Leu-OH or Ac-D-Leu-OH.

62. The method of claim 59, wherein the Ag salt is Ag₂CO₃.

63. A method of alkynylation of the C7 position of non-, C5, or C6-substituted polycyclic aza-arene having a hydrogen atom disposed on the 7-position thereof, comprising i) mixing the polycyclic aza-arene with the template compound of Formula XII of claim 41, the template chaperone compound of compound of Formula IV, and Pd catalyst; and ii) addition of a Pd catalyst, an N-acylamino acid, an alkynyl functional group, an Ag salt, and a Cu salt.

64. The method of claim 63, wherein the Pd catalyst is Pd(OAc)₂.

65. The method of claim 63, wherein the N-acylamino acid is Ac-L-Leu-OH or Ac-D-Leu-OH.

66. The method of claim 63, wherein the Ag salt is Ag₂CO₃.

67. The method of claim 63, wherein the Cu salt is Cu(OH)₂.

68. The method of claim 63, wherein the alkynyl functional group is triisopropylsilyl acetylene bromide.

69. The method of claim 63, wherein the Pd catalyst is Pd(OAc)₂, the N-acylamino acid is Ac-L-Leu-OH or Ac-D-Leu-OH, the Ag salt is Ag₂CO₃, the Cu salt is Cu(OH)₂, and the alkynyl functional group is triisopropylsilyl acetylene bromide.

70. The method of any one of claims 13-30, 34-40, 43-57, and 59-69 wherein the polycyclic aza-arene has the structure of any one of Formulae XV-XXIX: 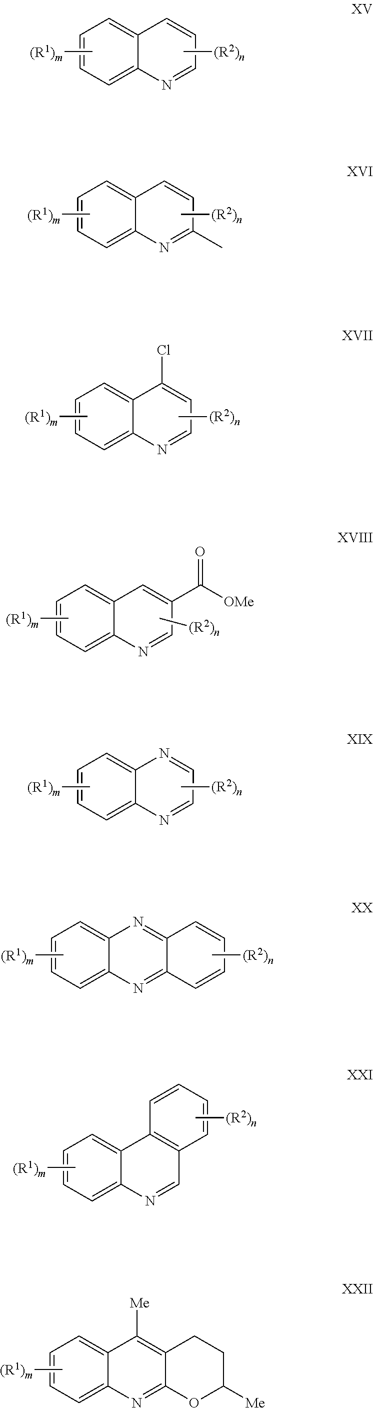 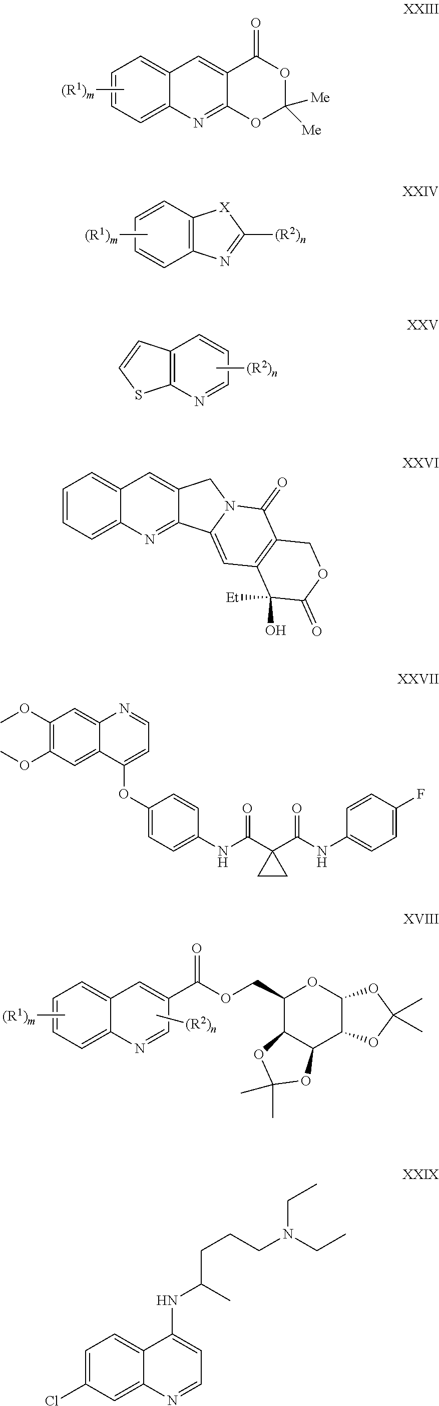
wherein:
each R¹ and R² is independently selected from H, F, Cl, C₁-C₆ alkyl, C₃-C₆ cycloalkyl, 3,5-dimethylC₆H₃, O(C₁-C₆ alkyl), CF₃, C(═O)OH, C₁-C₆ alkylC(═O)OH, C(═O)OC₁-C₆ alkyl) and C₁-C₆ alkylC(═O)OC₁-C₆ alkyl;
m is 0, 1, 2, or 3;
n is 0, 1, 2, or 3; and
X is S, O, NR², or C(R²)₂.

71. The method of any one of claims 13-30, 34-40, 43-57, and 59-70 wherein the polycyclic aza-arene is an optionally substituted bicyclic aza-arene.

72. The method of any one of claims 13-30, 34-40, 43-57, and 59-70 wherein the polycyclic aza-arene is an optionally substituted tricyclic aza-arene.

73. The method of any one of claims 13-30, 34-40, 43-57, and 59-70 wherein the polycyclic aza-arene is an optionally substituted quinoline.

74. The method of any one of claims 13-30, 34-40, 43-57, and 59-70 wherein the polycyclic aza-arene is optionally substituted 3-methylquinoline.

75. The method of any one of claims 13-30, 34-40, 43-57, and 59-70 wherein the polycyclic aza-arene is an optionally substituted tetracyclic or pentacyclic quinoline.

76. The method of any one of claims 13-30, 34-40, 43-57, and 59-70 wherein the polycyclic aza-arene is an optionally substituted quinoxaline.

77. The method of any one of claims 13-30, 34-40, 43-57, and 59-70 wherein the polycyclic aza-arene is an optionally substituted benzothiophene.

78. The method of any one of claims 13-30, 34-40, 43-57, and 59-70 wherein the polycyclic aza-arene is an optionally substituted phenazine.

79. The method of any one of claims 13-30, 34-40, 43-57, and 59-70 wherein the polycyclic aza-arene is an optionally substituted thieno[2,3-b]pyridine.

80. A process for iterative C7 and C6 C—H activation and selective substitution of polycyclic aza-arenes comprising i) mixing the polycyclic aza-arene with the C7 directing template Formula of XII, Formula XIVa or Formula XIVb, and the template chaperone compound of Formula IV in the presence of Pd(OAc)₂, the first functional group to be added at position C7, with Pd(OAc)₂, Ag(OAc)₂, and Ac-DL-Phe-OH, Ac-D-Leu-OH, or Ac-L-Leu-OH and ii) mixing the polycyclic aza-arene product of step i) with the template compound of Formula I and the template chaperone compound of Formula IV in the presence of Pd(OAc)₂, the second functional group to be added at position C6, with Pd(OAc)₂, Ag(OAc)₂, and Ac-Gly-OH.

81. The process of claim 80, wherein the first functional group is an olefin and the second functional group is an olefin.

82. The process of claim 80, wherein the first functional group is an olefin and the second functional group is an allyl in the additional presence of Cu(OH)₂.

83. The process of claim 80, wherein the first functional group is an olefin and the second functional group is an alkyne in the additional presence of Cu(OH)₂.

84. The process of claim 80, wherein the first functional group is an alkyne in the additional presence of Cu(OH)₂ and the second functional group is an olefin.

85. The process of claim 80, wherein the first functional group is an alkyne in the additional presence of Cu(OH)₂ and the second functional group is an allyl in the additional presence of Cu(OH)₂.

86. The process of claim 80, wherein the first functional group is an alkyne in the additional presence of Cu(OH)₂ and the second functional group is an alkyne in the additional presence of Cu(OH)₂.

87. A process for iterative C7 and C6 C—H activation and selective olefination of polycyclic aza-arenes comprising i) mixing the polycyclic aza-arene with the C7 directing template Formula of XII and the template chaperone compound of Formula IV in the presence of Pd(OAc)₂, the first olefin functional group to be added at position C7, and Ac-DL-Phe-OH, Ac-D-Leu-OH, or Ac-L-Leu-OH, and Ag(OAc)₂, and ii) mixing the polycyclic aza-arene product of step i) with the template compound of Formula I and the template chaperone compound of Formula IV in the presence of Pd(OAc)₂, the second olefin functional group to be added at position C6, Ag(OAc)₂, and Ac-Gly-OH.

88. A process for iterative C7 and C6 C—H activation and selective alkynylation of polycyclic aza-arenes comprising i) mixing the polycyclic aza-arene with the C7 directing template Formula of XII and the template chaperone compound of Formula IV in the presence of Pd(OAc)₂, the first alkynyl functional group to be added at position C7, and Ac-DL-Phe-OH, Ac-D-Leu-OH, or Ac-L-Leu-OH, Ag(OAc)₂, and Cu(OH)₂ and ii) mixing the polycyclic aza-arene product of step i) with the template compound of Formula I and the template chaperone compound of Formula IV in the presence of Pd(OAc)₂, the second alkynyl functional group to be added at position C6, Ag(OAc)₂, Cu(OH)₂ and Ac-Gly-OH.

89. Any palladium-coordinating template compound, palladium-coordinating template chaperone compound, template palladium complex, template chaperone palladium complex, method of functionalization including, but not limited to, olefination, allylation, alkynylation, arylation, iodination and cyanation, or process for iterative C7, C6 or related positional C—H activation and substitution of polycyclic aza-arenes as herein described.
